SEQUENCE LISTING (237 sequences)

SEQ ID NO: 1 - protein, 12 aa, Xenorhabdus sp.
  source 1..12: note = strain NBAII XenSa04
WVNAFANWSKAL

SEQ ID NO: 2 - protein, 12 aa, Xenorhabdus stockiae
  source 1..12: note = strain DSM 17904
WVNAFGNWSKSL

SEQ ID NO: 3 - protein, 12 aa, Xenorhabdus sp.
  source 1..12: note = strain BG5
WVNAFANWSKSF

SEQ ID NO: 4 - protein, 12 aa, Kosakonia cowanii
  source 1..12: note = strain pasteuri
WVNAFARWGKSF

SEQ ID NO: 5 - protein, 12 aa, Yersinia sp.
  source 1..12: note = strain Marseille-Q3913
WVNAFARWGRAF

SEQ ID NO: 6 - protein, 12 aa, Yersinia kristensenii
  source 1..12: note = strain IP6945
WVNAFVNWPKSF

SEQ ID NO: 7 - protein, 12 aa, Yersinia bercovieri
  source 1..12: note = strain 127/84
WINAFARWGRAF

SEQ ID NO: 8 - protein, 12 aa, Serratia marcescens
  source 1..12: note = strain CAV1761
WVNAFARWSKSF

SEQ ID NO: 9 - protein, 12 aa, Yersinia enterocolitica
  source 1..12: note = strain PS23
WVNAFVNWTKSF

SEQ ID NO: 10 - protein, 12 aa, Xenorhabdus bovienii
  source 1..12: note = strain CS03
WVNVFARWDKAI

SEQ ID NO: 11 - protein, 12 aa, Erwinia sp.
WVNAFANWTKRI

SEQ ID NO: 12 - protein, 12 aa, Yersinia aleksiciae
WVNAFLRWGKSF

SEQ ID NO: 13 - protein, 12 aa, Erwinia toletana
  source 1..12: note = strain DAPP-PG 735
WINAFANWTKRI

SEQ ID NO: 14 - protein, 12 aa, Photorhabdus heterorhabditis
  source 1..12: note = strain ETL
WVNAFAKWTKRI

SEQ ID NO: 15 - protein, 12 aa, Salmonella enterica
WVNFFAKFTKSF

SEQ ID NO: 16 - protein, 12 aa, Yersinia aldovae
  source 1..12: note = strain IP23238
WVNAFLNWSRSF

SEQ ID NO: 17 - protein, 12 aa, Erwinia sp.
  source 1..12: note = strain E602
WVNAFANWPKRF

SEQ ID NO: 18 - protein, 12 aa, Yersinia frederiksenii
  source 1..12: note = strain RS-42
WVNAFLNWPRSF

SEQ ID NO: 19 - protein, 12 aa, Aeromonas jandaei
  source 1..12: note = strain CN17A0119
WVNAFANWTKRF

SEQ ID NO: 20 - protein, 12 aa, Vibrio sagamiensis
  source 1..12: note = strain NBRC 104589
WVNAFARFTKRF

SEQ ID NO: 21 - protein, 12 aa, Xenorhabdus japonica
  source 1..12: note = strain DSM 16522
WINVFARWNRAI

SEQ ID NO: 22 - protein, 12 aa, Providencia huaxiensis
  source 1..12: note = strain Pvs2
WVNVFARWDKQI

SEQ ID NO: 23 - protein, 12 aa, Sodalis sp.
  source 1..12: note = strain dw_96
WVNAFARWDKKF

SEQ ID NO: 24 - protein, 12 aa, Xenorhabdus bovienii
  source 1..12: note = strain oregonense
WLNVFVRWDRAI

SEQ ID NO: 25 - protein, 12 aa, Photorhabdus australis
  source 1..12: note = strain DSM 17609
WVNAYARWTNRF

SEQ ID NO: 26 - protein, 12 aa, Photorhabdus heterorhabditis
  source 1..12: note = strain SF41
WVNAYARWTKRF

SEQ ID NO: 27 - protein, 14 aa, Yersinia mollaretii
  source 1..14: note = strain SCPM-O-B-7610
AGWINAFGNWTKSF

SEQ ID NO: 28 - protein, 14 aa, Yersinia mollaretii
AGWINAFANWTKSF

SEQ ID NO: 29 - protein, 14 aa, Yersinia kristensenii
AGWIKAFGNWSRSF

SEQ ID NO: 30 - protein, 12 aa, Serratia marcescens
  source 1..12: note = strain 90-166
WVNAFARWSRRW

SEQ ID NO: 31 - protein, 14 aa, Yersinia mollaretii
  source 1..14: note = strain SCPM-O-B-7598
AGWINAFANWTRSF

SEQ ID NO: 32 - protein, 13 aa, Xenorhabdus nematophila
  source 1..13: note = strain SC 0516
WINAFGNWERAFH

SEQ ID NO: 33 - protein, 14 aa, Yersinia enterocolitica
  source 1..14: note = strain E701
AGWIKVFGNWSRSF

SEQ ID NO: 34 - protein, 12 aa, Serratia marcescens
  source 1..12: note = strain ID149856
WVNVFARWSRRW

SEQ ID NO: 35 - protein, 14 aa, Serratia sp.
  source 1..14: note = strain DD3
AGWIRAFANWSRSF

SEQ ID NO: 36 - protein, 13 aa, Mixta theicola
  source 1..13: note = strain QC88-366
GWFRAYLRWSRSF

SEQ ID NO: 37 - protein, 12 aa, Gilliamella sp.
  source 1..12: note = strain Lep-s5
WWRAYARWRRSF

SEQ ID NO: 38 - protein, 12 aa, synthetic construct
WVNAFARWSKRW

SEQ ID NO: 39 - protein, 12 aa, synthetic construct
WVNAFARWSKRF

SEQ ID NO: 40 - protein, 11 aa, Photorhabdus laumondii
GDRWLKWIKNH

SEQ ID NO: 41 - protein, 11 aa, Kosakonia cowanii
  source 1..11: note = strain pasteuri
DGRWLQWIKNH

SEQ ID NO: 42 - protein, 7 aa, Yersinia sp.
WVNAFLN

SEQ ID NO: 43 - protein, 13 aa, Bordetella sp.
  source 1..13: note = genomosp. 11 strain AU8856
VGGFANASWPKSF

SEQ ID NO: 44 - protein, 13 aa, Bordetella bronchialis
  source 1..13: note = strain AU17976
VGGFANATWSKSF

SEQ ID NO: 45 - protein, 13 aa, Bordetella sp.
  source 1..13: note = genomosp. 9 strain AU14267
VGGFANATWPKSF

SEQ ID NO: 46 - protein, 17 aa, Providencia rettgeri
  source 1..17: note = strain 2020EL-00052
KSEAAGGWVNFQWKNSW

SEQ ID NO: 47 - protein, 12 aa, Pandoraea oxalativorans
NVFVNATWSRAM

SEQ ID NO: 48 - protein, 12 aa, Erythrobacter sp.
WSRTVFNRVRPV

SEQ ID NO: 49 - protein, 15 aa, Sodalis sp.
  source 1..15: note = strain dw_96
AGNDGWVKFGWKKKF

SEQ ID NO: 50 - protein, 14 aa, Kosakonia cowanii
  source 1..14: note = strain pasteuri
RGEGWVRAYWAKRF

SEQ ID NO: 51 - protein, 14 aa, Bartonella sp.
RGQGYVRFIFRRSF

SEQ ID NO: 52 - protein, 15 aa, Photorhabdus heterorhabditis
KPGEGWVNFTWNKSF

SEQ ID NO: 53 - protein, 16 aa, Erwinia sp.
WVNAFANRTMGFLFKL

SEQ ID NO: 54 - protein, 16 aa, Xenorhabdus griffiniae
  source 1..16: note = strain VH1
ASTAETWFKLDWKKSF

SEQ ID NO: 55 - protein, 16 aa, Xenorhabdus griffiniae
  source 1..16: note = strain VH1
SSDDDGIFFKTTWDRR

SEQ ID NO: 56 - protein, 20 aa, Burkholderia sp.
ADSQPKARAWFANASFSKRF

SEQ ID NO: 57 - protein, 20 aa, Trinickia sp.
VESQSKPRAWFANSSFSKRF

SEQ ID NO: 58 - protein, 21 aa, Burkholderia sp.
ASSQANSRGWFANATWSKAWR

SEQ ID NO: 59 - protein, 12 aa, Pandoraea norimbergensis
NAFVNATWSRAM

SEQ ID NO: 60 - protein, 12 aa, Pandoraea terrigena
  source 1..12: note = strain LMG 31013
NVFVNATWSRAI

SEQ ID NO: 61 - protein, 392 aa, Xenorhabdus nematophila
MTTSKSEKIKHLEIILKISERCNINCSYCYVFNMGNSLATDSPPVISLDNVLALRGFFERSAAENEIEVIQVDFHGGEPLMMKKDRFDQMCDILRQGDYSGSRLELALQTNGILIDDEWISLFEKHKVHASISIDGPKHINDRYRLDRKGKSTYEGTIHGLRMLQNAWKQGRLPGEPGILSVANPTANGAEIYHHFANVLKCQHFDFLIPDAHHDDDIDGIGIGRFMNEALDAWFADGRSEIFVRIFNTYLGTMLSNQFYRVIGMSANVESAYAFTVTADGLLRIDDTLRSTSDEIFNAIGHLSELSLSGVLNSPNVKEYLSLNSELPSDCADCVWNKICHGGRLVNRFSRANRFNNKTVFCSSMRLFLSRAASHLITAGIDEETIMKNIQK

SEQ ID NO: 62 - protein, 405 aa, Yersinia kristensenii
MEVITGSEGRVMLNLLIEKNIRHLEIILKISERCNINCDYCYVFNKGNSAADDSPARLSNKNIHHLVCFLQRACQEYKIGTVQIDFHGGEPLLMKKENFTDMCIQLISGNYCGSNIRLALQTNATLIDNEWIAIFEKYSVNVSISIDGPKHINDRHRLDTKGRSTYESTVRGLRILQNAYQQGRLPSDPGILCVTNAQANGAEIYRHFVDELGVYSFDFLIPDDSYKDAHPDAVGIGRFLNEALDEWVKDNNAKIFVRLFQTHIASLLGQKNSGVLGHTPNITGVYALTVSSDGFVRVDDTLRSTSDRMFNPIGHLSEVNLSNVFASPQFQEYSSIGQSLPTECEGCIWENICAGGRIVNRFSTEDRFKHKSIYCYSMRTFLSRSSAHLLNMGIKEERIMAAIRA

SEQ ID NO: 63 - protein, 392 aa, Erwinia toletana
MTQLKGEKIKHLEIILKISERCNINCTYCYVFNMGNTLATDSTPVISLDNVYALRGFFERSAAENDIEVIQVDFHGGEPLMMKKDRFDRMCQILLQGNYRSSKFELALQTNGILIDDEWIALFEKHQVHASISVDGPKHINDRHRLDRKGKSTYEGTITGLRLLQNAWQQGRLPGEPGILSVANANANGAEIYRHFADTLQCQRFDFLIPDDHHDDSPDGEGVGRFLNEALDAWFADGRPEIFIRIFNTYLGTMLNSQFNRVLGMSANVESAYAFTVTADGMLRIDDTLRSTSDEIFNAVGHVSELSLARVLETSCVKEYLALSSNLPTVCAECVWNNICHGGRLVNRFSRTNRFNNKTVFCKSMRLFLSRAASHLMASGVDEKEIMKNIQK

SEQ ID NO: 64 - protein, 768 aa, Micromonospora sp.
MAPGPARAALTEFVLKVHARCDLACDHCYVYEHADQSWRRRPVRMTPEVLRTAAGRIAEHAAAHDLPDVTVILHGGEPLLLGAERLGEVLADLRRVIDPVTRLRLGMQTNGVLLSERLCDLLAEHDVAVGVSLDGDRAANDRHRRFRSGAGSYDQVLRAIGLLRRPAYRRIYSGLLCTVDVRNDPIAVYESLLTQEPPRIDFLLPHATWDDPPWRPAGGGTAYAGWLRAVYDRWLADGRPVSVRLFDSLLSTAAGGPSGTEWLGLDPVDLAVVETDGEWEQADSLKTAYDGAPATGMTVFSHAADDVAASPLLARRRSGRAGLSDECRRCPVVDQCGGGLFAHRYGAGHFDHPSVYCADLKELIVHVNENPPAPVRLDAGLPDDFIDRLAALTGDRVAIGRLVEAQIAIVRALLAEVADRLPAGGAGADGWEALTALDRSAPESVARIAAHPYVRAWAVDCLAGSGTGARQGPDYLSALAVAAALDAGTPVRLDVPVRSGRLHLPTVGTVLLPEVGDGAARVETGPGSLRVAAGDVTVAIRPGTPGDAPRWWPTRVLAAPDVSVLLEDGDPHRDCHRLPAGDRLDDAGAARWAETFAAAWQVIRDEVPGHAEELRAGLRAVVPLRRSGAGVSEASTARQAFGGVAATETDAGSLAVLLVHEFQHSKMNALLDICDLVDGTRPIDITVGWRPDPRPAEAVLHGIYAHAAVADIWRIRADRQVDGAQAVYRRYRDWTAEAIGALQRADALTPAGSRLVRQVARSMSGWPS

SEQ ID NO: 65 - protein, 395 aa, Oscillatoriales cyanobacterium
MINPTLLNPEKIDISKFGPINLVVIQATSFCNLNCDYCYLPNRDLKNTLSLDLIEPIFKNIFNSPFVGDEFTICWHAGEPLAVPISFYESAFQLIQAADQKYNQKQAKIWHSVQTNATYINQKWCDFIQEHNICVGVSLDGPEFIHDAHRQTRKGTGSHAQTMRGISFLQKNNIPFYVISVVTQDSLNYADEIFNFFRENGIYDVGFNLEEIEGVNQSSTLEAVGTSEKYRAFMQRFWELTSEVQGEFNLREFEAICGLIYSNTRLTQTDMNNPFVLINIDYQGNFSTFDPELLSVNIKPYGNFILGNVLTDSFESVCDTEKFQKIYTDMQEGIKLCRETCEYFGVCGGGAGSNKYWENGTFACSETMACRYRIKVVTDIILDKLENSLGLVENC

SEQ ID NO: 66 - protein, 405 aa, Lyngbya sp.
MTISKMNLPVQTDNFRASSTLDLSAFGPINLVVIQSTSFCNLNCDYCYLRDRQSKNRLSLDLIEPILKTVLTSPFVGCDFTILWHAGEPLAMPISFYDSATALIREAERQYKTQPIQIFQSIQTNATLINQAWCDCFRRNEIYVGVSLDGPAFLHDAHRQTYKGTGTHAATMRGISLLQKNEIPFNVICVLTQDSLDYPDEIFNFFRSNRITEVGFNMEEAEGVHQHSTLDQQGTEERYRAFMQRFWDLTVQAKGEFKLREFETICTLAYTGDRLGYTDMNQPFVIVNFDHQGNFSTFDPELLSFKIKEYGDFVLGNVLHNTLESVCQTEKFQKIYQDMAAGVVQCRQSCEYFGLCGGGAGSNKYWENGTFNCTETKACRYRIKVIADIVLEGLENSLELANSIS

SEQ ID NO: 67 - protein, 392 aa, Geminocytis sp.
MSIVTSKPVINFKNTANFGPISLIIIQPNSFCNLDCDYCYLPDRHLQNKLSLDLIDPIFKSIFTSPFLGCDFGVCWHAGEPLTMPVSFYKSAFQLIEEANTKYNKSEYSFYHSYQTNGTLINQGWCDLWQEYPVHVGVSIDGPAFLHDVHRKNRKGGNSHDLTMRGIRYLQKNNIPYNTISVITEESLNYPDEMFNFFAENEIYDLAFNMEETEGVNELTSLNGIEIEHKYSQFIKRFWQLVTESKLPFIVREFEILISLIYSGNRLTNTDMNKPFVIVNFDYQGNFSTFDPELLSVKTDKYGDFIFGNVLKDSLESICETEKFKTIYKDINDGVKLCSDNCSYFGICGGGAGSNKYWENGTFASMETQACRYRIKILTDVLVSTIENSLGL

SEQ ID NO: 68 - protein, 375 aa, Micromonospora sp.
  source 1..375: note = strain 375
MAPGPARAALTEFVLKVHARCDLACDHCYVYEHADQSWRRRPVRMTPEVLRTAAGRIAEHAAAHDLPDVTVILHGGEPLLLGAERLGEVLADLRRVIDPVTRLRLGMQTNGVLLSERLCDLLAEHDVAVGVSLDGDRAANDRHRRFRSGAGSYDQVLRAIGLLRRPAYRRIYSGLLCTVDVRNDPIAVYESLLTQEPPRIDFLLPHATWDDPPWRPAGGGTAYAGWLRAVYDRWLADGRPVSVRLFDSLLSTAAGGPSGTEWLGLDPVDLAVVETDGEWEQADSLKTAYDGAPATGMTVFSHAADDVAASPLLARRRSGRAGLSDECRRCPVVDQCGGGLFAHRYGAGHFDHPSVYCADLKELIVHVNENPPAPV

SEQ ID NO: 69 - protein, 8 aa, Xenorhabdus nematophila
CNINCSYC

SEQ ID NO: 70 - protein, 10 aa, Xenorhabdus nematophila
CADCVWNKIC

SEQ ID NO: 71 - protein, 10 aa, synthetic construct
WIXAFXNWXX

SEQ ID NO: 72 - protein, 10 aa, synthetic construct
WINAFGNWER

SEQ ID NO: 73 - protein, 10 aa, synthetic construct
WIKAFGNWSR

SEQ ID NO: 74 - protein, 10 aa, synthetic construct
WINAFANWTK

SEQ ID NO: 75 - protein, 13 aa, synthetic construct
WINAFGNWERAFH

SEQ ID NO: 76 - protein, 14 aa, synthetic construct
AGWIKAFGNWSRSF

SEQ ID NO: 77 - protein, 12 aa, synthetic construct
WINAFANWTKRI

SEQ ID NO: 78 - protein, 47 aa, synthetic construct
GAWGNGGGRGGWINRGGGGSWGNGGSWRNGGGWRNGWGDGGRFINSR

SEQ ID NO: 79 - protein, 93 aa, synthetic construct
GGGFTQGGRRGVATGPRGGNFYNAHPNYGRVGGPVGVGRGAAWADGGGFYNGTYQDGGSFVNGSDGGAAFKNGTYGAGGFVNGSQGGAGFRNW

SEQ ID NO: 80 - protein, 44 aa, synthetic construct
GFANGGGGFANRVGPGGFLNDNGGGGFLNNRGWGDGGGGFLNRR

SEQ ID NO: 81 - protein, 10 aa, synthetic construct
IPAAKFSSFI

SEQ ID NO: 82 - protein, 51 aa, Xenorhabdus sp.
  source 1..51: note = strain NBAII XenSa04
MSKLQREIAENKAQVTNSDKNKTQSKELVDNLLDTVSGGWVNAFANWSKAL

SEQ ID NO: 83 - protein, 51 aa, Xenorhabdus stockiae
  source 1..51: note = strain DSM 17904
MSKLQREIAENKSQIVNSDKNKTQRKELVDGLLDTVSGGWVNAFGNWSKSL

SEQ ID NO: 84 - protein, 51 aa, Xenorhabdus sp.
  source 1..51: note = strain BG5
MSKLQREIVENKTQVTNSDKNKAQRKELVDSLLDTVSGGWVNAFANWSKSF

SEQ ID NO: 85 - protein, 51 aa, Kosakonia cowanii
  source 1..51: note = strain pasteuri
MSKLSKEIAKNQAEVITSKDRNEEKKALAQSMLDSISGGWVNAFARWGKSF

SEQ ID NO: 86 - protein, 51 aa, Yersinia sp.
  source 1..51: note = strain Marseille-Q3913
MSRLKKEIIATKTVVNVSEAKRNQPQRLAEDVLEQVAGGWVNAFARWGRAF

SEQ ID NO: 87 - protein, 51 aa, Yersinia kristensenii
  source 1..51: note = strain IP6945
MSRLQKEINETKTVINICNTKKSQPQHLADSILDKIAGGWVNAFVNWPKSF

SEQ ID NO: 88 - protein, 51 aa, Yersinia bercovieri
  source 1..51: note = strain 127/84
MSSLKKEIMATKTVVNVSEAKRNHPQRLAEDVLEQIAGGWINAFARWGRAF

SEQ ID NO: 89 - protein, 51 aa, Serratia marcescens
  source 1..51: note = strain CAV1761
MSKLAKEINMNKAAVTVAADKKDARKALAQSMLDSVSGGWVNAFARWSKSF

SEQ ID NO: 90 - protein, 51 aa, Yersinia enterocolitica
  source 1..51: note = strain PS23
MSRLQKEINETKTVINICNTKKSQPQHLADSILDKIAGGWVNAFVNWTKSF

SEQ ID NO: 91 - protein, 51 aa, Xenorhabdus bovienii
  source 1..51: note = strain CS03
MRKLQREIALNNAKVINNSEKKQERKVLVENLMDSVSGGWVNVFARWDKAI

SEQ ID NO: 92 - protein, 51 aa, Erwinia sp.
MSKLQREITSNKAQLVNADARKMQRKVLVDSLLDTVSGGWVNAFANWTKRI

SEQ ID NO: 93 - protein, 51 aa, Yersinia aleksiciae
MSRLKKEITATKTVINVSEVKKNQPQRLAEDVLEQISGGWVNAFLRWGKSF

SEQ ID NO: 94 - protein, 51 aa, Erwinia toletana
  source 1..51: note = strain DAPP-PG 735
MSKLQHEIASNKARLNNADDKKAQRKILVDSLLDTVSGGWINAFANWTKRI

SEQ ID NO: 95 - protein, 51 aa, Photorhabdus heterorhabditis
  source 1..51: note = strain ETL
MSSLKREIAETKTEIKGTKVKNNQPQPLTEDLLDQISGGWVNAFAKWTKRI

SEQ ID NO: 96 - protein, 52 aa, Salmonella enterica
MSKLMKEIEKQNAKVTVNNKDKVASRKELTDAVLDSITGGWVNFFAKFTKSF

SEQ ID NO: 97 - protein, 51 aa, Yersinia aldovae
  source 1..51: note = strain IP23238
MSRLKKEITETKTAIGTNKAKKNQPQHLADDLLDQIAGGWVNAFLNWSRSF

SEQ ID NO: 98 - protein, 53 aa, Erwinia sp.
  source 1..53: note = strain E602
MKTLKREIERNNCQLTDVDVVTKKAERKALVDGLLDTVSGGWVNAFANWPKRF

SEQ ID NO: 99 - protein, 51 aa, Yersinia frederiksenii
  source 1..51: note = strain RS-42
MSRLKKEITETKTAIGSNKAKKNQPQHLADDLLDQIAGGWVNAFLNWPRSF

SEQ ID NO: 100 - protein, 51 aa, Aeromonas jandaei
  source 1..51: note = strain CN17A0119
MSKLQREIALNKTKLINADDKKVERKVLVDSLLDTVSGGWVNAFANWTKRF

SEQ ID NO: 101 - protein, 55 aa, Vibrio sagamiensis
  source 1..55: note = strain NBRC 104589
MSKLEKEITINNASVSLNKEVKPEKNKDKNELVQSMLDSVSGGWVNAFARFTKRF

SEQ ID NO: 102 - protein, 51 aa, Xenorhabdus japonica
  source 1..51: note = strain DSM 16522
MSELQREIALNNAQVINSSEKKQERKELVENLMDSVSGGWINVFARWNRAI

SEQ ID NO: 103 - protein, 51 aa, Providencia huaxiensis
  source 1..51: note = strain Pvs2
MSKLSKEIKENNANVKLASNERSSRETLVKSMLESVSGGWVNVFARWDKQI

SEQ ID NO: 104 - protein, 51 aa, Sodalis sp.
  source 1..51: note = strain dw_96
MSKLIKEINFNKAAVTIVADNKNAKKALTQAMLDSISGGWVNAFARWDKKF

SEQ ID NO: 105 - protein, 51 aa, Xenorhabdus bovienii
  source 1..51: note = strain oregonense
MSKLQREIDLNNAQVINSSEKKQERKELVENMMDSVSGGWLNVFVRWDRAI

SEQ ID NO: 106 - protein, 56 aa, Photorhabdus australis
  source 1..56: note = strain DSM 17609
MEESFMSNLKKEIAETKTEIKGTKVKNNQPQPLTEDLLDQISGGWVNAYARWTNRF

SEQ ID NO: 107 - protein, 51 aa, Photorhabdus heterorhabditis
  source 1..51: note = strain SF41
MSSLKKEIAETKTEIKGTKVKNNQPQPLTEDLLDQISGGWVNAYARWTKRF

SEQ ID NO: 108 - protein, 53 aa, Yersinia mollaretii
  source 1..53: note = strain SCPM-O-B-7610
MSRLKKEITATKTVINVNEVKKSQPQRLAEDALEQITGGAGWINAFGNWTKSF

SEQ ID NO: 109 - protein, 53 aa, Yersinia mollaretii
MSRLKKEITATKTVINVNEVKKSQPQRLAEETLEQIAGGAGWINAFANWTKSF

SEQ ID NO: 110 - protein, 53 aa, Yersinia kristensenii
MSRLQKEIIETKTVIDVSGAKKSQPQRLTEDVLEQIAGGAGWIKAFGNWSRSF

SEQ ID NO: 111 - protein, 51 aa, Serratia marcescens
  source 1..51: note = strain 90-166
MSKLAKEISMNKAAVIIDGDKKDIRRALTQSMLDSISGGWVNAFARWSRRW

SEQ ID NO: 112 - protein, 53 aa, Yersinia mollaretii
  source 1..53: note = strain SCPM-O-B-7598
MSRLKKEITATKTVINVSDVKKSQPQRLAEDALEQIAGGAGWINAFANWTRSF

SEQ ID NO: 113 - protein, 52 aa, Xenorhabdus nematophila
  source 1..52: note = strain SC 0516
MSKLQREIAANKAQLSHEDKKKTQHKELVDSLLDTVSGGWINAFGNWERAFH

SEQ ID NO: 114 - protein, 50 aa, Yersinia enterocolitica
  source 1..50: note = strain E701
MKKEIIETKTVIDVSDTKKNRPQHLAEDVLEQIAGGAGWIKVFGNWSRSF

SEQ ID NO: 115 - protein, 51 aa, Serratia marcescens
  source 1..51: note = strain ID149856
MSKLAKEISMNKAAVIIDGDKKDVRRALTQSMLDSVSGGWVNVFARWSRRW

SEQ ID NO: 116 - protein, 53 aa, Serratia sp.
  source 1..53: note = strain DD3
MTRLKKEIIETKTMIDVNSVKNNQPQHLTEDVLDQISGGAGWIRAFANWSRSF

SEQ ID NO: 117 - protein, 54 aa, Mixta theicola
  source 1..54: note = strain QC88-366
MNFTINDLKKLLLNTEENRSPSVAKETIEELSNDDLTNVGGGWFRAYLRWSRSF

SEQ ID NO: 118 - protein, 54 aa, Gilliamella sp.
  source 1..54: note = strain Lep-s5
MFFSKKTIEQRLRDTEAKRKNVPNAKAMEELAAQYLDEVNGGWWRAYARWRRSF

SEQ ID NO: 119 - protein, 48 aa, Photorhabdus laumondii
MSKLAKEIKENKTTVTTKKSADQKAMAQSLLDNVCGGGDRWLKWIKNH

SEQ ID NO: 120 - protein, 48 aa, Kosakonia cowanii
  source 1..48: note = strain pasteuri
MKKLAKEVKQNGVSVNTAKNKAQKKFSQSLLDDVQGGDGRWLQWIKNH

SEQ ID NO: 121 - protein, 46 aa, Yersinia sp.
MSRLKKEITETKTAIGTNKAKKNQPQHLADDLLDQIAGGWVNAFLN

SEQ ID NO: 122 - protein, 53 aa, Bordetella sp.
  source 1..53: note = genomosp. 11 strain AU8856
MMTKEIIQHLEQVQRNAAEEEKTVEEISQSELDQICGAGGVGGFANASWPKSF

SEQ ID NO: 123 - protein, 53 aa, Bordetella bronchialis
  source 1..53: note = strain AU17976
MTTKEIIQHLEQVQRNAAQEEKQMEEISQEELEKICGAGGVGGFANATWSKSF

SEQ ID NO: 124 - protein, 53 aa, Bordetella sp.
  source 1..53: note = genomosp. 9 strain AU14267
MMTKEIIQHLEQVQHNAAEEEKPIEEISQSELDQICGAGGVGGFANATWPKSF

SEQ ID NO: 125 - protein, 50 aa, Providencia rettgeri
  source 1..50: note = strain 2020EL-00052
MKKFKTVIQENSANLKIKKDSDVSKLLEHIRGGKSEAAGGWVNFQWKNSW

SEQ ID NO: 126 - protein, 52 aa, Pandoraea oxalativorans
MTTKAFIEQLAKKQKAANEAGSIKEIPASELERISGARGGNVFVNATWSRAM

SEQ ID NO: 127 - protein, 45 aa, Erythrobacter sp.
MAKNKTPKTEAKAQSKSLESLIDAQLDSIVVGGWSRTVFNRVRPV

SEQ ID NO: 128 - protein, 45 aa, Sodalis sp.
  source 1..45: note = strain dw_96
MDKLRDAIKNNTKTPLAKDTGDLLKSIRGGAGNDGWVKFGWKKKF

SEQ ID NO: 129 - protein, 49 aa, Kosakonia cowanii
  source 1..49: note = strain pasteuri
MSKLAKEIASNKATVTTPTAKAAHVANLLDNVQGGRGEGWVRAYWAKRF

SEQ ID NO: 130 - protein, 50 aa, Bartonella sp.
MSKLKSEINTNNHNNAADDLVELSEATIKKLDAAGGRGQGYVRFIFRRSF

SEQ ID NO: 131 - protein, 48 aa, Photorhabdus heterorhabditis
MKELQKAIQKNSANLKNQKAKEASNLLDAVRGGKPGEGWVNFTWNKSF

SEQ ID NO: 132 - protein, 55 aa, Erwinia sp.
MSKLQREITSNKAQLVNADVRKMQRKVFVDSLLDTVSGGWVNAFANRTMGFLFKL

SEQ ID NO: 133 - protein, 49 aa, Xenorhabdus griffiniae
  source 1..49: note = strain VH1
MKELQKIIHENSANLKNQKGQKASELLDFVRGGASTAETWFKLDWKKSF

SEQ ID NO: 134 - protein, 49 aa, Xenorhabdus griffiniae
  source 1..49: note = strain VH1
MKELQKVIQENSANLKNQKGQKASELLDAVRGGSSDDDGIFFKTTWDRR

SEQ ID NO: 135 - protein, 56 aa, Burkholderia sp.
MDLHVFKKEMMAGAQQEERELLAEIDPELLALVGGGADSQPKARAWFANASFSKRF

SEQ ID NO: 136 - protein, 56 aa, Trinickia sp.
MDLHVFKKEMMAGAQQVEREMPAELDPEFLALVGGGVESQSKPRAWFANSSFSKRF

SEQ ID NO: 137 - protein, 57 aa, Burkholderia sp.
MDLHAFKNEMMVGAQQVEREAPVELDSELLALVGGGASSQANSRGWFANATWSKAWR

SEQ ID NO: 138 - protein, 392 aa, Xenorhabdus sp.
  source 1..392: note = strain NBAII XenSa04
MAIVKNEKIKHIEIILKISERCNINCTYCYVFNMGNTLAADSTPIISLDNVAALRGFFERSVIENEIEVIQVDFHGGEPLMMKKERFNRMCEILREGNYGSSRLVLALQTNGILIDDEWIALFEKHQVHASISIDGPKHINDRHRLDQKGKSTYEGTVKGLRMLQNAWAQGRIPVEPGILSVANAKANGEEIYHHFSKELKCQRFDFLIPDDQHTDGIDAEGIGRFLNEALDAWFADGQPNIFVRIFNTYLGTMLNNQFSRVLGISANVESAYAFTVTSDGLLRIDDTLRSTSDKIFNSIGHVSKLTLASVLESSNVREYLSLSDELPDACCGCIWSKVCHGGRLVNRFSQTNRFHNKTVFCPSMRLFLSRAASHLIAAGISEETIIENIQK

SEQ ID NO: 139 - protein, 392 aa, Xenorhabdus stockiae
  source 1..392: note = strain DSM 17904
MAIIKNEKIKHLEIILKVSERCNINCTYCYVFNMGNTLAADSAPIISLDNIAALRGFFERSVIENHIEVIQVDFHGGEPLMMKKERFNQMCEILREGNYGNSQLVLALQTNGILIDDEWIALFEKHQVHASISIDGPKHINDRHRLDRKGKSTYEGTVNGLRMLQNAWAQGRIPAEPGILSVANANANGGEIYHHFSKELKCQRFDFLIPDDQHADSTDAEGIGRFLNEALDAWFADGQPNIFVRIFNTYLGTMLNSQFHRIIGISANVESVYAFTVTSDGLLRIDDTLRSTSDKIFNPIGHVRELTLSSVLESTNAKEYSSLNSELPEDCNDCIWSKICHGGRLVNRFSPTNRFHNKTVFCPSMRVFLSRAASHLIEAGVSEETIIKNIQQ

SEQ ID NO: 140 - protein, 392 aa, Xenorhabdus sp.
  source 1..392: note = strain BG5
MAIVKDGKVKHLEVILKISERCNINCTYCYVFNMGNTLAADSAPVISLDTVASLREFFERSVVENEIEVIQVDFHGGEPLMMKKERFNRMCEILREGNYGRSRLVLALQTNGILIDNEWISIFEKHQIHVSVSIDGPKHINDRYRLDRKGKSTYEGTVNGLRMLQNAWTQGRLSGEPGILSVANAKANGEEIYRHFTKELKCQRFDFLIPDDQHADSIDVEGIGRFLNEALDAWFADGQPKIFIRIFNTYLGTMLNNQFSRVLGMSANVESAYAFTVTADGQLRVDDTLRSTSDQIFSAIGHVSELTLARVLESPNVKEYLSLSSELPDACCGCVWSKICHGGRLVNRFSRANRFHNKTVFCLSMRLFLSRAASHLIAAGVSEETIIENIQK

SEQ ID NO: 141 - protein, 396 aa, Kosakonia cowanii
  source 1..396: note = strain pasteuri
MKNWSQNDLKKIKHLEIILKVSERCNINCSYCYMYNLGNNISIKSKPVIPFSVVKDLRNFFEQATKEHEIETIQVDFHGGEPLMMGKERFEVACDELAKGHYKNTKLNMACQTNATLIDDEWIEVFSKYNISVGISIDGPKHINDKHRLDKKGRSTYDKKVNGLKMLQKAWQEGKLADEPGILCVANQSVNGAEIYRHFVDDLKSKKFDFLIPDESHDTCSNPDGLSKFYCDAMDEFFSDANKNVYVRYFHTHMQSMLSQEFRPVMGISKSNDDILAFTVCSNGDIYIDDTLRATNDSIFTPIGNIKNLTLSDALSSWQMKKYILIKKTLPENCTDCVWKKICGGGRHIQRYSKDDDFNRETVFCPSIRKIMSRAASHLISSGIPEEKIMMNLEII

SEQ ID NO: 142 - protein, 394 aa, Yersinia sp.
  source 1..394: note = strain Marseille-Q3913
MVNISSKKNIQHLEVILKISERCNINCDYCYVFNKGNSISDNSPARISSENINQLVYFLQRACLEYDIATLQIDFHGGEPLLMKKENFARMCDQLVTADYGGSNINLALQTNGTLVDDEWISLFEKYSVNASVSIDGPKHINDRHRLDTKGRSTYEGTVRGLRMLQKAYQQGRIPSEPGILCVADASVDGAEIYRHFVDELGVYSFDFLIPDDCYKDTHVDAIGMGRFLNEALDEWVKDDNPKVFVRLFQTHIASLLGQMNSGVLGHNPNVTGIYALTVSSDGLVRVDDTLRSTSDSMFNPIGHMSEISLLDVFDSQQFREYSLIGQSLPTECTGCIWENICAGGRIVNRFSPEDRFNRKSTYCYSMRSFLSRASAHLLNMGIKEERIMAAISQ

SEQ ID NO: 143 - protein, 394 aa, Yersinia kristensenii
  source 1..394: note = strain IP6945
MVNQLNIQSIQHLEIILKISERCNINCDYCYVFNKGNPAANNSPARLSDRNINDLAEFLHTACREYKIGTLQIDFHGGEPLLMKKENFAKMCERLLTGRYSKTNIRFALQTNGTLIDEEWISLFEKYSVNASISIDGPKHINDRHRLDTKGRSTYEATVRGLRILQHAHKQGRIPSAPGVLCVANAQANGAEIYRHFVDELKVYGFDFLVPDDCYHDTNIDPVGISRFLNEALDEWFKDSNPNIFVRLFQTHLAHLLGTKHQGILGHSPSATGAYAFTVGSDGFIRVDDTLRATSDRIFNPIGHVSEISLTDALNSPQFQEYASVGQALPHECNGCIWENVCAGGRIMNRFSPETRFDRKSVYCYSMRSFLSRAAAHLLNMGIKEERIMTAIGR

SEQ ID NO: 144 - protein, 394 aa, Yersinia bercovieri
  source 1..394: note = strain 127/84
MVNISSKKSIQHLEIILKISERCNINCDYCYVFNKGNSIADNSPARISNKNIEQLVYFLQRACLEYDIATLQIDFHGGEPLLMKKENFASMCDQLTTADYGSSNISLALQTNGTLIDDEWISLFEQYLVYVSISIDGPKHINDRHRLDTKGRSTYEGTVRGLRMLQNAYKQGRLQAEPGILCVANPQANGAEIYRHFVDDLGVYGFDILIPDDAYNDTYADPVSMGRFLNEALDEWMKDDNPKIFVRLFQTHIATLLGAKKVGVLGHTPEVTGTYACTVGSDGLIRVDDTLRSTSDRIFNAIGHVSEINLSDVINSPQFQEYVSIGKSLPTECTGCIWENVCAGGRIMNRFSPEERFNRKSVYCYSMRSFLSRASAHLLNMGIKEERIMAAISQ

SEQ ID NO: 145 - protein, 392 aa, Serratia marcescens
  source 1..392: note = strain CAV1761
MTNKKKIKHLEIILKVSERCNINCTYCYVFNLGNDLAINSKPIISHKIIEDLRGFFERACQEYEIETVQVDFHGGEPLMMGKERFDNACKELISGDYNGARLNLACQTNAILIDNEWIDIFSKYNISVGISIDGPKHINDRHRLDRKGRSTYEGTVKGLEMLQVAWKAGRLIDEPGILCVANPSVKGAEIYRHFVDVLKCKKFDFLIPDESHDTCTDPDGLADFYCSALDEFFLDADKEVYVRYFHTHIQSMLSSEFNPVMGVSKAGNDTLAFTVSSDGELYVDDTLRATNDPIFTPIGNIQHLILSDTLASWQMTKYMAVNSQLPTVCGDCVWQKVCGGGRHIQRYSTADDFNRETVFCPSVRKIMSRAASHLIESGVAEDIIMKNLEVNS

SEQ ID NO: 146 - protein, 305 aa, Yersinia enterocolitica
  source 1..305: note = strain PS23
MVNQLNMQSIQHLEIILKISERCNINCDYCYVFNKGNPAANNSPARLSDKNINALAELLHTACREYKIGTLQIDFHGGEPLLMKKENFAKMCERLPAGKYSKTNVRFALQTNGTLIDEEWISLFEKYSVNASISIDGPKHINGRHRLDTKGRSTYEATVRGLRILQHAHKQGRIPSAPGVLCVANAQANGAEIYRHFVDDTLRATSDRIFNPIGHVSEISLTDALNSPQFQEYTSIGQSLPHECNGCIWENVCAGGRIMNRFSPETRFDRKSVYCYSMRSFLSRTAAHLLNMGIKEERIMAAIQA

SEQ ID NO: 147 - protein, 392 aa, Xenorhabdus bovienii
  source 1..392: note = strain CS03
MITKKKIKHLEIILKVSERCNINCTYCYVFNLGNEISINSKPIISHDIIKVLRAFFEQASQEYDIETIQVDFHGGEPLMMGKEKFENACNEFISGSYNKTKFNLACQTNAILIDNEWIDIFSKYNVSVGISIDGPKHINDKHRLDRKGRSTYEGTVRGLVMLQEAWSAGRLIDQPGILCVANPSVKGAEIYRHFVDVLKCKKFDFLIPDESHDTCTNPDGLSDFYCSAIDEFFSDADQDVYVRYFLTHMQSMLSSEFSPVMGLSKSGSDTIALTVSSEGDIYVDDTLRSTNDPIFTPIGNVLNLTLSETIASWQMQKYMTVNNQLPTACTDCIWKKVCGGGRHIQRYSKADDFKRESVFCPSIRKIMSRAASHLIESGISEDIIMKNLGIKS

SEQ ID NO: 148 - protein, 392 aa, Erwinia sp.
MRLIKGEKIKHLEIIFQVSERCNISCTYCYVFNMGNTLAADSHPTISLNNVIALRGFFERSTAENEIEVIQVDFHGGEPLMMKKDRFDQMCHILLQGDYGNSRIELALQTHGILVDEEWITLFEKYKVHASISVDGPKHINDRHRLDRKGKSTYEGTINGLRLLQNAWQQGRLPAEPGILSVANAKANGADIYHHFVDVLKCQRFDFLIPDDHHDDITDSEGIGRFLNEALDAWFADGRAELFVRIFNTYLGTLLDKQFSRVLGMSANVESAYAFTVTADGLLRIDDTLRSTSDEIFNPVGHVRDLSLAGVLKNTAVEEYLSLSNTLPEGCKDCVWNNVCHGGRLVNRFSQANRFNNKTVFCSSMRIFLSRGASHLMATGIDERTIMANIQG

SEQ ID NO: 149 - protein, 393 aa, Yersinia aleksiciae
MVNISSEKRIKHLEIILKISERCNINCDYCYVYNKGNTIADNSPARISNKNILQLVDFLQRACREYSIGTLQIDLHGGEPLLMKKENFASMCELLMMADYCGSNINLALQTNGTLVDDEWISLFEKYSIHVSISIDGPKHINDRHRLDTKGRSTYEGTVRGLRRLQHAHQQGRLRAAPGILCVANPQASGTEIYRHFVDDLGVYGFDLLIPDDAYSDDHVDPISMGRFLNEALDEWVKDDNPKIFVRLFQTHIATLLGAKVGVLGHTPEVTGAYACTVGSDGFIRVDDTLRATSDRIFDPIGHVSDISLSEVLDSPQFQEYTLIGQSLPTECENCIWAKVCAGGRIMNRFSPEDRFNRKSVYCYSMRSFLSRASAHLLNMGIKEERIMAAISQ

SEQ ID NO: 150 - protein, 392 aa, Erwinia toletana; identical to SEQ ID NO: 63
  source 1..392: note = strain DAPP-PG 735

SEQ ID NO: 151 - protein, 394 aa, Photorhabdus heterorhabditis
  source 1..394: note = strain ETL
MVNSLVKKKIQHLEVILKISERCNINCDYCYVFNKGNSAANDSPARISHANIDYLVDFFQRGSQEYDIDTLQIDFHGGEPLMMKKQQFASMCDRLASGNYHGSNIKFALQTNGILIDDEWISLFEKYSVSVSVSIDGPKHINDRHRLDRKGRSTYEGTVRGLRKLQEAYQAGRLPSDPGILCVANAKASGAEIYRHFVDNLGVYGFDFLVPDDCYTDALVDPVGVGRFLNEALDEWVNDNNPKIFVRLFNTHIASLLGAENAGFLGHNPSVAGIYAFTIGSDGSVRIDDTLRSTSDRIFDIIGHISEISLSEVLNSPQFQEYVSIGQSLPTECEDCIWAKICAGGRIVNRFSHEERFKRKSVYCYSMRSLLGRVSAHLLNMGIEEDRIMKAISR

SEQ ID NO: 152 - protein, 401 aa, Salmonella enterica
MPPFKGGLLMNKEKFNFLEIVLKVSERCNINCDYCYMYNCGNELSINSRPLINDETVYNLKKLLENAASEFEIGTIQVDFHGGEPLMLGKRKFSEACDILLSGNYHNSYFILSCQTNGTLIDEEWVDIFYKYNVRIGISIDGPKHINDKHRLDHKGKSTYERTVKGIKMINSAWKKGIMTNEPSILCVINPKVSGKEIYRHFVDDLECKSFDLLIPDENHDTCENTKAVGLYLNEAVDEFFNDSNKEIEVRIIATHMKSLMLKEFTPVIGISKGDINSAVFVITSEGDIYIDDALRVTNDILFSPIGNLRNVKFKNLLESWQLKQYMNINNTLPSSCYDCIWKNSCFGGRALNRFSKVNRFDNKTVFCDSMRIFLSRLTSHIIESGVDIKLIEENLGVNEL

SEQ ID NO: 153 - protein, 394 aa, Yersinia aldovae
  source 1..394: note = strain IP23238
MGHLLTKKRIKHFEIILKISERCNINCDYCYVFNKGNSDADNNPARISNKNIGHLANFLQRACLEYEIDTLQIDFHGGEPLLMKKEHFANMCIQLISGNYCGSNIRLALQTNGILIDDEWISLFEKYSVNVSLSIDGPKHINDRHRLDTKGRSTYEGTVRGLRLLQSAYQQGRLPSAPGILCVANAQANDAEIYRHFVDDLGVYGFDFLIPDDSYNDVNIDPIGIGRFLNEALDEWVKDNNPKIFVRHFQTHFASLLGVKNIGILGQSSNITGVYAFTVSSDGSIRVDDTLRSTSDRIFNTIGHISEINLSDVLNSPQAQEYSSIGQCLPNECKGCIWENICTGGRLVNRFSSEERFKHKSVYCYSIRSFLSRASAHLLNMGIKEERIMTSICQ

SEQ ID NO: 154 - protein, 396 aa, Erwinia sp.
  source 1..396: note = strain E602
MGANKEKIKHLEIILKISERCNINCDYCYVFNMGNQLATESNPVISMSNILSLRGFFERSVKEYEINVLQVDFHGGEPLMIKKSRFDEMCEILKGGNYSNSKLELALQTNGILIDEEWIVLFEKHKVHVSISVDGPKHINDRHRLDRKGKSTYEGTIKGFRLLQDAWESGRIPGEPGILSVANAKANGAEIYRHFVDVLDCKRIDFLIPDDHHNDEVDSQGIGMFLTEALDEWFSDGNSGVFVRIFNTYLGTMLNHQFSRVLGMSANVESAYAFTVTSDGIIRIDDTLRSTSDKIFDALGHVDEMSLSDVFEHNNFKEYIYLNAVLPAGCHGCLWSNICHGGRLVNRFSLDGRFNNKTIFCSSMKIFLSRAVAHLLASGIEEETIIKNIEKKEISV

SEQ ID NO: 155 - protein, 394 aa, Yersinia frederiksenii
  source 1..394: note = strain RS-42
MDNLLTKKRIKHFEIILKISERCNINCDYCYVFNKGNSDADNNPARISNTNISHLANFLQRACFEYEIDTLQIDFHGGEPLLMKKEHFANMCIQLISGNYRGSSIRLALQTNGTLIDDEWISLFEKYSVNVSISIDGPKHINDRHRLDTKGRSTYEGTVRGLRLLQSAYRQGRLPSAPGILCVANARANGAEIYRHFVDDLGVYGFDFLIPDDSYNDVNIDPIGIGRFLNEALDEWVKDNNPKIFVRHFQTHFASLLGVRNIGVLGQSSNITGVYAFTVGSDGSIRVDDTLRSTSDRIFNTIGHISEINLSDVLNSPQAQEYSSIGQCLPNECKGCIWENICTGGRLVNRFSSEERFKHKSVYCYSIRSFLSRASAHLLDMGIKEERIMAAISQ

SEQ ID NO: 156 - protein, 392 aa, Aeromonas jandaei
  source 1..392: note = strain CN17A0119
MTLIKGEKIKHLEIILKISERCNISCTYCYVFNMGNSLAADSSPVMSLDNVLALRGFFERSASENEIEVIQVDFHGGEPLMMKKNRFDQMCNILLQGNYGNSRLELALQTNGILIDEEWITLFEKHKVHTSISVDGPKHINDRHRLDRKGKSTYEGTINGLRLLQKAWEQGRLPGEPGILSVANAKANGAEIYRHFVDVLKCQRFDFLIPDDHHDDNTDNEGVGKFLNEALDAWFADGRPELFVRIFNTYLGTMLDNQFSRVLGMSANVESAYAFTVTADGLLRIDDTLRSTSDEIFNAVGHVRDLSLKSVLKNSSVKEYLSLSGELPNDCVDCVWNNVCHGGRLVNRFSKANRFNNKTVFCSSMRVFLSRAAAHLMATGIDERAIMENIQK

SEQ ID NO: 157 - protein, 391 aa, Vibrio sagamiensis
  source 1..391: note = strain NBRC 104589
MIRKKIKHLEIILKVSERCNINCTYCYVFNLGNDIAINSKPIISHQNIKHLKHFFERATREYEIESLQVDFHGGEPLMMGKERFKAACKELMSGDYQNSRLSLACQTNAILIDDEWIDIFSKYDVSVGISIDGPKHINDKHRIDRKGRGTYDDTVAGLKKLQAAWEEGKIADEPGILCVANPSVKGADIYRHFVDVLGCKKFDFLIPDESHDTCEDPHSLAEFYCSALDELFNDADKDIYVRYFHTHIHSMLASNFNPVMGMSKSTNDTIAYTVSSEGELYIDDTLRATNDNIFTSIGNIKDLTLSESINSWQMQKYMQVNNQTPEPCSECIWKNICGGGRHIQRYSKEDDFNRNSVYCPSIRKIMSRTASHLISSGIPEEKILTNLGVHN

SEQ ID NO: 158 - protein, 395 aa, Xenorhabdus japonica
  source 1..395: note = strain DSM 16522
MLTMIKKKKIKHLEIILKVSERCNINCTYCYVFNLGNEISINSKPIISHSTIKDLRAFFEQASQEYDIETIQVDFHGGEPLMMGKEKFENACNEFISGGYNKTKLNLACQTNAILIDNEWIDIFSKYNVSVGISIDGPKHINDKYRLDRKGRSTYEGTVRGLVMLQEAWNAGRLIDQPGILCVANPSVKGAEIYRHFVDVLKCKKFDFLIPDESHDTCANPDGLSDFYCSVIDAFFSDADQDVYVRYFLTHMQSMLSSEFSPVMGLNKSGNDTIALTVSSEGDIYVDDTLRSTNAPIFTSIGNILNLTLSETIASWQMQKYMTVNNQLPTACTDCIWKKVCGGGRHIQRYSKADDFKRESVFCPSIRKIMSRAASHLIESGISEDIIMKNLGIKS

SEQ ID NO: 159 - protein, 389 aa, Providencia huaxiensis
  source 1..389: note = strain Pvs2
MDKIKHLEVILKVSERCNINCTYCYVFNLGNEVAINSKPIISSEIINHLVEFFEQATTEYDIESIQVDFHGGEPLMMGKKRFIAACQKLISGNYNNTKLYLACQTNAILIDPDWIDIFSKYSISIGVSIDGPKHINDKHRLDTKGRSTYDNTIKGFKLLQNAWREGKLKDQPGILCVANPNVSGKDIYRHFVDELECTKFDFLIPDETHDTCIDPTHLSEFYCSALDEFFLDSNNDIYIRYFHTNIQSMLKSDFTPTMGVSKTSNDIIALTISSEGDVYIDDTLRGTNDDIFSVIGNIKKTKFRETLSSWQMEKYMQINSQLPSDCVNCIWKKTCSGGRHIQRYSKADNFNRKSVFCPSIKKILSRAASHLLESGVPEELIMDNLGIKS

SEQ ID NO: 160 - protein, 387 aa, Sodalis sp.
  source 1..387: note = strain dw_96
MIKIKHLEIILKVSERCNINCTYCYVFNLGNDISINSKPIISHDIIKDLTGFLERASHEYDIETIQIDFHGGEPLMMGKEKFDSACRDFLSGNYKKSRLQLACQTNAMLIDEEWIDIFSNNNISVGVSIDGPKHINDKHRLDRKGRSTYEGTVKGLVMLQDAWQAGRLIDEPGILCVANSLVNGAEIYRHFVDVLHCKKIDFLIPDETHDTCKDPEGLSDFYCSAIDEFFSDADSNVYIRFFYTHIQSMLNSDLSPVLGLSKSESDTLAFTVGSEGELYVDDTLRATNDPIFTSIGNVRNLSLSETIASWQMQKYMAVNNNLPLVCTDCIWQKICGGGRHIQRYSKADDFNRETVFCPSIRKIMSRAASHLLDCGVSENTIMKNLDS

SEQ ID NO: 161 - protein, 394 aa, Xenorhabdus bovienii
  source 1..394: note = strain oregonense
MITMIAKKKIKHLEIILKVSERCNINCTYCYVFNLGNEISINSKPIISHNTIKDLRAFFEQASQEYDIETIQVDFHGGEPLMMGREKFENACNEFISGSYNKTKLNLACQTNAILIDNEWIDIFSKYNVSVGISIDGPKHINDKYRLDRKGRSTYEGTVRGLVMLQEAWNAGRLIDQPGILCVANPSVKGAEIYRHFVDVLKCKKFDFLIPDESHDTCANPDGLSDFYCSVIDEFFSDADQDVYVRYFFTHMQSMISSEFSPVMGLSKSGSDTIALTVSSEGDIYVDDTLRATNDPIFTPIGNILNLTLSETIASWQMQKYMTVNNQLPTACTDCIWKKVCGGGRHIQRYSKADDFKRESVFCPSIRKIMSRAASHLIESGISEDIIMKNLGIK

SEQ ID NO: 162 - protein, 394 aa, Photorhabdus australis
  source 1..394: note = strain DSM 17609
MVNSLVKKKIQHLEVILKISERCNINCDYCYVFNRGNSAANDSPARISHANIDYLVDFFQRGSQEYDIDTLQIDFHGGEPLMMKKPQFASMCERLASGNYHGSKIRFALQTNGILIDDEWISLFEKYSVSVSVSIDGPKHINDRHRLDRKGRSTYEGTIRGLRKLQEAYQAGRLPSDPGILCVANAKASGAEIYRHFVDNLGVYGFDFLVPDDCYTDAQVDPDGVGRFLNEALDEWVNDNNPKIFVRLFNTHIASLLGAENAGFLGHNPSVAGIYAFTIGSDGFVRVDDTLRSTSDRIFDIIGHISEISLSEVLNSPQFQEYASIGESLPTECEDCIWAKVCAGGRIVNRFSHEERFKRKSVYCYSMRSLLSRVSAHLLNMGIEEDRIMKAIGR

SEQ ID NO: 163 - protein, 394 aa, Photorhabdus heterorhabditis
  source 1..394: note = strain SF41
MVNSLVKKKIQHLEVILKISERCNINCDYCYVFNRGNSAANDSPARISHANIDYLVDFFQRGSQEYDIDTLQIDFHGGEPLMMKKQQFASMCERLASGNYYGANIRFALQTNGILIDDEWISLFEKYSVSVSVSIDGPKHINDRHRLDRKGRSTYEGTVRGLRKLQEAYQEGRLPSDPGILCVANAKASGAEIYRHFVDNLGVYGFDFLVPDDCYTDAQVDPVGVGRFLNEALDEWVNDNNPKIFVRLFNTHIASLLGAENAGFLGHNPSVAGIYAFTIGSDGSVRVDDTLRSTSDRIFDIIGHISEISLSEVLNSPQFQEYSSIGESLPTECEDCIWAKVCAGGRIVNRFSNEERFKRKSVYCYSMRSLLGRVSAHLLNMGIEEDRIMKAIGR

SEQ ID NO: 164 - protein, 394 aa, Yersinia mollaretii
  source 1..394: note = strain SCPM-O-B-7610
MVELLINKRIRHLEIILKISERCNINCDYCYVFNKGNSAANDSPARISDKNIHHFVNFLERASQEYQIGTLQIDLHGGEPLLMKKENFANMCIQFMSGHYCGSNIRLALQTNGTLIDEEWIALFERYSVNVSVSIDGPKHINDRHRLDTKGRSTYEGTVRGLRMLQQAYQQGRLPSAPGILCVANAKVNGAEIYRHFVDDLGVYSFDFLIPDDCYKDADVDSLGLGRFLNEALDEWVKDDNPKIFVRLFQTHIATLLGQKNSGILGHNPSVTGVYALTVSSDGFVRVDDTLRSTSDSMFNPIGHTSEVSLSEVFDSPQFREYTSVGQSLPTECTGCIWENICAGGRIVNRFSPEDRFDRKSAYCYSMRSFLSRASAHLINMGIKEERIMAAISQ

SEQ ID NO: 165 - protein, 394 aa, Yersinia mollaretii
MVELLIDKRIRHLEIILKISERCNINCDYCYVFNKGNSAANDSPARISDKNIHHFINFLERASQEYQIGTLQIDLHGGEPLLMKKENFANMCIQFMSGHYCGSNIRLALQTNGTLIDEEWIALFEKYSVNVSVSIDGPKHINDRHRLDTKGRSTYEGTVRGLRMLQQAYQQGRLPSAPGILCVANAKVNGAEIYRHFVDDLGVYSFDFLIPDDCYKDADVDALGLGRFLNEALDEWVKDDNPKIFVRLFQTHIATLLGQKNSGILGHNPSVTGVYALTVSSDGFVRVDDTLRSTSDSMFNPIGHTSEVSLSEVFDSPQFREYTSVGQSLPTECTGCIWENICAGGRIVNRFSPEDHFDRKSAYCYSMRSFLSRASAHLINMGIKEERIMAAISQ

SEQ ID NO: 166 - protein, 394 aa, Yersinia kristensenii
MLNLLIEKNIRHLEIILKISERCNINCDYCYVFNKGNSAADDSPARLSNKNIHHLVCFLQRACQEYKIGTVQIDFHGGEPLLMKKENFTDMCIQLISGNYCGSNIRLALQTNATLIDNEWIAIFEKYSVNVSISIDGPKHINDRHRLDTKGRSTYESTVRGLRILQNAYQQGRLPSDPGILCVTNAQANGAEIYRHFVDELGVYSFDFLIPDDSYKDAHPDAVGIGRFLNEALDEWVKDNNAKIFVRLFQTHIASLLGQKNSGVLGHTPNITGVYALTVSSDGFVRVDDTLRSTSDRMFNPIGHLSEVNLSNVFASPQFQEYSSIGQSLPTECEGCIWENICAGGRIVNRFSTEDRFKHKSIYCYSMRTFLSRSSAHLLNMGIKEERIMAAIRA

SEQ ID NO: 167 - protein, 392 aa, Serratia marcescens
  source 1..392: note = strain 90-166
MANKEKIKHLEIILKVSERCNINCTYCYVFNLGNDLAINSKPIISHGVIKNLREFFERACREYEIETVQVDFHGGEPLMMGKDRFDNACKELVSGDYNGTRLNLACQTNAILIDNEWIDIFSKYNMSVGISIDGPKHINDRHRLDRKGRSTYEGTVKGLEMLQVAWRAGRLIDEPGILCVANPSVKGAEIYRHFVDVLKCKKFDFLIPDESHDTCTDPEGLSDFYCSALDEFFLDADKEVYVRYFHTHIQSMLSSEFSPVMGVSKAGSDTLAFTVSSDGELYVDDTLRSTNDSIFTPIGNLHSLTLSEALMSWQMQKYLSVDNQLPKVCIDCVWKKLCGGGRHIQRYSSNDDFNRETVFCPSIRKIMSRAASHLIESGVSEDVIMKNLEVNS

SEQ ID NO: 168 - protein, 394 aa, Yersinia mollaretii
  source 1..394: note = strain SCPM-O-B-7598
MVETLIDKRIRHLEIILKISERCNINCDYCYVFNKGNSAANDSPARISDKNIRHFVDFLERASQEYQIGTLQIDLHGGEPLLMKKENFANMCIQFMSGYYCGSNIRLALQTNDTLIDEEWIALFGKYSVNVSVSIDGPKHINDRHRLDTKGRSTYEGTVRGLRMLQQAYQQGRLPSAPGILCVANANVNGAEIYRHFIDELGVYSFDFLIPDDCYKDTYVDAVGMARFLNEALDEWVKDNNPKIFVRLFQTHIATLLGQKNSGILGHNPSVTGVYALTVSSDGFVRVDDTLRSTSDPMFNPIGHTSEVSLSEVFNSPQFQEYSSIGQSLPTECAGCIWENICAGGRIVNRFSPEDRFDRKSAYCYSMRSFLSRASAHLINMGIKEERIMAAISQ

SEQ ID NO: 169 - protein, 392 aa, Xenorhabdus nematophila; identical to SEQ ID NO: 61
  source 1..392: note = strain SC 0516

SEQ ID NO: 170 - protein, 394 aa, Yersinia enterocolitica
  source 1..394: note = strain E701
MLNLLIEKKIRHLEIILKVSERCNINCDYCYVFNKGNSAADDSPARISNKNIHHLVYFLQRACQEYQIDTIQIDFHGGEPLLMKKESFTNMCIQLISGNYCGSQLRLALQTNATLIDNEWIAIFEKYSVNVSISIDGPKHINDRHRLDTKGRSTYEGTVRGLRILQHAYKQGQLPSDPGILCVANAQANGAEIYRHFVDELGVYSFDFLIPDDSYKDAHTDAIGIGRFLNEALDEWIKDNNAKIFVRLFQTHIASLLGQKNSGVLGHTPNVTGIYALTVSSDGFVRVDDTLRSTSDRMFNPIGHLSEVNLSNVFASPQFQEYSSIGQSLPTECEGCIWENICAGGRIVNRFSTKDRFKRKSIYCYSMRTFLSRSSAHLLNMGIKEERIMAAIQA

SEQ ID NO: 171 - protein, 392 aa, Serratia marcescens
  source 1..392: note = strain ID149856
MANKEKIKHLEIILKVSERCNINCTYCYVFNLGNDLAINSKPIISHGTIKNLRGFFERACQEYEIETVQVDFHGGEPLMIGKDRFDNACKELVSGDYNGTRLNLACQTNAILIDNEWIDIFSKHNISVGISIDGPKHINDRHRLDRKGRSTYEGTVKGLEMLQAAWRAGRLIDEPGILCVANPSVKGAEIYRHFVDVLKCKKFDFLIPDESHDTCTDPEGLSDFYCSALDEFFLDADKEVYVRYFHTHIQSMLSLEFSPVMGVSKAGSDTLAFTVSSDGELYVDDTLRSTNDSIFTPIGHIQSLTLSEALTSWQMQKYLSVDNQLPEVCIDCIWKKLCGGGRHIQRYSSADDFNRETVFCPSIRKIMSRAASHLIESGVTEDIIMKNLEVNS

SEQ ID NO: 172 - protein, 394 aa, Serratia sp.
  source 1..394: note = strain DD3
MVNLLNKKHIKHLEIILKISERCNINCDYCYVFNKGNSASNDSPARLSDKNVNHLVDFFQRACLEYEIGTLQIDFHGGEPLLMKKENFDRMCDRLVTGNYCGSNIRLALQTNGMLVDDEWLALFEKHSVNVSISIDGPKHINDRHRLDTKGRSTYEGTVRGLRKLQHAYQQGRLPSDPGILCVANAQANGAEIYRHFVDDLNVRSFDFLIPDDCYKDTHVDPVGLGRFLNEALDEWVKDDNAKIFVRLFQTHIASLLGKENVGVLGHTPSITSVYALTVSSDGFVRVDDTLRSTSDRMFNTIGHLSEINLSDVFDSPQFQEYASIGQSLPTECKGCIWENICAGGRIMNRFSTEERFKRKSVYCYSMRSFLSRASAHLLNMGIKEERIMEAINR

SEQ ID NO: 173 - protein, 393 aa, Mixta theicola
  source 1..393: note = strain QC88-366
MAKKIDILEIILKVTECCNIACRYCYYFEGDNRDFADKPRVMNKKTVIQLANYLKETVVAHQIETLRIDIHGGEPLMMGKKRLGELLLILSDALKKICKLEFVLQCNGTLIDDDWINIFAKYQVAASVSVDGDAVTHNLNRIDRRGKGTYHRVMAGLSKLIAASKDNKVPYPGVLCVINPDKNGKVIFRHFVEQNKTPYISFIEPDFTIDEASKQRVDGIGNFLLDVYQEWEKNNSPKINRHMSLRVFNDLLSVLMVSGTEYENMKTINYVVITIRSDGYINPDDILRNTHPELFNESYHLASSTLEEFITSEDIRELYRGIFTLPVQCQECGVRKLCRNGFCFGSLPHRYSKKNGMNNTNLFCKFYREICIRLCNYAVNKGKTFAEIEKAVY

SEQ ID NO: 174 - protein, 395 aa, Gilliamella sp.
  source 1..395: note = strain Lep-s5
MSNSIKVDILEVILKITECCNIACRYCYFFRGGNIDFDERPNVIKKDTIHALASFLKEAILANEIKLLRLDFHGGEPLMMGKKRFVEMVELFDTELSQLVDLEYVLQSNGTLIDDEWVEIFSKYNVAASVSLDGDQAIHDANRIDKKGRGTYVRATEGLKKLICAARSNKVVFPGIISVINDSSDTKITFKHFLDDLESPFISFVELDLTIDELNQETVEKISNNLLAVYNEWERINTPTIVHDISVRNFNDILKQLVLSGTEADKKEKRKYVSLTIRSDGSLNPDDILRNIYPYLFTNEYNIKNNTLSDYLSDEKLKDLYRKLFTLPEKCNECGVKKICRNGWGFGSIPHRYSKENDMNNVNALCGVYHEISLRLCDLVIQQGKSYDSIKHNLF

SEQ ID NO: 175 - protein, 307 aa, Photorhabdus laumondii
MKKIKHLEIIAKVSERCNINCTYCYVFNMGNDLAINSKPVISLKTVSNLKRFLERSLTEYNIESIQVDLHGGEPLMLNRERFSRMCEELMSGDYKGAKFSIACQTNATLIDDEWIDIFSKYNISVSVSIDGPKHINDKNRIDNKGKGTYDATVSGLFKLQSAWKDGKLPSAPGVLCVANPNSNGAEVYRHFVDVLNCKSFDFLIPDESHDNCKNPYGISDFFCSAVDEFFSDADKKIIVRYFYATIQGMLNPGIFHVAGMGKMNNDIVAFTMGSEGNIHVDDILRSSNDDIFTAIGNVNELSLNNVI

SEQ ID NO: 176 - protein, 391 aa, Kosakonia cowanii
  source 1..391: note = strain pasteuri
MKSIEHLEIIVKISERCNIDCTYCYVFNKGNDLAINSQTIIKKNTINSFRDFLESASKGFDIKTIQIDFHGGEPLLLKKDRFNFLCKTLREGDYRGSRLVLSCQSNGVLIDDEWIDIFHKWDVGVSVSMDGPKHIHDAARIDKNGKGTYDQVVAGFRKLQDAWKENKISTQPGILCVANTNLKGVEIYRHFIDDLQCKGFDFLIPDETHDSNIDASKLYDFYESVIDEYFIDADIDIKFRYLKVLIQGMLNPGTYAIAGLNAVNNDIVALTMGANGDIYIDDTLRSTSDKAFSKIINISSGSLGDILSSWQYLEYTKFANTLPIECETCTWKKLCGGGGLVQRYSKEQRFNGKSVYCHSLKKIYGRVASHLIESGIDETHILKSLGCNDGN

SEQ ID NO: 177 - protein, 394 aa, Yersinia sp.
MGHLLTKKRIKHFEIILKISERCNINCDYCYVFNKGNSDADNNPARISNKNIGHLANFLQRACLEYEIDTLQIDFHGGEPLLMKKEHFANMCIQLISGNYCGSNIRLALQTNGILIDDEWISLFEKYSVNVSLSIDGPKHINDRHRLDTKGRSTYEGTVRGLRLLQSAYQQGRLPSAPGILCVANAQANGAEIYRHFVDDLGVYGFDFLIPDDSYNDVNIDPIGIGRFLNEALDEWVKDNNPKIFVRHFQTHFASLLGVKNIGILGQSSNITGVYAFTVGSDGSIRVDDTLRSTSDRIFNTIGHISEINLSDVLNSPQAQEYSSIGQCLPNECKGCIWENICTGGRLVNRFSSEERFKHKSVYCYSIRSFLSRASAHLLNMGIKEERIMTSICQ

SEQ ID NO: 178 - protein, 390 aa, Bordetella sp.
  source 1..390: note = genomosp. 11 strain AU8856
MHYIEIILKVAERCNLNCTYCYFFNKENKDFEDHPALISPDTVRQLVQFLRTSSHEISETVFQIDIHGGEPLLLGPRRFSEMVSIIENGLQDAKEVRFTVQTNAVLINDAWLDVFSRHKVFVGVSVDGPKDRHDANRIDRRGRGTFDSMVPKIAALKQATSEARIPGFGSISVVSPESNGRATYTCLTQELGFSKLQFLFPDDTHDSANPANAGRFISFVDDLFECWEEDNSRDVRIKFIDQTLVALLQNKHYIQRGRRVNPAFEGVVFTVSSAGDIGHDDTLRNVAPELFKSGMNVANAKFPEFIAWHNMVSGILVSPDLPAPCASCAWNNICEHVTGSYTPLHRMKNGTADQPSVYCEALKVAYQRGAEYLAKRGHPIHQISKNLNPA

SEQ ID NO: 179 - protein, 391 aa, Bordetella bronchialis
  source 1..391: note = strain AU17976
MHYVEIILKVSERCNLNCTYCYFFNKENRDFEGHPALISPNTVRHLVRFLRTSPHQISETVFQVDIHGGEPLLLGPKRFSEIVSIIENGLSDAKEVRFTVQTNAVLINEAWIDVFAQHKIFVGVSVDGPKGQHDANRIDRRGRGTFDSMVPKIAALKQAALERRIPGFGSISVVSPALDGRATYICLTKELHFAHLQFLFPDDTHDSTNPALAEGFAKFVEDLFASWQSDGNDNIHIKLIDQTLLGFLQDKQYIDGGRRISPAVGRVVFTVSSAGDIGHDDTLRNVAPELFKSGMNVSDANYAEFIVWHNRVSKILFPRDLAPPCASCAWNNICEHVTRSYTPLHRMKDGRVDQPSVYCEALKTAYRNGAEYLAKRGLPIREISKNLNPDY

SEQ ID NO: 180 - protein, 405 aa, Bordetella sp.
  source 1..405: note = genomosp. 9 strain AU14267
MAINHGEHATMPYVEIILKVAERCNLNCKYCYFFNKENRDFEDNPALISPNTVRQLVQFLRTSSHEISETVFQIDIHGGEPLLLGPRRFSEMVSIIENGLHDAKEVRFTVQTNAALINDAWLDVFSRHKVFVGVSVDGPKDQHDANRIDRRGRGTFDTMVPKIAALSQATSQGRIPGFGSISVVSPESDGRATYMCLTKELRFSKLQFLFPDDTHDSANTKNAGRFIKFVGDLFECWENDNNRDVRIKLIDQTLAAFLQDKHYVEAGRRVNSAAQGVVFTVSSAGEIGHDDTLRNVAQELFRSGMNVADAKYPEFLAWHNMISGMLVPRDLPPPCASCAWNNICEHVTGSYTPLHRMKNGTADQPSVYCEALKIAYRRGAEHLAKRGVPIHRISKNLTPVQRATS

SEQ ID NO: 181 - protein, 393 aa, Providencia rettgeri
  source 1..393: note = strain 2020EL-00052
MLKIKHFEVILKISERCNLNCTYCYIFNMGSELALNSAPVISNTTIVELKNFLERVADEVEHNVIQVDLHGGEPLMLKKKRFIYLCETLRSGDYKGAEFRIGLQTNATLIDDEWLEIFEKYNISVSISIDGPKHINDRYRLDHKGRSSYEATMNGYQALYSAAENRKIIPTPPILSVINPDASGKELFEYFYHDMKCRKFDFLLPDNNYVNTVDTEGIKRFLVDICDAWFAQNDPECDIRILSAYLRILTGAEDYIVLGVTPQNELHQTIAITVTSTGYIYVDDTLRSTLSDIFVPICHIRDASYQKIITSFPMRELSKIESFLPDDCHGCIWKAVCAGGRPINRYSQDNAFKNKTIYCDAMQSFLSRGAAYLINLGINSNEIAKNIGIDKNA

SEQ ID NO: 182 - protein, 389 aa, Pandoraea oxalativorans
MKQYVEVILKVSERCNIDCKYCYFFNKENKDYASNPPYMTQQTAEDFVTFLRSSPNLRETTFQIDLHGGEPLMMKRERFEALVTTLKNGLSDAESVQFTVQTNAMLVDEAWLDLFSRLGVYIGVSIDGPKIYHDENRVDKQGMGTYDRTVEKIALIKAAADTGLISGFGAICVMNPKFDARLVYDTLTRTLGIYNLQFLLPDESHDSVRTADVMALKWFTQALFDCWADDPRGTVRIRSIDRMLDAILADEPRKDVIWRDARSSVVFTLSSGGDIGHDDTLRNVIPDVFYARMNVASSTFSEFLAWHATVSAMLARRTTAVACRTCLWREICEIATRSDTPLHRCKNGVADQHTVYCECLKANYEKGAEYLALSGVAIEEISRNFVEVD

SEQ ID NO: 183 - protein, 406 aa, Erythrobacter sp.
MFDVEARLARPGRRHVSVVLKVAERCNLACTYCYFFFGGDDSYLKHPALISSDRVSDVARFLGEAAIKHRLERIEIALHGGEPLLLKPDRMGALVETIRAAVPDSCEVDILLQTNGVLVDETWIALFEQHSIGIGVSLDGPRAVNDIARLDKKGRSSFDATIAGWGLLKKAAADGRISEPGILSVIAPTTDAETLSFFIDELGAHSLNFLLPDMFFDNPETQPEDVARIGETMIAIFEEWRRRADPGLHIRFVNDALLPMIVAIPAESTHHCREDLSHAMTIASDGTIYVEDTIRSAFADRFDETLNVASATLADVFAHPHWQSIARAAEQPAGPCTSCRYGEICQGGPLISRYSSDRGFDNPSLYCSALFAFHRHVEREVSATGRLLPSPRFAADPLFPARKEVA

SEQ ID NO: 184 - protein, 395 aa, Sodalis sp.
  source 1..395: note = strain dw_96
MKDKQPKHLEIILKVSERCNLNCSYCYVFNMGSDLALNSAPVISRATINSLKNFLERSVREYSIDVIQIDLHGGEPLMLKKERMAVLCALIREGDYNGASVQIGIQTNATLIDEEWIEIFSRYHVSVSISIDGPKHVNDIHRLDHQGRSSYEKTLRGYKLLSTRSTDGKKEINAPVLSVLTPKANGSELFSHLYDVMGCRNFDFLLPDCNYDNPIDTAAIGRSLIEICDKWYAQNDPDCVVRIVNAHMAHLAGNKKNVVLGVTNVNKNALALAFTVTSQGEIYVDDTLRSTHSDIFTSIGNITHTSLEEIFASRQLIALNIIQDTIPRECSECVWRNICAGGRPINRYSSIDGFTGKTIYCDAMKMFLGRCASILNEMGVSIEELVINLGIENDK

SEQ ID NO: 185 - protein, 388 aa, Kosakonia cowanii
  source 1..388: note = strain pasteuri
MRTKIKHLEIILKVSERCNINCTYCYVFNLGNELAINSKPVISASTIGDLRRFLENAAIEHGIETLVIDFHGGEPLMMGKKKFAAACEVFRSGNYGNGELHLACQTNGILIDDEWIDLFSKYGVGVGVSIDGPKHINDKHRLDHKGRSTYEGTVKGFRLLQAAYAAGKLELEPGILSVANPFVKGSEIYRHFVDTLNCKRFDLLIPDESHFSCKNPNEIADFYCSAIDEFFFDGNPDINIRYINTHVQAIVSNNHAQTLGVSKSTSDAIAITVMSDGDIYIDDTLRSTNDELFSPIGNVREISFSGVKESWQFKKSAHIANNPPADCKDCLWKKVCGGGSMIQRYSKEEGFERKSVYCPSIKKIFSRMTSHLISAGIPEEKISKNLEG

SEQ ID NO: 186 - protein, 392 aa, Bartonella sp.
MSNVASKLNVLEIILKLTERCNLNCTYCYVFNKGDYDETSSQALISDNSVNDVIDFVLNAIESYELKLVRIIFHGGEPLLYPKKKFDNLCNSLKALESVDTSITLSLQTNGVLIDETWVEIFSRHDVTVGISLDGNKEMNDQYRLDKKGRSSYERSIKGLRLLQESYNQNKFSHSPSILMVANCENDIDTLYDHVFNNLGVSSFDILLPDDNYLDESRPSDDLMGKYFTRLLDLYLNDERDVFIRLFDAPIYILNSNSMDFLGFSARVHKMMVSLTINTDGLLYVNDVLKPTGAYLASAIGNIKDFKLEDFMASQQYKMYISATEYVPSECQDCIWRNPCSGGALQNRYSKENGFSNKTIYCGTNRSILSRVSEYLIIKGVDESKIMSNIGL

SEQ ID NO: 187 - protein, 393 aa, Photorhabdus heterorhabditis
MPKIKHFEVILKISERCNLNCSYCYVFNMGSELALNSAPVISHNTIIELKYFLERVAEETTPDVIQIDLHGGEPLMLKKERFVYLCETLRSGDYKNAEFRLGLQTNATLIDDEWIEIFEKFEVAVSISIDGPKHINDKYRIDHKGRSSYEATLNGYQALYTAAKKRNILPLPPVLSVIDPEANGKELFEHLYHDMQCRKFDFLLPDYNYENPTNTEGIKRFLTAICDAWFEQNDPACDVRILSAHLTRLMGTTGHVILGVTPQIESYKAVAITVTSTGDIYIDDSLRSTLSKIFTPIGNIKNTSYAQIVNSPPMRELSKIEASLPDDCQGCIWKTICAGGRPINRYSRDNAFNNKTIYCDAMQAFLGRGAAYLVELGLSENEIEKNIGIAEHE

SEQ ID NO: 188 - protein, 392 aa, Erwinia sp.
MRLIKGEKIKHLEIIFQVSKRCNISCSYCQVFIMGNTLAADSHPTKSLNNVIALRGFFERSTAENEIEVIQVDFHGGKPLMMKKDRFDQMCHILLQGDYGNSRIELALQTHGILVDEEWITLFEKYKVQASIPVDGLRHSNNRHRPDRTGESTYKGTINGLRLLQNAWQQGRLPAEPGILSVANAKANGADIYHHFVDVLKCQRFDFLIPDDHHDDITDSEGIGRFLNEALDAWFADGRPELFVRIFNTYLGTLLDKQFSRVLGMSANVESAYAFTVTADGLLRIDDTLRSTSDEIFNPVGHVRDLSLAGVLKNTAVEEYLSLSNTLPEGCKDCVWNNVCHGGRLVNRFSQANRFNNKTVFCSSMRIFLSRGASHLMATGIDERTIMANIQG

SEQ ID NO: 189 - protein, 392 aa, Xenorhabdus griffiniae
  source 1..392: note = strain VH1
MNKINHLEVILKISERCNLNCSYCYVFNMGSDIALNSAPVISHNTIIGLKGFLERVAEDVNPDVIQIDLHGGEPLMLKKERLIYLCETLNSGDYKGAELRFALQTNATLINNEWIAIFEKFNISVNISIDGPKHINDKYRIDHKGRSSYEATLNGYKALCTAAKERNILNYPSILSVIDPEASGKELFDHFYHDMQCKRFDFLLPDSNYENTTNTEGVKRFLIDVCDAWFEQSDPNCDVRILSSYFTRLAGSSKYIVLGVTPPTEGFEALAITVTSTGDIYIDDTLRSTVSEIFTPIGNIADATYAQIVNSQPMREFHKIESSLPVDCQGCIWQKICAGGKPVNRYSRDNAFNNKTIYCDTMAALLGRGAAYLVELGLSENELAKNIGIAEL

SEQ ID NO: 190 - protein, 392 aa, Xenorhabdus griffiniae; identical to SEQ ID NO: 189
  source 1..392: note = strain VH1

SEQ ID NO: 191 - protein, 395 aa, Burkholderia sp.
MIEHDKINRLEVILKVTERCNIDCTYCYYFNGNNRDYMGQPPYLTVDTAKSLAVYLRNAACSHSIDEIRIDLHGGEPLLMKKAKMSAVLEILRSGVADFTDLTICIQTNATLLDEEWISIFEKYSVSVGVSLDGSPDENDLYRVDKKGKGTHSVVVKAIELLKAANKKSEGIFAGIICVVNPDFDGKKIYRHFVDDLGVERIHFLKANQTRDGADIKLVAGTRKFLLGALNEWINDGNFNIYVRQFTEPLKQLCTSSAPSPCSDRYVAMTVRANGDIAIDDDFRNTLPSLFNLGLNISDSALADFLDRPGVADFHRACGEVSPSCLQCGAREICKNGTGLAESVLHRYSFINKFRNASLFCESHQAIIIRLGQFAISRGVPWSTIERNMAGIRNN

SEQ ID NO: 192 - protein, 441 aa, Trinickia sp.
MLIRLVIQKTPHFLVRNFRGCSTHQCFPKCIEPESSSCVLINNWRRNDGARKINRLEVIVKVTERCNIDCTYCYYFNGENGDYANQPPYLTVDTARSLAIYLHNASRSHSIDEIRIDLHGGEPLLMKKTRMSVMLEIFRSSIPDSTDLTICIQTNAILLDEEWISIFAKYNVSVGVSLDGPPRENDLYRVDKKGRGTHSAIAKAIEMLKKANKKCAGVFAGVICVVNPDFDGRKVYRHFVDDLGIERIHFLKPNQTRDGADIKLVEGTSKFLLDALNEWINDSNPNIYVRQFTDPIRRLCASGPSSPFSDRYVAVTVRANGEIAIDDDFRNTLPSLFNLELNVADSALADFLNHPGVFDFHQACAEVPPSCLQCGANGICQSGIGLNESVLHRYSFINKFRNASLFCQSHQAIIIRLGQFAISHGVPWSTIEKNMIRIHDN

SEQ ID NO: 193 - protein, 437 aa, Burkholderia sp.
MFISFSTKSHVTSLLARKLAPRNDASLGHQFWTESTLLKISKEMKNIDKINRLEVILKVTERCNIDCTYCYYFNGSNHDYTSQPPYLNIDTAKSLAGYLRDATRAHSIDEIQIDLHGGEPLLMKKSRMSDMLEIFRNSISDQTDLRISIQTNATLLDEEWLSIFAKYNVSVGVSLDGPPRENDLHRVDKKGNGTHSAVSKAIAMLIEKNKTCEGVFAGVICVINPDFDGSKTYRHFVDDLGIERIHFLKPNQTRDAADIKLTEGTSKFLLDTLSEWINDSDRNIYVRQFTDPLKRICASDASESPPHRFVAMTVRANGEIAVDDDFRNTLPSLFNLGLNVSNSTLADFINHPKVADFHRACDEVPPFCSQCGAKGICQSGAGLGESVLHRYSFINKFRNASLFCTSHQAVIIELGKFALSHGMPWATIEENMTGNRI

SEQ ID NO: 194 - DNA, 1395 bp, synthetic construct
  source 1..1395: mol_type = other DNA
agcaaattacagcgtgaaattgcagcaaacaaagctcaactgagccatgaagacaagaagaaaacgcagcacaaagagcttgttgacagcctgctggatactgtctctggtggttggataaacgcttttggaaactgggagagagcctttcattaatacactgccgggggaggttttcttcccccttctctttcttcattctggcgaataatgataatgacgacatcaaagagtgagaagatcaaacatcttgagatcattctcaaaattagtgaacgatgcaatatcaattgctcctattgctatgtattcaatatgggtaactcactggctaccgatagtcctccggtcatatcgcttgataacgtgctggcgttgaggggattctttgagcgctccgcagcagaaaacgagattgaagttatccaagtcgattttcacggtggtgaaccactgatgatgaaaaaagaccgtttcgatcaaatgtgtgacattcttcggcagggtgactatagcggttcccggcttgaattagcattacagactaacggtattctgattgatgatgaatggatttcactgtttgaaaaacataaagtccatgccagcatatcaatcgatggaccaaaacatatcaatgaccgctatcggttggaccgaaaaggaaaaagcacttacgaaggaacaattcacggcttgcgcatgctccagaatgcgtggaagcaagggcgactcccgggagagcccggcattctctctgtggcaaaccccacagcgaatggtgcagagatttatcaccactttgcaaacgtcctcaaatgtcagcacttcgatttcctcatacccgacgctcaccatgatgatgatattgatggcataggtattggcagattcatgaatgaagcgcttgacgcatggtttgctgacggtcggtcagagatttttgttcgaatctttaacacataccttggcacgatgctaagtaaccagttttaccgggttattggcatgagcgcgaatgtagaatctgcttatgctttcacggtaactgccgacggcctgctccgtattgatgatactttgcgttccacctctgatgaaatattcaatgccattgggcatctcagtgaattgtcactctccggcgtactcaattcacctaatgtcaaagaatatctttcactaaatagtgaactgccaagtgattgtgcagattgtgtgtggaacaaaatctgtcacggtggccgcttggtcaatcgcttttcacgggcaaaccgtttcaataataaaaccgtgttctgttcatcaatgaggcttttccttagtcgcgcggcttcacacctgattacggctggtattgatgaagaaacaataatgaaaaatattcagaaatag

SEQ ID NO: 195 - DNA, 4263 bp, synthetic construct
  source 1..4263: mol_type = other DNA
gaaaaaatcaatttctggttatcaaagttttcatgtgccgccctcgctatttgttgtacatcttgccttgctgactcgggaaattcggtaacacttaagctgaattatgacaaatatttcacgcctcatgcaactttcatcattaatggccacccggtaaatatgatgattgatacaggttcttcgaagggcttttatcttcaagagcctcaactaaaaaaaatacaaggcctcaaaaaagaaagcacttattacagtactaatatcaccgggaaaagacaggagaacacagagtatctcgccgcttctctcgacatgaatggccttaaattaaaaaacgtaaccgtgatcccatttaaacaatggggagcgctgatttctaacacaggtaaattgccggatggccctgttgtcggtctcgatgcgtttaaagataaacaaattatgctggattttgtgtctcattcattcacgatgagcgacagttttatccataacatgccggttccgaaaggctttaacgcattcactttccatatgtctcctgatggcatggtttttgatgttgatcagtctggacacacataccatttgattctggacaccggtgccactgcgtctgtgatttggcgtgaaagacttaaacagtatgaacccaaaagctgcctgctggtcgatccgaagatggataacgaaggatgccaggccactctgctcacaattaaatcaaaaactggaaatccccagcattttggtgcggttgttgttgtcggaaattttaaacacatgggcaacgttgatggccttttagggaataacttcctcagaaatcgaaaggtacttatagactttaaaaacaagaaggtttttatttccgatgagcaccgaaacagaaaagaatgacaactcaatctttcgtgccgaggctttgcaacacaaacgagaaggttggctcggcgcttctcgtttgcatataccgtcagcgctctctatttgttgcctgacaatccttgttattttctttttcatcatattgataattgcatttggttcgtacagtgaacggataaatgtcatcggaaccgtggtttataagccgcctgcggtatcactgattgcacaaagcagtggaatcattacgcattcactggcattagagcaaacaagagttaagcgcaacgagagcattttttctatcagtggtgacactcagacaaatctgggtgccaccaatgttgaaacggtagaacttttaaataagcaacgtaacgcgctgtctaaaaagcttgatattgcggccaatgaatcaaaagcaaacaagatttatctcagcgaaaaaattaaaaataaacaacaggaaatagaaagtctgcaaaacctgatagaaacttcagaaaaacagcaagcgtggttcgagaaaaaatcaaacctgtatgcgaattttaagaagaaaggcattgcgcttgatgctgaatggataaacagaaagaaagattattacgcatccacattaagcatttcttctgcaaaggtcaaagtgatagccctgctgggagagttgcaggatctgaaaaatgacgtttcggttatcgacaggaaactcgacaaagaaacagcatctctcactgtcgaaatagccgatatagcacaaaaaatactgattacagaaaaacaaaaagagtatttaatcgtcgcgccgtttgatggaatgataaccagtgttacagcccatatcggtgaaagagtgactgccggccagcaaatagccgtgctgataccacaaggtgcgacagaaaaggttgagttgttttcaccgtctgattctctcggtgaagtgaccagcggacagcaagtcagaatgagagtctcggcatacccttaccagtggtatggaaagattgcaggcatcatagaaacgatatcggcagcaccggtcaatgtcacctcacagatgcagatgaaaggtgaagaggtaaaaaaggggctttttcggattgtcgtacaaccaaaattgaccggacaacaaacaaacatttcccttctacccggcatggaagtggaaacagagatctatgtgaaaacccgaaaattgtacgaatggttatttatccccattaaaggggcatatgaacgggcgacagacagtacggaataaatatgcagtataagatgagtgattttttcgagtttttcgtcaaaaaactcccggtgataatacaaacagagaccacagaatgcgggttggcatgtctggccatgattgctgcctggtatggccgtgagactgatatctacagcatgagaaaggtttttgacgtgtcaaacaatggcatgacattaaggcagatcatcacggcggccgggcgaataaacatgaataccagagctgtgcggctggaactcaacgaactcagcagtgtcaggcttccgtgcatcttgcactggtcctttaatcattttgtcgtgttaaaaaaattcacaaaaaaaggggcagtcatccatgatcccgccttgggaaaaagaactgtcactctgaaagaactctcaaataagtttacgggcatcgctctggaagtctggccccagacggagtttaaaaaggaaaaggtcagtgaaagcataaccatcacggatatgtttcgcggtgttgccggccttaagaatacgctgtttaaaatcattctgttgtcgctctttattgaagtactggcactttccatccctctcagctctcaattcattattgatgttgttctacggtccagtgacctcagtatgctgaatttcattgtcattggaatcgttcttctgctctccctgcgcgctgctttcagtattgtgcgcgcctgggctcttatggcaatgcgttactcacttggcatacagtggagttccggtttttttaaccggttactcagattgccggtcactttttttgaaaaacgtcacgtaggtgatatcgcctccagattgacatcgttgagcgaagttcaagaagcctttacagcagaaatgctgacttcgttacttgatgtacttattctcataacgctggctgtgctcatgttctgttacagccctcttctgacccttctcccgctactcatgactaccgtttatcttggggtcaaatttgctttttatgacagatacatgggagcaaaagtagaagcaattacgcatgaagcgcagcaatcatcctactttctcgaaacaatacgaggcgtagcgtgcgtgaaagtatttggcctgacagaattccgacgtatcacatggcttaaccgggtgattgatactgccaatgcccgggcccatttatttaagatagacctcatcagccaaacgctttcaggtttcctgacggggctatcatcggcggccattttgtttatggggagtcatctcacagaacgcggcctgatcactgccggcattctgtttgcttttctgctctataccgatatgtttctgacacgttcagtgaaggtaataaattcactgtttgcttttcgccttatttcgatacacacgcaccgattgaccgatattgcaacagcccagacagaaaatgcatggaacccggaagatcccgtcacactcgataatgtaaaaggccggataacactgaacaatctcacatatcggtacggagaaactgaaccctgtattttcgactgtatcgacatggaaattaatgctggtgagagtgtggcgatcgtaggtccgtcaggttgcggtaaatcgacacttctccgggtcatggccggcctggttctccctcagtcaggcgatgtgtcaattgatgatgtcagtgtgaaaaaaatgggtattgacgaatatcgcagacacacggcgtttgtcatgcaagatgataagctttttgctgcctcattgatggataacatatccgcttttgatccacagccaaatattgattggatacatgaatgcgctaaggcggcggcaatacacgatgaaattatgactatgccgatgcagtacgaaaccatggtgggtgacatggggagcattctttcaggcggacaaaaacagcgtgtatcccttgcacgggcactttacaagtgtccgcgtatcctctttcttgatgaggccaccagccatctcgacgtttttaatgaacgcaagataaatgaggctgtaaagcagatgccgattacgcgtgtatttgtggctcatcggccagaaatgatcgctgtcgcagaccgagtttataacctgagggataagacctttacaacgtaa

SEQ ID NO: 196 - DNA, 5457 bp, synthetic construct
  source 1..5457: mol_type = other DNA
atgacgacatcaaagagtgagaagatcaaacatcttgagatcattctcaaaattagtgaacgatgcaatatcaattgctcctattgctatgtattcaatatgggtaactcactggctaccgatagtcctccggtcatatcgcttgataacgtgctggcgttgaggggattctttgagcgctccgcagcagaaaacgagattgaagttatccaagtcgattttcacggtggtgaaccactgatgatgaaaaaagaccgtttcgatcaaatgtgtgacattcttcggcagggtgactatagcggttcccggcttgaattagcattacagactaacggtattctgattgatgatgaatggatttcactgtttgaaaaacataaagtccatgccagcatatcaatcgatggaccaaaacatatcaatgaccgctatcggttggaccgaaaaggaaaaagcacttacgaaggaacaattcacggcttgcgcatgctccagaatgcgtggaagcaagggcgactcccgggagagcccggcattctctctgtggcaaaccccacagcgaatggtgcagagatttatcaccactttgcaaacgtcctcaaatgtcagcacttcgatttcctcatacccgacgctcaccatgatgatgatattgatggcataggtattggcagattcatgaatgaagcgcttgacgcatggtttgctgacggtcggtcagagatttttgttcgaatctttaacacataccttggcacgatgctaagtaaccagttttaccgggttattggcatgagcgcgaatgtagaatctgcttatgctttcacggtaactgccgacggcctgctccgtattgatgatactttgcgttccacctctgatgaaatattcaatgccattgggcatctcagtgaattgtcactctccggcgtactcaattcacctaatgtcaaagaatatctttcactaaatagtgaactgccaagtgattgtgcagattgtgtgtggaacaaaatctgtcacggtggccgcttggtcaatcgcttttcacgggcaaaccgtttcaataataaaaccgtgttctgttcatcaatgaggcttttccttagtcgcgcggcttcacacctgattacggctggtattgatgaagaaacaataatgaaaaatattcagaaatagtggagccggacaatggaaaaaatcaatttctggttatcaaagttttcatgtgccgccctcgctatttgttgtacatcttgccttgctgactcgggaaattcggtaacacttaagctgaattatgacaaatatttcacgcctcatgcaactttcatcattaatggccacccggtaaatatgatgattgatacaggttcttcgaagggcttttatcttcaagagcctcaactaaaaaaaatacaaggcctcaaaaaagaaagcacttattacagtactaatatcaccgggaaaagacaggagaacacagagtatctcgccgcttctctcgacatgaatggccttaaattaaaaaacgtaaccgtgatcccatttaaacaatggggagcgctgatttctaacacaggtaaattgccggatggccctgttgtcggtctcgatgcgtttaaagataaacaaattatgctggattttgtgtctcattcattcacgatgagcgacagttttatccataacatgccggttccgaaaggctttaacgcattcactttccatatgtctcctgatggcatggtttttgatgttgatcagtctggacacacataccatttgattctggacaccggtgccactgcgtctgtgatttggcgtgaaagacttaaacagtatgaacccaaaagctgcctgctggtcgatccgaagatggataacgaaggatgccaggccactctgctcacaattaaatcaaaaactggaaatccccagcattttggtgcggttgttgttgtcggaaattttaaacacatgggcaacgttgatggccttttagggaataacttcctcagaaatcgaaaggtacttatagactttaaaaacaagaaggtttttatttccgatgagcaccgaaacagaaaagaatgacaactcaatctttcgtgccgaggctttgcaacacaaacgagaaggttggctcggcgcttctcgtttgcatataccgtcagcgctctctatttgttgcctgacaatccttgttattttctttttcatcatattgataattgcatttggttcgtacagtgaacggataaatgtcatcggaaccgtggtttataagccgcctgcggtatcactgattgcacaaagcagtggaatcattacgcattcactggcattagagcaaacaagagttaagcgcaacgagagcattttttctatcagtggtgacactcagacaaatctgggtgccaccaatgttgaaacggtagaacttttaaataagcaacgtaacgcgctgtctaaaaagcttgatattgcggccaatgaatcaaaagcaaacaagatttatctcagcgaaaaaattaaaaataaacaacaggaaatagaaagtctgcaaaacctgatagaaacttcagaaaaacagcaagcgtggttcgagaaaaaatcaaacctgtatgcgaattttaagaagaaaggcattgcgcttgatgctgaatggataaacagaaagaaagattattacgcatccacattaagcatttcttctgcaaaggtcaaagtgatagccctgctgggagagttgcaggatctgaaaaatgacgtttcggttatcgacaggaaactcgacaaagaaacagcatctctcactgtcgaaatagccgatatagcacaaaaaatactgattacagaaaaacaaaaagagtatttaatcgtcgcgccgtttgatggaatgataaccagtgttacagcccatatcggtgaaagagtgactgccggccagcaaatagccgtgctgataccacaaggtgcgacagaaaaggttgagttgttttcaccgtctgattctctcggtgaagtgaccagcggacagcaagtcagaatgagagtctcggcatacccttaccagtggtatggaaagattgcaggcatcatagaaacgatatcggcagcaccggtcaatgtcacctcacagatgcagatgaaaggtgaagaggtaaaaaaggggctttttcggattgtcgtacaaccaaaattgaccggacaacaaacaaacatttcccttctacccggcatggaagtggaaacagagatctatgtgaaaacccgaaaattgtacgaatggttatttatccccattaaaggggcatatgaacgggcgacagacagtacggaataaatatgcagtataagatgagtgattttttcgagtttttcgtcaaaaaactcccggtgataatacaaacagagaccacagaatgcgggttggcatgtctggccatgattgctgcctggtatggccgtgagactgatatctacagcatgagaaaggtttttgacgtgtcaaacaatggcatgacattaaggcagatcatcacggcggccgggcgaataaacatgaataccagagctgtgcggctggaactcaacgaactcagcagtgtcaggcttccgtgcatcttgcactggtcctttaatcattttgtcgtgttaaaaaaattcacaaaaaaaggggcagtcatccatgatcccgccttgggaaaaagaactgtcactctgaaagaactctcaaataagtttacgggcatcgctctggaagtctggccccagacggagtttaaaaaggaaaaggtcagtgaaagcataaccatcacggatatgtttcgcggtgttgccggccttaagaatacgctgtttaaaatcattctgttgtcgctctttattgaagtactggcactttccatccctctcagctctcaattcattattgatgttgttctacggtccagtgacctcagtatgctgaatttcattgtcattggaatcgttcttctgctctccctgcgcgctgctttcagtattgtgcgcgcctgggctcttatggcaatgcgttactcacttggcatacagtggagttccggtttttttaaccggttactcagattgccggtcactttttttgaaaaacgtcacgtaggtgatatcgcctccagattgacatcgttgagcgaagttcaagaagcctttacagcagaaatgctgacttcgttacttgatgtacttattctcataacgctggctgtgctcatgttctgttacagccctcttctgacccttctcccgctactcatgactaccgtttatcttggggtcaaatttgctttttatgacagatacatgggagcaaaagtagaagcaattacgcatgaagcgcagcaatcatcctactttctcgaaacaatacgaggcgtagcgtgcgtgaaagtatttggcctgacagaattccgacgtatcacatggcttaaccgggtgattgatactgccaatgcccgggcccatttatttaagatagacctcatcagccaaacgctttcaggtttcctgacggggctatcatcggcggccattttgtttatggggagtcatctcacagaacgcggcctgatcactgccggcattctgtttgcttttctgctctataccgatatgtttctgacacgttcagtgaaggtaataaattcactgtttgcttttcgccttatttcgatacacacgcaccgattgaccgatattgcaacagcccagacagaaaatgcatggaacccggaagatcccgtcacactcgataatgtaaaaggccggataacactgaacaatctcacatatcggtacggagaaactgaaccctgtattttcgactgtatcgacatggaaattaatgctggtgagagtgtggcgatcgtaggtccgtcaggttgcggtaaatcgacacttctccgggtcatggccggcctggttctccctcagtcaggcgatgtgtcaattgatgatgtcagtgtgaaaaaaatgggtattgacgaatatcgcagacacacggcgtttgtcatgcaagatgataagctttttgctgcctcattgatggataacatatccgcttttgatccacagccaaatattgattggatacatgaatgcgctaaggcggcggcaatacacgatgaaattatgactatgccgatgcagtacgaaaccatggtgggtgacatggggagcattctttcaggcggacaaaaacagcgtgtatcccttgcacgggcactttacaagtgtccgcgtatcctctttcttgatgaggccaccagccatctcgacgtttttaatgaacgcaagataaatgaggctgtaaagcagatgccgattacgcgtgtatttgtggctcatcggccagaaatgatcgctgtcgcagaccgagtttataacctgagggataagacctttacaacgtaa

SEQ ID NO: 197 - DNA, 1397 bp, synthetic construct
  source 1..1397: mol_type = other DNA
tctaaattagccaaagaaattaacatgaataaagcagccgtcaccgttgcagctgataaaaaagacgcacgaaaagcactggctcaatctatgctggatagcgtttctggcggttgggtcaacgcctttgcgcgttggtccaaaagcttctaattgaccttggtgcagggtgggagaccgccctgcactttctcctttgttgaacagtggtacgggcaatgacgaataagaaaaaaataaagcatcttgaaataattttaaaggttagtgaacgatgcaacattaactgcacgtattgctatgtattcaacctgggcaatgatttggcaataaattcaaaaccaattatttctcataaaatcattgaagatttgagaggttttttcgagcgggcctgccaggagtatgaaatagaaacggttcaggttgactttcatggcggcgaaccgttaatgatggggaaagagcgtttcgacaatgcctgcaaagagcttatctcaggtgactataatggcgccaggctcaaccttgcctgtcagacaaacgctatccttattgataatgagtggattgatattttctcgaaatataatatcagcgtggggatttctattgatggccccaagcacattaacgacaggcaccgcctggatagaaagggacgcagcacctacgaaggtacggtaaaagggctggagatgctgcaggttgcctggaaagcgggccgattgatcgatgaacccggcatcctgtgcgtcgccaatccttcggtaaaaggcgctgaaatctatcgtcattttgtcgatgtactgaaatgcaaaaaatttgatttcctcattccggatgaaagccatgacacctgcacggatccggacggactggcggatttttattgctcggcgctggacgagttctttttggacgcggataaagaggtgtatgtgcgctacttccatacgcacatccaatccatgttgagttcagaattcaatccggtaatgggagtaagcaaagccgggaacgatactctcgctttcacggtgagttccgatggtgaactgtatgtggatgatacgctgagagcaaccaatgaccctatatttacgcctattggtaatattcaacatttaatactgtcagacactctcgcctcatggcagatgacaaagtatatggctgtgaatagtcagcttcctaccgtttgcggtgactgtgtctggcaaaaagtttgtggcggagggcgtcatattcagcgttattctacagccgatgattttaaccgtgaaaccgttttttgtccgtcggtaagaaagatcatgagccgtgcggcttcgcatttgattgaatcgggcgtggcagaggatataatcatgaaaaacttagaggttaactcatga

SEQ ID NO: 198 - DNA, 4210 bp, synthetic construct
  source 1..4210: mol_type = other DNA
atcaagcggctatccttattggcgttcttgttttccggcatcagcatggcgagtcttcccgctgattttgggcggttgcggtatgatgaacgtggactgccgttaattgatgtccggatcgataatcgtcttcataccttaatgttggataccggcagcggggaggggatgcatctttataaacacgatcttgacaacttagtggctaatcctggcctgcaggcgaccgaacaagcccctcgccggttgatggatgtttcagggggtgaaaataaagtttcctcatggaagattaatcgattacttatttccaatattcctttcgataatgttgaagcggtaagttttaaaccatggggattaagcatcggcggtgatgtccctatgaatgaagtgatggggttggggctttttcgagaacgcagagtgctgatggattttaaaaacgatcggttaaaaatattggccgacttgccatctgacataaagaaatggtcatcgtaccccatcgaaccaaccgcatcgggattgcgcgttaccgcctccgcaggcggtatgcctttgcatttgattgtcgatactgcggccagccattctctgctgttttcagaccgtttgccgccgggcctccttttctctgggtgccgcgacattgagccggaagcgtcgaatctggattgccgggtgacaaaaatcgcttttacggatcgcgaaggtaaggctcgtgatgaccaggccgtcgttgcctctggtgccacgcccccggaactggattttgacggtcttttggggatgaagtttatgcggggacatcaggtgatcatcgatatgcctgaacgcctgctctatatcagccgttagcgtgatggacaaagaaaactcgtttttccgccaggaggcgttgcagcataaaaaaaatgcctggctgggcgattttaccgtttcggcgccatcagtgttgcccatcgcgttatggagcgccgttggcgttttgctgttggctacccttctgttattcaccacttatgccaaaagagtccccgtgaccgggcgagtcatctatacgccttccgctgctgaggcggtgtttaaccatgacgggattatcggccgcatcgaagtgcaccaaggggaaagggttaagaaaggggatgtcatcgcgacgttttcacgcgatgtcgcctatgtcgggggaggcatgaatcaggcattgcaagatgcggcgcagcgccagcttaccgagttgcaaaagcgcgcgggagagcggcgtaaagagggagaagaagagcgcttgcgtttacgtgagaaagtcagcgccaaagaacgggaaatggtggcgattcaagctgcggccgaagccgaatcggagcacatcgtcggtttgaagaagcggatggcgctttatcaacagctgttactgaaaggtattacgaccgtacaagagaaaattgagcgggagaacgaatatcataattctattgcacagctgaacacgcatcgaatcaatatcgcgcgggtgaaaggagagctgctgcaattcgaggatgagctggctcgctctgaatcgcaagaaaaacagtctattactgacattcaacagcagaaggtcacgctgcaacagcaggtgattaatgcctctgcggtcgtggagtctcgggttgtggctccgcttgatggcgtcgtcgcttcaatgagcattttggaaggacagagagtgaccgccggcgcagttgccgcagtggtggtgccggaaaatgcacgtccgttcgttgaaatgtggatcccgccctctgcgctgcaggaggtgaaagcgggtcagcatgttttcatgcgcgtcgcatccttgccgtgggagtggtttgggaaagtgtccggcacggttgccgccgtcagcgagagtcctgaggcgctgacgggaaataatcgacgttttcgcgtgctgatcgcgcccgatgtcggaacgcgagcgctgcctgcgggagtggacgttgaggccgacatattgacgacgcatcggcgcatctgggaatggctcttcttaccattaaaacaaagtattaaccgcatgacggctgagagttgacacatgcttttttcctggcaaaaaacaccgctgattctacagtcggaaacgaatgagtgtgggttggcctgtttggccatgatggccggttatttcggcaaacgcatcgatcttgcttcggcgcgtacccttcacgggatcggcagccacgggatgacgctgcgagatctcattacggcgtttgaacgtgtggggatgacggctcgtgcttcgcgcgtagagctggatgaactgcgttctctcagccgccctgcgattcttcactggtcattcaatcatttcgtggtgctggtgaaagtgacgcgtcggggcgcggtgatcctggatcctgccattggtcgccgcagcatttcattgcgtgaactgtcggataaatttaccggcgttttggtggaagcatggcctgcggagaccttcgataagaaagcgctggaaatgaatgtcaccgtatccgatctttttcgtggcgtacggggcttaagacgcatttttaccggcgttctgatgctttcggtcttggtggaactgctctccattgcggtacccgccgcgtcacaatttactatcgatacgttagtgcgttcatcagaccgcgaaggaatattttttgtcggtatcgtggtcatttccgcattgctgattaagtccgccttttcggtggtgcgtgcctggattttgatgaatctgcgctatacgctcggcgtgaaatgggctgaaatgttctttaaccggcttatcaaacttacgctgtcattttttgagaagcggcacaccggcgatatcgcgtcgcgcttccagtcgttgaccgccattcaggaagcgtttacggccgatatggttgcctctctcttggatgcgattgtgattgtcatttcaatggcgatcatttttacctattcacctgtgctggccatcggccccctgatcgccgcctgcgcctatgccgccttgaaggcgggcctgttctcgacctaccgcaatcgtaaaattgaacatatcgccttcgaagcggtgcaatcctcccacttccttgaaaccgtcagagcgatcggcgcgatcaaaatgttgaacctgacgccggttcgtcggcgcgaatgggtcaaccatgtggtcaacagcacgcatgcggggaaccagctgtttaaactcgatctgctgaccaacacggcggccgtgctgctggtgggattttccgggattttcgtgcttagcgtcggggccatcggatttgataaaggcattacgactggcgccttgctggccgtgatgctgtatgccgatatggtgattacccgcacggtgaagttagtcaatgcggtttctgatttttgcctggtatccatgcacagtcagcgtttgactgacgtggctgtttcacccgtggaacgggatgagggagaacaagtgtcgccacagctgaatgggcatatcgtgatccgcaacttagcgttccgccattcccagaccgaacgcaacatcttcgaggggatcaatcttgagatcatgccaggggaaaacgtcgcgatcgtcgggccgtccgggtgtggtaagtcaacattcctccatgtgctggcggggttgtacgaatctaccgaaggggatgttttcattaacaacgtggggatgtctggcatgggcaaacgagacattcgtgaacatgtcgcttttgtcatgcaggacgacaaactcttggctggaaccatacagcagaatattaccggttttaccgcgtcccccgatgtggaacgcatggctgaatgcgccaatcatgccgcgattgacgaagaaatcagcgcatttccacagggatatgagtcgatgatcggtgatattggtagcacgctttctggcgggcaacgccagcgtatttctatcgccagagcgctataccggcaacctcgtgtgctgctgcttgatgaggcaaccagcgatcttgatatcgataacgagaaaaagatcactcgcgccatcgggcaattgccgataacccgcatttttgttgctcatcgcccagaaatgatcaagtcagcggatcgggtctttaatcttcatctgaatgcctgggtgaagcaggaaaatcgggggggcgctacaatgttgatcgccgacaaggttcacataagctga

SEQ ID NO: 199 - DNA, 1389 bp, synthetic construct
  source 1..1389: mol_type = other DNA
agcaaattacagcatgaaatcgcgtcaaacaaagcccgcctgaataatgctgacgataaaaaagcacagcgtaaaatccttgttgatagcctgctggatactgtctctggcggctggataaatgcctttgctaactggactaagcgtatctaattgagactgcacgggggagatttccacccccgtgttttcccatggaggaggatacacatgacacagttaaaaggcgaaaaaataaagcatcttgaaataattttaaaaattagtgaacgctgcaatattaattgtacttactgctatgtattcaatatgggtaatacactggcaaccgatagcacgccggtaatttctctggataacgtatacgcgctgaggggattttttgaacgatcggctgccgaaaatgacattgaggttattcaggtagactttcacggtggcgaaccgctgatgatgaaaaaagaccgtttcgatcgcatgtgccagattctcttgcagggtaactaccgcagttcaaaatttgaactggcattacaaaccaatggcattttgattgatgacgagtggattgcgctttttgaaaaacatcaggtgcatgccagtatatcggtcgacggaccaaaacatatcaatgaccgtcatcggttagaccgtaaggggaagagcacttacgagggcacaattaccggtttacgcctgctgcaaaatgcgtggcagcaagggcgtctgccaggtgaaccaggcatactttcagtggccaacgccaatgcaaatggtgcggagatttatcgccactttgccgatactctccagtgccagcgtttcgattttcttataccagacgatcatcacgacgatagccctgatggcgaaggtgtaggccgatttctgaacgaggcactggatgcatggtttgctgatgggcggccagaaatctttattcgaatctttaatacttatctcggcaccatgctaaacagccagtttaatcgggtgcttggtatgagtgctaatgttgagtccgcctatgcctttacagtaacagccgacggcatgctgcgtattgatgacacattgcgttcgacatctgatgagatattcaatgccgttgggcatgtcagtgaattatcgctggcgagggtacttgaaacatcttgtgttaaagaatatctcgcgttaagcagcaatctgccgacagtgtgcgcagaatgcgtatggaataatatctgccacggcggccgtctggtaaatcgtttttcacgcactaatcgtttcaacaataaaaccgttttctgcaaatcgatgagattatttcttagtcgcgctgcatcgcatcttatggcatcgggcgtggatgaaaaagaaatcatgaaaaacattcaaaaatag

SEQ ID NO: 200 - DNA, 4278 bp, synthetic construct
  source 1..4278: mol_type = other DNA
aagatgataataacctggttattaaaccgcttatattttgtattcgcctttagcacgacactatcctttgctgatatggaaaaatccgtaaccttaacgctgagctttgatcagcttgccaccccgcatgcaaatttcgtcatcaatggcaccccggtctatgccatggttgatacgggttcttcatttggtttccatctttatcaaaatcaacttaataaaatcaaaggattaaaaaaagaacgtacatatcgtagtactgatggaaaaggtaaagttcaggaaaatatagcgtatctggctaaatctctcgatatgaatgggttgaaattaagagatgtccccgtcactccatttaagcagtgggggctgatgatctctggcgaaggtgaattgccgcagagccaggtcgtggggttaggtgcatttaaagataaacaaatattactggattataaggggaaatcactcaccattggcgacaacatcgcttctgaatcgcaaatcaaagaaaattttcaggaatattcttttcaaatgtcttccgatggcatgatctttcaagccgagcaatccgggcataagtatcatctgattatggatacaggttccaccgtttccataatctggcgtgagagacttaaatccagacaacctgagagctgtcttattgtcgatcctgagatggataatgaaggatgcgaggcactgatgctggaaacgaaatcgaagaatggcaaaatcgagcattttggcgcggtcattgtagccggtgactttgaacatatgggcaatattgatggacttataggtaacaacttcctcaaaagcagaaagctattgatagattttaaaaataataaggtttttatttccgatgacaacagaaaaggatgatgagtcagtctttcgtgccgaggcattgcaacataagcgtgagggatggtttggcccttcccgtctgcatgtcccgtcaggtctcactatttttctgataaccggcctgataaccggcattttcactgtatccattattacgtttggttcgtacagcgaacggataaacgtcaccggaatggtggcttatgatcctccagcggtggcgttaatggcactacgtgatgggataataacccgttcctctgcatttgagggaacaatcataaaacgcggccagctggttttcacggtaagcagtgatattcataccaaccttggccctgccaacgttgaaatgatggcgctgttaaaaaagcaacgtgatgcactgtctaaaaagcttgagatcaccattagcaatgctcaaaaaaatagtctctatctggccagtaaaactaaaataaaacagcaggaaattaacagcctggaagcgttgatacaagaaagcgaaattcagaaggaatggttcgcagaaaaatccaggctgtatacccacttaagaaaaaaaggcatcgcgcttgattcggatctgatagacaggcgaaaagattattatttatcagcagaaagtttatcttcatcgaaggtaaggcggatcactctgcaaggtgagttgctggagttacagaaacaagcgtcatctgtagacagggatttaaatgaaaaaaaagaatcctttattatagaactggcaaccattgatcaaaggattcttgatgctgagaaaaacaaagaatatttaattgtcgccccctttgatggcgtcataaccagcgtaagcgcacatattggtgaaagggtaacagctggacagagaatagctgtgcttgtgccgcaaggcgcaacggcaaaagttgagctactttcgccttctgattcaattggtgaagtcgtcagagggttgcaagtaaaaatgagagtggccgcatacccttatcagtggtatgggaaaatccgtggcgcgatagaagcgatatcggtagcaccagtcaatatgacatccccggcacaggcaaagagtgattatagcggcaaaggactttttcgcatcattgtcacaccagagctgacagagcagcaattgaatatttcgcttttacctggcatggaggtcgaagcggaaatatatgttaaaaccagaaaagtttaccaatggttatttatacctgtcaggcgggcatatgaacgtgcaacggacagcatggaatagagatgcaatataatatcagcgcattttttcagtcttttagcaaaaggctaccggtaataatgcaaacagaggttactgagtgcggattagcttgcctggcaatgatagccgcatggtatggtcgcaagacagatatttacgggatgcgaaaactttttgacgtctcaagtaacggcatgacattaaggcaaataatgacagccgcaggacgaataaacctgaatgcccgtgcagtgcggcttgagctggaggagctgagcagcactaaacttccgtgtattttgcactggtcattcaaccatttcgtggtgttgaaaaagataagcaaaaaaggcgctatcatccatgaccccgcatccggaaagagaattatcagcatcaatgaactgtccaataaatttaccggcatcgctctggaagtgtggcctcaggccgaatttaaaaaagaaaaaatcagcgagagtattactgtcagcgatatgtttcgcggcgtagacggacttgggcgtgtgctgtgtaaaattcttctgttatcactgtttatcgagattctggccctttctgttcctcttgcctctcaatttattattgatattgcgttaaaggcaagcgacctcaacatgttgaattttattataactggcgtcgtttttctgcttatcctgcgtgcgattcttagtatggttcgcgcctggacgcttatggcgatacgttattcacttggcatccagtggagcgccggattttttaaccgcctgctaaagctgccggtggccttttttgaaaagcgccatgtcggagatattgcctcgaggctgacttcgctaaatgaggtgcaggaagcatttacggcagaaatgcttacttctctgctcgacgtacttattctgctggcgctgatcgcgctgatgttcgcttacagcccatttttggccatcatatccctgctgatggccgctgtttatctgggggtgaaattaatgttctatgacacctgcatgggggcgaaagttgaggcgatagcgcatgaagcccagcaatcatcccactttctggagactgtgcgcggcgtggcagcggtaaaagtgtttgatttagctgaataccggcgtaacgcatggcttaaccgggttattgataccgcgaatgcacgcgctcatctgttaaagatagatcttattaaccagacgctttcggctctgctgacgggtctctcatcggcagcgatcctgtttatcggcggcagcctgatggaagcgggcataatgaccgcgggtattctgttggcttttctgctctatgcagatatgttccttacccgttcagtgaaggtgataaattcgctgtttgattttcgtctgatctcgatccacacgcagcgcctgacagatattgctgcaaccgaaacagaaagtgcatggaatccgctaaatcctgtacggcttgagaacgtatccggccagctaaccctgagtgcgctttcatttcgctacagtgaggcggaaccctttattttcgaagggatagatatggagatcaaaccgggcgagagcgtagcgattatcggcccatcaggctgtggtaaatcgacgcttctcaatgttatggcgggtctgactcttccgcattcaggagagatatttattgatggcgttagtgtccgccagactggtattgacgaataccgtcggcacacggcgtttgtcatgcaggatgataaattatttgcagcctcactcatggataacatcacttcttttaccccacagcctgatattgactggatgcatgaatgcgccacggcagcggcaatccatgatgagattatggcgatgccgatgcaatacgaaacgatggtgggtgacatgggaagtattctttctagcggacaaaaacagcgcgtgtcgctcgccagggcgctgtacaagcgtccccgcattctgtttcttgatgaggccaccagtgacctggacgttattaacgagcggaagatcaatgaagcggtaaaacagatgcctgttacacgggtattcgtggctcaccggccagagatgattgctgtcgccgatcgggtttataacctgagagataaaacttttgtgccatcaggctatgaggttacagattaa

SEQ ID NO: 201 - DNA, 1479 bp, synthetic construct
  source 1..1479: mol_type = other DNA
tctaacttgaaaaaagaaatcgctgaaactaaaactgaaattaaaggtactaaagttaaaaataatcaacctcaacctctaacagaagatctgctcgaccaaatctctggtggttgggtgaatgcttacgcaagatggacaaaccgcttttaaattcagtagattaaagtcagggggcttaattgcccccatttgattctttcgagctgagcaatgttcgtagttggaacttaacctgccattttcgtattactggcatagggtctaacaaagtaaaaaatggagcttcgagtgatggttaattcattagttaagaaaaaaattcaacatcttgaagtaatattaaagataagcgagcgatgtaatatcaattgtgactattgttacgtattcaatagaggaaattcagcggctaatgatagccccgccaggatctctcatgcgaatattgattacctggtggatttctttcagcggggaagtcaagaatatgatattgacactctgcaaattgattttcatggaggagaacctctcatgatgaaaaagccgcagtttgccagtatgtgtgagcgactagcctcaggtaattaccatggttcgaaaatcagatttgcattacagactaatggcatccttattgatgatgaatggatatctttattcgaaaaatattctgtcagtgtgagtgtctccattgatggaccgaagcatattaatgatcgtcatcgcttagacagaaaagggcgtagtacttacgaaggtactatacggggtctccgtaaacttcaagaagcttatcaagcaggtcggctgccgtcagatccgggtattttgtgtgtcgcgaatgctaaagcaagcggggctgaaatatatcgacactttgttgataacctgggcgtttatggctttgattttctggtacctgacgactgttacactgatgcccaggttgatccagatggcgttggacgtttcctaaatgaggcgttagatgaatgggtgaatgacaataaccccaagatttttgtgcgtctttttaatacccatattgccagtcttcttggcgcggaaaatgcggggtttttggggcataacccaagcgtagctggaatatatgcatttaccattggttcagatggttttgtccgtgtcgatgataccttgagatcgacatctgaccgtattttcgacatcattggtcacatttctgaaatcagcctatctgaagtattaaatagcccacagtttcaggaatatgcgtctataggggaatcgttaccaacagaatgtgaagactgtatttgggcaaaagtttgtgccggtgggcgcatagttaatcgcttctcgcatgaagagagatttaaacgcaagtcagtatattgttattcaatgagaagccttcttagccgcgtttcagctcatcttctcaatatggggattgaggaagatcgcattatgaaagcgattggccggtaa

SEQ ID NO: 202 - DNA, 4212 bp, synthetic construct
  source 1..4212: mol_type = other DNA
ccagtaggcgcctcagtttggacaataatagcgcttgttattattgtcagccttgttgtgttcatgataataggcacttacacacagaaggttcggctaatgggggaaattatctacgagcctgcggttgcgagaatagaagcaacgggtaacggaaccattgtccgtagttttgctgttgaagggaaagaagttcgcgctggagatgttatttttatcgttaacatggaaactcaaaccgaatatgggcgtacaagtcatgaaattacttctgccctcaagtcacaaaaaaccgctattgaacgagagatcatgctgaaatcagaggcgtctgatcaagaaagtgattttcttacccagcgtcttaagaataaggaagcggaaattcaagaattagacaacctgatcacaaaatcaaccgaacaagtcgcgtggctatttgacaaagctcagcttttcaataaattagttgggaaaggaatcgcacttgaaatagatcatatagaacgccgctctgattattatactgcttctgttcaactggcggcttacaaacgagaaaaggttaagttacagggtgaatctctcgatatcagggcgaggttggcgacaatccacattggacttgaaacttcacgtgaaacattacgtcgagatattgcacggctagatcaagacttagtctctacggcagaacgaagggaactctatataacgtctccaattgacggtaagttaacgggaattactggattagttggcaaaagaattcgctcgtcccaggaattagcgagtgttgtacctacttcgggccgccccaaagtagaaatcttttccacttctgaagttattggagaattacgcgagggacaatctgtaaaattacggtttgatgcttatccataccagtggtttgggcagcatgatggtattgttactgcaatttccacgacttcagttgaagggagtttaggaataaaggatgaaaataatcagcaacagaaacggtattttcaggttcatatccgtcctaaaagcgacggtgtactcttagcgggaaatatgcatcctttacggcccggaatgggggtcgaaacagacatttttataagaaaaaggccaatctacgaatggattttgttacctctaaaaagaattcatgtcgcgactcaaggtaaacctggagatgatgtatgaatgtcacaatgaaaggctactttgaagcattcaggcaccatcttcctgtagtgatgcaaacagaggctacggaatgtggactcgcttgtgtcgctatgattgcaggttattatggacttaatatggatctgcaagcgcttcgcaaatattatcaggtgtctttaaaaggtatgaacctgcgcgatattatcgtattagctgatcgcctctcattagcgtctcgtccaattcgagctgatcttgattctttaagtcaggtaaaaacgccttgtattttgcattggtcttttaatcactttgttgtattaaagaaattttcacgccgtggggtcgttattcacgatccggcaaaaggcgagagaagaatttctatcgatgagttatctaaaaaatttacgggtattgcacttgagctttggccaaataaagactttcagaaacgtactgaaaagaaaacaattcgactgctggatatgtttaaaaacgtttctggattatctcgggctttagttcaagtattggctttatcattttgtattgagcttcttgctatggccgtgccgatggcagctcaattcacgatagatatggctttgaggtctagcgatattgatcttgtctctgtgattgtgtgcggaattattggcttattaatattcagagccttgcttagtattgtgcgttcatggtctgttatgtcgattaagtatactttgggtattcaatggagctctgggctttttagtcatatgatccgattacctacttcatactttgaaaagcgtcatattggtgacgtcacttcgcgatttaactctttatcggcagtacaagatgccttcaccgcggatatgatagcttcactcttagacattgttgtggtgattggactcttctttttaatgtgggtttacaatggttatcttgctgtcgtggtcatttcgatatccattgtatacgcatcgctaaaattctttctttttcgagcctatcgttcggctaatctcgaggcgatagcccatgaatctcagcaacagtcacacttccttgaaacagtacgcggcatcacttgcgttaaaatttttgacttagccgatcgcagacgatccgattggctcaatcttgttattgatgaagccaatgcaaaaatatacctctttaaaattgacctggtgacacagactgcggcacagcttttaattggtcttacttctgcatccatattatggttaggcgctaaattgattgatggcggcgcgttaaccacaggtatgctttttgccttcttgatttactctgatatgtacgtaaatcgaaccatacgagtggttgactcgattattaaacttcgcttgatcgatatgcatagcgaacgactgtcagaagtggctttagccgaacctgaacataatgaaggggatgctgttctatcatgtcctgaaacaatttcaggcagtattgaaattaaaagcctgagttatcgttatggcgatggcgaacccgctatatttgagaatgtttttctgtctattaaggctggtgaaagtatcgctatagttgggccgtcaggttgtggtaaatcgacactgcttaagacaatcggtggattagtctcgccagaaagtggctttatttatttggacggagttgatgtgcggagattaggacttggggcctaccgtagccatatcgcttgtgtcttacaagaggacagattatttgcgggatcgctattggataatattagttcattcgacgttaagcctgaccatgaatgggtatatgagtgtgctcgtcttgcttcaattcacgctgaaatagaagagatgccaatgaaatatgaaacaatggttggagacatgggcagtgctctgtcaggtggacaacggcagcgtatttctcttgccagggcattgtacaaacgtccaaagatattatttcttgatgaagcaacgagtgatctggatatcgataacgaagcaaaaattaatgactcaatacgagaactaaagattaccagggtatttgtagcccatcgtccgacaatgatcgcaatggcggatagggtttttgatctaagtatgaacgcagaagtggagaacccccatgcatttttctctaagtaaacatatcaaggtgaccgcatttgttgctttttcttccatgatgtcattatttgttgcaaattctatggccgctgaaaaagtcatgcatatcaattttcaatttgatgaatttgctctaccgatagcaaatcttgaaattgatggaaaaactcaaaatcttatgatcgatacgggttcaactataggtctccatttatctaaaaacctgatgtcgaaaatttccggcttagttatcgaacctgaaaaagcgcgttctactgaccttacgggtaagacttttttaaatgacaaatttaatattccacggctttcgataaatggcatgatgtttaaagatgttaaaggggtttcattaacaccatggggaatgaaattaattggagacaatgatcttccttcctcaatggtaattggccttgatttattcaagggaaaggtggttcttattgattataaaagccggaaattatcagtttctgatcgtttgcaagcgttgggagtcaatgtggataatggttggataaaattgccgctgagactgactaaagaaggcattgctgtcaaagtttcacaaaactttaaaagctacaacatggtattggatactggcgcatcggtttcgattttttggaaagaaagattgaaatctcctccggttaacatttcttgccaggctgtggttaaagagatggacaatgaagggtgtgttgcatcgacgtttcagcttgacgaaatgggcgttaagggagttaagctgaattcggtattggttgatgggggatttaatcagttaaatactgatggattaatcgggaataatttctttaataaatacgcagtattaatcgacttccctggtaagagattattcattaaagagaactcgtag

SEQ ID NO: 203 - DNA, 5435 bp, synthetic construct
  source 1..5435: mol_type = other DNA
gctaacaaagaaaaaatcaaacacctggaaatcatcctgaaagtttctgaacgttgcaacatcaactgcacctactgctacgttttcaacctgggtaacgacctggctatcaactctaaaccgatcatctctcacggtaccatcaaaaacctgcgtggtttcttcgaacgtgcttgccaggaatacgaaatcgaaaccgttcaggttgacttccacggtggtgaaccgctgatgatcggtaaagaccgtttcgacaacgcttgcaaagaactggtttctggtgactacaacggtacccgtctgaacctggcttgccagaccaacgctatcctgatcgacaacgaatggatcgacatcttctctaaacacaacatctctgttggtatctctatcgacggtccgaaacacatcaacgaccgtcaccgtctggaccgtaaaggtcgttctacctacgaaggtaccgttaaaggtctggaaatgctgcaggctgcttggcgtgctggtcgtctgatcgacgaaccgggtatcctgtgcgttgctaacccgtctgttaaaggtgctgaaatctaccgtcacttcgttgacgttctgaaatgcaaaaaattcgacttcctgatcccggacgaatctcacgacacctgcaccgacccggaaggtctgtctgacttctactgctctgctctggacgaattcttcctggacgctgacaaagaagtttacgttcgttacttccacacccacatccagtctatgctgtctctggaattctctccggttatgggtgtttctaaagctggttctgacaccctggctttcaccgtttcttctgacggtgaactgtacgttgacgacaccctgcgttctaccaacgactctatcttcaccccgatcggtcacatccagtctctgaccctgtctgaagctctgacctcttggcagatgcagaaatacctgtctgttgacaaccagctgccggaagtttgcatcgactgcatctggaaaaaactgtgcggtggtggtcgtcacatccagcgttactcttctgctgacgacttcaaccgtgaaaccgttttctgcccgtctatccgtaaaatcatgtctcgtgctgcttctcacctgatcgaatctggtgttaccgaagacatcatcatgaaaaacctggaagttaactcttaatggagccggacaatggaaaaaatcaatttctggttatcaaagttttcatgtgccgccctcgctatttgttgtacatcttgccttgctgactcgggaaattcggtaacacttaagctgaattatgacaaatatttcacgcctcatgcaactttcatcattaatggccacccggtaaatatgatgattgatacaggttcttcgaagggcttttatcttcaagagcctcaactaaaaaaaatacaaggcctcaaaaaagaaagcacttattacagtactaatatcaccgggaaaagacaggagaacacagagtatctcgccgcttctctcgacatgaatggccttaaattaaaaaacgtaaccgtgatcccatttaaacaatggggagcgctgatttctaacacaggtaaattgccggatggccctgttgtcggtctcgatgcgtttaaagataaacaaattatgctggattttgtgtctcattcattcacgatgagcgacagttttatccataacatgccggttccgaaaggctttaacgcattcactttccatatgtctcctgatggcatggtttttgatgttgatcagtctggacacacataccatttgattctggacaccggtgccactgcgtctgtgatttggcgtgaaagacttaaacagtatgaacccaaaagctgcctgctggtcgatccgaagatggataacgaaggatgccaggccactctgctcacaattaaatcaaaaactggaaatccccagcattttggtgcggttgttgttgtcggaaattttaaacacatgggcaacgttgatggccttttagggaataacttcctcagaaatcgaaaggtacttatagactttaaaaacaagaaggtttttatttccgatgagcaccgaaacagaaaagaatgacaactcaatctttcgtgccgaggctttgcaacacaaacgagaaggttggctcggcgcttctcgtttgcatataccgtcagcgctctctatttgttgcctgacaatccttgttattttctttttcatcatattgataattgcatttggttcgtacagtgaacggataaatgtcatcggaaccgtggtttataagccgcctgcggtatcactgattgcacaaagcagtggaatcattacgcattcactggcattagagcaaacaagagttaagcgcaacgagagcattttttctatcagtggtgacactcagacaaatctgggtgccaccaatgttgaaacggtagaacttttaaataagcaacgtaacgcgctgtctaaaaagcttgatattgcggccaatgaatcaaaagcaaacaagatttatctcagcgaaaaaattaaaaataaacaacaggaaatagaaagtctgcaaaacctgatagaaacttcagaaaaacagcaagcgtggttcgagaaaaaatcaaacctgtatgcgaattttaagaagaaaggcattgcgcttgatgctgaatggataaacagaaagaaagattattacgcatccacattaagcatttcttctgcaaaggtcaaagtgatagccctgctgggagagttgcaggatctgaaaaatgacgtttcggttatcgacaggaaactcgacaaagaaacagcatctctcactgtcgaaatagccgatatagcacaaaaaatactgattacagaaaaacaaaaagagtatttaatcgtcgcgccgtttgatggaatgataaccagtgttacagcccatatcggtgaaagagtgactgccggccagcaaatagccgtgctgataccacaaggtgcgacagaaaaggttgagttgttttcaccgtctgattctctcggtgaagtgaccagcggacagcaagtcagaatgagagtctcggcatacccttaccagtggtatggaaagattgcaggcatcatagaaacgatatcggcagcaccggtcaatgtcacctcacagatgcagatgaaaggtgaagaggtaaaaaaggggctttttcggattgtcgtacaaccaaaattgaccggacaacaaacaaacatttcccttctacccggcatggaagtggaaacagagatctatgtgaaaacccgaaaattgtacgaatggttatttatccccattaaaggggcatatgaacgggcgacagacagtacggaataaatatgcagtataagatgagtgattttttcgagtttttcgtcaaaaaactcccggtgataatacaaacagagaccacagaatgcgggttggcatgtctggccatgattgctgcctggtatggccgtgagactgatatctacagcatgagaaaggtttttgacgtgtcaaacaatggcatgacattaaggcagatcatcacggcggccgggcgaataaacatgaataccagagctgtgcggctggaactcaacgaactcagcagtgtcaggcttccgtgcatcttgcactggtcctttaatcattttgtcgtgttaaaaaaattcacaaaaaaaggggcagtcatccatgatcccgccttgggaaaaagaactgtcactctgaaagaactctcaaataagtttacgggcatcgctctggaagtctggccccagacggagtttaaaaaggaaaaggtcagtgaaagcataaccatcacggatatgtttcgcggtgttgccggccttaagaatacgctgtttaaaatcattctgttgtcgctctttattgaagtactggcactttccatccctctcagctctcaattcattattgatgttgttctacggtccagtgacctcagtatgctgaatttcattgtcattggaatcgttcttctgctctccctgcgcgctgctttcagtattgtgcgcgcctgggctcttatggcaatgcgttactcacttggcatacagtggagttccggtttttttaaccggttactcagattgccggtcactttttttgaaaaacgtcacgtaggtgatatcgcctccagattgacatcgttgagcgaagttcaagaagcctttacagcagaaatgctgacttcgttacttgatgtacttattctcataacgctggctgtgctcatgttctgttacagccctcttctgacccttctcccgctactcatgactaccgtttatcttggggtcaaatttgctttttatgacagatacatgggagcaaaagtagaagcaattacgcatgaagcgcagcaatcatcctactttctcgaaacaatacgaggcgtagcgtgcgtgaaagtatttggcctgacagaattccgacgtatcacatggcttaaccgggtgattgatactgccaatgcccgggcccatttatttaagatagacctcatcagccaaacgctttcaggtttcctgacggggctatcatcggcggccattttgtttatggggagtcatctcacagaacgcggcctgatcactgccggcattctgtttgcttttctgctctataccgatatgtttctgacacgttcagtgaaggtaataaattcactgtttgcttttcgccttatttcgatacacacgcaccgattgaccgatattgcaacagcccagacagaaaatgcatggaacccggaagatcccgtcacactcgataatgtaaaaggccggataacactgaacaatctcacatatcggtacggagaaactgaaccctgtattttcgactgtatcgacatggaaattaatgctggtgagagtgtggcgatcgtaggtccgtcaggttgcggtaaatcgacacttctccgggtcatggccggcctggttctccctcagtcaggcgatgtgtcaattgatgatgtcagtgtgaaaaaaatgggtattgacgaatatcgcagacacacggcgtttgtcatgcaagatgataagctttttgctgcctcattgatggataacatatccgcttttgatccacagccaaatattgattggatacatgaatgcgctaaggcggcggcaatacacgatgaaattatgactatgccgatgcagtacgaaaccatggtgggtgacatggggagcattctttcaggcggacaaaaacagcgtgtatcccttgcacgggcactttacaagtgtccgcgtatcctctttcttgatgaggccaccagccatctcgacgtttttaatgaacgcaagataaatgaggctgtaaagcagatgccgattacgcgtgtatttgtggctcatcggccagaaatgatcgctgtcgcagaccgagtttataacctgaggga

SEQ ID NO: 204 - DNA, 299 bp, synthetic construct
  source 1..299: mol_type = other DNA
tctaaactggctaaagaaatctctatgaacaaagctgctgttatcatcgacggtgacaaaaaagacgttcgtcgtgctctgacccagtctatgctggactctgtttctggtggttgggttaacgcattcgctcgttggtctaaacgttggtaaaattcgagctcggcgcgcctgcaggtcgacaagcttgcggccgcataatgcttaagtcgaacagaacccaagaccaggggggctcgccacgttggctaatcctggtacatcttgtaatcaatattcagtagaaaatttgtgttaga

SEQ ID NO: 205 - DNA, 300 bp, synthetic construct
  source 1..300: mol_type = other DNA
tctaaactggctaaagaaatctctatgaacaaagctgctgttatcatcgacggtgacaaaaaagacgttcgtcgtgctctgacccagtctatgctggactctgtttctggtggttgggttaacgcattcgctcgttggtctaaacgtttctaaaattcgagctcggcgcgcctgcaggtcgacaagcttgcggccgcataatgcttaagtcgaacagaacccaagaccaggggggctcgccacgttggctaatcctggtacatcttgtaatcaatattcagtagaaaatttgtgttagaa

SEQ ID NO: 206 - DNA, 1585 bp, synthetic construct
  source 1..1585: mol_type = other DNA
ggtcatcacatcatcatcatcatcacagctctggattagtgccgcgcggtagtcatatgtctcgcttacaaaaagaaatcaatgaaactaagacagtcattaacatttgtaatactaaaaagagtcaacctcagcatcttgcagacagtattctcgacaagatagcaggcggttgggtgaatgcttttgtaaactggccaaaaagtttttaagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggtcaatcaattaaacattcaaagcatccaacaccttgaaataatattaaaaataagcgaacgctgtaatattaattgtgattattgctatgtattcaataaaggtaatccggcggctaataacagccccgccagattgtcagatagaaacattaatgacttagctgaatttcttcacacagcatgtcgggaatataaaatcggtaccctacaaattgatttccacgggggggaaccgttattgatgaaaaaagaaaacttcgccaaaatgtgtgagcgattactgacaggaagatactcgaagactaatatcagattcgcattgcaaactaacggcacacttattgatgaagaatggatatcactatttgaaaaatattctgtgaacgcaagtatttctattgatggcccgaaacatattaatgacaggcatcgtttagataccaaagggcgtagcacttacgaggcgacagtgcgtggtttgcgtatactccaacatgctcataagcaaggccgtattccatcggcaccgggggttttatgtgtcgcgaatgctcaagcaaatggtgctgagatatatcgtcattttgtggacgaattaaaggtttatggttttgattttctggtgccagacgattgttatcatgacactaatattgaccctgttggtattagccgcttcctaaatgaagctttggatgaatggttcaaggacagcaaccctaatatttttgtccgcctttttcaaacacacttagctcatttgctcggtacaaagcatcaaggaattttagggcattcacccagcgccactggggcatacgcattcaccgtgggttcagatggttttattcgtgtggatgataccttacgcgccacatcagacagaattttcaatcccattggtcatgtttctgaaatcagcctaactgatgcacttaatagccctcagttccaggagtacgcgtcagtcggccaagctctgccccatgaatgcaacggttgcatttgggaaaacgtctgtgctggaggtcgtattatgaatcgtttttcacctgaaacccgcttcgaccgcaagtctgtttattgctattccatgagaagtttcctcagccgcgccgctgcacacctactcaatatgggcatcaaggaagagcgcattatgacagcaattgggcgataa

SEQ ID NO: 207 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggttaacgctttcgttaactggccgaaatctttctaa

SEQ ID NO: 208 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggttaacgctttcgctaactggtctaaatctttctaa

SEQ ID NO: 209 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggttaacgctttcgctcgttgggacaaaaaattctaa

SEQ ID NO: 210 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggttaacgctttcgctaactggaccaaacgtttctaa

SEQ ID NO: 211 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggttaacgttttcgctcgttgggacaaacagatctaa

SEQ ID NO: 212 - DNA, 153 bp, synthetic construct
  source 1..153: mol_type = other DNA
agcaaattacagcgtgaaattgcagcaaacaaagctcaactgagccatgaagacaagaagaaaacgcagcacaaagagcttgttgacagcctgctggatactgtctctggtggttgggtaaacgccttcgcacgcttcacgaagcgcttctga

SEQ ID NO: 213 - protein, 13 aa, Yersinia kristensenii
CNINCDYCYVFNK

SEQ ID NO: 214 - protein, 10 aa, Yersinia kristensenii
CEGCIWENIC

SEQ ID NO: 215 - protein, 8 aa, Erwinia toletana
CNINCTYC

SEQ ID NO: 216 - protein, 10 aa, Erwinia toletana
CAECVWNNIC

SEQ ID NO: 217 - protein, 8 aa, Micromonospora sp.
CDLACDHC

SEQ ID NO: 218 - protein, 10 aa, Micromonospora sp.
CRRCPVVDQC

SEQ ID NO: 219 - protein, 8 aa, Oscillatoriales cyanobacterium
CNLNCDYC

SEQ ID NO: 220 - protein, 11 aa, Oscillatoriales cyanobacterium
CRETCEYFGVC

SEQ ID NO: 221 - protein, 8 aa, Lyngbya sp.
CNLNCDYC

SEQ ID NO: 222 - protein, 11 aa, Lyngbya sp.
CRQSCEYFGLC

SEQ ID NO: 223 - protein, 8 aa, Geminocytis sp.
CNLDCDYC

SEQ ID NO: 224 - protein, 11 aa, Geminocytis sp.
CSDNCSYFGIC

SEQ ID NO: 225 - protein, 26 aa, Xenorhabdus nematophila
  source 1..26: note = strain SC 0516
ELVDSLLDTVSGGWINAFGNWERAFH

SEQ ID NO: 226 - protein, 25 aa, Serratia marcescens
  source 1..25: note = strain CAV1761
ALAQSMLDSVSGGWVNAFARWSKSF

SEQ ID NO: 227 - protein, 25 aa, Erwinia toletana
  source 1..25: note = strain DAPP-PG 735
ILVDSLLDTVSGGWINAFANWTKRI

SEQ ID NO: 228 - protein, 30 aa, Photorhabdus australis
  source 1..30: note = strain DSM 17609
NNQPQPLTEDLLDQISGGWVNAYARWTNRF

SEQ ID NO: 229 - protein, 51 aa, synthetic construct
MSKLAKEISMNKAAVIIDGDKKDVRRALTQSMLDSVSGGWVNAFARWSKRW

SEQ ID NO: 230 - protein, 51 aa, synthetic construct
MSKLAKEISMNKAAVIIDGDKKDVRRALTQSMLDSVSGGWVNAFARWSKRF

SEQ ID NO: 231 - protein, 52 aa, Pandoraea sp.
  source 1..52: note = strain PE-S2R-1
MTKQALIAQLTEKQNAAKDDGSIKEIPQSELEQISGAGRGNAFVNATWSRAM

SEQ ID NO: 232 - protein, 50 aa, synthetic construct
MTTKAFIEQLAKKQKAANEAGSIKEIPASELERISGARGKFANATWSKSF

SEQ ID NO: 233 - protein, 10 aa, Bordetella bronchialis
  source 1..10: note = strain AU17976
FANATWSKSF

SEQ ID NO: 234 - protein, 13 aa, Pandoraea sp.
  source 1..13: note = strain PE-S2R-1
GNAFVNATWSRAM

SEQ ID NO: 235 - protein, 25 aa, synthetic construct
ELVDSLLDTVSGGWVNAFARWSKSF

SEQ ID NO: 236 - protein, 14 aa, synthetic construct
AAHVANLLDNVQGG

SEQ ID NO: 237 - protein, 12 aa, synthetic construct
FSQSLLDDVQGG

(19) United States
(12) Patent Application Publication
(10) Pub. No.: US 2026/0049104 A1
(43) Pub. Date: Feb. 19, 2026

(54) PEPTIDES WITH ANTIMICROBIAL PROPERTIES

(71) Applicant: National University of Singapore, Singapore (SG)

(72) Inventors: Brandon Isamu Morinaka, Singapore (SG); Ryosuke Sugiyama, Singapore (SG); Ziwei Yao, Singapore (SG); Pui Lai Rachel Ee, Singapore (SG); Dai Thien Nhan Tram, Singapore (SG); Yohei Morishita, Singapore (SG); Chin-Soon Phan, Singapore (SG); Joel Lim, Singapore (SG)

(73) Assignee: National University of Singapore, Singapore (SG)

(21) Appl. No.: 19/099,025

(22) PCT Filed: Jul. 27, 2023
(86) PCT No.: PCT/SG2023/050524

§ 371 Date: Jan. 27, 2025

(30) Foreign Application Priority Data
Jul. 27, 2022 (SG) 10202250612J

(51) Int. Cl. C07K 7/08; A61K 38/00; A61P 31/04; C07K 7/06; C07K 14/195; C12N 15/70

(52) U.S. Cl. CPC C07K 7/08; A61P 31/04; C07K 7/06; C07K 14/195; C12N 15/70; A61K 38/00

(57) ABSTRACT

The present disclosure concerns a polypeptide comprising a first three residue motif (from a N-terminus) and a second three residue motif, the first and second three residue motif optionally separated by 1 to 3 amino acid residue, and at least two C-terminus residues. The three residue motif is each represented by X₁-X₂-X₃. Each X₁ is a residue independently selected from tryptophan, phenylalanine, tyrosine, histidine, an unnatural aromatic amino acid residue or a derivative thereof. Each X₂ and X₃ are independently any amino acid residue. X₁ and X₃ in each motif are connected to form a cyclophane moiety. At least one of the two C-terminus residues is an aromatic residue. The present disclosure also concerns a method of producing the polypeptide.

### SEQUENCE LISTING

The present application contains a Sequence Listing which has been submitted electronically as an XML document in the ST.26 format and is hereby incorporated by reference in its entirety. Said XML copy, created on 28 Oct. 2025, is named S61018249_Peptides_with_Antimicrobial_Properties.xml and is 288 KB in size.

### TECHNICAL FIELD

The present invention relates, in general terms, to peptides with antimicrobial properties and the methods of synthesising the peptides thereof.

### BACKGROUND

The CDC and WHO classify Carbapenem-resistant Enterobacteriaceae (CRE) which include the Gram-negative bacteria Klebsiella pneumoniae and Escherichia coli as two of the highest priority pathogens for which new antibiotics are urgently needed. CRE are an immediate threat because of their resistance to any carbapenem and their 50% increase over the last 5 years. Extended-spectrum p-lactamase-producing Enterobacterales (ESBL-E) account for a greater number of cases and more deaths compared to CRE but may still be treated with selected carbapenem antibiotics. The increased use of carbapenems, along with transmission of various resistance mechanisms have likely contributed to the rise in CRE. Both CRE and ESBL-E can lead to severe and deadly infections in hospital and nursing home patients via pneumonia, bloodstream infections, urinary tract infections, wound infections, and meningitis. New antibiotics able to treat both types of infections would reduce the mortality rate and decrease the spread of resistance mechanisms.

Ribosomally synthesized and posttranslationally modified peptides (RiPPs) are a rapidly growing family of natural products with potential antibiotic activities against a broad range of pathogens. RiPPs may be biosynthesized from a ribosomally synthesized precursor, posttranslationally modified, cleaved, then exported to give the mature RiPP. For example, RiPP pathways involving radical S-adenosylmethionine (rSAM) enzymes in their biosynthesis are of particular interest due to their ability to catalyze distinct chemically-demanding reactions leading to unique and bioactive RiPP natural products. The structural diversity and antibiotic activities are demonstrated by several RiPP families including lasso peptides, plantazolicins, lanthipeptides, thiopeptides, and sactipeptides. RIPP biosynthetic gene clusters (BGCs) are attractive for genome mining and synthetic biology due to their compact size and ease of genetic manipulation. For chemically-guided discovery, RiPP pathways are particularly appealing because a single posttranslational modifying enzyme can create unique, structurally complex, and bioactive peptides. Since RiPP biosynthesis is determined by a logic rather than genetically tractable features, their true number and diversity remains enigmatic and a promising source for new peptide scaffolds and antibiotics.

It would be desirable to overcome or ameliorate at least one of the above-described problems.

### SUMMARY

The present invention provides a polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof; wherein each X₂ and X₃ are independently any amino acid residue; wherein X₁ and X₃ in each motif are connected to form a cyclophane moiety; wherein at least one of the two C-terminus residues is an aromatic residue.

In some embodiments, the first and second three residue motifs are separated by 1 to 3 amino acid residue.

In some embodiments, the first three residue motif is not fused with the second three residue motif via the cyclophane moieties.

In some embodiments, the first X₁ is a residue selected from tryptophan, phenylalanine or a derivative thereof and the second X₁ is a residue selected from phenylalanine, tyrosine or a derivative thereof.

In some embodiments, X₂ is an amino acid residue, the amino acid independently selected from I, G, E, Y, V, L, A, D, S, T, N or Q.

In some embodiments, X₃ is an amino acid residue, the amino acid independently selected from N, R, S, D, Q or K.

In some embodiments, at least one of the two C-terminus residues is a polar and/or basic residue.

In some embodiments, at least one of the two C-terminus residues is an aromatic residue.

In some embodiments, the polypeptide comprises a third three residue motif.

In some embodiments, when the polypeptide comprises a third three residue motif, X₃ of the first motif and X₁ of the second motif are separated by 1 amino acid residue, and X₃ of the second motif and X₁ of the third motif are covalently bonded to each other via an amide bond.

In some embodiments, the third X₁ is a residue independently selected from tryptophan, phenylalanine or a derivative thereof.

In some embodiments, the polypeptide is represented by Formula (I):

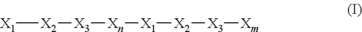

wherein each X₁ is an amino acid residue, the amino acid independently selected from tryptophan, phenylalanine or a derivative thereof;
wherein each X₂ is an amino acid residue, the amino acid independently selected from leucine, isoleucine, valine, alanine, proline, serine, lysine, asparagine, phenylalanine, aspartic acid or a derivative thereof;
wherein each X₃ is an amino acid residue, the amino acid independently selected from lysine, glutamine, asparagine, arginine or a derivative thereof;
wherein Xₙ is an amide bond or 1 to 3 amino acid residue; and
wherein Xₘ is at least two C-terminus residues.

In some embodiments, the polypeptide is represented by Formula (II):

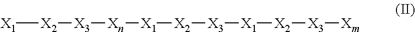

wherein each X₁ is an amino acid residue, the amino acid independently selected from tryptophan, phenylalanine, tyrosine or a derivative thereof;
wherein each X₂ is an amino acid residue, the amino acid independently selected from valine, isoleucine, phenylalanine, tryptophan, alanine, leucine, glycine, serine, proline, threonine, aspartic acid, asparagine, glutamic acid, arginine or a derivative thereof;
wherein each X₃ is an amino acid residue, the amino acid independently selected from arginine, lysine, asparagine or a derivative thereof;
wherein Xₙ is an amide bond or 1 to 3 amino acid residue; and
wherein Xₘ is at least two C-terminus residues.

In some embodiments, X₁ and X₃ in the second motif are connected via phenylene to form a cyclophane moiety.

In some embodiments, the polypeptide is represented by Formula (Ia), (IIa), (Id) or (IId):

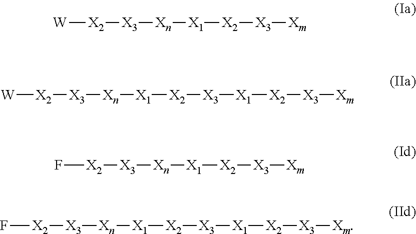

In some embodiments, when X₁ is W, X₁ is connected to X₃ via a 3,6 or 3,7 substituted indolylene moiety. It was found that the 3,6 or 3,7 substitution is advantageous for providing an antibacterial effect.

In some embodiments, the polypeptide is represented by Formula (Tb), (IIb), (Ie) or (IIe):

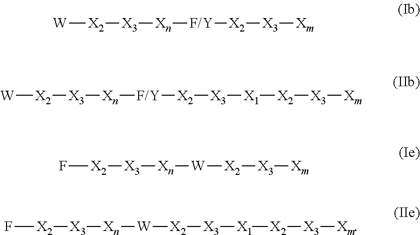

In some embodiments, when X₁ is F or Y, X₁ is connected to X₃ via a 1,3 or 1,4 disubstituted phenylene moiety. In some embodiments, when X₁ is F or Y, X₁ is connected to X₃ via a 1,3 disubstituted phenylene moiety.

In some embodiments, the polypeptide is represented by Formula (IIc):

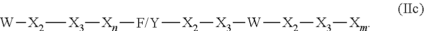

In some embodiments, the polypeptide is selected from:

| (SEQ ID 19) | |
|---|---|
| | WVNAFANWTKRF |
| (SEQ ID 17) | |
| | WVNAFANWPKRF |
| (SEQ ID 13) | |
| | WINAFANWTKRI |
| (SEQ ID 37) | |
| | WWRAYARWRRSF |
| (SEQ ID 4) | |
| | WVNAFARWGKSF |
| (SEQ ID 36) | |
| | GWFRAYLRWSRSF |
| (SEQ ID 25) | |
| | WVNAYARWTNRF |
| (SEQ ID 14) | |
| | WVNAFAKWTKRI |
| (SEQ ID 26) | |
| | WVNAYARWTKRF |
| (SEQ ID 22) | |
| | WVNVFARWDKQI |
| (SEQ ID 15) | |
| | WVNFFAKFTKSF |
| (SEQ ID 30) | |
| | WVNAFARWSRRW |
| (SEQ ID 8) | |
| | WVNAFARWSKSF |
| (SEQ ID 34) | |
| | WVNVFARWSRRW |
| (SEQ ID 35) | |
| | AGWIRAFANWSRSF |
| (SEQ ID 23) | |
| | WVNAFARWDKKF |
| (SEQ ID 20) | |
| | WVNAFARFTKRF |
| (SEQ ID 10) | |
| | WVNVFARWDKAI |
| (SEQ ID 24) | |
| | WLNVFVRWDRAI |
| (SEQ ID 21) | |
| | WINVFARWNRAI |
| (SEQ ID 32) | |
| | WINAFGNWERAFH |
| (SEQ ID 3) | |
| | WVNAFANWSKSF |
| (SEQ ID 1) | |
| | WVNAFANWSKAL |
| (SEQ ID 2) | |
| | WVNAFGNWSKSL |
| (SEQ ID 16) | |
| | WVNAFLNWSRSF |
| (SEQ ID 12) | |
| | WVNAFLRWGKSF |
| (SEQ ID 7) | |
| | WINAFARWGRAF |
| (SEQ ID 33) | |
| | AGWIKVFGNWSRSF |
| (SEQ ID 9) | |
| | WVNAFVNWTKSF |
| (SEQ ID 18) | |
| | WVNAFLNWPRSF |
| (SEQ ID 29) | |
| | AGWIKAFGNWSRSF |
| (SEQ ID 6) | |
| | WVNAFVNWPKSF |
| (SEQ ID 28) | |
| | AGWINAFANWTKSF |
| (SEQ ID 31) | |
| | AGWINAFANWTRSF |
| (SEQ ID 27) | |
| | AGWINAFGNWTKSF |
| (SEQ ID 5) | |
| | WVNAFARWGRAF |
| (SEQ ID 38) | |
| | WVNAFARWSKRW |
| (SEQ ID 39) | |
| | WVNAFARWSKRF |
| (SEQ ID 50) | |
| | RGEGWVRAYWAKRF |
| (SEQ ID 52) | |
| | KPGEGWVNFTWNKSF |
| (SEQ ID 46) | |
| | KSEAAGGWVNFQWKNSW |
| (SEQ ID 49) | |
| | AGNDGWVKFGWKKKF |
| (SEQ ID 54) | |
| | ASTAETWFKLDWKKSF |
| (SEQ ID 41) | |
| | DGRWLQWIKNH |
| (SEQ ID 40) | |
| | GDRWLKWIKNH |
| (SEQ ID 44) | |
| | VGGFANATWSKSF |
| (SEQ ID 43) | |
| | VGGFANASWPKSF |
| (SEQ ID 45) | |
| | VGGFANATWPKSF |
| (SEQ ID 59) | |
| | NAFVNATWSRAM |
| (SEQ ID 47) | |
| | NVFVNATWSRAM |
| (SEQ ID 60) | |
| | NVFVNATWSRAI |
| (SEQ ID 55) | |
| | SSDDDGIFFKTTWDRR |

In some embodiments, the polypeptide is selected from:

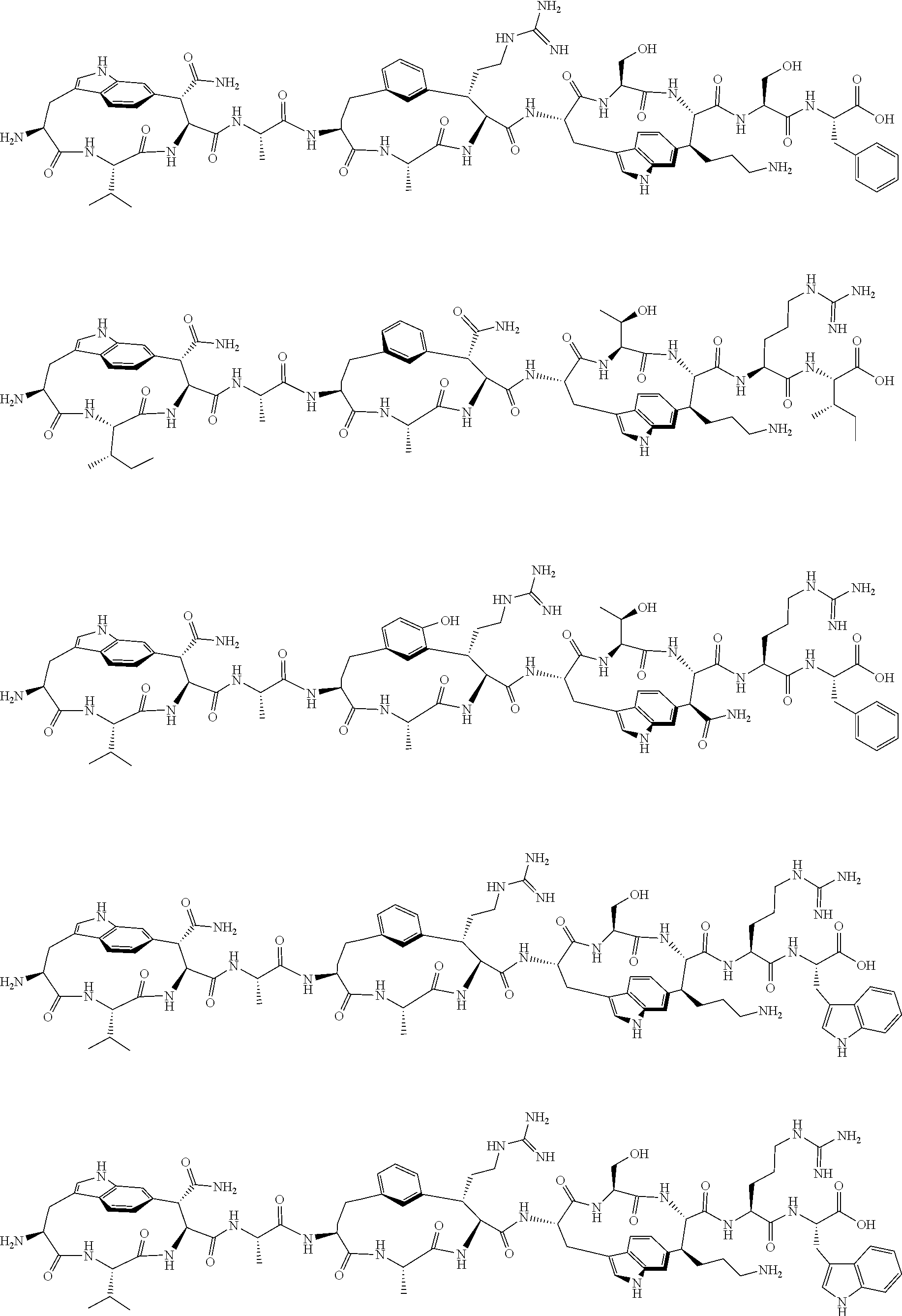

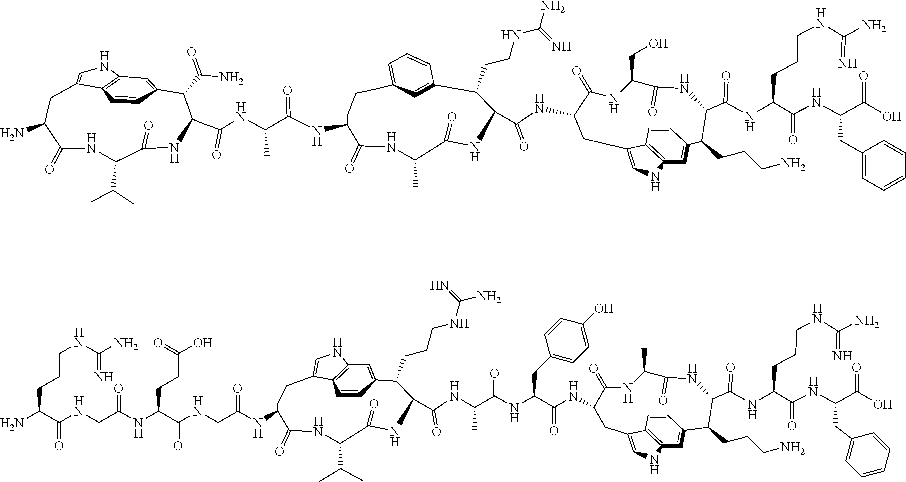

In some embodiments, the polypeptide is an isolated polypeptide.

In some embodiments, the polypeptide is characterised by an antibacterial activity. In some embodiments, the polypeptide is characterised by an antibacterial activity against Gram-negative bacteria. In some embodiments, the polypeptide is characterised by an antibacterial activity against drug-resistant bacteria.

In some embodiments, the polypeptide is characterised by a minimal inhibitory concentration (MIC) of about 2 μg/mL to about 10 μg/mL.

The present invention also provides a composition comprising a polypeptide as disclosed herein.

The present invention also provides a method of producing a polypeptide in a host cell, the method comprising:
a) introducing to the host cell one or more nucleic acid molecules, the nucleic acid molecules configured to express a precursor polypeptide (A), a rSAM/SPASM maturase (B), a protease (C), a transporter (D) and a protease/transporter (E);
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide in the host cell to form a modified precursor polypeptide with a cyclophane moiety connecting the X₁ and X₃ residues in each motif;
wherein the protease, transporter and protease/transporter are capable of cleaving the modified precursor polypeptide from the rSAM/SPASM maturase to form a cleaved modified polypeptide and exporting the cleaved modified polypeptide out from the host cell.

In some embodiments, at least the nucleic acid molecule configured to express A is derived from a Xye maturase system.

In some embodiments, the nucleic acid molecules configured to express A and B are from one Xye species and the nucleic acid molecules configured to express C, D and E are from another Xye species.

In some embodiments, at least the nucleic acid molecules configured to express C, D and E are fused.

In some embodiments, the nucleic acid molecules configured to express A and B are fused.

In some embodiments, the nucleic acid molecules configured to express B, C, D and E are fused.

In some embodiments, the nucleic acid molecules configured to express A, B, C, D and E are fused.

In some embodiments, the nucleic acid molecule configured to express A is at least 70% identical to and derived from a bacterial species selected from Serratia marcescens (smc), Erwinia toletana (etc), Photorhabdus australis (pac), Xenorhabdus nematophila (xnc), Xenorhabdus griffiniae VH1 (xgc), Pandoraea sp. PE-S2R-1 (psc), Pandoraea oxalativorans DSM 23570 (poc), Photorhabdus heterorhabditis Q614 (phc), Kosakonia cowanii pasteuri (kcc2 and kcc1), Bordetella bronchialis AU17976 (bbc) and Photorhabdus laumondii BOJ-47 (plc).

In some embodiments, the nucleic acid molecules configured to express C, D and E are at least 70% identical to and derived from Xenorhabdus nematophila (xnc).

In some embodiments, the rSAM/SPASM maturase has an amino acid sequence that is at least 70% identical to one of the following:

XncB:
(SEQ ID NO: 61)
MTTSKSEKIKHLEIILKISERCNINCSYCYVFNMGNSLATDSPPVISLDNVLALRGFFERSAAENEI
EVIQVDFHGGEPLMMKKDRFDQMCDILRQGDYSGSRLELALQTNGILIDDEWISLFEKHKVHASISI
DGPKHINDRYRLDRKGKSTYEGTIHGLRMLQNAWKQGRLPGEPGILSVANPTANGAEIYHHFANVLK
CQHFDFLIPDAHHDDDIDGIGIGRFMNEALDAWFADGRSEIFVRIFNTYLGTMLSNQFYRVIGMSAN
VESAYAFTVTADGLLRIDDTLRSTSDEIFNAIGHLSELSLSGVLNSPNVKEYLSLNSELPSDCADCV
WNKICHGGRLVNRFSRANRFNNKTVFCSSMRLFLSRAASHLITAGIDEETIMKNIQK
YkcB:
(SEQ ID NO: 62)
MEVITGSEGRVMLNLLIEKNIRHLEIILKISERCNINCDYCYVFNKGNSAADDSPARLSNKNIHHLV
CFLQRACQEYKIGTVQIDFHGGEPLLMKKENFTDMCIQLISGNYCGSNIRLALQTNATLIDNEWIAI
FEKYSVNVSISIDGPKHINDRHRLDTKGRSTYESTVRGLRILQNAYQQGRLPSDPGILCVTNAQANG
AEIYRHFVDELGVYSFDFLIPDDSYKDAHPDAVGIGRFLNEALDEWVKDNNAKIFVRLFQTHIASLL
GQKNSGVLGHTPNITGVYALTVSSDGFVRVDDTLRSTSDRMFNPIGHLSEVNLSNVFASPQFQEYSS
IGQSLPTECEGCIWENICAGGRIVNRFSTEDRFKHKSIYCYSMRTFLSRSSAHLLNMGIKEERIMAA
IRA
EtcB:
(SEQ ID NO: 63)
MTQLKGEKIKHLEIILKISERCNINCTYCYVFNMGNTLATDSTPVISLDNVYALRGFFERSAAENDI
EVIQVDFHGGEPLMMKKDRFDRMCQILLQGNYRSSKFELALQTNGILIDDEWIALFEKHQVHASISV
DGPKHINDRHRLDRKGKSTYEGTITGLRLLQNAWQQGRLPGEPGILSVANANANGAEIYRHFADTLQ
CQRFDFLIPDDHHDDSPDGEGVGRFLNEALDAWFADGRPEIFIRIFNTYLGTMLNSQFNRVLGMSAN
VESAYAFTVTADGMLRIDDTLRSTSDEIFNAVGHVSELSLARVLETSCVKEYLALSSNLPTVCAECV
WNNICHGGRLVNRFSRTNRFNNKTVFCKSMRLFLSRAASHLMASGVDEKEIMKNIQK
MscB
(SEQ ID NO: 64)
MAPGPARAALTEFVLKVHARCDLACDHCYVYEHADQSWRRRPVRMTPEVLRTAAGRIAEHAAAHDLP
DVTVILHGGEPLLLGAERLGEVLADLRRVIDPVTRLRLGMQTNGVLLSERLCDLLAEHDVAVGVSLD
GDRAANDRHRRFRSGAGSYDQVLRAIGLLRRPAYRRIYSGLLCTVDVRNDPIAVYESLLTQEPPRID
FLLPHATWDDPPWRPAGGGTAYAGWLRAVYDRWLADGRPVSVRLFDSLLSTAAGGPSGTEWLGLDPV
DLAVVETDGEWEQADSLKTAYDGAPATGMTVFSHAADDVAASPLLARRRSGRAGLSDECRRCPVVDQ
CGGGLFAHRYGAGHFDHPSVYCADLKELIVHVNENPPAPVRLDAGLPDDFIDRLAALTGDRVAIGRL
VEAQIAIVRALLAEVADRLPAGGAGADGWEALTALDRSAPESVARIAAHPYVRAWAVDCLAGSGTGA
RQGPDYLSALAVAAALDAGTPVRLDVPVRSGRLHLPTVGTVLLPEVGDGAARVETGPGSLRVAAGDV
TVAIRPGTPGDAPRWWPTRVLAAPDVSVLLEDGDPHRDCHRLPAGDRLDDAGAARWAETFAAAWQVI
RDEVPGHAEELRAGLRAVVPLRRSGAGVSEASTARQAFGGVAATETDAGSLAVLLVHEFQHSKMNAL
LDICDLVDGTRPIDITVGWRPDPRPAEAVLHGIYAHAAVADIWRIRADRQVDGAQAVYRRYRDWTAE
AIGALQRADALTPAGSRLVRQVARSMSGWPS
OscB:
(SEQ ID NO: 65)
MINPTLLNPEKIDISKFGPINLVVIQATSFCNLNCDYCYLPNRDLKNTLSLDLIEPIFKNIFNSPFV
GDEFTICWHAGEPLAVPISFYESAFQLIQAADQKYNQKQAKIWHSVQTNATYINQKWCDFIQEHNIC
VGVSLDGPEFIHDAHRQTRKGTGSHAQTMRGISFLQKNNIPFYVISVVTQDSLNYADEIFNFFRENG
IYDVGFNLEEIEGVNQSSTLEAVGTSEKYRAFMQRFWELTSEVQGEFNLREFEAICGLIYSNTRLTQ
TDMNNPFVLINIDYQGNFSTFDPELLSVNIKPYGNFILGNVLTDSFESVCDTEKFQKIYTDMQEGIK
LCRETCEYFGVCGGGAGSNKYWENGTFACSETMACRYRIKVVTDIILDKLENSLGLVENC
LscB:
(SEQ ID NO: 66)
MTISKMNLPVQTDNFRASSTLDLSAFGPINLVVIQSTSFCNLNCDYCYLRDRQSKNRLSLDLIEPIL
KTVLTSPFVGCDFTILWHAGEPLAMPISFYDSATALIREAERQYKTQPIQIFQSIQTNATLINQAWC
DCFRRNEIYVGVSLDGPAFLHDAHRQTYKGTGTHAATMRGISLLQKNEIPFNVICVLTQDSLDYPDE
IFNFFRSNRITEVGFNMEEAEGVHQHSTLDQQGTEERYRAFMQRFWDLTVQAKGEFKLREFETICTL
AYTGDRLGYTDMNQPFVIVNFDHQGNFSTFDPELLSFKIKEYGDFVLGNVLHNTLESVCQTEKFQKI
YQDMAAGVVQCRQSCEYFGLCGGGAGSNKYWENGTFNCTETKACRYRIKVIADIVLEGLENSLELAN
SIS
GscB
(SEQ ID NO: 67)
MSIVTSKPVINFKNTANFGPISLIIIQPNSFCNLDCDYCYLPDRHLQNKLSLDLIDPIFKSIFTSPF
LGCDFGVCWHAGEPLTMPVSFYKSAFQLIEEANTKYNKSEYSFYHSYQTNGTLINQGWCDLWQEYPV
HVGVSIDGPAFLHDVHRKNRKGGNSHDLTMRGIRYLQKNNIPYNTISVITEESLNYPDEMFNFFAEN
EIYDLAFNMEETEGVNELTSLNGIEIEHKYSQFIKRFWQLVTESKLPFIVREFEILISLIYSGNRLT
NTDMNKPFVIVNFDYQGNFSTFDPELLSVKTDKYGDFIFGNVLKDSLESICETEKFKTIYKDINDGV
KLCSDNCSYFGICGGGAGSNKYWENGTFASMETQACRYRIKILTDVLVSTIENSLGL
MscB-375
(SEQ ID NO: 68)
MAPGPARAALTEFVLKVHARCDLACDHCYVYEHADQSWRRRPVRMTPEVLRTAAGRIAEHAAAHDLP
DVTVILHGGEPLLLGAERLGEVLADLRRVIDPVTRLRLGMQTNGVLLSERLCDLLAEHDVAVGVSLD
GDRAANDRHRRFRSGAGSYDQVLRAIGLLRRPAYRRIYSGLLCTVDVRNDPIAVYESLLTQEPPRID
FLLPHATWDDPPWRPAGGGTAYAGWLRAVYDRWLADGRPVSVRLFDSLLSTAAGGPSGTEWLGLDPV
DLAVVETDGEWEQADSLKTAYDGAPATGMTVFSHAADDVAASPLLARRRSGRAGLSDECRRCPVVDQ
CGGGLFAHRYGAGHFDHPSVYCADLKELIVHVNENPPAPV.

In some embodiments, the rSAM/SPASM maturase is characterised by a rSAM domain and a SPASM domain;
wherein the rSAM domain is selected from CNINCSYC (SEQ ID NO: 69), CNINCDYCYVFNK (SEQ ID NO: 213), CNINCTYC (SEQ ID NO: 215), CDLACDHC (SEQ ID NO: 217), CNLNCDYC (SEQ ID NO: 219), CNLNCDYC (SEQ ID NO: 221), and CNLDCDYC (SEQ ID NO: 223); and
wherein the SPASM domain is selected from CADCVWNKIC (SEQ ID NO: 70), CEGCIWENIC (SEQ ID NO: 214), CAECVWNNIC (SEQ ID NO: 216), CRRCPVVDQC (SEQ ID NO: 218), CRETCEYFGVC (SEQ ID NO: 220), CRQSCEYFGLC (SEQ ID NO: 222), and CSDNCSYFGIC (SEQ ID NO: 224).

In some embodiments, the nucleic acid molecules are introduced into the host cell via a pET28a(+) vector, pCDFduet-1 vector, pACYCDuet-1 vector, pETDuet-1 vector, pCOLADuet-1 vector, pRSFDuet-1 vector, pBAD vector, or a combination thereof.

In some embodiments, the host cell is E. coli NiCo21(DE3), BL21(DE3), BL21-AI, BL21 Star™ (DE3) pLysS, Rosetta™ (DE3), or a combination thereof.

The present invention also provides a method of producing a polypeptide, the method comprising:
a) expressing a precursor polypeptide and a rSAM/SPASM maturase;
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide to form a polypeptide with a cyclophane moiety connecting the X₁ and X₃ residues in each motif.

The present invention also provides a method of synthesising a polypeptide as disclosed herein, the method comprising:
(a) coupling a pre-sequence peptide to a support, wherein said pre-sequence peptide comprises amino acid residues having side chain functionalities which are, if necessary, protected during the synthesis;
(b) coupling one or more N-protected amino acids to the N-terminus of the pre-sequence peptide to form a precursor polypeptide, wherein each coupling is performed in stepwise fashion and under conditions in which each of the amino acids of the target peptide is coupled and subsequently N-deprotected;
c) cleaving said precursor polypeptide from the support; and
d) synthetically or enzymatically connecting the X₁ and X₃ in each motif to form a cyclophane moiety.

The present invention also provides a method of modifying a precursor polypeptide, the precursor polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue; and
wherein at least one of the two C-terminus residues is an aromatic residue; the method comprising:
enzymatically connecting the X₁ and X₃ residues in each motif to form a cyclophane moiety.

In some embodiments, the enzyme is rSAM/SPASM maturase.

The present invention also provides a method of treating a bacterial infection, comprising administering an effective amount of a polypeptide as disclosed herein to subject in need thereof.

In some embodiments, the bacterial infection is a Gram-negative bacterial infection. In some embodiments, the bacterial infection is characterised by a drug-resistance.

In some embodiments, the bacterial infection is caused by a Gram-negative bacteria selected from Escherichia coli, Pseudomonas aeruginosa, Candidatus Liberibacter, Agrobacterium tumefaciens, Acinetobactor baumannii, Moraxella catarrhalis, Citrobacterdi versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baurmannii, Salmonella enteriditis, Fusobacterium nucleatum, Veillonella parvula, Actinobacillus actinomycetemcomitans, Aggregatibacter actinomycetemcomitans, Porphyromonas gingivalis, Helicobacter pylori, Francisella tularensis, Yersinia pestis, Vibrio cholera, Morganella morganii, Edwardsiella tarda, Campylobacter jejuni, Haemophilus influenza, Enterobacter cloacae, or a combination thereof.

### BRIEF DESCRIPTION OF THE DRAWINGS

Embodiments of the present invention will now be described, by way of non-limiting example, with reference to the drawings in which:

FIG. 1. Biosynthesis and types of Xenorceptides.

FIG. 2. Chemically-guided workflow for RiPP antibiotic discovery (GEnSyBER-A). Genomic enzymology identifies sequence-function space of a RiPP family based on posttranslational modifying enzyme. Synthetic biology provides the targeted natural products. Structure elucidation unveils the chemical structure. Antibacterial assays reveal any bioactivity against pathogens of interest. Sequence similarity network containing SPASM/Twitch proteins (Alignment score=45) taken from RadicalSAM.org.

FIG. 3. Production of Xenorceptides. a, Coexpression of His₆-SmcA+SmcB. b, Production of natural product using a 2-vector system, His₆-AB/pET28+CDE/pCDFDuet-1. EICs show cleaved leader (left) and natural product (right) detected only when coexpressed with SmcCDE. HR-MS for 2 is shown. c, Summary of constructs used to produce 2-4. Coexpressions with XncCDE provide increased production of natural product.

FIG. 4. Source BGCs/strains, structures, and NOESY correlations. a, Structures of xenorceptide A1 (1), xenorceptide A2 (2), xenorceptide A3 (3), and xenorceptide A4 (4). b, Key NOESY correlations used to assign the substitution and conformation of Phe- and Tyr-derived cyclophanes.

FIG. 5. Biological evaluation of xenorceptide A2 (2). a, Time-kill kinetics of xenorceptide A2 (2) against E. coli M6 over 24 h. Colistin at 2×MIC was tested as a positive control. Black dotted lines indicate the limit of detection (50 CFU/mL). Experiments were repeated on three biologically independent samples. Data are presented as geometric mean±SE. b, SEM images of E. coli M6 cells either untreated or after treatment with 8×MIC xenorceptide A2 (2) for 2 h. For each sample slide, at least five independent fields were imaged to ensure representativeness. Magnification=20,000×. c, the development of resistance of E. coli M6 against xenorceptide A2 (2) was monitored using serial passage over 14 days. Experiments were repeated on three independent starting cultures.

FIG. 6. Test expression of xnc genes. a, Test expression for precursor and rSAM/SPASM by coexpression of His₆-XncA+XncB. EICs show modified fragment. HR-MS for the modified fragment is shown. b, Coexpression using a 2-vector system, His₆-xncAB/pET28+xncCDE/pCDFDuet-1. EICs show cleaved leader, suggesting peptidase cleaves precursor peptide.

FIG. 7. xye BGCs from Serratia marcescens, Erwinia toletana, and Photorhabdus australis.

FIG. 8. Production of xenorceptide A3. a, Test expression for precursor and rSAM/SPASM by coexpression of His₆-EtcA+EtcB. b, Production of natural product using a 2-vector system, His₆-etcAB/pET28+etcCDE/pCDFDuet-1. EICs show cleaved leader (left) only when coexpressed with EtcCDE, while natural product is not detected (right).

FIG. 9. Production of xenorceptide A4. a, Test expression for precursor and rSAM/SPASM by coexpression of His₆-EtcA+EtcB. b, Production of natural product using a 2-vector system, His₆-pacAB/pET28+pacCDE/pCDFDuet-1. EICs show cleaved leader (left) only when coexpressed with PacCDE, while natural product is not detected (right).

FIG. 10. RiPP cyclophane natural products: darobactin, dynobactin, and triceptides. a, Chemical structures for darobactin, dynobactin and xenorceptide A1 from the dar, dyn, and xnc BGCs respectively. Xenorceptide A1 is a representative xenorceptide. b, Canonical cyclophanes from each class. c, Schematic showing location of Cys residues corresponding to three Fe-S clusters in DarE, DynA, and 3-CyFE maturases. The CX3CX2C motif for the rSAM Fe-S cluster and the CX2-3CX4-6C motif with additional Cys for Aux II are commonly conserved in all groups while 3-CyFEs lack the Cys residues corresponding to Aux I cluster. d, Sequence-function space of rSAM/SPASM proteins containing 3-CyFEs (n=13,151; AS=75; 40% representative nodes). Nodes are based on maturase type. XncB, DarE, and DynA are annotated.

FIG. 11. Summary of xenorceptide biosynthesis, precursor types, phylogeny of maturases, and representative BGCs. a, A phylogenetic tree made by Clustal Omega summarizing gene sequences encoding rSAM/SPASM XyeB proteins associated with a type A XyeA precursor. Sequence logos are shown for XyeA core sequences of each genus. b, Representative xye BGCs from each genus.

FIG. 12. Synthetic biology for the production of xenorceptides. a, Production of natural product using strategy 2, engineered His₆-A/pET28a(+)+BCDE/pCDFDuet-1 (strategy 2). The precursor constituted of His-tagged XncA leader and YkcA core sequence (His₆-XncA_{L}-YkcA_{C}) is co-expressed with XncBCDE. This strategy gave a better yield of the ykc natural product (5) than strategy 1. b, Summary of xenorceptides named xenorceptides A2-A10 (2-10) produced in this study. Characteristic motifs/residues are highlighted in red. Products 9 and 10 could not be isolated due to the low yield.

FIG. 13. Biological evaluation of xenorceptide A2 (2). a, Time-kill kinetics of xenorceptide A2 (2) against E. coli M6 over 24 h was determined by agar colony count. Colistin at 2×MIC was tested as a positive control. Black dotted lines indicate the limit of detection (50 CFU/mL). Experiments were repeated on three biologically independent samples. Data are presented as geometric mean±SE. b, The development of resistance of E. coli M6 against xenorceptide A2 (2) was monitored using serial passage over 14 days. Experiments were repeated three times with different starting bacteria cultures. c, SEM images of E. coli M6 after treatment with xenorceptide A2 (2) at 4× or 8×MIC for 2 h. For each sample slide, at least five independent fields were imaged to ensure representativeness. Magnification=25,000×. Scale bar=1 μm. d, Experiment schematics of the mouse peritonitis model infected with E. coli M6 for evaluating the in vivo efficacy of xenorceptide A2 (2). e, Bacteria burden in the peritoneal fluid, blood, liver, spleen, and kidney of C57BL/6NTac mice (n=5 mice per treatment group) collected 5 h after treatment with 5 mg/kg xenorceptide A2 (2), 50 mg/kg xenorceptide A2 (2), 5 mg/kg colistin, or saline (vehicle control). Samples were plated onto LB agar and incubated for 18-20 h at 37° C. before colony count. Colony counts of organ tissues were normalized against the average mass of the respective mouse organs. Statistical significance of differences between data groups were evaluated using one-way analysis of variance (ANOVA) followed by Turkey post-hoc test (ns: p>0.05, *: p≤0.05, **: p≤0.01).

FIG. 14. Synthetic biology for the production of 11 by co-expression of His₆-A/pET28a(+)+BCDE/pCDFDuet-1 (strategy 2).

FIG. 15. Synthetic biology for the production of 12 by co-expression of His₆-A/pET28a(+)+BCDE/pCDFDuet-1 (strategy 2).

FIG. 16. Synthetic biology for the production of 13 by co-expression of His₆-A/pET28a(+)+BCDE/pCDFDuet-1 (strategy 2).

FIG. 17. Summary of Xye Type B and Type D biosynthetic gene clusters and the corresponding sequence of the precursor.

FIG. 18. LC-MS analysis of coexpression of His6-XgcA1B and full cluster expression His6-XgcA1B+DEC full-length precursors. (a) XgcA1 sequence with His6-tag. (b) Blue fill shows the truncated leader only existed in full-cluster expression. (c) MS of truncated leader from GG. *A1BDEC=Full-cluster expression, A1B=XgcA1B only.

FIG. 19. LC-MS analysis of coexpression of His6-PlcAB digested with trypsin and full cluster expression His6-PlcAB+PlcCDE full-length precursors. (a) PlcA sequence with His6-tag. (b-e) LC-MS analysis of PlcAB and PlcAB+PlcCDE full-length precursors. (b) Blue fill shows the truncated leader only existed in full-cluster expression. (c, d) MS of truncated leader from GG. (e) LC-MS of extracted ion chromatogram (EIC) data of PlcAB and PlcAB+PlcCDE tryptic fragment, the red arrows indicating that the plc precursor in Plc full cluster expression cleavage at GG (red arrow), while PlcAB only expression does not exhibit this cleavage. *ABCDS=Full-cluster expression, AB=PlcAB only

FIG. 20. The xgc biosynthetic gene cluster, the protein sequence of XgcA1 and XgcA2 are given at right side.

FIG. 21. The phc biosynthetic gene cluster, the protein sequence of PhcA is given at right side.

FIG. 22. (a) The kcc2 and kcc1 biosynthetic gene clusters, the protein sequence of Kcc2A and Kcc1A are given at right side. (b) LC-MS analysis of SPE elute fraction of Kcc2AB+Kcc2CDE, with 24-26 indicating Kcc2 products. (c) LC-MS analysis of SPE elute fraction of Kcc1AB+Kcc2CDE, with 27-29 indicating Kcc1 products.

FIG. 23. LC-MS analysis of variants. (a) Co-expression of XgcA2(G-1K) and XgcB, followed by trypsin digestion leads to the formation of compound 22. (b) Co-expression of Kcc1(G-1E) and Kcc1B, followed by GluC digestion leads to the formation of compound 27 and 28. (c) Co-expression of Poc_leader/Bbc_core_(G-1K) fusion precursor and PocB, followed by trypsin digestion leads to the formation of compound 30 and 31. For 31, b&y ions in MS data suggested the −2D modification is localized to the WSK motif. (d) Co-expression of Poc(G-1R) and PocB, followed by trypsin digestion leads to the formation of compound 32 and 33. For 33, b&y ions in MS data suggested the −2D modification is localized to the WSR motif.

FIG. 24. Structure of compound 24. Peptide sequences for compound 24 (top), and structure of residues +5 to +12 of fragment (bottom). Blue connectors in the core peptide sequences indicate modifications (−2 Da) detected and localized by LC-MS/MS.

FIG. 25. Key features of Kcc2-4D HMBC (a) and COSY (b), showing the correlation between Trp5-C6 and Arg7β and Trp10-C6 and Lys12p C—C bond formation.

FIG. 26. Structure elucidation of xenorceptide A2 (2). a, Key 2D NMR correlation of 2. b, Conformational analysis and NOE correlations for WVN (left), FAR (center), and WSK (right) motifs.

FIG. 27. Structure elucidation of xenorceptide A3 (3). a, Key 2D NMR correlation of 3. b, Conformational analysis and NOE correlations for WVN (left), FAN (center), and WTK (right) motifs.

FIG. 28. Structure elucidation of xenorceptide A4 (4). a, Key 2D NMR correlation of 4. b, Conformational analysis and NOE correlations for WVN (left), YAR (center), and WTK (right) motifs.

FIG. 29. 1H NMR spectrum of xenorceptide A2. Acquired at 800 MHz in DMSO-d6 at 298 K.

FIG. 30. TOCSY xenorceptide A2. Acquired at 800 MHz in DMSO-d6 at 298 K.

FIG. 31. Phase-sensitive NOESY spectrum of xenorceptide A2. Acquired at 800 MHz in DMSO-d6 at 298 K.

FIG. 32. HSQC spectrum of xenorceptide A2. Acquired at 800 MHz in DMSO-d6 at 298 K.

FIG. 33. HMBC spectrum of xenorceptide A2. Acquired at 800 MHz in DMSO-d6 at 298 K.

FIG. 34. 1H NMR spectrum of xenorceptide A3. Acquired at 400 MHz in DMSO-d6+0.3% TFA-d at 298 K.

FIG. 35. COSY spectrum of xenorceptide A3. Acquired at 400 MHz in DMSO-d6+0.3% TFA-d at 298 K.

FIG. 36. TOCSY spectrum of xenorceptide A3. Acquired at 400 MHz in DMSO-d6+0.3% TFA-d at 298 K.

FIG. 37. Phase-sensitive NOESY spectrum of xenorceptide A3. Acquired at 400 MHz in DMSO-d6+0.3% TFA-d at 298 K.

FIG. 38. Edited-HSQC spectrum of xenorceptide A3. Acquired at 400 MHz in DMSO-d6+0.3% TFA-d at 298 K.

FIG. 39. HMBC spectrum of xenorceptide A3. Acquired at 400 MHz in DMSO-d6+0.3% TFA-d at 298 K.

FIG. 40. 1H NMR spectrum of xenorceptide A4. Acquired at 400 MHz in DMSO-d6+0.2% TFA-d at 298 K.

FIG. 41. COSY spectrum of xenorceptide A4. Acquired at 400 MHz in DMSO-d6+0.2% TFA-d at 298 K.

FIG. 42. TOCSY spectrum of xenorceptide A4. Acquired at 400 MHz in DMSO-d6+0.2% TFA-d at 298 K.

FIG. 43. Phase-sensitive NOESY spectrum of xenorceptide A4. Acquired at 400 MHz in DMSO-d6+0.2% TFA-d at 298 K.

FIG. 44. Edited-HSQC spectrum of xenorceptide A4. Acquired at 400 MHz in DMSO-d6+0.2% TFA-d at 298 K.

FIG. 45. HMBC spectrum of xenorceptide A4. Acquired at 400 MHz in DMSO-d6+0.2% TFA-d at 298 K.

FIG. 46. 1H spectrum of product xenorceptide D1. Acquired at 400 MHz in DMSO at 298 K.

FIG. 47. COSY spectrum of product xenorceptide D1. Acquired at 400 MHz in DMSO at 298 K.

FIG. 48. TOSCY spectrum of product xenorceptide D1. Acquired at 400 MHz in DMSO at 298 K.

FIG. 49. HSQC spectrum of product xenorceptide D1. Acquired at 400 MHz in DMSO at 298 K.

FIG. 50. HMBC spectrum of product xenorceptide D1. Acquired at 400 MHz in DMSO at 298 K.

FIG. 51. TOSCY spectrum of product xenorceptide D1. Acquired at 400 MHz in DMSO at 298 K.

### DETAILED DESCRIPTION

The term “cyclophane group” or “cyclophane” may be used interchangeably to refer to a macrocycle or ring consisting of an aromatic unit (aryl or heteroaryl) and an optionally substituted aliphatic chain that forms a bridge between two non-adjacent positions of the aromatic ring. For example, the “cyclophane group” or “cyclophane” can refer to a macrocycle or ring formed when an aromatic unit in an aromatic amino acid X₁ (such as W, F, Y or H) in a peptide comprising a 3 residue motif X₁-X₂-X₃ is joined to a Cβ in X₃ via a carbon to carbon bond.

The terms “polypeptide”, “peptides” and “protein” are used interchangeably and include any polymer of amino acids (dipeptide or greater) linked through peptide bonds or modified peptide bonds, whether produced naturally or synthetically. The polypeptides of the invention may comprise non-peptidic components, such as carbohydrate or fatty acid groups.

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refer to compounds that have the same basic chemical structure as a naturally occurring amino acid, by way of example, an a carbon that is bound to a hydrogen, a carboxyl group, an amino group, and an R group. Such analogs may have modified R groups (by way of example, norleucine) or may have modified peptide backbones, while still retaining the same basic chemical structure as a naturally occurring amino acid. Non-limiting examples of amino acid analogs include homoserine, norleucine, methionine sulfoxide, methionine methyl sulfonium. The amino acid as referred to herein may be a D or L amino acid. The amino acid may also be a β-amino acid. The term “amino acid” can include D-amino acids, α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid, non-conventional amino acids such as compounds which have an amine and carboxyl functional group separated in a 1,3 or larger substitution pattern, such as β-alanine, y-amino butyric acid, Freidinger lactam, the bicyclic dipeptide (BTD), amino-methyl benzoic acid and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art are also included.

A “conservative amino acid substitution” is one in which the amino acid residue is replaced with an amino acid residue having a similar side chain. Families of amino acid residues having similar side chains have been defined in the art, which can be generally sub-classified as follows:

TABLE 1
Amino Acid Subclassification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine, Histidine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary and Preferred Amino Acid Substitutions

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Unnatural amino acids may include amino acids which are not in the L conformation. These can include non-a amino acids such as P amino acids and D amino acids. Unnatural amino acids incorporated into peptides may include 1) a ketone reactive group (as found in para or meta acetyl-phenylalanine) that can be specifically reacted with hydrazines, hydroxylamines and their derivatives (Addition of the keto reactive group to the genetic code of Escherichia coli. Wang L, Zhang Z, Brock A, Schultz P G. Proc Natl Acad Sci USA. 2003 Jan. 7; 100(1):56-61; Bioorg Med Chem Lett. 2006 Oct. 15; 16(20):5356-9. Genetic introduction of a diketone-containing amino acid into proteins. Zeng H, Xie J, Schultz P G), 2) azides (as found in p-azido-phenylalanine) that can be reacted with alkynes via copper catalysed “click chemistry” or strain promoted (3+2) cyloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124(31):9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1; 125(39):11782-3), or azides that can be reacted with aryl phosphines, via a Staudinger ligation (Selective Staudinger modification of proteins containing p-azidophenylalanine. Tsao M L, Tian F, Schultz P C. Chembiochem. 2005 December; 6(12):2147-9), to form the corresponding amides, 4) Alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15(5):1521-4), 5) Boronic acids (boronates) than can be specifically reacted with compounds containing more than one appropriately spaced hydroxyl group or undergo palladium mediated coupling with halogenated compounds (Angew Chem Int Ed Engl. 2008; 47(43):8220-3. A genetically encoded boronate-containing amino acid, Brustad E, Bushey M L, Lee J W, Groff D, Liu W, Schultz P G), 6) Metal chelating amino acids, including those bearing bipyridyls, that can specifically co-ordinate a metal ion (Angew Chem Int Ed Engl. 2007; 46(48):9239-42. A genetically encoded bidentate, metal-binding amino acid. Xie J, Liu W, Schultz P G).

The majority of strains on the WHOs Priority Pathogens List for R&D of new antibiotics belong to the family Enterobactericiae and include Klebsiella pneumoniae, Escherichia coli, Enterobacter spp., Serratia spp., Proteus spp., Providencia spp., and Morganella spp. These strains are multi-drug resistant and lead to severe and deadly infections in hospitals and nursing homes. The discovery of new antibiotics with the ability to treat these infections will have significant impact in the clinic and can save thousands of lives annually.

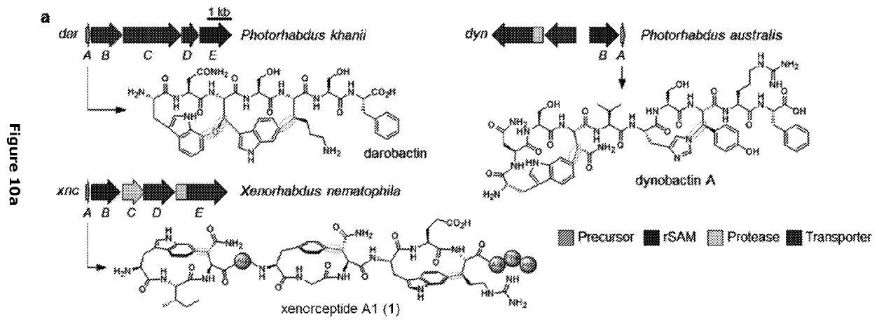

The present invention is predicated on the understanding that RiPP cyclophane-containing natural products may be a source of antibiotics against Gram-negative pathogens. For example, Darobactin was isolated from Photorhabdus khanii in efforts targeting animal associated symbionts as a promising source of new antibiotics. The structure of darobactin is composed of two fused three-residue cyclophanes and an ether linkage (FIG. 10a). Homologues of the maturase DarE, have also been characterized to install an ether which is a characteristic feature for this class of maturases and products (FIG. 10b). Dynobactin was recently reported by a research group by expanding on this class of natural products bioinformatically and optimizing the purification protocol by testing of purified fractions. Dynobactin contains one four-residue and one three-residue cyclophane with the latter incorporating an imidazole via Nε2 linkage (FIG. 10a). Sequence comparison of DynA precursors shows the 4-residue cyclophane is likely conserved while the second cyclophane appears to be formed between two aromatic residues (FIG. 10b).

In an alternative approach to natural products drug discovery, the inventors pursued identification of a new RiPP family prior to knowledge of the bioactivity of the natural products. The rationale was that new RiPP families will contain new products for screening platforms and biosynthetic enzymes that could be applied for making drug-like molecules. To do this the inventors systematically characterized three unique TIGRFAMs annotated as rSAM/SPASM maturases (Xye, TIGR04996: Grr, TIGR04261; and Fxs, TIGR04269) and found they are unified in their ability to catalyze 3-residue cyclophane formation. Cyclophane formation occurs via a C(sp²)-Cβ(sp³) bond between an aromatic ring and β-position on 3-residue Ω1-X2-X3 motifs where all aromatic residues (Phe, Trp, Tyr, and His) appear at the Ω1 position (FIG. 10b). Collectively, the maturases is referred to as 3-residue cyclophane forming enzymes (3-CyFEs). 3-CyFEs can be differentiated from DarE, DynA, and other radical SAM/SPASM maturases by the lack of Cys residues that bind auxiliary cluster 1 of the SPASM domain (FIG. 10c). BGCs that contain at least one 3-CyFE define a new family of RiPPs are termed as triceptides. 3-CyFEs were localized within a region of rSAM/SPASM sequence-function space and analysis of this biosynthetic landscape allowed the identification of ˜4000 triceptide precursors which are broadly distributed in bacteria (FIG. 10d). With a new RiPP family identified the inventors focused on a specific maturase system for antibiotic discovery.

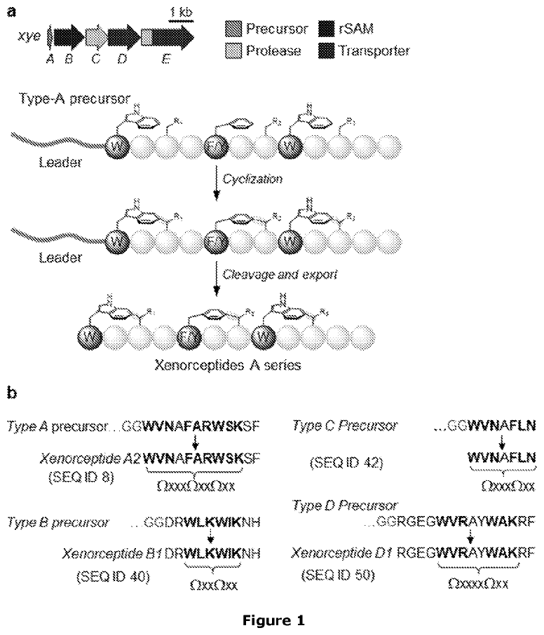

As the activity and function for triceptides was unknown, the Xye maturase systems (GenProp1090) as a source of potential antibiotics for several reasons. First, xye BGCs are reminiscent of Class I bacteriocins, a well-known source of antibacterial peptides. Shared biosynthetic features include precursors encoding a Gly-Gly motif that separates the leader and core peptide, and protease/transporter proteins that cleave and export the mature RIPP (FIGS. 10a and 1a). Second, most xye BGC-containing bacteria are isolated from human or animal microbiomes. Since these end products are likely secreted and act in a biological environment similar to that experienced by clinically used antibiotics, the inventors hypothesize that these molecules would have evolved ideal drug-like features. Third, the inventors previously demonstrated production of xenorceptide A1, as a representative from the Xye maturase system. To their knowledge, xenorceptide A1 is the first characterized triceptide natural product. The inventors collectively refer to the triceptides derived from the Xye maturase systems as xenorceptides. Although xenorceptide A1 was not active when tested against several bacterial strains, the inventors believed that the production of xenorceptide A1 provided an entry point to produce and study this subfamily further. The inventors hypothesized that the diversity in bacterial and core sequences within XyeA precursors had the potential to generate peptide antibiotics.

The bioinformatic analysis and synthetic biology enabled production of xenorceptides is now disclosed herein. Screening of the natural products against Gram-negative and Gram-positive pathogens revealed xenorceptide A2 which was subjected to further biological evaluation. This study adds Xenorceptides to the RIPP cyclophane antibiotic class, and identified xenorceptide A2 as an antibiotics candidate.

The present invention provides a polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein X₁ and X₃ in each motif are connected to form a cyclophane moiety;
wherein at least one of the two C-terminus residues is an aromatic residue.

The present invention provides a polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₂-X₂-X₃;
wherein each X₁ is a residue independently selected from tryptophan, phenylalanine, tyrosine, histidine, or an unnatural aromatic amino acid residue;
wherein each X₂ and X₃ are independently any amino acid residue;
wherein X₁ and X₃ in each motif are connected to form a cyclophane moiety;
wherein at least one of the two C-terminus residues is an aromatic residue; and
wherein X₁ and X₃ in the second motif are connected via phenylene to form a cyclophane moiety.

A cyclophane is a hydrocarbon consisting of an aromatic unit and a chain that forms a bridge between two non-adjacent positions of the aromatic ring.

When the polypeptide comprises two three residue motifs, the two three residue motifs may be referred to as a first three residue motif (from the N-terminus) and a second three residue motif (following the first motif).

The three residue motif may be each represented by X₁-X₂-X₃.

The polypeptide is modified such that X₁ and X₃ in each motif are linked. The linkage may be via W, F, Y or H to form imidazolylene, indolylene or phenylene-bridged cyclophanes. The modified polypeptide may, for example, display restricted rotation of the aromatic ring and induce planar chirality in the asymmetric indole bridge. In some embodiments, X₁ and X₃ are connected via phenylene or indolylene to form a cyclophane moiety. In some embodiments, X₁ and X₃ in the second motif are connected via phenylene to form a cyclophane moiety.

In some embodiments, X₁ is each X₁ is a residue independently selected from tryptophan, phenylalanine, tyrosine, histidine, an unnatural aromatic amino acid residue or a derivative thereof. In some embodiments, the first X₁ is a residue selected from tryptophan, phenylalanine, tyrosine, histidine, an unnatural aromatic amino acid residue or a derivative thereof. In some embodiments, the first X₁ is a residue selected from tryptophan, phenylalanine, tyrosine, histidine or a derivative thereof. In some embodiments, the first X₁ is a residue selected from tryptophan, phenylalanine or a derivative thereof. In some embodiments, the second X₁ is a residue selected from tryptophan, phenylalanine, tyrosine, histidine, an unnatural aromatic amino acid residue or a derivative thereof. In some embodiments, the second X₁ is a residue selected from tryptophan, phenylalanine, tyrosine, histidine or a derivative thereof. In some embodiments, the second X₁ is a residue selected from tryptophan, phenylalanine, tyrosine or a derivative thereof. In some embodiments, the second X₁ is a residue selected from phenylalanine, tyrosine or a derivative thereof.

X₂ and X₃ may each independently be any amino acid. In some embodiments, X₂ is I, G, E, Y, V, L, A, D, S, T, N or Q. X₃ may be a non-aromatic amino acid. In some embodiments, X₃ is an amino acid that is not W, F, Y or H. In some embodiments, X₃ is N, R, S, D, Q or K. In some embodiment, X₃ is N, R or K.

In some embodiments, X₂ is I, G, E, Y, V, L, A, D, S, T, N or Q, and X₃ is N, R, S, D or K. In some embodiments, X₂ is I, G, E, Y, V, L, A, D, S, T, N or Q, and X₃ is N, R or K.

In some embodiments, the first and second three residue motifs are separated by 0 amino acid residue. In some embodiments, the first and second three residue motifs are separated by 1 to 3 amino acid residue. In some embodiments, the two three residue motifs are separated by 1 to 2 amino acid residue. In some embodiments, the two three residue motifs is separated by 1, 2 or 3 amino acid residue.

The first and second three residue motifs may be separated by any type of amino acid residue, natural or non-natural. In some embodiments, the two three residue motifs is separated by a residue selected from A, V, Y, F, T, Q, G, L, D, or S. In some embodiments, the two three residue motifs is separated by A.

In some embodiments, the first three residue motif is not fused with the second three residue motif other than via 1-3 amino acid residues or an amide bond. In other embodiments, the cyclophane moiety in the first three residue motif is not fused to the cyclophane moiety in the second three residue motif. In some embodiments, the cyclophane moieties connecting X₁ and X₃ in each motif are not fused to each other. In this regard, in contrast to darobactin for example, the polypeptide of the present invention does not comprise linked three-residue cyclophanes. The polypeptide of the present invention also does not comprise an ether linkage between the three-residue cyclophanes motifs.

The C-terminus comprises at least two residues. These residues do not form part of the three residue motif. In some embodiments, the C-terminus comprises at least three residues, or at least four residues. In other embodiments, the C-terminus comprises 2 to S residues, 2 to 7 residues, 2 to 6 residues, 2 to 5 residues, or 2 to 4 residues. In some embodiments, the C-terminus comprises at least three residues.

At least one of the two C-terminus residues is an aromatic residue. For example, at least one of the C-terminus residue may be tryptophan, tyrosine, phenylalanine, or histidine. In some embodiments, at least one of the two C-terminus residues is a polar and/or basic residue. In some embodiments, the C-terminus comprises an aromatic residue and a polar and/or basic residue.

It was found that having at least an aromatic residue at the C-terminus improves the anti-bacterial property of the polypeptide.

In some embodiments, the polypeptide comprises at least three three residue motifs. In this regard, the three three residue motifs may be referred to as a first motif (from the N-terminus), a second motif (following the first motif), and a third motif (following the second motif and in proximity to the C-terminus).

In some embodiments, the third X₁ is a residue independently selected from tryptophan, phenylalanine, tyrosine, histidine, an unnatural aromatic amino acid residue or a derivative thereof. In some embodiments, the third X₁ is a residue independently selected from tryptophan, phenylalanine, tyrosine, histidine or a derivative thereof. In some embodiments, the third X₁ is a residue independently selected from tryptophan, phenylalanine or a derivative thereof.

In some embodiments, when the polypeptide comprises a third three residue motifs, X₃ of the second motif (from the N-terminus) and X₁ of the third motif are covalently bonded to each other via an amide bond. Accordingly, the second motif and the third motif are not separated by any residue.

In one embodiment, the polypeptide is a linear polypeptide. The polypeptide may be of any sequence length, having any number of residues at the N-terminus or C-terminus as long as it comprises at least two three residue motif optionally separated by 1 to 3 amino acid residue and at least two C-terminus residues.

In some embodiments, the polypeptide is represented by Formula (I):

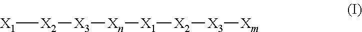

wherein each X₁ is a residue independently selected from tryptophan, phenylalanine, tyrosine, histidine or an unnatural aromatic amino acid residue;
wherein each X₂ and X₃ are independently any amino acid residue;
wherein Xₙ is an amide bond or 1 to 3 amino acid residue; and
wherein Xₘ is at least two C-terminus residues.

In some embodiments, the polypeptide is represented by Formula (I′):

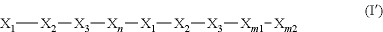

wherein Xₘ₁ is a first C-terminus residue; and
Xₘ₂ is a second C-terminus residue.

In some embodiments, each X₂ is an amino acid residue, the amino acid independently selected from leucine, isoleucine, valine, alanine, proline, serine, lysine, asparagine, phenylalanine, aspartic acid or a derivative thereof.

In some embodiments, each X₃ is an amino acid residue, the amino acid independently selected from lysine, glutamine, asparagine, arginine or a derivative thereof. In some embodiments, each X₃ is an amino acid residue, the amino acid independently selected from lysine, asparagine, arginine or a derivative thereof.

In some embodiments, the polypeptide is represented by Formula (II):

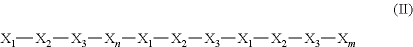

wherein each X₁ is a residue independently selected from tryptophan, phenylalanine, tyrosine, histidine or an unnatural aromatic amino acid residue;
wherein each X₂ and X₃ are independently any amino acid residue;
wherein Xₙ is an amide bond or 1 to 3 amino acid residue; and
wherein Xₘ is at least two C-terminus residues.

In some embodiments, the polypeptide is represented by Formula (II′):

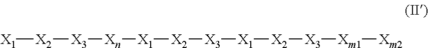

wherein Xₘ₁ is a first C-terminus residue; and
Xₘ₂ is a second C-terminus residue.

In some embodiments, each X₂ is an amino acid residue, the amino acid independently selected from valine, isoleucine, phenylalanine, tryptophan, alanine, leucine, glycine, serine, proline, threonine, aspartic acid, asparagine, glutamic acid, arginine or a derivative thereof.

In some embodiments, each X₃ is an amino acid residue, the amino acid independently selected from arginine, lysine, asparagine or a derivative thereof.

In some embodiments, X₁ and X₃ in the first motif are connected via indolylene to form a cyclophane moiety. In some embodiments, X₁ and X₃ in the second motif are connected via phenylene to form a cyclophane moiety.

In some embodiments, the polypeptide is represented by Formula (Ia) or (IIa):

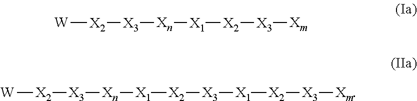

In some embodiments, X₁ is W. In some embodiments, X₁ of the first motif is W. In some embodiments, when X₁ is W, X₁ (or W) is connected to X₃ via a 3,6 or 3,7 disubstituted indolylene moiety. This may for example be represented pictorially as follows:

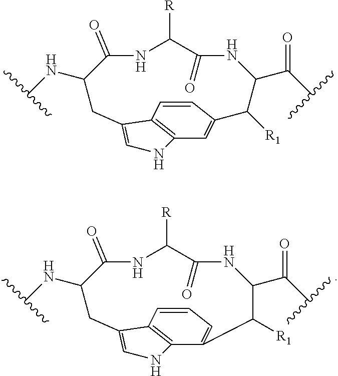

In some embodiments, the polypeptide is represented by Formula (Ia′) or (IIa′):

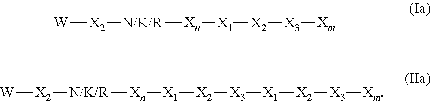

In some embodiments, the polypeptide is represented by Formula (Ib) or (IIb):

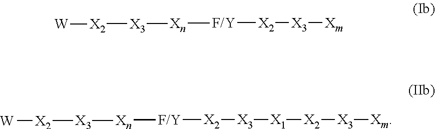

In some embodiments, X₁ is F or Y. In some embodiments, X₁ of the second motif is F or Y. In some embodiments, when X₁ is F or Y, X₁ (being F or Y) is connected to X₃ via a 1,3 or 1,4 disubstituted phenylene moiety. The 1,4 disubstituted phenylene moiety may for example be represented pictorially as follows:

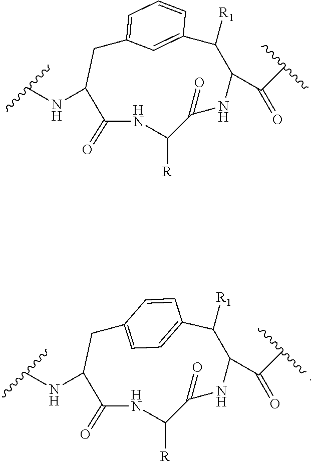

In some embodiments, the polypeptide is represented by Formula (Ib′) or (IIb′):

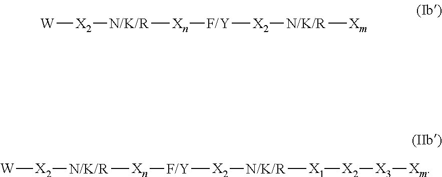

In some embodiments, the polypeptide is represented by Formula (IIc):

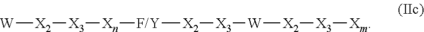

In some embodiments, the polypeptide is represented by Formula (IIc):

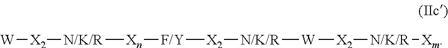

In some embodiments, when X₁ in the first motif is F, the polypeptide is represented by Formula (Id) or (IId):

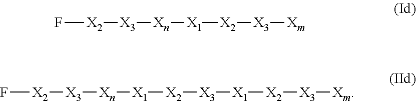

Such polypeptides may be Type D peptides.

In some embodiments, the polypeptide is represented by Formula (Id′) or (IId′):

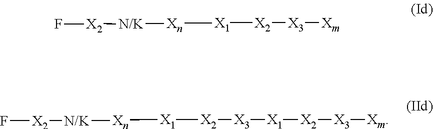

In some embodiments, the polypeptide is represented by Formula (Ie) or (IIe):

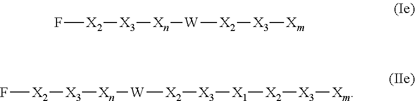

In some embodiments, the polypeptide comprises 3 three residue motifs, wherein X₁ of the second three residue motif is F, X₃ of the second and third three residue motifs are independently basic amino acid residues, and at least one of the two C-terminus residues is an aromatic residue.

In some embodiments, the polypeptide is selected from Table 3:

TABLE 3
Xenorceptides

| | | | | | | MIC |
|---|---|---|---|---|---|---|
| SEQ | | xenor- | | Core | | (E. |
| ID | Type^{e} | ceptide^{f} | Bacterial strain | Sequence^{ª} | Length^{d} | coli)^{b} |
| 1 | A | | Xenorhabdus sp. | WVNAFANWSKAL | 51 | |
| | | | NBAII XenSa04 | | | |
| 2 | A | | Xenorhabdus stockiae | WVNAFGNWSKSL | 51 | |
| | | | DSM 17904 | | | |
| 3 | A | A6 (6) | Xenorhabdus sp. BG5 | WVNAFANWSKSF | 51 | |
| 4 | A | | Kosakonia cowanii | WVNAFARWGKSF | 51 | |
| | | | pasteuri | | | |
| 5 | A | | Yersinia sp. Marseille- | WVNAFARWGRAF | 51 | |
| | | | Q3913 | | | |
| 6 | A | A5 (5) | Yersinia kristensenil | WVNAFVNWPKSF | 51 | |
| | | | IP6945 | | | |
| 7 | A | | Yersinia bercovieri | WINAFARWGRAF | 51 | |
| | | | 127/84 | | | |
| 8 | A | A2 (2) | Serratia marcescens | WVNAFARWSKSF | 51 | |
| | | | CAV1761 | | | |
| 9 | A | | Yersinia enterocolitica | WVNAFVNWTKSF | 51 | |
| | | | PS23 | | | |
| 10 | A | | Xenorhabdus bovienii | WVNVFARWDKAI | 51 | |
| | | | CS03 | | | |
| 11 | A | | Erwinia | WVNAFANWTKRI | 51 | |
| 12 | A | | Yersinia aleksiciae | WVNAFLRWGKSF | 51 | |
| 13 | A | A3 (3) | Erwinia toletana DAPP- | WINAFANWTKRI | 51 | 8 |
| | | | PG 735 | | | |
| 14 | A | | Photorhabdus | WVNAFAKWTKRI | 51 | |
| | | | heterorhabditis ETL | | | |
| 15 | A | | Salmonella enterica | WVNFFAKFTKSF | 52 | |
| 16 | A | | Yersinia aldovae | WVNAFLNWSRSF | 51 | |
| | | | IP23238 | | | |
| 17 | A | | Erwinia sp. E602 | WVNAFANWPKRF | 53 | |
| 18 | A | | Yersinia frederiksenii | WVNAFLNWPRSF | 51 | |
| | | | RS-42 | | | |
| 19 | A | A8 (8) | Aeromonas jandaei | WVNAFANWTKRF | 51 | |
| | | | CN17A0119 | | | |
| 20 | A | A10 (10) | Vibrio sagamiensis | WVNAFARFTKRF | 55 | |
| | | | NBRC 104589 | | | |
| 21 | A | | Xenorhabdus japonica | WINVFARWNRAI | 51 | |
| | | | DSM 16522 | | | |
| 22 | A | A9 (9) | Providencia huaxiensis | WVNVFARWDKQI | 51 | |
| | | | Pvs2 | | | |
| 23 | A | A7 (7) | Sodalis sp. dw_96 | WVNAFARWDKKF | 51 | |
| 24 | A | | Xenorhabdus bovienii | WLNVFVRWDRAI | 51 | |
| | | | str. oregonense | | | |
| 25 | A | A4 (4) | Photorhabdus australis | WVNAYARWTNRF | 56 | |
| | | | DSM 17609 | | | |
| 26 | A | | Photorhabdus | WVNAYARWTKRF | 51 | 8 |
| | | | heterorhabditis SF41 | | | |
| 27 | A | | Yersinia mollaretil | AGWINAFGNWTK | 53 | |
| | | | SCPM-O-B-7610 | SF | | |
| 28 | A | | Yersinia mollaretil | AGWINAFANWTK | 53 | |
| | | | | SF | | |
| 29 | A | | Yersinia kristensenii | AGWIKAFGNWSR | 53 | |
| | | | | SF | | |
| 30 | A | A11 (11) | Serratia marcescens | WVNAFARWSRRW | 51 | 1 |
| | | | 90-166 | | | |
| 31 | A | | Yersinia mollaretii | AGWINAFANWTR | 53 | |
| | | | SCPM-O-B-7598 | SF | | |
| 32 | A | A1 (1) | Xenorhabdus | WINAFGNWERAF | 52 | 64 |
| | | | nematophila SC 0516 | H | | |
| 33 | A | | Yersinia enterocolitica | AGWIKVFGNWSR | 50 | |
| | | | E701 | SF | | |
| 34 | A | | Serratia marcescens | WVNVFARWSRRW | 51 | |
| | | | ID149856 | | | |
| 35 | A | | Serratia sp. DD3 | AGWIRAFANWSR | 53 | 4^{c} |
| | | | | SF | | |
| 36 | A | | Mixta theicola QC88- | GWFRAYLRWSRS | 54 | |
| | | | 366 | F | | |
| 37 | A | | Gilliamella sp. Lep-s5 | WWRAYARWRRSF | 54 | |
| 38 | A | A12-1 (12) | Engineered sequence | WVNAFARWSKRW | 52 | 2 |
| | | | of A-34 | | | |
| 39 | A | A12-2 (13) | Engineered sequence | WVNAFARWSKRF | 52 | 1 |
| | | | of A-34 | | | |
| 40 | B | B1 | Photorhabdus | GDRWLKWIKNH | 48 | |
| | | | laumondii | | | |
| 41 | B | | Kosakonia cowanii | DGRWLQWIKNH | 48 | |
| | | | pasteuri | | | |
| 42 | C | | Yersinia | WVNAFLN | 46 | |
| 43 | D | | Bordetella genomo sp. | VGGFANASWPKS | 53 | |
| | | | 11 AU8856 | F | | |
| 44 | D | | Bordetella bronchialis | VGGFANATWSKS | 53 | |
| | | | AU17976 | F | | |
| 45 | D | | Bordetella genomo sp. | VGGFANATWPKS | 53 | |
| | | | 9 AU14267 | F | | |
| 46 | D | | Providencia rettgeri | KSEAAGGWVNFQ | 50 | |
| | | | 2020EL-00052 | WKNSW | | |
| 47 | D | | Pandoraea | NVFVNATWSRAM | 52 | |
| | | | oxalativorans | | | |
| 48 | D | | Erythrobacter | WSRTVFNRVRPV | 45 | |
| 49 | D | | Sodalis sp. dw_96 | AGNDGWVKFGWK | 45 | |
| | | | | KKF | | |
| 50 | D | D1 | Kosakonia cowanii | RGEGWVRAYWAK | 49 | |
| | | | pasteuri | RF | | |
| 51 | D | | Bartonella | RGQGYVRFIFRR | 50 | |
| | | | | SF | | |
| 52 | D | | Photorhabdus | KPGEGWVNFTWN | 48 | |
| | | | heterorhabditis | KSF | | |
| 53 | D | | Erwinia | WVNAFANRTMGF | 55 | |
| | | | | LFKL | | |
| 54 | D | | Xenorhabdus griffiniae | ASTAETWFKLDW | 49 | |
| | | | VH1 | KKSF | | |
| 55 | D | D2 | Xenorhabdus griffiniae | SSDDDGIFFKTT | 49 | |
| | | | VH1 | WDRR | | |
| 56 | D | | Burkholderia | ADSQPKARAWFA | 56 | |
| | | | | NASFSKRF | | |
| 57 | D | | Trinickia | VESQSKPRAWFA | 56 | |
| | | | | NSSFSKRF | | |
| 58 | D | | Burkholderia | ASSQANSRGWFA | 57 | |
| | | | | NATWSKAWR | | |
| 59 | D | | Pandoraea | NAFVNATWSRAM | | |
| | | | norimbergensis | | | |
| 60 | D | | Pandoraea terrigena | NVFVNATWSRAI | | |
| | | | LMG 31013 | | | |

^{a}Bold residues indicate aromatic amino acids predicted to be in cyclophane
^{b}MIC (μg/mL) indicates the product has been produced and tested against E. coli.
^{c}Represents the Serraceptide product, aka Serraceptide.
^{d}length of a representative precursor encoding each core peptide
^{e}Precursor Type and Series of xenorceptide A-D
^{f}Xenorceptide compound numbers and abbreviated numbers used in FIGURES (in brackets)

In some embodiments, the polypeptide is selected from:

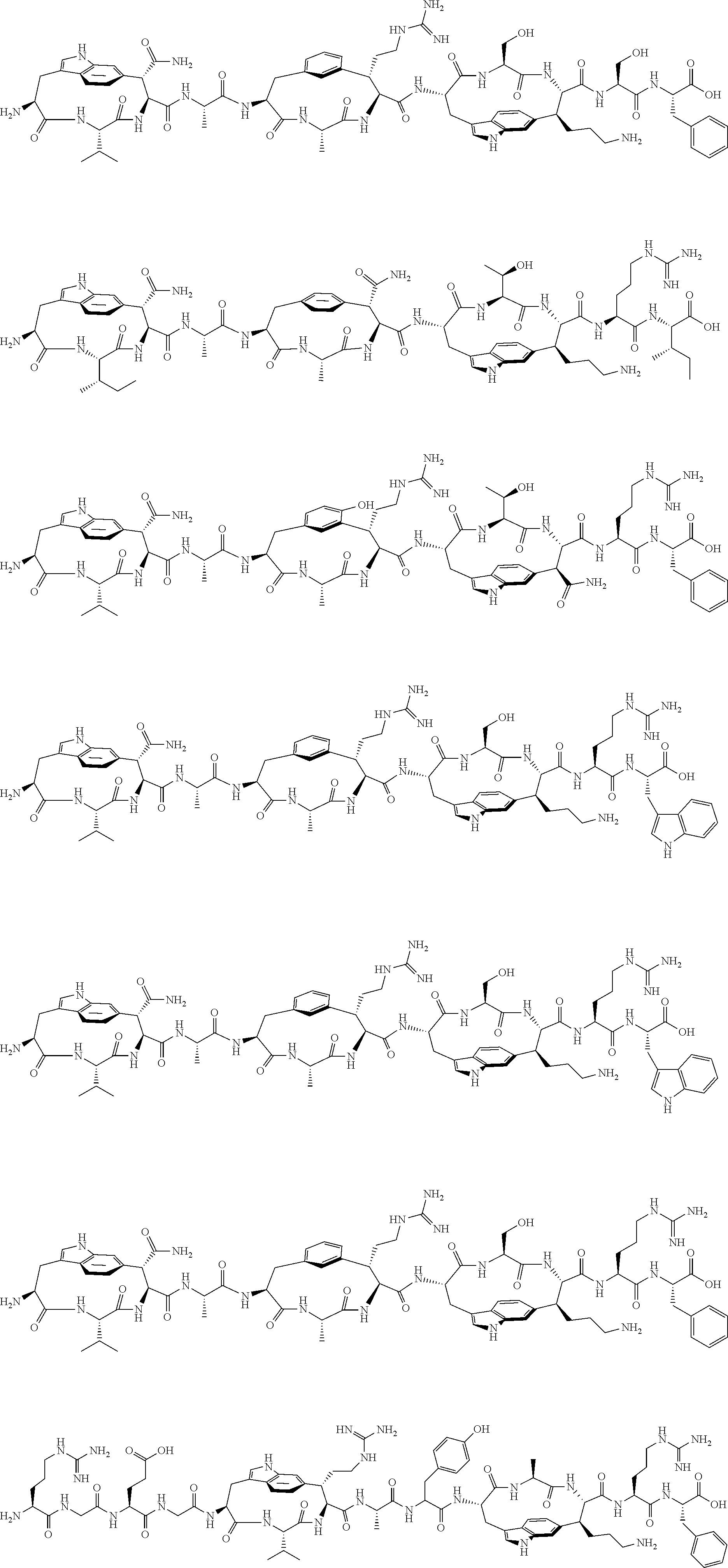

In some embodiments, the polypeptide is selected from WVNAFARWSKSF (2, SEQ ID 8), WINAFANWTKRI (3, SEQ ID 13) and WVNAYARWTKRF (4, SEQ ID 25). The cyclophane is formed between W and N, F and R, F and N, Y and R, and W and K. In some embodiments, the polypeptide is selected from:

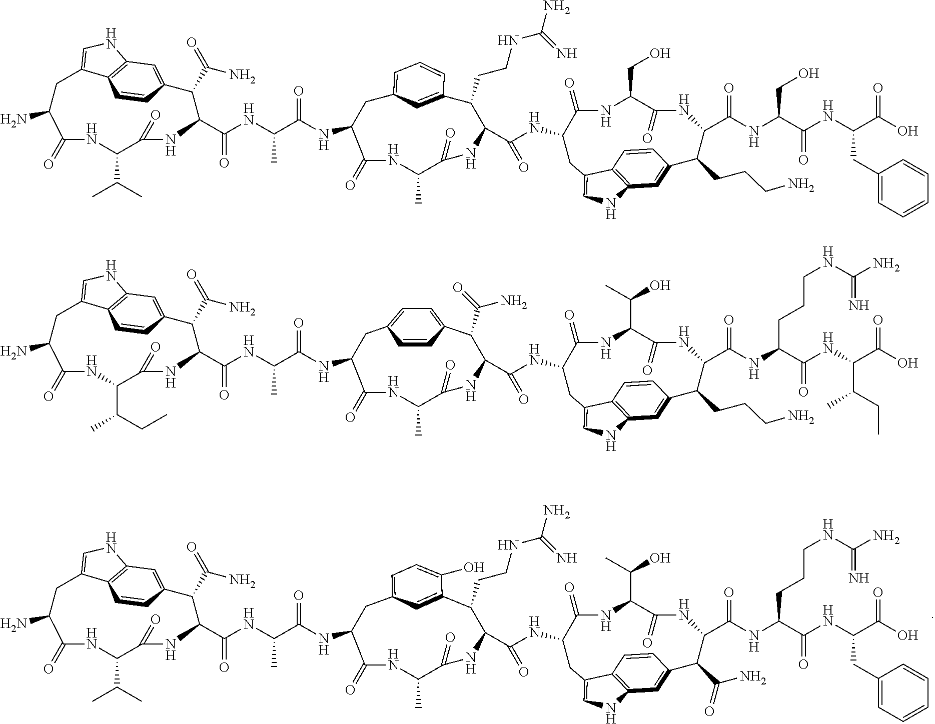

For simplicity, the above three polypeptide can be represented pictorially as follows:

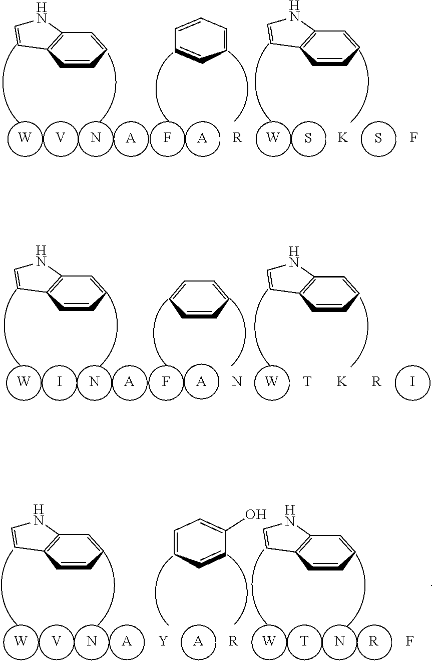

In some embodiments, the polypeptide is characterised by an antibacterial activity. In some embodiments, the polypeptide is characterised by an antibacterial activity against Gram-negative bacteria. The Gram-negative bacteria may be of the Enterobacteriaceae family. In some embodiments, the polypeptide is characterised by an antibacterial activity against drug-resistant bacteria. In some embodiments, the polypeptide shows antibacterial activity against Escherichia coli, Klebsiella pneumonia, Morganella mnorganii, Pseudomonas aeruginosa, Acinetobacter baumanii, Enterobacter cloacae, Salmonella typhimuriumn, Salmonella entereditis, Shigella flexneri, or a combination thereof. In some embodiments, the polypeptide shows antibacterial activity against Escherichia coli, Klebsiella pneumonia, Enterobacter cloacae, Salmonella typhimurium, Salmonella entereditis, Shigella flexneri, or a combination thereof.

It is believed that the varying activities of the peptides is due to different affinities to target proteins.

In some embodiments, the polypeptide is characterised by a minimal inhibitory concentration (MIC) of about 2 μg/mL to about 10 μg/mL. In other embodiments, the MIC is less than about 90 μg/mL, about 80 μg/mL, about 70 μg/mL, about 60 μg/mL, about 50 μg/mL, or about 40 μg/mL.

In some embodiments, the polypeptide is an isolated polypeptide. “Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The polypeptide may be present within a cell, present in the cellular medium, or prepared in various forms, such as lysates or isolated preparations. The polypeptide is then separated from its native medium in order to form the isolated polypeptide.

In some embodiments, the polypeptide is synthetically produced. In this regard, the polypeptide can be formed via recombinant methods, phage systems, biological systems and/or via chemical synthesis. For example, solid-phase peptide synthesis can be used. The polypeptide may be synthesised by providing the corresponding nucleic acid sequence to a host cell and the polypeptide produced and modified in vivo.

The present invention also provides a method of producing a polypeptide in a host cell, the method comprising:
a) introducing to the host cell one or more nucleic acid molecules, the nucleic acid molecules configured to express a precursor polypeptide (A), a rSAM/SPASM maturase (B), a protease (C), a transporter (D) and a protease/transporter (E);
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid or a derivative thereof;
wherein each X₂ and X₃ are independently any amino acid residue;
wherein at least one of the two C-terminus residues is an aromatic residue;
wherein the rSAM/SPASM maturase (B) is capable of modifying the precursor polypeptide (A) in the host cell to form a modified precursor polypeptide with a cyclophane moiety connecting the X₁ and X₃ residues in each motif;
wherein the protease (C), transporter (D) and protease/transporter (E) are capable of cleaving the modified precursor polypeptide from the rSAM/SPASM maturase (A) to form a cleaved modified polypeptide and exporting the cleaved modified polypeptide out from the host cell.

The nucleic acid molecule is a polynucleotide. In some embodiments, at least the nucleic acid molecule configured to express the precursor polypeptide (A) is derived from a Xye species. In some embodiments, at least the nucleic acid molecule configured to express the precursor polypeptide (A) and the nucleic acid molecule configured to express the rSAM/SPASM maturase (B) is derived from a Xye species.

In some embodiments, the nucleic acid molecule configured to express the precursor polypeptide (A) is from one Xye species while the nucleic acid molecules configured to express the rSAM/SPASM maturase (B), the protease (C), the transporter (D) and the protease/transporter (E) are from another Xye species. In some embodiments, the nucleic acid molecule configured to express the rSAM/SPASM maturase (B) is from one Xye species while the nucleic acid molecules configured to express the precursor polypeptide (A), the protease (C), the transporter (D) and the protease/transporter (E) are from another Xye species. In some embodiments, the nucleic acid molecule configured to express the protease (C) is from one Xye species while the nucleic acid molecules configured to express the precursor polypeptide (A), the rSAM/SPASM maturase (B), the transporter (D) and the protease/transporter (E) are from another Xye species. In some embodiments, the nucleic acid molecules configured to express the transporter (D) is from one Xye species while the nucleic acid molecules configured to express the precursor polypeptide (A), the rSAM/SPASM maturase (B), the protease (C), and the protease/transporter (E) are from another Xye species. In some embodiments, the nucleic acid molecules configured to express the protease/transporter (E) is from one Xye species while the nucleic acid molecules configured to express the precursor polypeptide (A), the rSAM/SPASM maturase (B), the protease (C), and the transporter (D) are from another Xye species. In some embodiments, the nucleic acid molecules configured to express the precursor polypeptide (A) and the rSAM/SPASM maturase (B) are from one Xye species while the nucleic acid molecules configured to express the protease (C), the transporter (D) and the protease/transporter (E) are from another Xye species. In some embodiments, the nucleic acid molecules configured to express the precursor polypeptide (A), the rSAM/SPASM maturase (B), the protease (C), the transporter (D) and the protease/transporter (E) are from one Xye species.

In some embodiments, the nucleic acid molecule is derived from a Xenorhabdus, Yersinia and Erwinia (Xye) maturase system. The Xye maturase system is named after three bacterial genera where it is commonly found: Xenorhabdus, Yersinia, and Erwinia, but also includes other bacterial genus where it may also be found, such as Serratia and Photorhabdus. In some embodiments, the nucleic acid molecule configured to express the precursor polypeptide is derived from a bacterial species selected from Serratia marcescens (smc), Erwinia toletana (etc), Photorhabdus australis (pac) or Xenorhabdus nematophila (xnc) In some embodiments, the nucleic acid molecule configured to express the rSAM/SPASM maturase is derived from a bacterial species selected from Serratia marcescens (smc), Erwinia toletana (etc), Photorhabdus australis (pac) or Xenorhabdus nematophila (xnc). In some embodiments, the nucleic acid molecule configured to express the protease, transporter and protease/transporter are derived from Xenorhabdus nematophila (xnc).

In some embodiments, the nucleic acid molecules configured to express the precursor polypeptide is derived from a bacterial species selected from Xenorhabdus griffiniae VH1 (xgc), Pandoraea sp. PE-S2R-1 (psc), Pandoraea oxalativorans DSM 23570 (poc), Photorhabdus heterorhabditis Q614 (phc), Kosakonia cowanii pasteuri (kcc2 and kcc1kcc1), Bordetella bronchialis AU17976 (bbc) and Photorhabdus laumondii BOJ-47 (plc).

In some embodiments, only the nucleic acid molecules configured to express protease, transporter and protease/transporter are derived from Xenorhabdus Spp.

The nucleic acid molecules may each individually express a precursor polypeptide, a rSAM/SPASM maturase, a protease, a transporter and a protease/transporter. Alternatively, the nucleic acid molecules may be fused. In other words, the nucleic acid molecules are operably linked to a first promoter; i.e. the nucleic acid molecules are part of one expression unit. In some embodiments, at least the nucleic acid molecule expressing the protease, the nucleic acid molecule expressing the transporter and the nucleic acid molecule expressing the protease/transporter are fused. In some embodiments, the nucleic acid molecule expressing the precursor polypeptide and the nucleic acid molecule expressing the rSAM/SPASM maturase are fused. In some embodiments, the nucleic acid molecule expressing the rSAM/SPASM maturase, the nucleic acid molecule expressing the protease, the nucleic acid molecule expressing the transporter and the nucleic acid molecule expressing the protease/transporter are fused. In some embodiments, the nucleic acid molecule expressing the precursor polypeptide, the nucleic acid molecule expressing the rSAM/SPASM maturase, the nucleic acid molecule expressing the protease, the nucleic acid molecule expressing the transporter and the nucleic acid molecule expressing the protease/transporter are fused.

In some embodiments, the nucleic acid molecule expressing the precursor polypeptide and the nucleic acid molecule expressing the rSAM/SPASM maturase are fused or operably linked to a first promoter, and the nucleic acid molecule expressing the protease, the nucleic acid molecule expressing the transporter and the nucleic acid molecule expressing the protease/transporter are fused or operably linked to a second promoter.

In some embodiments, the nucleic acid molecule expressing the precursor polypeptide is operably linked to a first promoter, and the nucleic acid molecule expressing the rSAM/SPASM maturase, the nucleic acid molecule expressing the protease, the nucleic acid molecule expressing the transporter and the nucleic acid molecule expressing the protease/transporter are fused or operably linked to a second promoter.

When the nucleic acid molecules are fused or linked, they may be fused in any order. For example, the nucleic acid molecule expressing the precursor polypeptide (A), the nucleic acid molecule expressing the rSAM/SPASM maturase (B), the nucleic acid molecule expressing the protease (C), the nucleic acid molecule expressing the transporter (D) and the nucleic acid molecule expressing the protease/transporter (E) may be fused as BACDE, BADEC, BAECD, BADCE, BACED, BAEDC, ABCDE, ABDEC, ABECD, ABDCE, ABCED, or ABEDC. When C, D and E are fused, they may be fused as CDE, DEC, ECD, DCE, CED, or EDC. When A and B are fused, they may be fused as AB or BA.

In some embodiments, at least one motif comprises X₁ and X₃ connected via phenylene to form a cyclophane moiety. In some embodiments, at least one motif comprises X₁ and X₃ connected via indolylene to form a cyclophane moiety. In some embodiments, the two motifs separately comprises phenylene and indolylene.

The present invention also provides a method of producing a polypeptide in a host cell, the method comprising:
a) introducing to the host cell one or more nucleic acid molecules, the nucleic acid molecules configured to express a precursor polypeptide, a rSAM/SPASM maturase, a protease, a transporter and a protease/transporter;
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, or an unnatural aromatic amino acid residue;
wherein each X₂ and X₃ are independently any amino acid residue;
wherein at least one of the two C-terminus residues is an aromatic residue;
wherein the rSAM/SPASM maturase is capable of modifying the precursor polypeptide in the host cell to form a modified precursor polypeptide with a cyclophane moiety connecting the X₁ and X₃ residues in each motif;
wherein X₁ and X₃ in the second motif are connected via phenylene to form a cyclophane moiety;
wherein only the protease, transporter and protease/transporter are derived from Xenorhabdus Spp;
wherein the protease, transporter and protease/transporter are capable of cleaving the modified precursor polypeptide from the rSAM/SPASM maturase to form a cleaved modified polypeptide and exporting the cleaved modified polypeptide out from the host cell.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to synthesis a modified polypeptide of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

In some embodiments, the method further comprises a step of culturing the host cell under conditions suitable for the production of the polypeptide.

The precursor polypeptide may be of any sequence length, as long as it comprises at least two of the three residue motif optionally separated by 1 to 3 amino acid residue and at least two C-terminus residues. The precursor polypeptide, which does not comprise a cyclophane, is then modified by the rSAM/SPASM maturase to form a cyclophane containing modified precursor polypeptide. The modified precursor polypeptide may then be cleaved and transported out from the host cell by the protease, transporter and protease/transporter.

In some embodiments, the precursor polypeptide or the nucleic acid molecule configured to express the precursor polypeptide is derived from a bacterial strain as shown in Table 3. In some embodiments, the precursor polypeptide or the nucleic acid molecule configured to express the precursor polypeptide is derived from Serratia marcescens (smc), Erwinia toletana (etc), Photorhabdus australis (pac), Xenorhabdus nematophila (xnc), Xenorhabdus griffiniae VH1 (xgc), Pandoraea sp. PE-S2R-1 (psc), Pandoraea oxalativorans DSM 23570 (poc), Photorhabdus heterorhabditis Q614 (phc), Kosakonia cowanii pasteuri (kcc2 and kcc1), Bordetella bronchialis AU17976 (bbc) or Photorhabdus laumondii BOJ-47 (plc).

The precursor polypeptide and the rSAM/SPASM maturase (or the nucleic acid molecule configured to express the precursor polypeptide and rSAM/SPASM maturase) may be derived from the same bacterial strain, or may be of different bacterial strains. In some embodiments, the precursor polypeptide and rSAM/SPASM maturase (or the nucleic acid molecule configured to express the precursor polypeptide and rSAM/SPASM maturase) are derived from a bacterial strain as shown in Table 3. In some embodiments, the precursor polypeptide is fused to the rSAM/SPASM maturase. In some embodiments, the precursor polypeptide are transcribed and translated separately from the rSAM/SPASM maturase.

The amino acid sequence of the precursor polypeptide may be at least 70% identical to the amino acid sequence of SEQ ID NO: [XyeA](see Table 4 below). The amino acid sequence of the precursor polypeptide may be at least 70% identical to the amino acid sequence of SEQ ID NO: [SmcA], SEQ ID NO: [EtcA], SEQ ID NO: [PacA], SEQ ID NO: [XgcA], SEQ ID NO: [PscA], SEQ ID NO: [PocA], SEQ ID NO: [PhcA], SEQ ID NO: [Kcc2A]SEQ ID NO: Kcc1A, SEQ ID NO: [BbcA] or SEQ ID NO: [PlcA].

The amino acid sequence of the rSAM/SPASM maturase may be at least 70% identical to the amino acid sequence of SEQ ID NO: [XyeB](see Table 4 below).

The term “rSAM” refers to radical S-adenosylmethionine. The rSAM enzyme may be an rSAM enzyme of the Xenorhabdus, Yersinia and Erwinia (XYE) maturase system (Xye, TIGR04496, IPR030989), Glycine-rich repeat (Grr) maturase system (GrrM, TIGR04261, IPR026357) or the Fxs maturase system (FxsB, TIGR04269, IPR026335). In some embodiments, the rSAM/SPASM maturase is from a Xenorhabdus, Yersinia and Erwinia (XYE) maturase system.

The rSAM enzyme may also be an enzymatically active fragment of an rSAM enzyme of the Xenorhabdus, Yersinia and Erwinia (XYE) maturase system (XyeB, TIGR04496, IPR030989), Glycine-rich repeat (Grr) maturase system (GrrM, TIGR04261, IPR026357) or the Fxs maturase system (FxsB, TIGR04269, IPR026335). In some embodiments, the rSAM/SPASM maturase is an enzymatically active fragment from a Xenorhabdus, Yersinia and Erwinia (XYE) maturase system.

The rSAM enzyme may have an amino acid sequence that is at least 70% (or 75%, 80%, 85%, 90% or 95%) identical to the following sequences:

XncB (Xenorhabdus nematophila):
(SEQ ID NO: 61)
MTTSKSEKIKHLEIILKISERCNINCSYCYVFNMGNSLATDSPPVISLDN
VLALRGFFERSAAENEIEVIQVDFHGGEPLMMKKDRFDQMCDILRQGDYS
GSRLELALQTNGILIDDEWISLFEKHKVHASISIDGPKHINDRYRLDRKG
KSTYEGTIHGLRMLQNAWKQGRLPGEPGILSVANPTANGAEIYHHFANVL
KCQHFDFLIPDAHHDDDIDGIGIGRFMNEALDAWFADGRSEIFVRIFNTY
LGTMLSNQFYRVIGMSANVESAYAFTVTADGLLRIDDTLRSTSDEIFNAI
GHLSELSLSGVLNSPNVKEYLSLNSELPSDCADCVWNKICHGGRLVNRFS
RANRFNNKTVFCSSMRLFLSRAASHLITAGIDEETIMKNIQK
YkcB (Yersinia kristensenii):
(SEQ ID NO: 62)
MEVITGSEGRVMLNLLIEKNIRHLEIILKISERCNINCDYCYVFNKGNSA
ADDSPARLSNKNIHHLVCFLQRACQEYKIGTVQIDFHGGEPLLMKKENFT
DMCIQLISGNYCGSNIRLALQTNATLIDNEWIAIFEKYSVNVSISIDGPK
HINDRHRLDTKGRSTYESTVRGLRILQNAYQQGRLPSDPGILCVTNAQAN
GAEIYRHFVDELGVYSFDFLIPDDSYKDAHPDAVGIGRFLNEALDEWVKD
NNAKIFVRLFQTHIASLLGQKNSGVLGHTPNITGVYALTVSSDGFVRVDD
TLRSTSDRMFNPIGHLSEVNLSNVFASPQFQEYSSIGQSLPTECEGCIWE
NICAGGRIVNRFSTEDRFKHKSIYCYSMRTFLSRSSAHLLNMGIKEERIM
AAIRA
EtcB (Erwinia toletana):
(SEQ ID NO: 63)
MTQLKGEKIKHLEIILKISERCNINCTYCYVFNMGNTLATDSTPVISLDN
VYALRGFFERSAAENDIEVIQVDFHGGEPLMMKKDRFDRMCQILLQGNYR
SSKFELALQTNGILIDDEWIALFEKHQVHASISVDGPKHINDRHRLDRKG
KSTYEGTITGLRLLQNAWQQGRLPGEPGILSVANANANGAEIYRHFADTL
QCQRFDFLIPDDHHDDSPDGEGVGRFLNEALDAWFADGRPEIFIRIFNTY
LGTMLNSQFNRVLGMSANVESAYAFTVTADGMLRIDDTLRSTSDEIFNAV
GHVSELSLARVLETSCVKEYLALSSNLPTVCAECVWNNICHGGRLVNRFS
RTNRFNNKTVFCKSMRLFLSRAASHLMASGVDEKEIMKNIQK
MscB (Micromonospora sp.):
(SEQ ID NO: 64)
MAPGPARAALTEFVLKVHARCDLACDHCYVYEHADQSWRRRPVRMTPEVL
RTAAGRIAEHAAAHDLPDVTVILHGGEPLLLGAERLGEVLADLRRVIDPV
TRLRLGMQTNGVLLSERLCDLLAEHDVAVGVSLDGDRAANDRHRRFRSGA
GSYDQVLRAIGLLRRPAYRRIYSGLLCTVDVRNDPIAVYESLLTQEPPRI
DFLLPHATWDDPPWRPAGGGTAYAGWLRAVYDRWLADGRPVSVRLFDSLL
STAAGGPSGTEWLGLDPVDLAVVETDGEWEQADSLKTAYDGAPATGMTVF
SHAADDVAASPLLARRRSGRAGLSDECRRCPVVDQCGGGLFAHRYGAGHF
DHPSVYCADLKELIVHVNENPPAPVRLDAGLPDDFIDRLAALTGDRVAIG
RLVEAQIAIVRALLAEVADRLPAGGAGADGWEALTALDRSAPESVARIAA
HPYVRAWAVDCLAGSGTGARQGPDYLSALAVAAALDAGTPVRLDVPVRSG
RLHLPTVGTVLLPEVGDGAARVETGPGSLRVAAGDVTVAIRPGTPGDAPR
WWPTRVLAAPDVSVLLEDGDPHRDCHRLPAGDRLDDAGAARWAETFAAAW
QVIRDEVPGHAEELRAGLRAVVPLRRSGAGVSEASTARQAFGGVAATETD
AGSLAVLLVHEFQHSKMNALLDICDLVDGTRPIDITVGWRPDPRPAEAVL
HGIYAHAAVADIWRIRADRQVDGAQAVYRRYRDWTAEAIGALQRADALTP
AGSRLVRQVARSMSGWPS
OscB (Oscillatoriales cyanobacterium):
(SEQ ID NO: 65)
MINPTLLNPEKIDISKFGPINLVVIQATSFCNLNCDYCYLPNRDLKNTLS
LDLIEPIFKNIFNSPFVGDEFTICWHAGEPLAVPISFYESAFQLIQAADQ
KYNQKQAKIWHSVQTNATYINQKWCDFIQEHNICVGVSLDGPEFIHDAHR
QTRKGTGSHAQTMRGISFLQKNNIPFYVISVVTQDSLNYADEIFNFFREN
GIYDVGFNLEEIEGVNQSSTLEAVGTSEKYRAFMQRFWELTSEVQGEFNL
REFEAICGLIYSNTRLTQTDMNNPFVLINIDYQGNFSTFDPELLSVNIKP
YGNFILGNVLTDSFESVCDTEKFQKIYTDMQEGIKLCRETCEYFGVCGGG
AGSNKYWENGTFACSETMACRYRIKVVTDIILDKLENSLGLVENC
LscB (Lyngbya sp.):
(SEQ ID NO: 66)
MTISKMNLPVQTDNFRASSTLDLSAFGPINLVVIQSTSFCNLNCDYCYLR
DRQSKNRLSLDLIEPILKTVLTSPFVGCDFTILWHAGEPLAMPISFYDSA
TALIREAERQYKTQPIQIFQSIQTNATLINQAWCDCFRRNEIYVGVSLDG
PAFLHDAHRQTYKGTGTHAATMRGISLLQKNEIPENVICVLTQDSLDYPD
EIFNFFRSNRITEVGFNMEEAEGVHQHSTLDQQGTEERYRAFMQRFWDLT
VQAKGEFKLREFETICTLAYTGDRLGYTDMNQPFVIVNFDHQGNFSTFDP
ELLSFKIKEYGDFVLGNVLHNTLESVCQTEKFQKIYQDMAAGVVQCRQSC
EYFGLCGGGAGSNKYWENGTFNCTETKACRYRIKVIADIVLEGLENSLEL
ANSIS
GscB (Geminocytis sp.):
(SEQ ID NO: 67)
MSIVTSKPVINFKNTANFGPISLIIIQPNSFCNLDCDYCYLPDRHLQNKL
SLDLIDPIFKSIFTSPFLGCDFGVCWHAGEPLTMPVSFYKSAFQLIEEAN
TKYNKSEYSFYHSYQTNGTLINQGWCDLWQEYPVHVGVSIDGPAFLHDVH
RKNRKGGNSHDLTMRGIRYLQKNNIPYNTISVITEESLNYPDEMFNFFAE
NEIYDLAFNMEETEGVNELTSLNGIEIEHKYSQFIKRFWQLVTESKLPFI
VREFEILISLIYSGNRLTNTDMNKPFVIVNFDYQGNFSTFDPELLSVKTD
KYGDFIFGNVLKDSLESICETEKFKTIYKDINDGVKLCSDNCSYFGICGG
GAGSNKYWENGTFASMETQACRYRIKILTDVLVSTIENSLGL

In one embodiment, the rSAM enzyme is a C-terminal truncated MscB-375 enzyme with the following sequence:

(SEQ ID NO: 68)
MAPGPARAALTEFVLKVHARCDLACDHCYVYEHADQSWRRRPVRMTPEVL
RTAAGRIAEHAAAHDLPDVTVILHGGEPLLLGAERLGEVLADLRRVIDPV
TRLRLGMQTNGVLLSERLCDLLAEHDVAVGVSLDGDRAANDRHRRFRSGA
GSYDQVLRAIGLLRRPAYRRIYSGLLCTVDVRNDPIAVYESLLTQEPPRI
DFLLPHATWDDPPWRPAGGGTAYAGWLRAVYDRWLADGRPVSVRLFDSLL
STAAGGPSGTEWLGLDPVDLAVVETDGEWEQADSLKTAYDGAPATGMTVF
SHAADDVAASPLLARRRSGRAGLSDECRRCPVVDQCGGGLFAHRYGAGHF
DHPSVYCADLKELIVHVNENPPAPV.

The enzymes as referred to herein may comprise one or more conservative amino acid substitution.

In one embodiment, the rSAM enzyme is an enzymatically active fragment of any one of the above sequences. In one embodiment, the enzymatically active fragment is one that comprises the rSAM and SPASM domains (such as CNINCSYC (SEQ ID NO: 69) and CADCVWNKIC (SEQ ID NO: 70) in XncB). In one embodiment, the enzymatically active fragment is from YkcB, wherein the rSAM domain is CNINCDYCYVFNK (SEQ ID NO: 213) and the SPASM domain is CEGCIWENIC (SEQ ID NO: 214). In one embodiment, the enzymatically active fragment is from EtcB, wherein the rSAM domain is CNINCTYC (SEQ ID NO: 215), and the SPASM domain is CAECVWNNIC (SEQ ID NO: 216). In one embodiment, the enzymatically active fragment is from MscB, wherein the rSAM domain is CDLACDHC (SEQ ID NO: 217), and the SPASM domain is CRRCPVVDQC (SEQ ID NO: 218). In one embodiment, the enzymatically active fragment is from OscB, wherein the rSAM domain is CNLNCDYC (SEQ ID NO: 219), and the SPASM domain is CRETCEYFGVC (SEQ ID NO: 220). In one embodiment, the enzymatically active fragment is from LscB, wherein the rSAM domain is CNLNCDYC (SEQ ID NO: 221), and the SPASM domain is CRQSCEYFGLC (SEQ ID NO: 222). In one embodiment, the enzymatically active fragment is from GscB, wherein the rSAM domain is CNLDCDYC (SEQ ID NO: 223), and the SPASM domain is CSDNCSYFGIC (SEQ ID NO: 224).

The rSAM enzyme may be a XyeB, GrrM or FxsB rSAM enzyme from a bacterial genus listed in Tables 4-6.

TABLE 4
Precursor (XyeA, IPRO30990) and rSS (XyeB, IPR030989)
paired sequences from the UniProt database.

| Accession No. | | |
|---|---|---|
| Precursor | Accession No. | |
| (XyeA) | rSS (XyeB) | Strain |
| A0A1C0TZE6 | A0A1C0TZL9 | Photorhabdus australis |
| A0A1Q4P361 | A0A1Q4P3B6 | Serratia marcescens |
| A0A084A5U2 | A0A084A5U1 | Serratia sp. DD3 |
| A0A0B6XF00 | A0A0B6XFQ9 | Xenorhabdus bovienii |
| A0A077P0J4 | A0A077P0L0 | Xenorhabdus bovienii str. oregonense |
| A0A1I5BFB3 | A0A1I5BES0 | Xenorhabdus japonica |
| D3VF66 | D3VF67 | Xenorhabdus nematophila (strain ATCC 19061/ |
| | | DSM 3370/LMG 1036/NCIB 9965/AN6) |
| A0A0R4D012 | A0A0R4D0A6 | Xenorhabdus nematophila AN6/1 |
| N1NN13 | N1NM08 | Xenorhabdus nematophila F1 |
| A0A0A8NQW6 | A0A0A8NMB7 | Xenorhabdus nematophila str. Websteri |
| A0A2D0KYU9 | A0A2D0KZ85 | Xenorhabdus sp. KJ12.1 |
| A0A2D0K7T4 | A0A2D0K7L0 | Xenorhabdus sp. KK7.4 |
| A0A2D0KQ63 | A0A2D0KQJ1 | Xenorhabdus stockiae |
| A0A2G4TZ16 | A0A2G4TZ87 | Yersinia bercovieri |
| A0A0E1NG59 | A0A0EINDZ2 | Yersinia enterocolitica |
| A0A0T7NPU9 | A0A0T7NP34 | Yersinia enterocolitica |
| A0A0H3NSR9 | A0A0H3NRG2 | Yersinia enterocolitica subsp. palearctica |
| | | serotype O:3 (strain DSM 13030/CIP 106945/ |
| | | Y11) |
| F4MYR4 | F4MYR5 | Yersinia enterocolitica W22703 |
| A0A209AZF0 | A0A209AZP3 | Yersinia frederiksenii |
| A0A0T9N5M4 | A0A0T9N4P3 | Yersinia kristensenii |
| A0A0T9U1K9 | A0A0T9U1I2 | Yersinia kristensenii |
| A0A0U1HZP4 | A0A0U1HZK1 | Yersinia mollaretii |
| C4S8Z7 | C4S8Z6 | Yersinia mollaretii ATCC 43969 |

TABLE 5
Precursor (GrrA, IPR026356) and rSS (GrrM, IPR026357)
paired sequences from the UniProt database.

| Accession No. | Accession No. | |
|---|---|---|
| Precursor (GrrA) | rSAM (GrrM) | Strain |
| A0A1Q3KH01 | A0A1Q3KH56 | Alphaproteobacteria bacterium 65-37 |
| A0A2T1F2L2 | A0A2T1F219 | Aphanothece cf. minutissima CCALA 015 |
| A0A2T1LXR5 | A0A2T1LXR7 | Aphanothece hegewaldii CCALA 016 |
| G5J0Q7 | G5J0Q8 | Crocosphaera watsonii WH 0003 |
| G5J8Q7 | G5J0Q8 | Crocosphaera watsonii WH 0003 |
| G5J8Q8 | G5J0Q8 | Crocosphaera watsonii WH 0003 |
| T2IXQ8 | T2IYC6 | Crocosphaera watsonii WH 0005 |
| T2IXZ4 | T2IYC6 | Crocosphaera watsonii WH 0005 |
| T2J085 | T2IYC6 | Crocosphaera watsonii WH 0005 |
| T2JXQ3 | T2JW16 | Crocosphaera watsonii WH 0402 |
| T2JY88 | T2JW16 | Crocosphaera watsonii WH 0402 |
| T2JZD7 | T2JW16 | Crocosphaera watsonii WH 0402 |
| Q4BWP4 | Q4BWP2 | Crocosphaera watsonii WH 8501 |
| A0A1Z9JEB4 | A0A1Z9JEI5 | Cyanobacteria bacterium TMED177 |
| A0A1Z9JES1 | A0A1Z9JEI5 | Cyanobacteria bacterium TMED177 |
| A0A1Z9JIL3 | A0A1Z9JEI5 | Cyanobacteria bacterium TMED177 |
| A0A1Z9LF09 | A0A1Z9LEY5 | Cyanobacteria bacterium TMED188 |
| A0A1Z9LF10 | A0A1Z9LEY5 | Cyanobacteria bacterium TMED188 |
| K9Z5N8 | K9Z319 | Cyanobacterium aponinum (strain PCC |
| | | 10605) |
| A0A2G3PAN6 | A0A2G3P8V3 | Cyanobacterium aponinum IPPAS B-1201 |
| K9PAE0 | K9PBG1 | Cyanobium gracile (strain ATCC 27147/ |
| | | PCC 6307) |
| A0A2W6YZ82 | A0A2W6YZU4 | Cyanobium sp |
| A0A2W6ZHA8 | A0A2W7A6G1 | Cyanobium sp |
| A0A326QHT4 | A0A326QDC6 | Cyanobium sp |
| A0A2D6FEB5 | A0A2D6FEG4 | Cyanobium sp. ARS6 |
| A0A081GHK6 | A0A081GHK5 | Cyanobium sp. CACIAM 14 |
| A0A2E1IN00 | A0A2E1IQ77 | Cyanobium sp. MED843 |
| A0A2E1IQ42 | A0A2E1IQ77 | Cyanobium sp. MED843 |
| A0A2E1IQ50 | A0A2E1IQ77 | Cyanobium sp. MED843 |
| A0A2E0AN10 | A0A2E0AMN8 | Cyanobium sp. NAT70 |
| A0A182AQN3 | A0A182ASF1 | Cyanobium sp. NIES-981 |
| A0A182AU27 | A0A182ASU9 | Cyanobium sp. NIES-981 |
| B5IK36 | B5IK37 | Cyanobium sp. PCC 7001 |
| B5ILU6 | B5ILU5 | Cyanobium sp. PCC 7001 |
| A0A2E4LLZ3 | A0A2E4LLZ4 | Cyanobium sp. SAT1300 |
| A0A2P7MTB4 | A0A2P7MT91 | Cyanobium usitatum str. Tous |
| B1X121 | B1X120 | Cyanothece sp. (strain ATCC 51142) |
| B1X122 | B1X120 | Cyanothece sp. (strain ATCC 51142) |
| B7KDY1 | B7KDY3 | Cyanothece sp. (strain PCC 7424) |
| B7KDY2 | B7KDY3 | Cyanothece sp. (strain PCC 7424) |
| B8HSH4 | B8HSH5 | Cyanothece sp. (strain PCC 7425/ATCC |
| | | 29141) |
| B8HSH8 | B8HSH9 | Cyanothece sp. (strain PCC 7425/ATCC |
| | | 29141) |
| B8HV48 | B8HUF3 | Cyanothece sp. (strain PCC 7425/ATCC |
| | | 29141) |
| E0UHF6 | E0UHF5 | Cyanothece sp. (strain PCC 7822) |
| E0UHF7 | E0UHF5 | Cyanothece sp. (strain PCC 7822) |
| B7JUH9 | B7JUI0 | Cyanothece sp. (strain PCC 8801) |
| A3INK4 | A3INK3 | Cyanothece sp. CCY0110 |
| A3INK5 | A3INK3 | Cyanothece sp. CCY0110 |
| A0A3B8XXV7 | A0A3B8Y1T1 | Cyanothece sp. UBA12306 |
| A0A3B8XZG8 | A0A3B8Y6Z2 | Cyanothece sp. UBA12306 |
| A0A3B8Y4Z1 | A0A3B8Y1T1 | Cyanothece sp. UBA12306 |
| A0A1T4RKP1 | A0A1T4RK36 | Enhydrobacter aerosaccus |
| A0A2P8W4T2 | A0A2P8W4T3 | filamentous cyanobacterium CCT1 |
| A0A0D6AAG1 | A0A0D6AAL6 | Geminocystis sp. NIES-3708 |
| A0A0D6AAQ5 | A0A0D6AAL6 | Geminocystis sp. NIES-3708 |
| A0A0D6AVA7 | A0A0D6AVB2 | Geminocystis sp. NIES-3709 |
| A0A0D6AWJ4 | A0A0D6AVB2 | Geminocystis sp. NIES-3709 |
| A0A261KMH7 | A0A261KM11 | Hydrocoleum sp. CS-953 |
| A0A261KMK1 | A0A261KM12 | Hydrocoleum sp. CS-953 |
| A0A261KPG0 | A0A261KM13 | Hydrocoleum sp. CS-953 |
| A0A1L3EWS6 | A0A1L3EWP1 | Luteibacter rhizovicinus DSM 16549 |
| A0A2T5LGC6 | A0A2T5LG77 | Luteibacter sp. OK325 |
| A0YYD0 | A0YYD1 | Lyngbya sp. (strain PCC 8106) |
| A0A113WAQ4 | A0A1I3WAK9 | Methylocapsa palsarum |
| A0A2J7TE77 | A0A2J7TE75 | Methylocella silvestris |
| B8EQ29 | B8EQ28 | Methylocella silvestris (strain DSM 15510/ |
| | | CIP 108128/LMG 27833/NCIMB 13906/BL2) |
| A0A3E0LTQ3 | A0A2W4QF24 | Microcystis aeruginosa DA14 |
| L8NY47 | A0A2W6YZU4 | Microcystis aeruginosa DIANCHI905 |
| A0A3NOWKD4 | A0A2W7B0M0 | Microcystis aeruginosa FACHB-524 |
| A0A1V4BUU7 | A0A2Z6UYG4 | Microcystis aeruginosa KW |
| A0A0F6RM21 | A0A3E0LNV2 | Microcystis aeruginosa NIES-2549 |
| A0A2H6BTD4 | A0A3E0LRP7 | Microcystis aeruginosa NIES-298 |
| A0A0A1VYH5 | A0A3N0VP57 | Microcystis aeruginosa NIES-44 |
| A0A2H6KZG4 | A0A3N5J195 | Microcystis aeruginosa NIES-87 |
| A0A139GHJ6 | A0A3R7P7F6 | Microcystis aeruginosa NIES-88 |
| A0A1E4QIR2 | A0A3S1IS64 | Microcystis aeruginosa NIES-98 |
| A8YAG5 | A0A3S3KC59 | Microcystis aeruginosa PCC 7806 |
| I4GMR0 | A0A402AY08 | Microcystis aeruginosa PCC 7941 |
| I4FZ11 | A0A402DGT7 | Microcystis aeruginosa PCC 9443 |
| I4IUU0 | A0A402DKN0 | Microcystis aeruginosa PCC 9701 |
| I4FU32 | A0A429FKD6 | Microcystis aeruginosa PCC 9717 |
| I4GVW3 | A0A495Q9Z9 | Microcystis aeruginosa PCC 9806 |
| I4HD64 | A0A4P5VFP0 | Microcystis aeruginosa PCC 9807 |
| I4HZK0 | A0A4P5VNH3 | Microcystis aeruginosa PCC 9808 |
| I4HQP4 | A0A4P5Z922 | Microcystis aeruginosa PCC 9809 |
| A0A2Z6UMP5 | A0A4P6JJ41 | Microcystis aeruginosa Sj |
| S3JFW1 | A0A4P6JTC0 | Microcystis aeruginosa SPC777 |
| A0A3E0LWL6 | A0A4P6LF79 | Microcystis aeruginosa TA09 |
| L7E5P1 | A0A4P7ZWF9 | Microcystis aeruginosa TAIHU98 |
| A0A3E0LEJ9 | A0A4Q0QKH8 | Microcystis flos-aquae DF17 |
| A0A3E0L677 | A0A4R2MAC4 | Microcystis flos-aquae TF09 |
| A0A0K1S6M0 | A0A4V0YR58 | Microcystis panniformis FACHB-1757 |
| A0A2L2XVF6 | A0A510PMW7 | Microcystis sp. 0824 |
| A0A2P1UF64 | A0A521QRV3 | Microcystis sp. MC19 |
| I4IH33 | A0A525JRG1 | Microcystis sp. T1-4 |
| A0A3G9JV83 | A0A537IV48 | Microcystis viridis NIES-102 |
| A0A3E0LNP2 | A0A537WMI1 | Microcystis wesenbergii TW10 |
| A0A098TGT4 | A0A098TIF4 | Neosynechococcus sphagnicola sy1 |
| A0A1J5GLC7 | A0A1J5G9T5 | Oscillatoriales cyanobacterium |
| | | CG2_30_40_61 |
| A0A1J5GNK8 | A0A1J5G9T5 | Oscillatoriales cyanobacterium |
| | | CG2_30_40_61 |
| A0A2D5W495 | A0A2D5W441 | Pedosphaera sp |
| A0A1U7IQQ0 | A0A1U7IR09 | Phormidium ambiguum IAM M-71 |
| A0A1J1JHQ4 | A0A1J1JKY7 | Planktothrix agardhii |
| A0A2Z6CEF9 | A0A2Z6CEN3 | Planktothrix agardhii NIES-204 |
| A0A073CC77 | A0A073CPJ3 | Planktothrix agardhii NIVA-CYA 126/8 |
| A0A1J1K3H2 | A0A1J1K5L2 | Planktothrix paucivesiculata PCC 9631 |
| A0A1J1K4A6 | A0A1J1K5L2 | Planktothrix paucivesiculata PCC 9631 |
| A0A1J1L466 | A0A1J1L5D0 | Planktothrix rubescens |
| A0A1J1L4L1 | A0A1J1L5D0 | Planktothrix rubescens |
| A0A1T4ZP83 | A0A1T4ZPC2 | Planktothrix sp. PCC 11201 |
| A0A1T4ZPR1 | A0A1T4ZPC2 | Planktothrix sp. PCC 11201 |
| A0A354WB48 | A0A354WC37 | Planktothrix sp. UBA10369 |
| A0A1J1LRN3 | A0A1J1LPS2 | Planktothrix tepida PCC 9214 |
| A2C6R5 | A2C6R4 | Prochlorococcus marinus (strain MIT |
| | | 9303) |
| A2C6R6 | A2C6R4 | Prochlorococcus marinus (strain MIT |
| | | 9303) |
| Q7TUR4 | Q7V5N2 | Prochlorococcus marinus (strain MIT |
| | | 9313) |
| Q7V5N3 | Q7V5N2 | Prochlorococcus marinus (strain MIT |
| | | 9313) |
| A0A163MAY1 | A0A163MB05 | Prochlorococcus marinus str. MIT 1318 |
| A0A163MAY9 | A0A163MB05 | Prochlorococcus marinus str. MIT 1318 |
| A0A163UYZ9 | A0A163UYY0 | Prochlorococcus marinus str. MIT 1342 |
| A0A163UZ11 | A0A163UYY0 | Prochlorococcus marinus str. MIT 1342 |
| A0A0A2CVT9 | A0A0A2CSU8 | Prochlorococcus sp. MIT 0701 |
| A0A163G309 | A0A163G301 | Prochlorococcus sp. MIT 1303 |
| A0A163G370 | A0A163G301 | Prochlorococcus sp. MIT 1303 |
| A0A163CFK3 | A0A162EHT7 | Prochlorococcus sp. MIT 1306 |
| A0A163CFM9 | A0A162EHT7 | Prochlorococcus sp. MIT 1306 |
| A0A2W7AW46 | A0A2W7AZA2 | Pseudanabaena sp |
| A0A2W7BIW5 | A0A2W7AZA2 | Pseudanabaena sp |
| A0A1Q3UQZ1 | A0A1Q3URB4 | Rhodospirillales bacterium 69-11 |
| A0A1H8W476 | A0A1H8W4C7 | Rhodospirillales bacterium URHD0017 |
| U5D711 | U5DGM8 | Rubidibacter lacunae KORDI 51-2 |
| A0A2T6CYV8 | A0A2T6CYW6 | Spartobacteria bacterium LR76 |
| A0A140K716 | A0A140K7I7 | Stanieria sp. NIES-3757 |
| A0A354AYF2 | A0A354AYF1 | Synechococcales bacterium UBA10510 |
| K9RV97 | K9RVS0 | Synechococcus sp. (strain ATCC 27167/ |
| | | PCC 6312) |
| K9RWD4 | K9RVS0 | Synechococcus sp. (strain ATCC 27167/ |
| | | PCC 6312) |
| Q0I7K8 | Q0I7K7 | Synechococcus sp. (strain CC9311) |
| Q3AHW8 | Q3AHW7 | Synechococcus sp. (strain CC9605) |
| Q3AZB1 | Q3AZB2 | Synechococcus sp. (strain CC9902) |
| A5GNI4 | A5GNI5 | Synechococcus sp. (strain WH7803) |
| A4CQZ9 | A4CQZ8 | Synechococcus sp. (strain WH7805) |
| A4CR02 | A4CQZ8 | Synechococcus sp. (strain WH7805) |
| A0A0H4BED4 | A0A0H4B9G9 | Synechococcus sp. (strain WH8020) |
| Q7U8L1 | Q7U8L2 | Synechococcus sp. (strain WH8102) |
| A0A0H5PPM7 | A0A0H5Q5R5 | Synechococcus sp. (strain WH8103) |
| A0A2D6Y6K9 | A0A2D6Y6L1 | Synechococcus sp. ARS1019 |
| Q063T1 | Q063T0 | Synechococcus sp. BL107 |
| A0A2D5RBM0 | A0A2D5RBZ8 | Synechococcus sp. CPC100 |
| A0A2D4YV37 | A0A2D4YV84 | Synechococcus sp. CPC35 |
| A0A2D8TUV2 | A0A2D8TUV7 | Synechococcus sp. EAC657 |
| A0A076H3B2 | A0A076H4I8 | Synechococcus sp. KORDI-100 |
| A0A076H859 | A0A076H950 | Synechococcus sp. KORDI-49 |
| A0A076HIY6 | A0A076HGM3 | Synechococcus sp. KORDI-52 |
| A0A2D7JF21 | A0A2D7JF38 | Synechococcus sp. MED650 |
| A0A2D7JF48 | A0A2D7JF38 | Synechococcus sp. MED650 |
| A0A2E1IKX8 | A0A2E1IKT4 | Synechococcus sp. MED850 |
| A0A163XXP8 | A0A163XXR0 | Synechococcus sp. MIT S9504 |
| A0A2E0KHR0 | A0A2E0KJ42 | Synechococcus sp. NAT40 |
| A0A2E9IYA8 | A0A2E9IY90 | Synechococcus sp. NP17 |
| A3Z9D0 | A3Z9D6 | Synechococcus sp. RS9917 |
| A0A1J0P9N7 | A0A1J0PAS0 | Synechococcus sp. SynAce01 |
| A0A1Z8P5Z3 | A0A3R7P7F6 | Synechococcus sp. TMED20 |
| A0A1Z9MG24 | A0A1Z9MG09 | Synechococcus sp. TMED205 |
| A0A1Z9W1Y1 | A0A1Z9W225 | Synechococcus sp. TMED90 |
| A0A1Z9W204 | A0A1Z9W225 | Synechococcus sp. TMED90 |
| A3YUD7 | A3YUD8 | Synechococcus sp. WH 5701 |
| G4FNN6 | G4FNN7 | Synechococcus sp. WH 8016 |
| A0A316JQL6 | A0A316JNT0 | Synechococcus sp. XM-24 |
| A0A068MZG7 | A0A068MZ81 | Synechocystis sp. (strain PCC 6714) |
| A0A068MZS1 | A0A068MZ81 | Synechocystis sp. (strain PCC 6714) |
| P73641 | P73639 | Synechocystis sp. (strain PCC 6803/ |
| | | Kazusa) |
| P73642 | P73639 | Synechocystis sp. (strain PCC 6803/ |
| | | Kazusa) |
| A0A1G7JAL7 | A0A1G7JAI1 | Terriglobus roseus |
| A0A146G9H0 | A0A146GA35 | Terrimicrobium sacchariphilum |
| L8LYM3 | L8M110 | Xenococcus sp. PCC 7305 |

TABLE 6
Precursor (FxsA, IPR026334) and rSS (FxsB, IPR026335)
paired sequences from the UniProt database.

| Accession No | | |
|---|---|---|
| Precursor | Accession No | |
| (FxsA) | rSAM (FxsB) | Strain |
| A0A024YVT1 | A0A024YTX8 | Streptomyces sp. PCS3-D2 |
| A0A086GKG9 | A0A086GKG5 | Streptomyces scabiei |
| A0A086H3F5 | A0A086H3F6 | Streptomyces scabiei |
| A0A0B5DCU4 | A0A0B5D7B6 | Streptomyces nodosus |
| A0A0B5DFK9 | A0A0B5DGY8 | Streptomyces nodosus |
| A0A0C2AZ32 | A0A0C1XRC9 | Streptomyces sp. AcH 505 |
| A0A0C2JH84 | A0A0C2FG78 | Streptomonospora alba |
| A0A0D8BGK1 | A0A0D8BE63 | Frankia torreyi |
| A0A0F0HR20 | A0A0F0HQY3 | Saccharothrix sp. ST-888 |
| A0A0F2TMH1 | A0A0F2TLU9 | Streptomyces rubellomurinus (strain ATCC |
| | | 31215) |
| A0A0F2TP24 | A0A0F2TK09 | Streptomyces rubellomurinus (strain ATCC |
| | | 31215) |
| A0A0F7FYW7 | A0A0F7CPX4 | Streptomyces xiamenensis |
| A0A0F7VTY0 | A0A0F7VWL0 | Streptomyces leeuwenhoekii |
| A0A0G3UPS1 | A0A0G3UX52 | Streptomyces sp. Mg1 |
| A0A0H1ANZ2 | A0A0H1ATT0 | Streptomyces sp. KE1 |
| A0A0L0L3D8 | A0A0L0L3M2 | Streptomyces stelliscabiei |
| A0A0L8KXY1 | A0A0L8KXN5 | Streptomyces resistomycificus |
| A0A0L8N4S2 | A0A0L8N542 | Streptomyces virginiae |
| A0A0M4DX52 | A0A0M4DES0 | Streptomyces pristinaespiralis |
| A0A0M8UJ12 | A0A0M9Z7D0 | Streptomyces sp. H021 |
| A0A0M8X5P8 | A0A0M8X512 | Streptomyces sp. NRRL B-1140 |
| A0A0M8Z5Z8 | A0A0M8Z7D9 | Streptomyces sp. NRRL F-7442 |
| A0A0M9CUH5 | A0A0M9CUQ8 | Streptomyces sp, XY332 |
| A0A0M9X8N0 | A0A0M9X8Q2 | Streptomyces caelestis |
| A0A0N0N1U5 | A0A0N1GCD1 | Actinobacteria bacterium OK074 |
| A0A0N1GPU5 | A0A0N1NRU5 | Actinobacteria bacterium OV320 |
| A0A0N1GVW3 | A0A0N1GG97 | Actinobacteria bacterium OK074 |
| A0A0N1H1K8 | A0A0N1GVW6 | Actinobacteria bacterium OV450 |
| A0A0N6ZI00 | A0A0N6ZHQ7 | Streptomyces sp. CCM_MD2014 |
| A0A0Q1CC38 | A0A0Q0XVU4 | Frankia sp. ACN1ag |
| A0A0Q8P0V1 | A0A0Q8P0C1 | Kitasatospora sp. Root187 |
| A0A0S1UIU0 | A0A0S1UIV4 | Streptomyces sp. FR-008 |
| A0A0S4QS43 | A0A0S4QR97 | Frankia irregularis |
| A0A0T1TPK5 | A0A0T1TPF8 | Streptomyces sp. Root1310 |
| A0A0U3PLY0 | A0A0U3QPY8 | Streptomyces sp. CdTB01 |
| A0A0X3SAJ4 | A0A0X3S963 | Streptomyces sp. NRRL F-5122 |
| A0A0X7JP05 | A0A0X7JP10 | Streptomyces albus subsp. albus |
| A0A100JQ89 | A0A100JQ96 | Streptomyces scabiei |
| A0A100JSG9 | A0A100JSI9 | Streptomyces scabiei |
| A0A100JVX7 | A0A100JVX4 | Streptomyces scabiei |
| A0A101N4D8 | A0A124H9X5 | Streptomyces pseudovenezuelae |
| A0A101SUF2 | A0A124I2K5 | Streptomyces bungoensis |
| A0A117E9F8 | A0A117E9X1 | Streptomyces acidiscabies |
| A0A126Y013 | A0A126Y041 | Streptomyces albidoflavus |
| A0A162JNC9 | A0A166Q011 | Frankia sp. EI5c |
| A0A171DNJ8 | A0A171DNJ7 | Planomonospora sphaerica |
| A0A1A8ZLD1 | A0A1A8ZKQ9 | Micromonospora narathiwatensis |
| A0A1A9CJH0 | A0A1A9CLI2 | Streptomyces sp. OspMP-M45 |
| A0A1A9DPC8 | A0A1A9DPD0 | Streptomyces sp. Ncost-T6T-1 |
| A0A1C4HUF9 | A0A1C4HUC7 | Streptomyces sp. ScaeMP-e83 |
| A0A1C4L932 | A0A1C4L9L5 | Streptomyces sp. TverLS-915 |
| A0A1C4N8D6 | A0A1C4N823 | Streptomyces sp. DvalAA-14 |
| A0A1C4NZW7 | A0A1C4NZD7 | Streptomyces sp. BvitLS-983 |
| A0A1C4TA70 | A0A1C4T9T5 | Streptomyces sp. DvalAA-43 |
| A0A1C4TI64 | A0A1C4TI12 | Streptomyces sp. DfronAA-171 |
| A0A1C4U9B9 | A0A1C4U928 | Micromonospora chokoriensis |
| A0A1C4XM11 | A0A1C4XM63 | Micromonospora coriariae |
| A0A1C5CP40 | A0A1C5CPH1 | Streptomyces sp. Ncost-T10-10d |
| A0A1C5D1B7 | A0A1C5D1A6 | Streptomyces sp. Cmuel-A718b |
| A0A1C5FIC7 | A0A1C5FJB4 | Streptomyces sp. MnatMP-M17 |
| A0A1C5G7Q8 | A0A1C5G8S6 | Micromonospora echinofusca |
| A0A1C5GPW7 | A0A1C5GQK8 | Micromonospora zamorensis |
| A0A1C6NPX7 | A0A1C6NPH8 | Streptomyces sp. AmelKG-D3 |
| A0A1C6UQD4 | A0A1C6UQP0 | Micromonospora eburnea |
| A0A1C6VY14 | A0A1C6VY60 | Micromonospora peucetia |
| A0A1E5PVW4 | A0A1E5Q214 | Streptomyces subrutilus |
| A0A1E7N9W0 | A0A1E7NAH0 | Kitasatospora aureofaciens |
| A0A1E7N9W6 | A0A1E7NA64 | Kitasatospora aureofaciens |
| A0A1G5GGQ1 | A0A1G5GGI7 | Streptomyces sp. 136MFCol5.1 |
| A0A1G5JV31 | A0A1G5JVA0 | Streptomyces sp. 136MFCol5.1 |
| A0A1G6WPA2 | A0A1G6WPJ5 | Alloactinosynnema iranicum |
| A0A1G7C1E1 | A0A1G7C1R1 | Streptomyces jietaisiensis |
| A0A1G7LZV4 | A0A1G7M0C7 | Streptomyces jietaisiensis |
| A0A1G7XUG5 | A0A1G7XUG0 | Streptomyces jietaisiensis |
| A0A1G8WML1 | A0A1G8WMP2 | Nonomuraea maritima |
| A0A1G9DA01 | A0A1G9D9E5 | Nonomuraea jiangxiensis |
| A0A1G9PDZ7 | A0A1G9PD87 | Streptomyces wuyuanensis |
| A0A1H0D7U0 | A0A1H0D7N6 | Streptomyces wuyuanensis |
| A0A1H0WZZ7 | A0A1H0WZZ1 | Lentzea jiangxiensis |
| A0A1H2C4Q2 | A0A1H2C3L8 | Actinoplanes derwentensis |
| A0A1H2CWI0 | A0A1H2CVZ5 | Streptomyces sp. 2114.2 |
| A0A1H4TIP6 | A0A1H4TIA0 | Streptomyces sp. 2131.1 |
| A0A1H5MF42 | A0A1H5MGQ9 | Streptomyces sp. Ag109_O5-10 |
| A0A1H5MSX2 | A0A1H5MT11 | Streptomyces sp. Ag109_O5-10 |
| A0A1H5VHM3 | A0A1H5VJ45 | Streptomyces yanglinensis |
| A0A1H5XYE0 | A0A1H5XX26 | Actinomadura echinospora |
| A0A1H5ZY41 | A0A1H5ZVE5 | Actinomadura echinospora |
| A0A1H6YBE7 | A0A1H6Y914 | Xiangella phaseoli |
| A0A1H7G2N2 | A0A1H7G2Y5 | Streptacidiphilus jiangxiensis |
| A0A1H9WH15 | A0A1H9WGM3 | Actinokineospora terrae |
| A0A1H9WRT3 | A0A1H9WS35 | Streptomyces sp. yr375 |
| A0A1I0LMG3 | A0A1I0LMI5 | Nonomuraea wenchangensis |
| A0A1I2I7E5 | A0A1I215Q1 | Streptomyces alni |
| A0A1I2JTC6 | A0A1I2JW35 | Actinoplanes philippinensis |
| A0A1I3ZHI7 | A0A1I3ZIA4 | Streptosporangium canum |
| A0A1I4X566 | A0A1I4X4G5 | Streptomyces sp. cf124 |
| A0A1I5AVC1 | A0A1I5AVB1 | Streptomyces sp. cf124 |
| A0A1I6CRS4 | A0A1I6CS20 | Lentzea waywayandensis |
| A0A1I6D2T8 | A0A1I6D2V8 | Lentzea waywayandensis |
| A0A1I6UEE3 | A0A1I6UEC1 | Streptomyces harbinensis |
| A0A1K1VQJ3 | A0A1K1VQP5 | Streptomyces atratus |
| A0A1L7GCD1 | A0A1L7GQF0 | Streptomyces sp. TN58 |
| A0A1L7GJB8 | A0A1L7GRF4 | Streptomyces sp. TN58 |
| A0A1L9DLD7 | A0A1L9DXE1 | Streptomyces viridifaciens |
| A0A1L9DLD8 | A0A1L9DLG1 | Streptomyces viridifaciens |
| A0A1M5XAY4 | A0A1M5XB19 | Streptomyces sp. 3214.6 |
| A0A1M6SYF3 | A0A1M6SYI1 | Nocardiopsis flavescens |
| A0A1M6V6Y1 | A0A1M6V748 | Streptomyces paucisporeus |
| A0A1N7CYY2 | A0A1N7CYZ5 | Microbispora rosea |
| A0A1Q4XR29 | A0A1Q4XQY2 | Streptomyces sp. CB03911 |
| A0A1Q4XRD0 | A0A1Q4XQY2 | Streptomyces sp. CB03911 |
| A0A1Q4Y4D4 | A0A1Q4Y5E8 | Streptomyces sp. CB03578 |
| A0A1Q5BD81 | A0A1Q5BE10 | Streptomyces sp. MJM1172 |
| A0A1Q5E401 | A0A1Q5E343 | Streptomyces sp. CB01249 |
| A0A1Q5EUX8 | A0A1Q5EUW4 | Kitasatospora sp. CB01950 |
| A0A1Q5HGD5 | A0A1Q5HGB9 | Streptomyces sp. CB01580 |
| A0A1Q5KB04 | A0A1Q5K8H5 | Streptomyces sp. CB02460 |
| A0A1Q5LG09 | A0A1Q5LG54 | Streptomyces sp. CB03234 |
| A0A1Q5MNP9 | A0A1Q5MP57 | Streptomyces sp. CB02488 |
| A0A1Q5N2E5 | A0A1Q5N491 | Streptomyces sp. CB00455 |
| A0A1Q8UE70 | A0A1Q8UE52 | Streptomyces sp. MNU77 |
| A0A1Q9LP82 | A0A1Q9LPA1 | Actinokineospora bangkokensis |
| A0A1Q9UI73 | A0A1Q9UI65 | Actinomadura sp. CNU-125 |
| A0A1R3UXA7 | A0A1R3UU34 | Nocardiopsis sp. JB363 |
| A0A1S1QFV2 | A0A1S1QJP0 | Frankia sp. Cc1.17 |
| A0A1S1QTS7 | A0A1S1QQZ1 | Frankia sp. EUN1h |
| A0A1S1R984 | A0A1S1R2X2 | Frankia sp. EUN1h |
| A0A1S1RWC7 | A0A1S1RUL9 | Frankia sp. BMG5.36 |
| A0A1S2PZI1 | A0A1S2PWY7 | Streptomyces sp. MUSC 1 |
| A0A1T3NV05 | A0A1T3NV01 | Embleya scabrispora |
| A0A1U9P2I3 | A0A1U9P9Y3 | Streptomyces sp. fd1-xmd |
| A0A1V0ABT3 | A0A1V0ALM0 | Nonomuraea sp. ATCC 55076 |
| A0A1V0QZ43 | A0A1V0RBQ3 | Streptomyces sp. Sge12 |
| A0A1V0R6L6 | A0A1V0RCA9 | Streptomyces sp. Sge12 |
| A0A1V2IMT1 | A0A1V2IMT6 | Frankia sp. BMG5.30 |
| A0A1V2KR92 | A0A1V2KQT6 | Frankia sp. CcI49 |
| A0A1V2QLX0 | A0A1V2QLW7 | Saccharothrix sp. ALI-22-I |
| A0A1V2RG86 | A0A1V2RG00 | Streptomyces sp. MP131-18 |
| A0A1V9KL43 | A0A1V9KLA1 | Streptomyces sp. M41(2017) |
| A0A1V9WGR4 | A0A1V9WHG6 | Streptomyces sp. B9173 |
| A0A1W7CW67 | A0A1W7CV74 | Streptomyces sp. SCSIO 03032 |
| A0A1X1NKK3 | A0A1X1NKM4 | Streptomyces sp. CB03238 |
| A0A209CGC9 | A0A209CGU5 | Streptomyces sp. CS227 |
| A0A209CMP7 | A0A209CMS7 | Streptomyces sp. CS057 |
| A0A212SLW0 | A0A212SLC0 | Streptomyces sp. PgraA7 |
| A0A239B847 | A0A239B9P7 | Actinoplanes regularis |
| A0A239NIM8 | A0A239NHP3 | Actinomadura meyerae |
| A0A239P8P8 | A0A239P749 | Asanoa hainanensis |
| A0A249LUQ9 | A0A249LUL9 | Streptomyces sp. CLI2509 |
| A0A285QR51 | A0A285QM97 | Streptomyces sp. 1331.2 |
| A0A286EAG3 | A0A286EAI9 | Streptomyces sp. 1222.2 |
| A0A286ECT3 | A0A286ECS4 | Streptomyces sp. 1222.2 |
| A0A286EZA4 | A0A286EZ49 | Streptomyces sp. 1222.2 |
| A0A2A3GYD4 | A0A2A3GZ55 | Streptomyces sp. Tue6028 |
| A0A2A3I5U1 | A0A2A3I3N7 | Streptomyces sp. TLI_235 |
| A0A2A4KLS7 | A0A2A4KLL5 | Streptomyces sp. WZ.A104 |
| A0A2B8ATJ3 | A0A2B8B2U6 | Streptomyces sp. Ru87 |
| A0A2C9ZLR6 | A0A2C9ZLR9 | Streptosporangium minutum |
| A0A2D3U667 | A0A2D3UJJ6 | Streptomyces peucetius subsp. caesius ATCC |
| | | 27952 |
| A0A2G5IZM1 | A0A2G5J039 | Streptomyces sp. HG99 |
| A0A2G6XEV4 | A0A2G6XF34 | Micromonospora sp. CNZ299 |
| A0A2G7A2P2 | A0A2G7A0G6 | Streptomyces sp. 1121.2 |
| A0A2G7CIN7 | A0A2G7CIZ2 | Streptomyces sp. 61 |
| A0A2G7DAJ2 | A0A2G7D841 | Verrucosispora sp. CNZ293 |
| A0A2G9DPW9 | A0A2G9DPJ2 | Streptomyces sp. JV178 |
| A0A2H5B440 | A0A2H5B445 | Kitasatospora sp. MMS16-BH015 |
| A0A210SKU9 | A0A210SKT5 | Streptomyces populi |
| A0A2K8PCN9 | A0A2K8PFH7 | Streptomyces lavendulae subsp. lavendulae |
| A0A2L2MIY2 | A0A2L2MIX6 | Streptomyces dengpaensis |
| A0A2M9I333 | A0A2M9I3R2 | Streptomyces sp. TSRI0384-2 |
| A0A2M9K385 | A0A2M9K3V0 | Streptomyces sp. CB01635 |
| A0A2M9KAY5 | A0A2M9KAK8 | Streptomyces sp. CB02120-2 |
| A0A2M9KCW3 | A0A2M9KDT5 | Streptomyces sp. CB02120-2 |
| A0A2M9LGU6 | A0A2M9LGW6 | Streptomyces sp. CB02613 |
| A0A2N0FHQ9 | A0A2N0FHR4 | Streptomyces sp. 4121.5 |
| A0A2N0GTZ4 | A0A2N0GU84 | Streptomyces sp. Ag109_G2-1 |
| A0A2N0IYT9 | A0A2N0IYW6 | Streptomyces sp. 69 |
| A0A2N0JRS8 | A0A2N0JRS9 | Kitasatospora sp. OK780 |
| A0A2N3K0G0 | A0A2N3K0G5 | Streptomyces sp. EAG2 |
| A0A2N3UQP3 | A0A2N3UQM9 | Streptomyces sp. GP55 |
| A0A2N3VTJ9 | A0A2N3VTA9 | Streptomyces sp. TLI_146 |
| A0A2N3Y6P3 | A0A2N3Y6N8 | Saccharopolyspora spinosa |
| A0A2N3YZW9 | A0A2N3YZW5 | Micromonospora sp. CNZ309 |
| A0A2N7T251 | A0A2N7T260 | Verrucosispora sp. ts21 |
| A0A2N9B2G6 | A0A2N9B2E9 | Streptomyces chartreusis NRRL 3882 |
| A0A2P7PXG1 | A0A2P7PXA9 | Streptosporangium nondiastaticum |
| A0A2P7Z906 | A0A2P7Z8Y6 | Streptomyces sp. 111WW2 |
| A0A2P8BLH9 | A0A2P8BLG8 | Streptomyces sp. CS149 |
| A0A2P8I3F8 | A0A2P8I3H1 | Saccharothrix carnea |
| A0A2P8PWL1 | A0A2P8PWM4 | Streptomyces sp. A217 |
| A0A2P9EW35 | A0A2P9EW49 | Streptomyces sp. MA5143a |
| A0A2P9I985 | A0A2P9I9S2 | Actinomadura parvosata subsp. kistnae |
| A0A2R4FSX3 | A0A2R4FSZ2 | Plantactinospora sp. BB1 |
| A0A2R4JG02 | A0A2R4K067 | Streptomyces sp. P3 |
| A0A2R4SZB8 | A0A2R4TDW9 | Streptomyces lunaelactis |
| A0A2S1SQ83 | A0A2S1SQG2 | Streptomyces tirandamycinicus |
| A0A2S1YWM4 | A0A2S1YWL3 | Streptomyces spongiicola |
| A0A2S2FUZ4 | A0A2S2FUN9 | Streptomyces sp. SM17 |
| A0A2S2G322 | A0A2S2GHB9 | Streptomyces sp. SM18 |
| A0A2S3Y395 | A0A2S3Y362 | Streptomyces sp. ZL-24 |
| A0A2S4XWX5 | A0A2S4XX30 | Streptomyces sp. Ru73 |
| A0A2S4YJA9 | A0A2S4YJL5 | Streptomyces sp. Ru71 |
| A0A2S6PXE9 | A0A2S6PXF1 | Streptomyces sp. QL37 |
| A0A2S6WLF2 | A0A2S6WLA7 | Streptomyces sp. MH60 |
| A0A2S6WPG0 | A0A2S6WPF7 | Streptomyces sp. 46 |
| A0A2S9PN61 | A0A2S9PNB9 | Streptomyces sp. ST5x |
| A0A2T0SWN1 | A0A2T0SWM3 | Umezawaea tangerina |
| A0A2T7L4S6 | A0A2T7L4L8 | Streptomyces sp. CS131 |
| A0A2T7L5C6 | A0A2T7L5C0 | Streptomyces sp. CS014 |
| A0A2T7M489 | A0A2T7M3S8 | Streptomyces sp. CS090A |
| A0A2T7MNZ3 | A0A2T7MP23 | Streptomyces sp. CS147 |
| A0A2T7T7D5 | A0A2T7T7K1 | Streptomyces scopuliridis RB72 |
| A0A2V1NLR3 | A0A2V1NLH9 | Streptomyces sp. V2 |
| A0A2V2ATG9 | A0A2V2B402 | Streptomyces sp. CG 926 |
| A0A2V4NJ29 | A0A2V4P5V2 | Streptomyces tateyamensis |
| A0A2W2CFV4 | A0A2W2DMC0 | Jishengella endophytica |
| A0A2W2CGD1 | A0A2W2DGS8 | Micromonospora deserti |
| A0A2W2CK63 | A0A2W2CYC1 | Jishengella endophytica |
| A0A2W4QMB1 | A0A2W4NJL9 | Actinobacteria bacterium |
| A0A2W6CS80 | A0A2W6CMP0 | Pseudonocardiales bacterium |
| A0A2X2P9G4 | A0A2X2LZ37 | Streptomyces griseus |
| A0A2X3L6E8 | A0A2X3KTN6 | Frankia sp. Ea1.12 |
| A0A2Z3UI41 | A0A2Z3UJY5 | Streptosporangium sp. ‘caverna’ |
| A0A2Z4UYC8 | A0A2Z4V9U2 | Streptomyces sp. ICC1 |
| A0A2Z5JLA6 | A0A2Z5JIE4 | Streptomyces atratus |
| A0A2Z5JQL0 | A0A2Z5JQD6 | Streptomyces atratus |
| A0A316FCE1 | A0A316FAP2 | Actinoplanes xinjiangensis |
| A0A317D4S2 | A0A317D6Z3 | Micromonospora sp. 5R2A7 |
| A0A317LK75 | A0A317LL65 | Nocardiopsis sp. L17-MgMaSL7 |
| A0A317S413 | A0A317S3M3 | Actinokineospora mzabensis |
| A0A327TDH6 | A0A327TE11 | Kitasatospora sp. SolWspMP-SS2h |
| A0A327V4K6 | A0A327VFM8 | Streptomyces sp. KhCrAH-43 |
| A0A327ZKA7 | A0A327ZL08 | Actinoplanes lutulentus |
| A0A344TWD6 | A0A344TWD7 | Streptomyces globosus |
| A0A345T341 | A0A345T342 | Streptacidiphilus sp. DSM 106435 |
| A0A358SNX0 | A0A358SPK1 | Actinobacteria bacterium |
| A0A365H3K6 | A0A365H138 | Actinomadura sp. LHW63021 |
| A0A365HA33 | A0A365HAK1 | Actinomadura sp. LHW63021 |
| A0A365ZVQ5 | A0A365ZVT7 | Streptomyces sp. PT12 |
| A0A370B5U2 | A0A370B7F4 | Streptomyces corynorhini |
| A0A370BCA7 | A0A370BHZ7 | Streptomyces corynorhini |
| A0A370RH18 | A0A370RHA5 | Streptomyces sp. HB202 |
| A0A372GAG0 | A0A372G9I9 | Actinomadura sp. LHW52907 |
| A0A380MR20 | A0A380MR53 | Streptomyces griseus |
| A0A384I871 | A0A384IHN3 | Streptomyces sp. AC1-42W |
| A0A385DA15 | A0A385D9S2 | Streptomyces koyangensis |
| A0A388T029 | A0A388T3Z5 | Streptomyces spongiicola |
| A0A397QDY9 | A0A397QHI3 | Streptomyces sp. 19 |
| A0A397R4V6 | A0A397R8E8 | Streptomyces sp. 3211.1 |
| A0A399H7K0 | A0A399H577 | Streptomyces sp. YIM 130001 |
| A0A3A9WFN4 | A0A3A9VZM8 | Streptomyces sp. AZ1-7 |
| A0A3A9YX76 | A0A3A9YZ33 | Streptomyces hoynatensis |
| A0A3A9ZWF6 | A0A3A9ZZ57 | Micromonospora costi |
| A0A3D8NL33 | A0A3D8NL08 | Streptomyces sp. IB2014 011-12 |
| A0A3D9QTI2 | A0A3D9QR75 | Streptomyces sp. 3212.3 |
| A0A3D9SHU3 | A0A3D9SIG7 | Actinomadura umbrina |
| A0A3E0GN80 | A0A3E0GL89 | Streptomyces sp. 2221.1 |
| A0A3G4VQC1 | A0A3G4VVX0 | Streptomyces sp. ADI95-16 |
| A0A3L7BU08 | A0A3L7BU27 | Micromonospora sp. BL4 |
| A0A3L7BWZ6 | A0A3L7BWY8 | Micromonospora sp. CV4 |
| A0A3M8U363 | A0A3M8U433 | Streptomyces sp. NEAU-LD23 |
| A0A3N1HFV6 | A0A3N1HFV9 | Saccharothrix texasensis |
| A0A3N1LYD5 | A0A3N1M2N3 | Streptomyces ossamyceticus |
| A0A3N1SEW3 | A0A3N1SDZ1 | Streptomyces sp. 840.1 |
| A0A3N1SQ42 | A0A3N1SL56 | Streptomyces sp. 840.1 |
| A0A3N1T3X2 | A0A3N1TCT9 | Streptomyces sp. CEV 2-1 |
| A0A3N1U416 | A0A3N1TUF5 | Streptomyces sp. CEV 2-1 |
| A0A3N1UY22 | A0A3N1UZY1 | Streptomyces sp. 2132.2 |
| A0A3N1YVC4 | A0A3N1YYB0 | Kitasatospora cineracea |
| A0A3N4RIC0 | A0A3N4RXG5 | Kitasatospora niigatensis |
| A0A3N4SQP3 | A0A3N4SCI5 | Streptomyces sp. Ag109_O5-1 |
| A0A3N5AL06 | A0A3N5BB93 | Streptomyces sp. Ag109_G2-6 |
| A0A3N6DE32 | A0A3N6FXV8 | Streptomyces sp. ADI91-18 |
| A0A3N6F4K2 | A0A3N6G610 | Streptomyces sp. ADI96-02 |
| A0A3N6FQ75 | A0A3N6FLE5 | Streptomyces sp. ADI97-07 |
| A0A3N6FVN9 | A0A3N6EGY5 | Streptomyces sp. ADI96-15 |
| A0A3N6FX82 | A0A3N6GYK9 | Streptomyces sp. ADI95-17 |
| A0A3N6HTX2 | A0A3N6GKF1 | Streptomyces sp. ADI98-12 |
| A0A3N6I2F3 | A0A3N6GAD3 | Streptomyces sp. ADI95-17 |
| A0A3Q8W8A6 | A0A3Q8WA02 | Streptomyces sp. W1SF4 |
| A0A3R9UNN7 | A0A429RNX4 | Streptomyces sp. WAC06614 |
| A0A3R9UWE6 | A0A429RZ95 | Streptomyces sp. WAC05292 |
| A0A3R9XGC0 | A0A429T9N4 | Streptomyces sp. WAC07149 |
| A0A3R9XP27 | A0A429UH43 | Streptomyces sp. WAC05374 |
| A0A3S8Y671 | A0A3Q8W210 | Streptomyces sp. W1SF4 |
| A0A3T1AXX7 | A0A3T1AXT9 | Actinoplanes sp. OR16 |
| A0A401YSF5 | A0A401YSE7 | Embleya hyalina |
| A0A418N138 | A0A418N231 | Micromonospora radicis |
| A0A421BBS0 | A0A421BBP9 | Actinokineospora cianjurensis |
| A0A421LIK8 | A0A421LIK4 | Streptomyces sp. LaPpAH-201 |
| A0A423V0D6 | A0A423V0C4 | Streptomyces globisporus |
| A0A429F8V5 | A0A429F8W7 | Actinomadura sp. WAC 06369 |
| A0A429I9S6 | A0A429I9T4 | Streptomyces sp. WAC 06783 |
| A0A429INB7 | A0A429ING0 | Streptomyces sp. WAC 06725 |
| A0A429QRZ1 | A0A3R9VYX6 | Streptomyces sp. WAC07061 |
| A0A429T3K9 | A0A3R9XB12 | Streptomyces sp. WAC05950 |
| A0A429TAN1 | A0A3R9VNS4 | Streptomyces sp. WAC07149 |
| A0A429TSQ9 | A0A3R9VYA9 | Streptomyces sp. WAC04770 |
| A0A432N705 | A0A432N6W3 | Verrucosispora sp. FIM060022 |
| A0A495QKT5 | A0A495QL66 | Actinomadura pelletieri DSM 43383 |
| A0A495R149 | A0A495R032 | Actinomadura pelletieri DSM 43383 |
| A0A495TBA2 | A0A495TAE3 | Streptomyces sp. 1114.5 |
| A0A495W527 | A0A495W6M9 | Saccharothrix australiensis |
| A0A495XLA8 | A0A495XKM0 | Saccharothrix variisporea |
| A0A498B7J2 | A0A498B7I9 | Streptomyces sp. 57 |
| A0A4D4J478 | A0A4D4J7P2 | Gandjariella thermophila |
| A0A4D4MQX0 | A0A4D4MQ65 | Streptomyces avermitilis |
| A0A4P6TZ93 | A0A4P6U2L8 | Streptomyces seoulensis |
| A0A4Q6VCA6 | A0A4Q6VAZ3 | Streptomyces sp. SCA2-2 |
| A0A4Q7Z2M9 | A0A4Q7Z4B7 | Streptomyces sp. BK022 |
| A0A4Q7ZMV2 | A0A4Q7ZMV6 | Krasilnikovia cinnamomea |
| A0A4R0GS97 | A0A4R0GXB3 | Micromonospora zingiberis |
| A0A4R1CV15 | A0A4V2P0U2 | Frankia sp. BMG5.11 |
| A0A4R2AZ35 | A0A4R2AYK7 | Micromonospora sp. CNZ303 |
| A0A4R2J4A4 | A0A4V2S5U4 | Actinocrispum wychmicini |
| A0A4R2QP39 | A0A4R2QWF3 | Streptomyces sp. BK438 |
| A0A4R3BLI4 | A0A4R3BPX5 | Streptomyces sp. BK329 |
| A0A4R3CUB3 | A0A4R3CTY5 | Streptomyces sp. BK038 |
| A0A4R3D3G9 | A0A4V2U1S7 | Streptomyces sp. BK308 |
| A0A4R3DA40 | A0A4R3DC57 | Streptomyces sp. BK308 |
| A0A4R3ERL0 | A0A4V6NWQ2 | Streptomyces sp. BK674 |
| A0A4R3IQ37 | A0A4R3IL25 | Streptomyces sp. BK335 |
| A0A4R5C851 | A0A4R5CAU4 | Actinomadura sp. H3C3 |
| A0A4R5FID0 | A0A4R5FIL0 | Nonomuraea sp. 6K102 |
| A0A4R6VA88 | A0A4R6V497 | Actinorugispora endophytica |
| A0A4R7JEF4 | A0A4R7JBB6 | Streptomyces sp. BK447 |
| A0A4R8HAZ4 | A0A4R8HGB2 | Streptomyces sp. 25 |
| A0A4V1B1B4 | A0A4P7DFY5 | Streptomyces sp. S501 |
| A0A4V1VMT8 | A0A4Q4DFM2 | Streptomyces sp. L-9-10 |
| A0A4V2UM06 | A0A4R3IWV4 | Streptomyces sp. BK335 |
| A0A4V2XJX9 | A0A4R4NAH7 | Nonomuraea sp. KC201 |
| A0A4V3ELN6 | A0A4R7IS56 | Streptomyces sp. BK161 |
| A0A4V6Q5J2 | A0A4R7SBU6 | Streptomyces sp. KS 21 |
| A0A4Y8NTS5 | A0A4Y8NTZ5 | Streptomyces sp. ICN441 |
| A0A4Z1DGC7 | A0A4Z1DG56 | Streptomyces bauhiniae |
| A0A4Z1DQ17 | A0A4Z1DRE3 | Streptomyces griseoluteus |
| A0A504DIH5 | A0A504DH74 | Mesorhizobium sp. B2-3-3 |
| A0A505DEP4 | A0A505DJQ4 | Streptomyces sp. NEAU-SSA 1 |
| A0A540Q425 | A0A540Q472 | Streptomyces ipomoeae |
| A0A540Q7K4 | A0A540Q7Z5 | Streptomyces ipomoeae |
| A0A540Q9U8 | A0A540Q9E8 | Streptomyces ipomoeae |
| A0A540QPN3 | A0A540NYL6 | Streptomyces ipomoeae |
| A0A540W473 | A0A540W471 | Kitasatospora sp. MMS16-CNU292 |
| A0A542EYT7 | A0A542EYT6 | Micromonospora sp. A202 |
| A0A542HUG6 | A0A542HU89 | Streptomyces sp. SLBN-115 |
| A0A542Q0K0 | A0A542Q0N6 | Streptomyces sp. SLBN-118 |
| A0A543J3Y2 | A0A543J3Y7 | Thermopolyspora flexuosa |
| A0A543JMS0 | A0A543JMT3 | Saccharothrix saharensis |
| A0A552R3W3 | A0A552R3U5 | Streptomyces sp. 130 |
| A0A560A002 | A0A560A008 | Micromonospora sp. CNZ322 |
| A0A561ETU5 | A0A561ETV0 | Kitasatospora atroaurantiaca |
| A0A561RJY9 | A0A561RJY3 | Streptomyces argenteolus |
| A0A561UGB9 | A0A561UGB0 | Kitasatospora viridis |
| A0A561V213 | A0A561V244 | Streptomyces brevispora |
| A0A561VF89 | A0A561VFB1 | Micromonospora taraxaci |
| A0A5B8E034 | A0A5B8DYW9 | Streptomyces albidoflavus |
| A0A5C4QNY8 | A0A5C4QN11 | Micromonospora orduensis |
| A0A5C4W413 | A0A5C4W1S7 | Nonomuraea phyllanthi |
| A0A5C6IDZ1 | A0A5C6IHR2 | Streptomyces albidoflavus |
| A8M4S4 | A8M4S3 | Salinispora arenicola (strain CNS-205) |
| B5HLH5 | D6XBR5 | Streptomyces sviceus ATCC 29083 |
| B5HUD6 | B5HUD5 | Streptomyces sviceus ATCC 29083 |
| C7PXA6 | C7PXA7 | Catenulispora acidiphila (strain DSM 44928/ |
| | | NRRL B-24433/NBRC 102108/JCM 14897) |
| C9YT11 | C9YT10 | Streptomyces scabiei (strain 87.22) |
| C9Z6K5 | C9Z6K1 | Streptomyces scabiei (strain 87.22) |
| C9ZC34 | C9ZC33 | Streptomyces scabiei (strain 87.22) |
| C9ZCF5 | C9ZCF4 | Streptomyces scabiei (strain 87.22) |
| D2B797 | D2B794 | Streptosporangium roseum (strain ATCC 12428/ |
| | | DSM 43021/JCM 3005/NI 9100) |
| D3D356 | D3D355 | Frankia sp. EUN1f |
| D3D359 | D3D355 | Frankia sp. EUN1f |
| D6B6N6 | D6B6N7 | Streptomyces albidoflavus |
| D6EUL4 | D6EUL3 | Streptomyces lividans TK24 |
| D9VPL0 | D9VPL1 | Streptomyces sp. C |
| D9VYP9 | D9VYQ0 | Streptomyces sp. C |
| D9WR65 | D9WR66 | Streptomyces himastatinicus ATCC 53653 |
| E3JAZ0 | E3JAY9 | Frankia inefficax (strain DSM 45817/CECT |
| | | 9037/EuI1c) |
| E4NFH4 | E4NFH5 | Kitasatospora setae (strain ATCC 33774/DSM |
| | | 43861/JCM 3304/KCC A-0304/NBRC 14216/ |
| | | KM-6054) |
| E8W5K9 | E8W5L0 | Streptomyces pratensis (strain ATCC 33331/ |
| | | IAF-45CD) |
| F3NAU0 | F3NAU3 | Streptomyces griseoaurantiacus M045 |
| F3ND60 | F3ND61 | Streptomyces griseoaurantiacus M045 |
| F3NGR8 | F3NGR7 | Streptomyces griseoaurantiacus M045 |
| F3Z709 | F3Z708 | Streptomyces sp. Tu6071 |
| F4F3S7 | F4F3S8 | Verrucosispora maris (strain AB-18-032) |
| F8B685 | F8B684 | Frankia symbiont subsp. Datisca glomerata |
| G0Q517 | G0Q518 | Streptomyces sp. ACT-1 |
| I0H3J3 | I0H3J2 | Actinoplanes missouriensis (strain ATCC 14538/ |
| | | DSM 43046/CBS 188.64/JCM 3121/ |
| | | NCIMB 12654/NBRC 102363/431) |
| I0L5F6 | I0L5F7 | Micromonospora lupini str. Lupac 08 |
| J7LDH3 | J7LJ81 | Nocardiopsis alba (strain ATCC BAA-2165/ |
| | | BE74) |
| K0K089 | K0K5U7 | Saccharothrix espanaensis (strain ATCC 51144/ |
| | | DSM 44229/JCM 9112/NBRC 15066/NRRL |
| | | 15764) |
| L1KQP3 | L1KQE4 | Streptomyces ipomoeae 91-03 |
| L1L497 | L1L3D8 | Streptomyces ipomoeae 91-03 |
| L7ESL4 | L7ETG5 | Streptomyces turgidiscabies Car8 |
| L7FBZ3 | L7FD96 | Streptomyces turgidiscabies Car8 |
| L8EWX8 | L8F0S4 | Streptomyces rimosus subsp. rimosus (strain |
| | | ATCC 10970/DSM 40260/JCM 4667/NRRL |
| | | 2234) |
| M3D8F8 | M3ETS5 | Streptomyces bottropensis ATCC 25435 |
| M3ESS4 | M3D7E8 | Streptomyces bottropensis ATCC 25435 |
| M3EWW5 | M3FND2 | Streptomyces bottropensis ATCC 25435 |
| Q82BI9 | Q82BJ0 | Streptomyces avermitilis (strain ATCC 31267/ |
| | | DSM 46492/JCM 5070/NBRC 14893/NCIMB |
| | | 12804/NRRL 8165/MA-4680) |
| Q9F3J3 | Q9F3J2 | Streptomyces coelicolor (strain ATCC BAA-471/ |
| | | A3(2)/M145) |
| S2XSG9 | S2YU48 | Streptomyces sp. HGB0020 |
| V4IV16 | V4KJC0 | Streptomyces sp. PVA 94-07 |
| W7IT42 | W7IFD2 | Actinokineospora spheciospongiae |
| W9FQ90 | W9FMS1 | Streptomyces filamentosus NRRL 11379 |

In one embodiment, the rSAM enzyme or enzymatically active fragment has two Cys-rich domains that are critical or essential for activity. The two Cys-rich domains may include the rSAM binding domain in the N-terminus (CXXXCXXC) and the SPASM domain in the C-terminus (CXXXCXXXXXC) or CXXCXXXXXC, where X may be any amino acid).

The term “domain”, as used herein, refers to a part of a molecule or structure that shares common physicochemical features, such as, but not limited to, hydrophobic, polar, globular and helical domains or properties such as ligand-binding, membrane fusion, signal transduction, cell penetration and the like. Often, a domain has a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain. Domains may be co-extensive with regions or portions thereof; domains may also include distinct, non-contiguous regions of a molecule.

The rSAM enzyme may be a recombinant enzyme or is isolated from bacteria.

The term “recombinant” when used with reference to, e.g., polypeptide, enzyme, nucleic acid or cell refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

In some embodiments, the nucleic acid sequence which encodes a rSAM/SPASM maturase comprises Xye, Grr or Fxs. In other embodiments, the nucleic acid sequence comprises Xye.

In one embodiment, the maturase is an enzyme from the XYE maturase system. The enzyme may be a XyeB SPASM protein (e.g. xncB, ykcB or etcB) or an enzymatically active fragment of the enzyme. The polypeptide may be a polypeptide having at least 80% identity to a XyeA precursor peptide (e.g. xncA, ykcA and etcA), including an XyeA precursor peptide that is listed in Table 4. In one embodiment, the polypeptide comprises WIX₄AFX₅NWX₆X₇ (SEQ ID NO: 71), wherein X₄ is N or K, wherein X₅ is G or A, wherein X₆ is E, S or T and wherein X₇ is R or K. The polypeptide may comprise WINAFGNWER (SEQ ID NO: 72), WIKAFGNWSR (SEQ ID NO: 73) or WINAFANWTK (SEQ ID NO: 74), WINAFGNWERAFH (SEQ ID NO: 75), AGWIKAFGNWSRSF (SEQ ID NO: 76) or WINAFANWTKRI (SEQ ID NO: 77).

In one embodiment, the enzyme is an enzyme from the GRR maturase system. The enzyme may be an GrrM SPASM protein (e.g. oscB, lscB or gscB) or an enzymatically active fragment of the enzyme. The enzyme may, for example, act on a peptide having at least 80% identity to an GrrA precursor peptide (e.g. oscA, lscA and gscA), including a GrrA precursor peptide that is listed in Table 5. The polypeptide may comprise

(a)
(SEQ ID NO: 78)
GAWGNGGGRGGWINRGGGGSWGNGGSWRNGGGWRNGWGDGGRFINSR;
(b)
(SEQ ID NO: 79)
GGGFTQGGRRGVATGPRGGNFYNAHPNYGRVGGPVGVGRGAAWADGGGFY
NGTYQDGGSFVNGSDGGAAFKNGTYGAGGFVNGSQGGAGFRNW;
or
(c)
(SEQ ID NO: 80)
GFANGGGGFANRVGPGGFLNDNGGGGFLNNRGWGDGGGGFLNRR.

In one embodiment, the enzyme is an enzyme from the FXS maturase system. The enzyme may be an FxsB SPASM protein (e.g. mscB) or an enzymatically active fragment of the enzyme. The enzyme may, for example, act on a peptide having at least 80% identity to an FxsA precursor peptide (e.g. mscA), including a FxsA precursor peptide that is listed in Table 6. The polypeptide may comprise IPAAKFSSFI (SEQ ID NO: 81).

The terms “Percentage of sequence identity” and “percentage identity” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mo. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Nat/Acad Sci USA 89:10915). Exemplary determination of sequence alignment and % sequence identity can employ the BESTFIT or GAP programs in the GCG Wisconsin Software package (Accelrys, Madison Wis.), using default parameters provided.

The term “nucleic acid” includes a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The terms “nucleic acid”, “nucleic acid molecule”, “nucleic acid sequence” and polynucleotide etc. are used interchangeably herein unless the context indicates otherwise.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include a RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F.

### Sambrook, D. W. Russell, and N. Irwin, Cold Spring Harbor Laboratory Press, 2000.

By “control element” or “control sequence” is meant nucleic acid sequences (e.g., DNA) necessary for expression of an operably linked coding sequence in a particular host cell.

The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

In some embodiments, the precursor polypeptide and the rSAM enzyme are selected from the following Table 7.

TABLE 7
Combination of precursor polypeptide sequence and rSAM sequence.

| Product | Core | | | | Precursor | Precursor | rSAM | |
|---|---|---|---|---|---|---|---|---|
| name | sequence^{a} | MW^{b} | Genus | XyeCDE^{c} | ID^{d} | sequence^{d} | ID^{d} | rSAM sequence^{d} |
| | WVNAFANWSKAL | 1400.56 | Xenorhabdus | CDE | WP_072032494.1 | MSKLQREIA | WP_187650499.1 | MAIVKNEKIKHIEIILKISERCNINCT |
| | | | | | | ENKAQVTNS | | YCYVFNMGNTLAADSTPIISLDNVAAL |
| | | | | | | DKNKTQSKE | | RGFFERSVIENEIEVIQVDFHGGEPLM |
| | | | | | | LVDNLLDTV | | MKKERFNRMCEILREGNYGSSRLVLAL |
| | | | | | | SGGWVNAFA | | QTNGILIDDEWIALFEKHQVHASISID |
| | | | | | | NWSKAL | | GPKHINDRHRLDQKGKSTYEGTVKGLR |
| | | | | | | (SEQ ID | | MLQNAWAQGRIPVEPGILSVANAKANG |
| | | | | | | 82) | | EEIYHHFSKELKCQRFDFLIPDDQHTD |
| | | | | | | | | GIDAEGIGRFLNEALDAWFADGQPNIF |
| | | | | | | | | VRIFNTYLGTMLNNQFSRVLGISANVE |
| | | | | | | | | SAYAFTVTSDGLLRIDDTLRSTSDKIF |
| | | | | | | | | NSIGHVSKLTLASVLESSNVREYLSLS |
| | | | | | | | | DELPDACCGCIWSKVCHGGRLVNRFSQ |
| | | | | | | | | TNRFHNKTVFCPSMRLFLSRAASHLIA |
| | | | | | | | | AGISEETIIENIQK (SEQ ID 138) |
| | WVNAFGNWSKSL | 1402.53 | Xenorhabdus | CDE | WP_099120413.1 | MSKLQREIA | WP_099120414.1 | MAIIKNEKIKHLEIILKVSERCNINCT |
| | | | | | | ENKSQIVNS | | YCYVFNMGNTLAADSAPIISLDNIAAL |
| | | | | | | DKNKTQRKE | | RGFFERSVIENHIEVIQVDFHGGEPLM |
| | | | | | | LVDGLLDTV | | MKKERFNQMCEILREGNYGNSQLVLAL |
| | | | | | | SGGWVNAFG | | QTNGILIDDEWIALFEKHQVHASISID |
| | | | | | | NWSKSL | | GPKHINDRHRLDRKGKSTYEGTVNGLR |
| | | | | | | (SEQ ID | | MLQNAWAQGRIPAEPGILSVANANANG |
| | | | | | | 83) | | GEIYHHFSKELKCQRFDFLIPDDQHAD |
| | | | | | | | | STDAEGIGRFLNEALDAWFADGQPNIF |
| | | | | | | | | VRIFNTYLGTMLNSQFHRIIGISANVE |
| | | | | | | | | SVYAFTVTSDGLLRIDDTLRSTSDKIF |
| | | | | | | | | NPIGHVRELTLSSVLESTNAKEYSSLN |
| | | | | | | | | SELPEDCNDCIWSKICHGGRLVNRFSP |
| | | | | | | | | TNRFHNKTVFCPSMRVFLSRAASHLIE |
| | | | | | | | | AGVSEETIIKNIQQ (SEQ ID 139) |
| | WVNAFANWSKSF | 1450.58 | Xenorhabdus | CDE | WP_193850059.1 | MSKLQREIV | WP_193850057.1 | MAIVKDGKVKHLEVILKISERCNINCT |
| | | | | | | ENKTQVTNS | | YCYVFNMGNTLAADSAPVISLDTVASL |
| | | | | | | DKNKAQRKE | | REFFERSVVENEIEVIQVDFHGGEPLM |
| | | | | | | LVDSLLDTV | | MKKERFNRMCEILREGNYGRSRLVLAL |
| | | | | | | SGGWVNAFA | | QTNGILIDNEWISIFEKHQIHVSVSID |
| | | | | | | NWSKSF | | GPKHINDRYRLDRKGKSTYEGTVNGLR |
| | | | | | | (SEQ ID | | MLQNAWTQGRLSGEPGILSVANAKANG |
| | | | | | | 84) | | EEIYRHFTKELKCQRFDFLIPDDQHAD |
| | | | | | | | | SIDVEGIGRFLNEALDAWFADGQPKIF |
| | | | | | | | | IRIFNTYLGTMLNNQFSRVLGMSANVE |
| | | | | | | | | SAYAFTVTADGQLRVDDTLRSTSDQIF |
| | | | | | | | | SAIGHVSELTLARVLESPNVKEYLSLS |
| | | | | | | | | SELPDACCGCVWSKICHGGRLVNRFSR |
| | | | | | | | | ANRFHNKTVFCLSMRLFLSRAASHLIA |
| | | | | | | | | AGVSEETIIENIQK (SEQ ID 140) |
| | WVNAFARWGKSF | 1462.63 | Erwinia; | CDE | WP_133622747.1 | MSKLSKEIA | WP_133622746.1 | MKNWSQNDLKKIKHLEIILKVSERCNI |
| | | | Kosakonia; | | | KNQAEVITS | | NCSYCYMYNLGNNISIKSKPVIPFSVV |
| | | | Pantoea | | | KDRNEEKKA | | KDLRNFFEQATKEHEIETIQVDFHGGE |
| | | | | | | LAQSMLDSI | | PLMMGKERFEVACDELAKGHYKNTKLN |
| | | | | | | SGGWVNAFA | | MACQTNATLIDDEWIEVFSKYNISVGI |
| | | | | | | RWGKSF | | SIDGPKHINDKHRLDKKGRSTYDKKVN |
| | | | | | | (SEQ ID | | GLKMLQKAWQEGKLADEPGILCVANQS |
| | | | | | | 85) | | VNGAEIYRHFVDDLKSKKFDFLIPDES |
| | | | | | | | | HDTCSNPDGLSKFYCDAMDEFFSDANK |
| | | | | | | | | NVYVRYFHTHMQSMLSQEFRPVMGISK |
| | | | | | | | | SNDDILAFTVCSNGDIYIDDTLRATND |
| | | | | | | | | SIFTPIGNIKNLTLSDALSSWQMKKYI |
| | | | | | | | | LIKKTLPENCTDCVWKKICGGGRHIQR |
| | | | | | | | | YSKDDDFNRETVFCPSIRKIMSRAASH |
| | | | | | | | | LISSGIPEEKIMMNLEII (SEQ ID |
| | | | | | | | | 141) |
| | WVNAFARWGRAF | 1474.65 | Yersinia | DEC | WP_212585760.1 | MSRLKKEII | WP_212585759.1 | MVNISSKKNIQHLEVILKISERCNINC |
| | | | | | | ATKTVVNVS | | DYCYVFNKGNSISDNSPARISSENINQ |
| | | | | | | EAKRNQPQR | | LVYFLORACLEYDIATLQIDFHGGEPL |
| | | | | | | LAEDVLEQV | | LMKKENFARMCDQLVTADYGGSNINLA |
| | | | | | | AGGWVNAFA | | LQTNGTLVDDEWISLFEKYSVNASVSI |
| | | | | | | RWGRAF | | DGPKHINDRHRLDTKGRSTYEGTVRGL |
| | | | | | | (SEQ ID | | RMLQKAYQQGRIPSEPGILCVADASVD |
| | | | | | | 86) | | GAEIYRHFVDELGVYSFDFLIPDDCYK |
| | | | | | | | | DTHVDAIGMGRFLNEALDEWVKDDNPK |
| | | | | | | | | VFVRLFQTHIASLLGQMNSGVLGHNPN |
| | | | | | | | | VTGIYALTVSSDGLVRVDDTLRSTSDS |
| | | | | | | | | MFNPIGHMSEISLLDVFDSQQFREYSL |
| | | | | | | | | IGQSLPTECTGCIWENICAGGRIVNRF |
| | | | | | | | | SPEDRFNRKSTYCYSMRSFLSRASAHL |
| | | | | | | | | LNMGIKEERIMAAISQ (SEQ ID |
| | | | | | | | | 142) |
| | WVNAFVNWPKSF | 1488.67 | Yersinia | DEC | WP_072082693.1 | MSRLQKEIN | WP_050115763.1 | MVNQLNIQSIQHLEIILKISERCNINC |
| | | | | | | ETKTVINIC | | DYCYVFNKGNPAANNSPARLSDRNIND |
| | | | | | | NTKKSQPQH | | LAEFLHTACREYKIGTLQIDFHGGEPL |
| | | | | | | LADSILDKI | | LMKKENFAKMCERLLTGRYSKTNIRFA |
| | | | | | | AGGWVNAFV | | LQTNGTLIDEEWISLFEKYSVNASISI |
| | | | | | | NWPKSF | | DGPKHINDRHRLDTKGRSTYEATVRGL |
| | | | | | | (SEQ ID | | RILQHAHKQGRIPSAPGVLCVANAQAN |
| | | | | | | 87) | | GAEIYRHFVDELKVYGFDFLVPDDCYH |
| | | | | | | | | DTNIDPVGISRFLNEALDEWFKDSNPN |
| | | | | | | | | IFVRLFQTHLAHLLGTKHQGILGHSPS |
| | | | | | | | | ATGAYAFTVGSDGFIRVDDTLRATSDR |
| | | | | | | | | IFNPIGHVSEISLTDALNSPQFQEYAS |
| | | | | | | | | VGQALPHECNGCIWENVCAGGRIMNRF |
| | | | | | | | | SPETRFDRKSVYCYSMRSFLSRAAAHL |
| | | | | | | | | LNMGIKEERIMTAIGR (SEQ ID |
| | | | | | | | | 143) |
| | WINAFARWGRAF | 1488.67 | Yersinia | DEC | WP_071984901.1 | MSSLKKEIM | WP_054871968.1 | MVNISSKKSIQHLEIILKISERCNINC |
| | | | | | | ATKTVVNVS | | DYCYVFNKGNSIADNSPARISNKNIEQ |
| | | | | | | EAKRNHPQR | | LVYFLQRACLEYDIATLQIDFHGGEPL |
| | | | | | | LAEDVLEQI | | LMKKENFASMCDQLTTADYGSSNISLA |
| | | | | | | AGGWINAFA | | LQTNGTLIDDEWISLFEQYLVYVSISI |
| | | | | | | RWGRAF | | DGPKHINDRHRLDTKGRSTYEGTVRGL |
| | | | | | | (SEQ ID | | RMLQNAYKQGRLQAEPGILCVANPQAN |
| | | | | | | 88) | | GAEIYRHFVDDLGVYGFDILIPDDAYN |
| | | | | | | | | DTYADPVSMGRFLNEALDEWMKDDNPK |
| | | | | | | | | IFVRLFQTHIATLLGAKKVGVLGHTPE |
| | | | | | | | | VTGTYACTVGSDGLIRVDDTLRSTSDR |
| | | | | | | | | IFNAIGHVSEINLSDVINSPQFQEYVS |
| | | | | | | | | IGKSLPTECTGCIWENVCAGGRIMNRF |
| | | | | | | | | SPEERFNRKSVYCYSMRSFLSRASAHL |
| | | | | | | | | LNMGIKEERIMAAISQ (SEQ ID |
| | | | | | | | | 144) |
| Xenorceptide A | WVNAFARWSKSF | 1492.66 | Serratia | CDE | WP_071845309.1 | MSKLAKEIN | WP_047728930.1 | MTNKKKIKHLEIILKVSERCNINCTYC |
| | | | | | | MNKAAVTVA | | YVFNLGNDLAINSKPIISHKIIEDLRG |
| | | | | | | ADKKDARKA | | FFERACQEYEIETVQVDFHGGEPLMMG |
| | | | | | | LAQSMLDSV | | KERFDNACKELISGDYNGARLNLACQT |
| | | | | | | SGGWVNAFA | | NAILIDNEWIDIFSKYNISVGISIDGP |
| | | | | | | RWSKSF | | KHINDRHRLDRKGRSTYEGTVKGLEML |
| | | | | | | (SEQ ID | | QVAWKAGRLIDEPGILCVANPSVKGAE |
| | | | | | | 89) | | IYRHFVDVLKCKKFDFLIPDESHDTCT |
| | | | | | | | | DPDGLADFYCSALDEFFLDADKEVYVR |
| | | | | | | | | YFHTHIQSMLSSEFNPVMGVSKAGNDT |
| | | | | | | | | LAFTVSSDGELYVDDTLRATNDPIFTP |
| | | | | | | | | IGNIQHLILSDTLASWQMTKYMAVNSQ |
| | | | | | | | | LPTVCGDCVWQKVCGGGRHIQRYSTAD |
| | | | | | | | | DFNRETVFCPSVRKIMSRAASHLIESG |
| | | | | | | | | VAEDIIMKNLEVNS (SEQ ID 145) |
| | WVNAFVNWTKSF | 1492.66 | Yersinia | DEC | WP_219657009.1 | MSRLQKEIN | WP_219657008.1 | MVNQLNMQSIQHLEIILKISERCNINC |
| | | | | | | ETKTVINIC | | DYCYVFNKGNPAANNSPARLSDKNINA |
| | | | | | | NTKKSQPQH | | LAELLHTACREYKIGTLQIDFHGGEPL |
| | | | | | | LADSILDKI | | LMKKENFAKMCERLPAGKYSKTNVRFA |
| | | | | | | AGGWVNAFV | | LQTNGTLIDEEWISLFEKYSVNASISI |
| | | | | | | NWTKSF | | DGPKHINGRHRLDTKGRSTYEATVRGL |
| | | | | | | (SEQ ID | | RILQHAHKQGRIPSAPGVLCVANAQAN |
| | | | | | | 90) | | GAEIYRHFVDDTLRATSDRIFNPIGHV |
| | | | | | | | | SEISLTDALNSPQFQEYTSIGQSLPHE |
| | | | | | | | | CNGCIWENVCAGGRIMNRFSPETRFDR |
| | | | | | | | | KSVYCYSMRSFLSRTAAHLLNMGIKEE |
| | | | | | | | | RIMAAIQA (SEQ ID 146) |
| | WVNVFARWDKAI | 1498.71 | Xenorhabdus | CDE | WP_071839243.1 | MRKLQREIA | WP_046338175.1 | MITKKKIKHLEIILKVSERCNINCTYC |
| | | | | | | LNNAKVINN | | YVFNLGNEISINSKPIISHDIIKVLRA |
| | | | | | | SEKKQERKV | | FFEQASQEYDIETIQVDFHGGEPLMMG |
| | | | | | | LVENLMDSV | | KEKFENACNEFISGSYNKTKFNLACQT |
| | | | | | | SGGWVNVFA | | NAILIDNEWIDIFSKYNVSVGISIDGP |
| | | | | | | RWDKAI | | KHINDKHRLDRKGRSTYEGTVRGLVML |
| | | | | | | (SEQ ID | | QEAWSAGRLIDQPGILCVANPSVKGAE |
| | | | | | | 91) | | IYRHFVDVLKCKKFDFLIPDESHDTCT |
| | | | | | | | | NPDGLSDFYCSAIDEFFSDADQDVYVR |
| | | | | | | | | YFLTHMQSMLSSEFSPVMGLSKSGSDT |
| | | | | | | | | IALTVSSEGDIYVDDTLRSTNDPIFTP |
| | | | | | | | | IGNVLNLTLSETIASWQMQKYMTVNNQ |
| | | | | | | | | LPTACTDCIWKKVCGGGRHIQRYSKAD |
| | | | | | | | | DFKRESVFCPSIRKIMSRAASHLIESG |
| | | | | | | | | ISEDIIMKNLGIKS (SEQ ID 147) |
| Xenorceptide A3 | WVNAFANWTKRI | 1499.69 | Erwinia | CDE | WP_082262368.1 | MSKLQREIT | WP_168401143.1 | MRLIKGEKIKHLEIIFQVSERCNISCT |
| | | | | | | SNKAQLVNA | | YCYVFNMGNTLAADSHPTISLNNVIAL |
| | | | | | | DARKMQRKV | | RGFFERSTAENEIEVIQVDFHGGEPLM |
| | | | | | | LVDSLLDTV | | MKKDRFDQMCHILLQGDYGNSRIELAL |
| | | | | | | SGGWVNAFA | | QTHGILVDEEWITLFEKYKVHASISVD |
| | | | | | | NWTKRI | | GPKHINDRHRLDRKGKSTYEGTINGLR |
| | | | | | | (SEQ ID | | LLQNAWQQGRLPAEPGILSVANAKANG |
| | | | | | | 92) | | ADIYHHFVDVLKCQRFDFLIPDDHHDD |
| | | | | | | | | ITDSEGIGRFLNEALDAWFADGRAELF |
| | | | | | | | | VRIFNTYLGTLLDKQFSRVLGMSANVE |
| | | | | | | | | SAYAFTVTADGLLRIDDTLRSTSDEIF |
| | | | | | | | | NPVGHVRDLSLAGVLKNTAVEEYLSLS |
| | | | | | | | | NTLPEGCKDCVWNNVCHGGRLVNRFSQ |
| | | | | | | | | ANRFNNKTVFCSSMRIFLSRGASHLMA |
| | | | | | | | | TGIDERTIMANIQG (SEQ ID 148) |
| | WVNAFLRWGKSF | 1504.71 | Yersinia | DEC | WP_071840519.1 | MSRLKKEIT | WP_145595300.1 | MVNISSEKRIKHLEIILKISERCNINC |
| | | | | | | ATKTVINVS | | DYCYVYNKGNTIADNSPARISNKNILQ |
| | | | | | | EVKKNQPQR | | LVDFLQRACREYSIGTLQIDLHGGEPL |
| | | | | | | LAEDVLEQI | | LMKKENFASMCELLMMADYCGSNINLA |
| | | | | | | SGGWVNAFL | | LQTNGTLVDDEWISLFEKYSIHVSISI |
| | | | | | | RWGKSF | | DGPKHINDRHRLDTKGRSTYEGTVRGL |
| | | | | | | (SEQ ID | | RRLQHAHQQGRLRAAPGILCVANPQAS |
| | | | | | | 93) | | GTEIYRHFVDDLGVYGFDLLIPDDAYS |
| | | | | | | | | DDHVDPISMGRFLNEALDEWVKDDNPK |
| | | | | | | | | IFVRLFQTHIATLLGAKVGVLGHTPEV |
| | | | | | | | | TGAYACTVGSDGFIRVDDTLRATSDRI |
| | | | | | | | | FDPIGHVSDISLSEVLDSPQFQEYTLI |
| | | | | | | | | GQSLPTECENCIWAKVCAGGRIMNRFS |
| | | | | | | | | PEDRFNRKSVYCYSMRSFLSRASAHLL |
| | | | | | | | | NMGIKEERIMAAISQ (SEQ ID |
| | | | | | | | | 149) |
| | WINAFANWTKRI | 1513.72 | Erwinia | CDE | WP_017801003.1 | MSKLQHEIA | WP_017801004.1 | MTQLKGEKIKHLEIILKISERCNINCT |
| | | | | | | SNKARLNNA | | YCYVFNMGNTLATDSTPVISLDNVYAL |
| | | | | | | DDKKAQRKI | | RGFFERSAAENDIEVIQVDFHGGEPLM |
| | | | | | | LVDSLLDTV | | MKKDRFDRMCQILLQGNYRSSKFELAL |
| | | | | | | SGGWINAFA | | QTNGILIDDEWIALFEKHQVHASISVD |
| | | | | | | NWTKRI | | GPKHINDRHRLDRKGKSTYEGTITGLR |
| | | | | | | (SEQ ID | | LLQNAWQQGRLPGEPGILSVANANANG |
| | | | | | | 94) | | AEIYRHFADTLQCQRFDFLIPDDHHDD |
| | | | | | | | | SPDGEGVGRFLNEALDAWFADGRPEIF |
| | | | | | | | | IRIFNTYLGTMLNSQFNRVLGMSANVE |
| | | | | | | | | SAYAFTVTADGMLRIDDTLRSTSDEIF |
| | | | | | | | | NAVGHVSELSLARVLETSCVKEYLALS |
| | | | | | | | | SNLPTVCAECVWNNICHGGRLVNRFSR |
| | | | | | | | | TNRFNNKTVFCKSMRLFLSRAASHLMA |
| | | | | | | | | SGVDEKEIMKNIQK (SEQ ID 150) |
| | WVNAFAKWTKRI | 1513.76 | Photorhabdus | DEC | WP_172908095.1 | MSSLKREIA | WP_172908148.1 | MVNSLVKKKIQHLEVILKISERCNINC |
| | | | | | | ETKTEIKGT | | DYCYVFNKGNSAANDSPARISHANIDY |
| | | | | | | KVKNNQPQP | | LVDFFQRGSQEYDIDTLQIDFHGGEPL |
| | | | | | | LTEDLLDQI | | MMKKQQFASMCDRLASGNYHGSNIKFA |
| | | | | | | SGGWVNAFA | | LQTNGILIDDEWISLFEKYSVSVSVSI |
| | | | | | | KWTKRI | | DGPKHINDRHRLDRKGRSTYEGTVRGL |
| | | | | | | (SEQ ID | | RKLQEAYQAGRLPSDPGILCVANAKAS |
| | | | | | | 95) | | GAEIYRHFVDNLGVYGFDFLVPDDCYT |
| | | | | | | | | DALVDPVGVGRFLNEALDEWVNDNNPK |
| | | | | | | | | IFVRLFNTHIASLLGAENAGFLGHNPS |
| | | | | | | | | VAGIYAFTIGSDGSVRIDDTLRSTSDR |
| | | | | | | | | IFDIIGHISEISLSEVLNSPQFQEYVS |
| | | | | | | | | IGQSLPTECEDCIWAKICAGGRIVNRF |
| | | | | | | | | SHEERFKRKSVYCYSMRSLLGRVSAHL |
| | | | | | | | | LNMGIEEDRIMKAISR (SEQ ID |
| | | | | | | | | 151) |
| | WVNFFAKFTKSF | 1515.73 | Salmonella | CDE | WP_153789637.1 | MSKLMKEIE | WP_153789560.1 | MPPFKGGLLMNKEKFNFLEIVLKVSER |
| | | | | | | KQNAKVTVN | | CNINCDYCYMYNCGNELSINSRPLIND |
| | | | | | | NKDKVASRK | | ETVYNLKKLLENAASEFEIGTIQVDFH |
| | | | | | | ELTDAVLDS | | GGEPLMLGKRKFSEACDILLSGNYHNS |
| | | | | | | ITGGWVNFF | | YFILSCQTNGTLIDEEWVDIFYKYNVR |
| | | | | | | AKFTKSF | | IGISIDGPKHINDKHRLDHKGKSTYER |
| | | | | | | (SEQ ID | | TVKGIKMINSAWKKGIMTNEPSILCVI |
| | | | | | | 96) | | NPKVSGKEIYRHFVDDLECKSFDLLIP |
| | | | | | | | | DENHDTCENTKAVGLYLNEAVDEFFND |
| | | | | | | | | SNKEIEVRIIATHMKSLMLKEFTPVIG |
| | | | | | | | | ISKGDINSAVFVITSEGDIYIDDALRV |
| | | | | | | | | TNDILFSPIGNLRNVKFKNLLESWQLK |
| | | | | | | | | QYMNINNTLPSSCYDCIWKNSCFGGRA |
| | | | | | | | | LNRFSKVNRFDNKTVFCDSMRIFLSRL |
| | | | | | | | | TSHIIESGVDIKLIEENLGVNEL |
| | | | | | | | | (SEQ ID 152) |
| | WVNAFLNWSRSF | 1520.67 | Yersinia | DEC | WP_074006888.1 | MSRLKKEIT | WP_128450850.1 | MGHLLTKKRIKHFEIILKISERCNINC |
| | | | | | | ETKTAIGTN | | DYCYVFNKGNSDADNNPARISNKNIGH |
| | | | | | | KAKKNQPQH | | LANFLQRACLEYEIDTLQIDFHGGEPL |
| | | | | | | LADDLLDQI | | LMKKEHFANMCIQLISGNYCGSNIRLA |
| | | | | | | AGGWVNAFL | | LQTNGILIDDEWISLFEKYSVNVSLSI |
| | | | | | | NWSRSF | | DGPKHINDRHRLDTKGRSTYEGTVRGL |
| | | | | | | (SEQ ID | | RLLQSAYQQGRLPSAPGILCVANAQAN |
| | | | | | | 97) | | DAEIYRHFVDDLGVYGFDFLIPDDSYN |
| | | | | | | | | DVNIDPIGIGRFLNEALDEWVKDNNPK |
| | | | | | | | | IFVRHFQTHFASLLGVKNIGILGQSSN |
| | | | | | | | | ITGVYAFTVSSDGSIRVDDTLRSTSDR |
| | | | | | | | | IFNTIGHISEINLSDVLNSPQAQEYSS |
| | | | | | | | | IGQCLPNECKGCIWENICTGGRLVNRF |
| | | | | | | | | SSEERFKHKSVYCYSIRSFLSRASAHL |
| | | | | | | | | LNMGIKEERIMTSICQ (SEQ ID |
| | | | | | | | | 153) |
| | WVNAFANWPKRF | 1529.72 | Erwinia | CDE | WP_212410257.1 | MKTLKREIE | WP_212410258.1 | MGANKEKIKHLEIILKISERCNINCDY |
| | | | | | | RNNCQLTDV | | CYVFNMGNQLATESNPVISMSNILSLR |
| | | | | | | DVVTKKAER | | GFFERSVKEYEINVLQVDFHGGEPLMI |
| | | | | | | KALVDGLLD | | KKSRFDEMCEILKGGNYSNSKLELALQ |
| | | | | | | TVSGGWVNA | | TNGILIDEEWIVLFEKHKVHVSISVDG |
| | | | | | | FANWPKRF | | PKHINDRHRLDRKGKSTYEGTIKGFRL |
| | | | | | | (SEQ ID | | LQDAWESGRIPGEPGILSVANAKANGA |
| | | | | | | 98) | | EIYRHFVDVLDCKRIDFLIPDDHHNDE |
| | | | | | | | | VDSQGIGMFLTEALDEWFSDGNSGVFV |
| | | | | | | | | RIFNTYLGTMLNHQFSRVLGMSANVES |
| | | | | | | | | AYAFTVTSDGIIRIDDTLRSTSDKIFD |
| | | | | | | | | ALGHVDEMSLSDVFEHNNFKEYIYLNA |
| | | | | | | | | VLPAGCHGCLWSNICHGGRLVNRFSLD |
| | | | | | | | | GRFNNKTIFCSSMKIFLSRAVAHLLAS |
| | | | | | | | | GIEEETIIKNIEKKEISV (SEQ ID |
| | | | | | | | | 154) |
| | WVNAFLNWPRSF | 1530.71 | Yersinia | DEC | WP_072089902.1 | MSRLKKEIT | WP_050317896.1 | MDNLLTKKRIKHFEIILKISERCNINC |
| | | | | | | ETKTAIGSN | | DYCYVFNKGNSDADNNPARISNTNISH |
| | | | | | | KAKKNQPQH | | LANFLORACFEYEIDTLQIDFHGGEPL |
| | | | | | | LADDLLDQI | | LMKKEHFANMCIQLISGNYRGSSIRLA |
| | | | | | | AGGWVNAFL | | LQTNGTLIDDEWISLFEKYSVNVSISI |
| | | | | | | NWPRSF | | DGPKHINDRHRLDTKGRSTYEGTVRGL |
| | | | | | | (SEQ ID | | RLLQSAYRQGRLPSAPGILCVANARAN |
| | | | | | | 99) | | GAEIYRHFVDDLGVYGFDFLIPDDSYN |
| | | | | | | | | DVNIDPIGIGRFLNEALDEWVKDNNPK |
| | | | | | | | | IFVRHFQTHFASLLGVRNIGVLGQSSN |
| | | | | | | | | ITGVYAFTVGSDGSIRVDDTLRSTSDR |
| | | | | | | | | IFNTIGHISEINLSDVLNSPQAQEYSS |
| | | | | | | | | IGQCLPNECKGCIWENICTGGRLVNRF |
| | | | | | | | | SSEERFKHKSVYCYSIRSFLSRASAHL |
| | | | | | | | | LDMGIKEERIMAAISQ (SEQ ID |
| | | | | | | | | 155) |
| | WVNAFANWTKRF | 1533.71 | Aeromonas | DEC | WP_201910365.1 | MSKLQREIA | WP_201910362.1 | MTLIKGEKIKHLEIILKISERCNISCT |
| | | | | | | LNKTKLINA | | YCYVFNMGNSLAADSSPVMSLDNVLAL |
| | | | | | | DDKKVERKV | | RGFFERSASENEIEVIQVDFHGGEPLM |
| | | | | | | LVDSLLDTV | | MKKNRFDQMCNILLQGNYGNSRLELAL |
| | | | | | | SGGWVNAFA | | QTNGILIDEEWITLFEKHKVHTSISVD |
| | | | | | | NWTKRF | | GPKHINDRHRLDRKGKSTYEGTINGLR |
| | | | | | | (SEQ ID | | LLQKAWEQGRLPGEPGILSVANAKANG |
| | | | | | | 100) | | AEIYRHFVDVLKCQRFDFLIPDDHHDD |
| | | | | | | | | NTDNEGVGKFLNEALDAWFADGRPELF |
| | | | | | | | | VRIFNTYLGTMLDNQFSRVLGMSANVE |
| | | | | | | | | SAYAFTVTADGLLRIDDTLRSTSDEIF |
| | | | | | | | | NAVGHVRDLSLKSVLKNSSVKEYLSLS |
| | | | | | | | | GELPNDCVDCVWNNVCHGGRLVNRFSK |
| | | | | | | | | ANRFNNKTVFCSSMRVFLSRAAAHLMA |
| | | | | | | | | TGIDERAIMENIQK (SEQ ID 156) |
| | WVNAFARFTKRF | 1536.76 | Vibrio | DE | WP_083932216.1 | MSKLEKEIT | WP_039980110.1 | MIRKKIKHLEIILKVSERCNINCTYCY |
| | | | | | | INNASVSLN | | VFNLGNDIAINSKPIISHQNIKHLKHF |
| | | | | | | KEVKPEKNK | | FERATREYEIESLQVDFHGGEPLMMGK |
| | | | | | | DKNELVQSM | | ERFKAACKELMSGDYQNSRLSLACQTN |
| | | | | | | LDSVSGGWV | | AILIDDEWIDIFSKYDVSVGISIDGPK |
| | | | | | | NAFARFTKR | | HINDKHRIDRKGRGTYDDTVAGLKKLQ |
| | | | | | | F (SEQ | | AAWEEGKIADEPGILCVANPSVKGADI |
| | | | | | | ID 101) | | YRHFVDVLGCKKFDFLIPDESHDTCED |
| | | | | | | | | PHSLAEFYCSALDELFNDADKDIYVRY |
| | | | | | | | | FHTHIHSMLASNFNPVMGMSKSTNDTI |
| | | | | | | | | AYTVSSEGELYIDDTLRATNDNIFTSI |
| | | | | | | | | GNIKDLTLSESINSWQMQKYMQVNNQT |
| | | | | | | | | PEPCSECIWKNICGGGRHIQRYSKEDD |
| | | | | | | | | FNRNSVYCPSIRKIMSRTASHLISSGI |
| | | | | | | | | PEEKILTNLGVHN (SEQ ID 157) |
| | WINVFARWNRAI | 1539.76 | Xenorhabdus | CDE | WP_092519408.1 | MSELQREIA | WP_175486043.1 | MLTMIKKKKIKHLEIILKVSERCNINC |
| | | | | | | LNNAQVINS | | TYCYVFNLGNEISINSKPIISHSTIKD |
| | | | | | | SEKKQERKE | | LRAFFEQASQEYDIETIQVDFHGGEPL |
| | | | | | | LVENLMDSV | | MMGKEKFENACNEFISGGYNKTKLNLA |
| | | | | | | SGGWINVFA | | CQTNAILIDNEWIDIFSKYNVSVGISI |
| | | | | | | RWNRAI | | DGPKHINDKYRLDRKGRSTYEGTVRGL |
| | | | | | | (SEQ ID | | VMLQEAWNAGRLIDQPGILCVANPSVK |
| | | | | | | 102) | | GAEIYRHFVDVLKCKKFDFLIPDESHD |
| | | | | | | | | TCANPDGLSDFYCSVIDAFFSDADQDV |
| | | | | | | | | YVRYFLTHMQSMLSSEFSPVMGLNKSG |
| | | | | | | | | NDTIALTVSSEGDIYVDDTLRSTNAPI |
| | | | | | | | | FTSIGNILNLTLSETIASWQMQKYMTV |
| | | | | | | | | NNQLPTACTDCIWKKVCGGGRHIQRYS |
| | | | | | | | | KADDFKRESVFCPSIRKIMSRAASHLI |
| | | | | | | | | ESGISEDIIMKNLGIKS (SEQ ID |
| | | | | | | | | 158) |
| | WVNVFARWDKQI | 1555.76 | Providencia | D | WP_206277116.1 | MSKLSKEIK | WP_206277115.1 | MDKIKHLEVILKVSERCNINCTYCYVF |
| | | | | | | ENNANVKLA | | NLGNEVAINSKPIISSEIINHLVEFFE |
| | | | | | | SNERSSRET | | QATTEYDIESIQVDFHGGEPLMMGKKR |
| | | | | | | LVKSMLESV | | FIAACQKLISGNYNNTKLYLACQTNAI |
| | | | | | | SGGWVNVFA | | LIDPDWIDIFSKYSISIGVSIDGPKHI |
| | | | | | | RWDKQI | | NDKHRLDTKGRSTYDNTIKGFKLLQNA |
| | | | | | | (SEQ ID | | WREGKLKDQPGILCVANPNVSGKDIYR |
| | | | | | | 103) | | HFVDELECTKFDFLIPDETHDTCIDPT |
| | | | | | | | | HLSEFYCSALDEFFLDSNNDIYIRYFH |
| | | | | | | | | TNIQSMLKSDFTPTMGVSKTSNDIIAL |
| | | | | | | | | TISSEGDVYIDDTLRGTNDDIFSVIGN |
| | | | | | | | | IKKTKFRETLSSWQMEKYMQINSQLPS |
| | | | | | | | | DCVNCIWKKTCSGGRHIQRYSKADNFN |
| | | | | | | | | RKSVFCPSIKKILSRAASHLLESGVPE |
| | | | | | | | | ELIMDNLGIKS (SEQ ID 159) |
| Xenorceptide A4 | WVNAFARWDKKF | 1561.77 | Sodalis | CDE | WP_213989265.1 | MSKLIKEIN | WP_213989266.1 | MIKIKHLEIILKVSERCNINCTYCYVF |
| | | | | | | FNKAAVTIV | | NLGNDISINSKPIISHDIIKDLTGFLE |
| | | | | | | ADNKNAKKA | | RASHEYDIETIQIDFHGGEPLMMGKEK |
| | | | | | | LTQAMLDSI | | FDSACRDFLSGNYKKSRLQLACQTNAM |
| | | | | | | SGGWVNAFA | | LIDEEWIDIFSNNNISVGVSIDGPKHI |
| | | | | | | RWDKKF | | NDKHRLDRKGRSTYEGTVKGLVMLQDA |
| | | | | | | (SEQ ID | | WQAGRLIDEPGILCVANSLVNGAEIYR |
| | | | | | | 104) | | HFVDVLHCKKIDFLIPDETHDTCKDPE |
| | | | | | | | | GLSDFYCSAIDEFFSDADSNVYIRFFY |
| | | | | | | | | THIQSMLNSDLSPVLGLSKSESDTLAF |
| | | | | | | | | TVGSEGELYVDDTLRATNDPIFTSIGN |
| | | | | | | | | VRNLSLSETIASWQMQKYMAVNNNLPL |
| | | | | | | | | VCTDCIWQKICGGGRHIQRYSKADDFN |
| | | | | | | | | RETVFCPSIRKIMSRAASHLLDCGVSE |
| | | | | | | | | NTIMKNLDS (SEQ ID 160) |
| | WLNVFVRWDRAI | 1568.8 | Xenorhabdus | CDE | WP_071826505.1 | MSKLQREID | WP_196243385.1 | MITMIAKKKIKHLEIILKVSERCNINC |
| | | | | | | LNNAQVINS | | TYCYVFNLGNEISINSKPIISHNTIKD |
| | | | | | | SEKKQERKE | | LRAFFEQASQEYDIETIQVDFHGGEPL |
| | | | | | | LVENMMDSV | | MMGREKFENACNEFISGSYNKTKLNLA |
| | | | | | | SGGWLNVFV | | CQTNAILIDNEWIDIFSKYNVSVGISI |
| | | | | | | RWDRAI | | DGPKHINDKYRLDRKGRSTYEGTVRGL |
| | | | | | | (SEQ ID | | VMLQEAWNAGRLIDQPGILCVANPSVK |
| | | | | | | 105) | | GAEIYRHFVDVLKCKKFDFLIPDESHD |
| | | | | | | | | TCANPDGLSDFYCSVIDEFFSDADQDV |
| | | | | | | | | YVRYFFTHMQSMISSEFSPVMGLSKSG |
| | | | | | | | | SDTIALTVSSEGDIYVDDTLRATNDPI |
| | | | | | | | | FTPIGNILNLTLSETIASWQMQKYMTV |
| | | | | | | | | NNQLPTACTDCIWKKVCGGGRHIQRYS |
| | | | | | | | | KADDFKRESVFCPSIRKIMSRAASHLI |
| | | | | | | | | ESGISEDIIMKNLGIK (SEQ ID |
| | | | | | | | | 161) |
| | WVNAYARWTNRF | 1577.72 | Photorhabdus | DEC | WP_072023203.1 | MEESFMSNL | WP_036768348.1 | MVNSLVKKKIQHLEVILKISERCNINC |
| | | | | | | KKEIAETKT | | DYCYVFNRGNSAANDSPARISHANIDY |
| | | | | | | EIKGTKVKN | | LVDFFQRGSQEYDIDTLQIDFHGGEPL |
| | | | | | | NQPQPLTED | | MMKKPQFASMCERLASGNYHGSKIRFA |
| | | | | | | LLDQISGGW | | LQTNGILIDDEWISLFEKYSVSVSVSI |
| | | | | | | VNAYARWTN | | DGPKHINDRHRLDRKGRSTYEGTIRGL |
| | | | | | | RF (SEQ | | RKLQEAYQAGRLPSDPGILCVANAKAS |
| | | | | | | ID 106) | | GAEIYRHFVDNLGVYGFDFLVPDDCYT |
| | | | | | | | | DAQVDPDGVGRFLNEALDEWVNDNNPK |
| | | | | | | | | IFVRLFNTHIASLLGAENAGFLGHNPS |
| | | | | | | | | VAGIYAFTIGSDGFVRVDDTLRSTSDR |
| | | | | | | | | IFDIIGHISEISLSEVLNSPQFQEYAS |
| | | | | | | | | IGESLPTECEDCIWAKVCAGGRIVNRF |
| | | | | | | | | SHEERFKRKSVYCYSMRSLLSRVSAHL |
| | | | | | | | | LNMGIEEDRIMKAIGR (SEQ ID |
| | | | | | | | | 162) |
| | WVNAYARWTKRF | 1591.79 | Photorhabdus | DEC | WP_214085658.1 | MSSLKKEIA | WP_214085659.1 | MVNSLVKKKIQHLEVILKISERCNINC |
| | | | | | | ETKTEIKGT | | DYCYVFNRGNSAANDSPARISHANIDY |
| | | | | | | KVKNNQPQP | | LVDFFQRGSQEYDIDTLQIDFHGGEPL |
| | | | | | | LTEDLLDQI | | MMKKQQFASMCERLASGNYYGANIRFA |
| | | | | | | SGGWVNAYA | | LQTNGILIDDEWISLFEKYSVSVSVSI |
| | | | | | | RWTKRF | | DGPKHINDRHRLDRKGRSTYEGTVRGL |
| | | | | | | (SEQ ID | | RKLQEAYQEGRLPSDPGILCVANAKAS |
| | | | | | | 107) | | GAEIYRHFVDNLGVYGFDFLVPDDCYT |
| | | | | | | | | DAQVDPVGVGRFLNEALDEWVNDNNPK |
| | | | | | | | | IFVRLFNTHIASLLGAENAGFLGHNPS |
| | | | | | | | | VAGIYAFTIGSDGSVRVDDTLRSTSDR |
| | | | | | | | | IFDIIGHISEISLSEVLNSPQFQEYSS |
| | | | | | | | | IGESLPTECEDCIWAKVCAGGRIVNRF |
| | | | | | | | | SNEERFKRKSVYCYSMRSLLGRVSAHL |
| | | | | | | | | LNMGIEEDRIMKAIGR (SEQ ID |
| | | | | | | | | 163) |
| | AGWINAFGNWTKSF | 1592.73 | Yersinia | DEC | WP_072080131.1 | MSRLKKEIT | WP_050143454.1 | MVELLINKRIRHLEIILKISERCNINC |
| | | | | | | ATKTVINVN | | DYCYVFNKGNSAANDSPARISDKNIHH |
| | | | | | | EVKKSQPQR | | FVNFLERASQEYQIGTLQIDLHGGEPL |
| | | | | | | LAEDALEQI | | LMKKENFANMCIQFMSGHYCGSNIRLA |
| | | | | | | TGGAGWINA | | LQTNGTLIDEEWIALFERYSVNVSVSI |
| | | | | | | FGNWTKSF | | DGPKHINDRHRLDTKGRSTYEGTVRGL |
| | | | | | | (SEQ ID | | RMLQQAYQQGRLPSAPGILCVANAKVN |
| | | | | | | 108) | | GAEIYRHFVDDLGVYSFDFLIPDDCYK |
| | | | | | | | | DADVDSLGLGRFLNEALDEWVKDDNPK |
| | | | | | | | | IFVRLFQTHIATLLGQKNSGILGHNPS |
| | | | | | | | | VTGVYALTVSSDGFVRVDDTLRSTSDS |
| | | | | | | | | MFNPIGHTSEVSLSEVFDSPQFREYTS |
| | | | | | | | | VGQSLPTECTGCIWENICAGGRIVNRF |
| | | | | | | | | SPEDRFDRKSAYCYSMRSFLSRASAHL |
| | | | | | | | | INMGIKEERIMAAISQ (SEQ ID |
| | | | | | | | | 164) |
| | AGWINAFANWTKSF | 1606.76 | Yersinia | DEC | WP_071984814.1 | MSRLKKEIT | WP_050538194.1 | MVELLIDKRIRHLEIILKISERCNINC |
| | | | | | | ATKTVINVN | | DYCYVFNKGNSAANDSPARISDKNIHH |
| | | | | | | EVKKSQPQR | | FINFLERASQEYQIGTLQIDLHGGEPL |
| | | | | | | LAEETLEQI | | LMKKENFANMCIQFMSGHYCGSNIRLA |
| | | | | | | AGGAGWINA | | LQTNGTLIDEEWIALFEKYSVNVSVSI |
| | | | | | | FANWTKSF | | DGPKHINDRHRLDTKGRSTYEGTVRGL |
| | | | | | | (SEQ ID | | RMLQQAYQQGRLPSAPGILCVANAKVN |
| | | | | | | 109) | | GAEIYRHFVDDLGVYSFDFLIPDDCYK |
| | | | | | | | | DADVDALGLGRFLNEALDEWVKDDNPK |
| | | | | | | | | IFVRLFQTHIATLLGQKNSGILGHNPS |
| | | | | | | | | VTGVYALTVSSDGFVRVDDTLRSTSDS |
| | | | | | | | | MFNPIGHTSEVSLSEVFDSPQFREYTS |
| | | | | | | | | VGQSLPTECTGCIWENICAGGRIVNRF |
| | | | | | | | | SPEDHFDRKSAYCYSMRSFLSRASAHL |
| | | | | | | | | INMGIKEERIMAAISQ (SEQ ID |
| | | | | | | | | 165) |
| | AGWIKAFGNWSRSF | 1620.79 | Yersinia | DEC | WP_072088965.1 | MSRLOKEII | WP_050291264.1 | MLNLLIEKNIRHLEIILKISERCNINC |
| | | | | | | ETKTVIDVS | | DYCYVFNKGNSAADDSPARLSNKNIHH |
| | | | | | | GAKKSQPQR | | LVCFLQRACQEYKIGTVQIDFHGGEPL |
| | | | | | | LTEDVLEQI | | LMKKENFTDMCIQLISGNYCGSNIRLA |
| | | | | | | AGGAGWIKA | | LQTNATLIDNEWIAIFEKYSVNVSISI |
| | | | | | | FGNWSRSF | | DGPKHINDRHRLDTKGRSTYESTVRGL |
| | | | | | | (SEQ ID | | RILQNAYQQGRLPSDPGILCVTNAQAN |
| | | | | | | 110) | | GAEIYRHFVDELGVYSFDFLIPDDSYK |
| | | | | | | | | DAHPDAVGIGRFLNEALDEWVKDNNAK |
| | | | | | | | | IFVRLFQTHIASLLGQKNSGVLGHTPN |
| | | | | | | | | ITGVYALTVSSDGFVRVDDTLRSTSDR |
| | | | | | | | | MFNPIGHLSEVNLSNVFASPQFQEYSS |
| | | | | | | | | IGQSLPTECEGCIWENICAGGRIVNRF |
| | | | | | | | | STEDRFKHKSIYCYSMRTFLSRSSAHL |
| | | | | | | | | LNMGIKEERIMAAIRA (SEQ ID |
| | | | | | | | | 166) |
| | WVNAFARWSRRW | 1628.82 | Serratia | CD | WP_072056064.1 | MSKLAKEIS | WP_072056065.1 | MANKEKIKHLEIILKVSERCNINCTYC |
| | | | | | | MNKAAVIID | | YVFNLGNDLAINSKPIISHGVIKNLRE |
| | | | | | | GDKKDIRRA | | FFERACREYEIETVQVDFHGGEPLMMG |
| | | | | | | LTQSMLDSI | | KDRFDNACKELVSGDYNGTRLNLACQT |
| | | | | | | SGGWVNAFA | | NAILIDNEWIDIFSKYNMSVGISIDGP |
| | | | | | | RWSRRW | | KHINDRHRLDRKGRSTYEGTVKGLEML |
| | | | | | | (SEQ ID | | QVAWRAGRLIDEPGILCVANPSVKGAE |
| | | | | | | 111) | | IYRHFVDVLKCKKFDFLIPDESHDTCT |
| | | | | | | | | DPEGLSDFYCSALDEFFLDADKEVYVR |
| | | | | | | | | YFHTHIQSMLSSEFSPVMGVSKAGSDT |
| | | | | | | | | LAFTVSSDGELYVDDTLRSTNDSIFTP |
| | | | | | | | | IGNLHSLTLSEALMSWQMQKYLSVDNQ |
| | | | | | | | | LPKVCIDCVWKKLCGGGRHIQRYSSND |
| | | | | | | | | DFNRETVFCPSIRKIMSRAASHLIESG |
| | | | | | | | | VSEDVIMKNLEVNS (SEQ ID 167) |
| | AGWINAFANWTRSF | 1634.77 | Yersinia | DEC | WP_072079580.1 | MSRLKKEIT | WP_099466089.1 | MVETLIDKRIRHLEIILKISERCNINC |
| | | | | | | ATKTVINVS | | DYCYVFNKGNSAANDSPARISDKNIRH |
| | | | | | | DVKKSQPQR | | FVDFLERASQEYQIGTLQIDLHGGEPL |
| | | | | | | LAEDALEQI | | LMKKENFANMCIQFMSGYYCGSNIRLA |
| | | | | | | AGGAGWINA | | LQTNDTLIDEEWIALFGKYSVNVSVSI |
| | | | | | | FANWTRSF | | DGPKHINDRHRLDTKGRSTYEGTVRGL |
| | | | | | | (SEQ ID | | RMLQQAYQQGRLPSAPGILCVANANVN |
| | | | | | | 112) | | GAEIYRHFIDELGVYSFDFLIPDDCYK |
| | | | | | | | | DTYVDAVGMARFLNEALDEWVKDNNPK |
| | | | | | | | | IFVRLFQTHIATLLGQKNSGILGHNPS |
| | | | | | | | | VTGVYALTVSSDGFVRVDDTLRSTSDP |
| | | | | | | | | MFNPIGHTSEVSLSEVFNSPQFQEYSS |
| | | | | | | | | IGQSLPTECAGCIWENICAGGRIVNRF |
| | | | | | | | | SPEDRFDRKSAYCYSMRSFLSRASAHL |
| | | | | | | | | INMGIKEERIMAAISQ (SEQ ID |
| | | | | | | | | 168) |
| Xenorceptide A1 | WINAFGNWERAFH | 1641.77 | Xenorhabdus | CDE | WP_010848441.1 | MSKLQREIA | WP_010848442.1 | MTTSKSEKIKHLEIILKISERCNINCS |
| | | | | | | ANKAQLSHE | | YCYVFNMGNSLATDSPPVISLDNVLAL |
| | | | | | | DKKKTQHKE | | RGFFERSAAENEIEVIQVDFHGGEPLM |
| | | | | | | LVDSLLDTV | | MKKDRFDQMCDILRQGDYSGSRLELAL |
| | | | | | | SGGWINAFG | | QTNGILIDDEWISLFEKHKVHASISID |
| | | | | | | NWERAFH | | GPKHINDRYRLDRKGKSTYEGTIHGLR |
| | | | | | | (SEQ ID | | MLQNAWKQGRLPGEPGILSVANPTANG |
| | | | | | | 113) | | AEIYHHFANVLKCQHFDFLIPDAHHDD |
| | | | | | | | | DIDGIGIGRFMNEALDAWFADGRSEIF |
| | | | | | | | | VRIFNTYLGTMLSNQFYRVIGMSANVE |
| | | | | | | | | SAYAFTVTADGLLRIDDTLRSTSDEIF |
| | | | | | | | | NAIGHLSELSLSGVLNSPNVKEYLSLN |
| | | | | | | | | SELPSDCADCVWNKICHGGRLVNRFSR |
| | | | | | | | | ANRFNNKTVFCSSMRLFLSRAASHLIT |
| | | | | | | | | AGIDEETIMKNIQK (SEQ ID 169) |
| | AGWIKVFGNWSRSF | 1648.84 | Yersinia | C | WP_071881823.1 | MKKEIIETK | WP_042661398.1 | MLNLLIEKKIRHLEIILKVSERCNINC |
| | | | | | | TVIDVSDTK | | DYCYVFNKGNSAADDSPARISNKNIHH |
| | | | | | | KNRPQHLAE | | LVYFLORACQEYQIDTIQIDFHGGEPL |
| | | | | | | DVLEQIAGG | | LMKKESFTNMCIQLISGNYCGSQLRLA |
| | | | | | | AGWIKVFGN | | LQTNATLIDNEWIAIFEKYSVNVSISI |
| | | | | | | WSRSF | | DGPKHINDRHRLDTKGRSTYEGTVRGL |
| | | | | | | (SEQ ID | | RILQHAYKQGQLPSDPGILCVANAQAN |
| | | | | | | 114) | | GAEIYRHFVDELGVYSFDFLIPDDSYK |
| | | | | | | | | DAHTDAIGIGRFLNEALDEWIKDNNAK |
| | | | | | | | | IFVRLFQTHIASLLGQKNSGVLGHTPN |
| | | | | | | | | VTGIYALTVSSDGFVRVDDTLRSTSDR |
| | | | | | | | | MFNPIGHLSEVNLSNVFASPQFQEYSS |
| | | | | | | | | IGQSLPTECEGCIWENICAGGRIVNRF |
| | | | | | | | | STKDRFKRKSIYCYSMRTFLSRSSAHL |
| | | | | | | | | LNMGIKEERIMAAIQA (SEQ ID |
| | | | | | | | | 170) |
| | WVNVFARWSRRW | 1656.87 | Serratia | CDE | WP_103774054.1 | MSKLAKEIS | WP_103774053.1 | MANKEKIKHLEIILKVSERCNINCTYC |
| | | | | | | MNKAAVIID | | YVFNLGNDLAINSKPIISHGTIKNLRG |
| | | | | | | GDKKDVRRA | | FFERACQEYEIETVQVDFHGGEPLMIG |
| | | | | | | LTQSMLDSV | | KDRFDNACKELVSGDYNGTRLNLACQT |
| | | | | | | SGGWVNVFA | | NAILIDNEWIDIFSKHNISVGISIDGP |
| | | | | | | RWSRRW | | KHINDRHRLDRKGRSTYEGTVKGLEML |
| | | | | | | (SEQ ID | | QAAWRAGRLIDEPGILCVANPSVKGAE |
| | | | | | | 115) | | IYRHFVDVLKCKKFDFLIPDESHDTCT |
| | | | | | | | | DPEGLSDFYCSALDEFFLDADKEVYVR |
| | | | | | | | | YFHTHIQSMLSLEFSPVMGVSKAGSDT |
| | | | | | | | | LAFTVSSDGELYVDDTLRSTNDSIFTP |
| | | | | | | | | IGHIQSLTLSEALTSWQMQKYLSVDNQ |
| | | | | | | | | LPEVCIDCIWKKLCGGGRHIQRYSSAD |
| | | | | | | | | DFNRETVFCPSIRKIMSRAASHLIESG |
| | | | | | | | | VTEDIIMKNLEVNS (SEQ ID 171) |
| | AGWIRAFANWSRSF | 1662.83 | Serratia | DEC | WP_023489715.1 | MTRLKKEII | WP_037383507.1 | MVNLLNKKHIKHLEIILKISERCNINC |
| | | | | | | ETKTMIDVN | | DYCYVFNKGNSASNDSPARLSDKNVNH |
| | | | | | | SVKNNQPQH | | LVDFFQRACLEYEIGTLQIDFHGGEPL |
| | | | | | | LTEDVLDQI | | LMKKENFDRMCDRLVTGNYCGSNIRLA |
| | | | | | | SGGAGWIRA | | LQTNGMLVDDEWLALFEKHSVNVSISI |
| | | | | | | FANWSRSF | | DGPKHINDRHRLDTKGRSTYEGTVRGL |
| | | | | | | (SEQ ID | | RKLQHAYQQGRLPSDPGILCVANAQAN |
| | | | | | | 116) | | GAEIYRHFVDDLNVRSFDFLIPDDCYK |
| | | | | | | | | DTHVDPVGLGRFLNEALDEWVKDDNAK |
| | | | | | | | | IFVRLFQTHIASLLGKENVGVLGHTPS |
| | | | | | | | | ITSVYALTVSSDGFVRVDDTLRSTSDR |
| | | | | | | | | MFNTIGHLSEINLSDVFDSPQFQEYAS |
| | | | | | | | | IGQSLPTECKGCIWENICAGGRIMNRF |
| | | | | | | | | STEERFKRKSVYCYSMRSFLSRASAHL |
| | | | | | | | | LNMGIKEERIMEAINR (SEQ ID |
| | | | | | | | | 172) |
| | WFRAYLRWSRSF | 1668,88 | Mixta | DC | WP_165786503.1 | MNFTINDLK | WP_103059455.1 | MAKKIDILEIILKVTECCNIACRYCYY |
| | | | | | | KLLLNTEEN | | FEGDNRDFADKPRVMNKKTVIQLANYL |
| | | | | | | RSPSVAKET | | KETVVAHQIETLRIDIHGGEPLMMGKK |
| | | | | | | IEELSNDDL | | RLGELLLILSDALKKICKLEFVLQCNG |
| | | | | | | TNVGGGWFR | | TLIDDDWINIFAKYQVAASVSVDGDAV |
| | | | | | | AYLRWSRSF | | THNLNRIDRRGKGTYHRVMAGLSKLIA |
| | | | | | | (SEQ ID | | ASKDNKVPYPGVLCVINPDKNGKVIFR |
| | | | | | | 117) | | HFVEQNKTPYISFIEPDFTIDEASKQR |
| | | | | | | | | VDGIGNFLLDVYQEWEKNNSPKINRHM |
| | | | | | | | | SLRVFNDLLSVLMVSGTEYENMKTINY |
| | | | | | | | | VVITIRSDGYINPDDILRNTHPELFNE |
| | | | | | | | | SYHLASSTLEEFITSEDIRELYRGIFT |
| | | | | | | | | LPVQCQECGVRKLCRNGFCFGSLPHRY |
| | | | | | | | | SKKNGMNNTNLFCKFYREICIRLCNYA |
| | | | | | | | | VNKGKTFAEIEKAVY (SEQ ID |
| | | | | | | | | 173) |
| | WWRAYARWRRSF | 1734.95 | Gilliamella | DEC | WP_160406027.1 | MFFSKKTIE | WP_160406026.1 | MSNSIKVDILEVILKITECCNIACRYC |
| | | | | | | QRLRDTEAK | | YFFRGGNIDFDERPNVIKKDTIHALAS |
| | | | | | | RKNVPNAKA | | FLKEAILANEIKLLRLDFHGGEPLMMG |
| | | | | | | MEELAAQYL | | KKRFVEMVELFDTELSQLVDLEYVLQS |
| | | | | | | DEVNGGWWR | | NGTLIDDEWVEIFSKYNVAASVSLDGD |
| | | | | | | AYARWRRSF | | QAIHDANRIDKKGRGTYVRATEGLKKL |
| | | | | | | (SEQ ID | | ICAARSNKVVFPGIISVINDSSDTKIT |
| | | | | | | 118) | | FKHFLDDLESPFISFVELDLTIDELNQ |
| | | | | | | | | ETVEKISNNLLAVYNEWERINTPTIVH |
| | | | | | | | | DISVRNFNDILKQLVLSGTEADKKEKR |
| | | | | | | | | KYVSLTIRSDGSLNPDDILRNIYPYLF |
| | | | | | | | | TNEYNIKNNTLSDYLSDEKLKDLYRKL |
| | | | | | | | | FTLPEKCNECGVKKICRNGWGFGSIPH |
| | | | | | | | | RYSKENDMNNVNALCGVYHEISLRLCD |
| | | | | | | | | LVIQQGKSYDSIKHNLF (SEQ ID |
| | | | | | | | | 174) |
| | DRWLKWIKNH | 1391.6 | Photorhabdus | CDE | WP_181147865.1 | MSKLAKEIK | WP_219847460.1 | MKKIKHLEIIAKVSERCNINCTYCYVF |
| | | | | | | ENKTTVTTK | | NMGNDLAINSKPVISLKTVSNLKRFLE |
| | | | | | | KSADQKAMA | | RSLTEYNIESIQVDLHGGEPLMLNRER |
| | | | | | | QSLLDNVCG | | FSRMCEELMSGDYKGAKFSIACQTNAT |
| | | | | | | GGDRWLKWI | | LIDDEWIDIFSKYNISVSVSIDGPKHI |
| | | | | | | KNH (SEQ | | NDKNRIDNKGKGTYDATVSGLFKLQSA |
| | | | | | | ID 119) | | WKDGKLPSAPGVLCVANPNSNGAEVYR |
| | | | | | | | | HFVDVLNCKSFDFLIPDESHDNCKNPY |
| | | | | | | | | GISDFFCSAVDEFFSDADKKIIVRYFY |
| | | | | | | | | ATIQGMLNPGIFHVAGMGKMNNDIVAF |
| | | | | | | | | TMGSEGNIHVDDILRSSNDDIFTAIGN |
| | | | | | | | | VNELSLNNVI (SEQ ID 175) |
| | DGRWLQWIKNH | 1448.61 | Kosakonia | CDE | WP_180344379.1 | MKKLAKEVK | WP_139569738.1 | MKSIEHLEIIVKISERCNIDCTYCYVF |
| | | | | | | QNGVSVNTA | | NKGNDLAINSQTIIKKNTINSFRDFLE |
| | | | | | | KNKAQKKFS | | SASKGFDIKTIQIDFHGGEPLLLKKDR |
| | | | | | | QSLLDDVQG | | FNFLCKTLREGDYRGSRLVLSCQSNGV |
| | | | | | | GDGRWLQWI | | LIDDEWIDIFHKWDVGVSVSMDGPKHI |
| | | | | | | KNH (SEQ | | HDAARIDKNGKGTYDQVVAGFRKLQDA |
| | | | | | | ID 120) | | WKENKISTQPGILCVANTNLKGVEIYR |
| | | | | | | | | HFIDDLQCKGFDFLIPDETHDSNIDAS |
| | | | | | | | | KLYDFYESVIDEYFIDADIDIKFRYLK |
| | | | | | | | | VLIQGMLNPGTYAIAGLNAVNNDIVAL |
| | | | | | | | | TMGANGDIYIDDTLRSTSDKAFSKIIN |
| | | | | | | | | ISSGSLGDILSSWQYLEYTKFANTLPI |
| | | | | | | | | ECETCTWKKLCGGGGLVQRYSKEQRFN |
| | | | | | | | | GKSVYCHSLKKIYGRVASHLIESGIDE |
| | | | | | | | | THILKSLGCNDGN (SEQ ID 176) |
| | WVNAFLN | 858.95 | Yersinia | DEC | WP_072086462.1 | MSRLKKEIT | WP_050097262.1 | MGHLLTKKRIKHFEIILKISERCNINC |
| | | | | | | ETKTAIGTN | | DYCYVFNKGNSDADNNPARISNKNIGH |
| | | | | | | KAKKNQPQH | | LANFLORACLEYEIDTLQIDFHGGEPL |
| | | | | | | LADDLLDQI | | LMKKEHFANMCIQLISGNYCGSNIRLA |
| | | | | | | AGGWVNAFL | | LQTNGILIDDEWISLFEKYSVNVSLSI |
| | | | | | | N (SEQ ID | | DGPKHINDRHRLDTKGRSTYEGTVRGL |
| | | | | | | 121) | | RLLQSAYQQGRLPSAPGILCVANAQAN |
| | | | | | | | | GAEIYRHFVDDLGVYGFDFLIPDDSYN |
| | | | | | | | | DVNIDPIGIGRFLNEALDEWVKDNNPK |
| | | | | | | | | IFVRHFQTHFASLLGVKNIGILGQSSN |
| | | | | | | | | ITGVYAFTVGSDGSIRVDDTLRSTSDR |
| | | | | | | | | IFNTIGHISEINLSDVLNSPQAQEYSS |
| | | | | | | | | IGQCLPNECKGCIWENICTGGRLVNRF |
| | | | | | | | | SSEERFKHKSVYCYSIRSFLSRASAHL |
| | | | | | | | | LNMGIKEERIMTSICQ (SEQ ID |
| | | | | | | | | 177) |
| | FANASWPKSF | 1150.26 | Bordetella | CD | WP_176463924.1 | MMTKEIIQH | WP_176463923.1 | MHYIEIILKVAERCNLNCTYCYFFNKE |
| | | | | | | LEQVQRNAA | | NKDFEDHPALISPDTVRQLVQFLRTSS |
| | | | | | | EEEKTVEEI | | HEISETVFQIDIHGGEPLLLGPRRFSE |
| | | | | | | SQSELDQIC | | MVSIIENGLQDAKEVRFTVQTNAVLIN |
| | | | | | | GAGGVGGFA | | DAWLDVFSRHKVFVGVSVDGPKDRHDA |
| | | | | | | NASWPKSF | | NRIDRRGRGTFDSMVPKIAALKQATSE |
| | | | | | | (SEQ ID | | ARIPGFGSISVVSPESNGRATYTCLTQ |
| | | | | | | 122) | | ELGFSKLQFLFPDDTHDSANPANAGRF |
| | | | | | | | | ISFVDDLFECWEEDNSRDVRIKFIDQT |
| | | | | | | | | LVALLQNKHYIQRGRRVNPAFEGVVFT |
| | | | | | | | | VSSAGDIGHDDTLRNVAPELFKSGMNV |
| | | | | | | | | ANAKFPEFIAWHNMVSGILVSPDLPAP |
| | | | | | | | | CASCAWNNICEHVTGSYTPLHRMKNGT |
| | | | | | | | | ADQPSVYCEALKVAYQRGAEYLAKRGH |
| | | | | | | | | PIHQISKNLNPA (SEQ ID 178) |
| | FANATWSKSF | 1154.25 | Bordetella | CDE | WP_156770205.1 | MTTKEIIQH | WP_082993604.1 | MHYVEIILKVSERCNLNCTYCYFFNKE |
| | | | | | | LEQVQRNAA | | NRDFEGHPALISPNTVRHLVRFLRTSP |
| | | | | | | QEEKQMEEI | | HQISETVFQVDIHGGEPLLLGPKRFSE |
| | | | | | | SQEELEKIC | | IVSIIENGLSDAKEVRFTVQTNAVLIN |
| | | | | | | GAGGVGGFA | | EAWIDVFAQHKIFVGVSVDGPKGQHDA |
| | | | | | | NATWSKSF | | NRIDRRGRGTFDSMVPKIAALKQAALE |
| | | | | | | (SEQ ID | | RRIPGFGSISVVSPALDGRATYICLTK |
| | | | | | | 123) | | ELHFAHLQFLFPDDTHDSTNPALAEGF |
| | | | | | | | | AKFVEDLFASWQSDGNDNIHIKLIDQT |
| | | | | | | | | LLGFLQDKQYIDGGRRISPAVGRVVFT |
| | | | | | | | | VSSAGDIGHDDTLRNVAPELFKSGMNV |
| | | | | | | | | SDANYAEFIVWHNRVSKILFPRDLAPP |
| | | | | | | | | CASCAWNNICEHVTRSYTPLHRMKDGR |
| | | | | | | | | VDQPSVYCEALKTAYRNGAEYLAKRGL |
| | | | | | | | | PIREISKNLNPDY (SEQ ID 179) |
| | FANATWPKSF | 1164.29 | Bordetella | CDE | WP_157664463.1 | MMTKEIIQH | WP_086057504.1 | MAINHGEHATMPYVEIILKVAERCNLN |
| | | | | | | LEQVQHNAA | | CKYCYFFNKENRDFEDNPALISPNTVR |
| | | | | | | EEEKPIEEI | | QLVQFLRTSSHEISETVFQIDIHGGEP |
| | | | | | | SQSELDQIC | | LLLGPRRFSEMVSIIENGLHDAKEVRF |
| | | | | | | GAGGVGGFA | | TVQTNAALINDAWLDVFSRHKVFVGVS |
| | | | | | | NATWPKSF | | VDGPKDQHDANRIDRRGRGTFDTMVPK |
| | | | | | | (SEQ ID | | IAALSQATSQGRIPGFGSISVVSPESD |
| | | | | | | 124) | | GRATYMCLTKELRFSKLQFLFPDDTHD |
| | | | | | | | | SANTKNAGRFIKFVGDLFECWENDNNR |
| | | | | | | | | DVRIKLIDQTLAAFLQDKHYVEAGRRV |
| | | | | | | | | NSAAQGVVFTVSSAGEIGHDDTLRNVA |
| | | | | | | | | QELFRSGMNVADAKYPEFLAWHNMISG |
| | | | | | | | | MLVPRDLPPPCASCAWNNICEHVTGSY |
| | | | | | | | | TPLHRMKNGTADQPSVYCEALKIAYRR |
| | | | | | | | | GAEHLAKRGVPIHRISKNLTPVQRATS |
| | | | | | | | | (SEQ ID 180) |
| | WVNFQWKNSW | 1390.52 | Providencia | CDE | WP_210852630.1 | MKKFKTVIQ | WP_210852632.1 | MLKIKHFEVILKISERCNLNCTYCYIF |
| | | | | | | ENSANLKIK | | NMGSELALNSAPVISNTTIVELKNFLE |
| | | | | | | KDSDVSKLL | | RVADEVEHNVIQVDLHGGEPLMLKKKR |
| | | | | | | EHIRGGKSE | | FIYLCETLRSGDYKGAEFRIGLQTNAT |
| | | | | | | AAGGWVNFQ | | LIDDEWLEIFEKYNISVSISIDGPKHI |
| | | | | | | WKNSW | | NDRYRLDHKGRSSYEATMNGYQALYSA |
| | | | | | | (SEQ ID | | AENRKIIPTPPILSVINPDASGKELFE |
| | | | | | | 125) | | YFYHDMKCRKFDFLLPDNNYVNTVDTE |
| | | | | | | | | GIKRFLVDICDAWFAQNDPECDIRILS |
| | | | | | | | | AYLRILTGAEDYIVLGVTPQNELHQTI |
| | | | | | | | | AITVTSTGYIYVDDTLRSTLSDIFVPI |
| | | | | | | | | CHIRDASYQKIITSFPMRELSKIESFL |
| | | | | | | | | PDDCHGCIWKAVCAGGRPINRYSQDNA |
| | | | | | | | | FKNKTIYCDAMQSFLSRGAAYLINLGI |
| | | | | | | | | NSNEIAKNIGIDKNA (SEQ ID |
| | | | | | | | | 181) |
| | NVFVNATWSRAM | 1391.57 | Pandoraea | CDE | WP_157122607.1 | MTTKAFIEQ | WP_046290456.1 | MKQYVEVILKVSERCNIDCKYCYFFNK |
| | | | | | | LAKKQKAAN | | ENKDYASNPPYMTQQTAEDFVTFLRSS |
| | | | | | | EAGSIKEIP | | PNLRETTFQIDLHGGEPLMMKRERFEA |
| | | | | | | ASELERISG | | LVTTLKNGLSDAESVQFTVQTNAMLVD |
| | | | | | | ARGGNVFVN | | EAWLDLFSRLGVYIGVSIDGPKIYHDE |
| | | | | | | ATWSRAM | | NRVDKQGMGTYDRTVEKIALIKAAADT |
| | | | | | | (SEQ ID | | GLISGFGAICVMNPKFDARLVYDTLTR |
| | | | | | | 126) | | TLGIYNLQFLLPDESHDSVRTADVMAL |
| | | | | | | | | KWFTQALFDCWADDPRGTVRIRSIDRM |
| | | | | | | | | LDAILADEPRKDVIWRDARSSVVFTLS |
| | | | | | | | | SGGDIGHDDTLRNVIPDVFYARMNVAS |
| | | | | | | | | STFSEFLAWHATVSAMLARRTTAVACR |
| | | | | | | | | TCLWREICEIATRSDTPLHRCKNGVAD |
| | | | | | | | | QHTVYCECLKANYEKGAEYLALSGVAI |
| | | | | | | | | EEISRNFVEVD (SEQ ID 182) |
| | WSRTVFNRVRPV | 1512.74 | Erythrobacter | DEC | WP_212451268.1 | MAKNKTPKT | WP_212451270.1 | MFDVEARLARPGRRHVSVVLKVAERCN |
| | | | | | | EAKAQSKSL | | LACTYCYFFFGGDDSYLKHPALISSDR |
| | | | | | | ESLIDAQLD | | VSDVARFLGEAAIKHRLERIEIALHGG |
| | | | | | | SIVVGGWSR | | EPLLLKPDRMGALVETIRAAVPDSCEV |
| | | | | | | TVFNRVRPV | | DILLQTNGVLVDETWIALFEQHSIGIG |
| | | | | | | (SEQ ID | | VSLDGPRAVNDIARLDKKGRSSFDATI |
| | | | | | | 127) | | AGWGLLKKAAADGRISEPGILSVIAPT |
| | | | | | | | | TDAETLSFFIDELGAHSLNFLLPDMFF |
| | | | | | | | | DNPETQPEDVARIGETMIAIFEEWRRR |
| | | | | | | | | ADPGLHIRFVNDALLPMIVAIPAESTH |
| | | | | | | | | HCREDLSHAMTIASDGTIYVEDTIRSA |
| | | | | | | | | FADRFDETLNVASATLADVFAHPHWQS |
| | | | | | | | | IARAAEQPAGPCTSCRYGEICQGGPLI |
| | | | | | | | | SRYSSDRGFDNPSLYCSALFAFHRHVE |
| | | | | | | | | REVSATGRLLPSPRFAADPLFPARKEV |
| | | | | | | | | A (SEQ ID 183) |
| | AGNDGWVKFGWKKKF | 1764.02 | Sodalis | CDE | WP_213990087.1 | MDKLRDAIK | WP_213990088.1 | MKDKQPKHLEIILKVSERCNLNCSYCY |
| | | | | | | NNTKTPLAK | | VFNMGSDLALNSAPVISRATINSLKNF |
| | | | | | | DTGDLLKSI | | LERSVREYSIDVIQIDLHGGEPLMLKK |
| | | | | | | RGGAGNDGW | | ERMAVLCALIREGDYNGASVQIGIQTN |
| | | | | | | VKFGWKKKF | | ATLIDEEWIEIFSRYHVSVSISIDGPK |
| | | | | | | (SEQ ID | | HVNDIHRLDHQGRSSYEKTLRGYKLLS |
| | | | | | | 128) | | TRSTDGKKEINAPVLSVLTPKANGSEL |
| | | | | | | | | FSHLYDVMGCRNFDFLLPDCNYDNPID |
| | | | | | | | | TAAIGRSLIEICDKWYAQNDPDCVVRI |
| | | | | | | | | VNAHMAHLAGNKKNVVLGVTNVNKNAL |
| | | | | | | | | ALAFTVTSQGEIYVDDTLRSTHSDIFT |
| | | | | | | | | SIGNITHTSLEEIFASROLIALNIIQD |
| | | | | | | | | TIPRECSECVWRNICAGGRPINRYSSI |
| | | | | | | | | DGFTGKTIYCDAMKMFLGRCASILNEM |
| | | | | | | | | GVSIEELVINLGIENDK (SEQ ID |
| | | | | | | | | 184) |
| | RGEGWVRAYWAKRF | 1778.01 | Kosakonia | CDE | WP_139569744.1 | MSKLAKEIA | WP_139569743.1 | MRTKIKHLEIILKVSERCNINCTYCYV |
| | | | | | | SNKATVTTP | | FNLGNELAINSKPVISASTIGDLRRFL |
| | | | | | | TAKAAHVAN | | ENAAIEHGIETLVIDFHGGEPLMMGKK |
| | | | | | | LLDNVQGGR | | KFAAACEVFRSGNYGNGELHLACQTNG |
| | | | | | | GEGWVRAYW | | ILIDDEWIDLFSKYGVGVGVSIDGPKH |
| | | | | | | AKRF (SEQ | | INDKHRLDHKGRSTYEGTVKGFRLLQA |
| | | | | | | ID 129) | | AYAAGKLELEPGILSVANPFVKGSEIY |
| | | | | | | | | RHFVDTLNCKRFDLLIPDESHFSCKNP |
| | | | | | | | | NEIADFYCSAIDEFFFDGNPDINIRYI |
| | | | | | | | | NTHVQAIVSNNHAQTLGVSKSTSDAIA |
| | | | | | | | | ITVMSDGDIYIDDTLRSTNDELFSPIG |
| | | | | | | | | NVREISFSGVKESWQFKKSAHIANNPP |
| | | | | | | | | ADCKDCLWKKVCGGGSMIQRYSKEEGF |
| | | | | | | | | ERKSVYCPSIKKIFSRMTSHLISAGIP |
| | | | | | | | | EEKISKNLEG (SEQ ID 185) |
| | RGQGYVRFIFRRSF | 1785.04 | Bartonella | | WP_008038584.1 | MSKLKSEIN | WP_008038586.1 | MSNVASKLNVLEIILKLTERCNLNCTY |
| | | | | | | TNNHNNAAD | | CYVFNKGDYDETSSQALISDNSVNDVI |
| | | | | | | DLVELSEAT | | DFVLNAIESYELKLVRIIFHGGEPLLY |
| | | | | | | IKKLDAAGG | | PKKKFDNLCNSLKALESVDTSITLSLQ |
| | | | | | | RGQGYVRFI | | TNGVLIDETWVEIFSRHDVTVGISLDG |
| | | | | | | FRRSF | | NKEMNDQYRLDKKGRSSYERSIKGLRL |
| | | | | | | (SEQ ID | | LQESYNQNKFSHSPSILMVANCENDID |
| | | | | | | 130) | | TLYDHVFNNLGVSSFDILLPDDNYLDE |
| | | | | | | | | SRPSDDLMGKYFTRLLDLYLNDERDVF |
| | | | | | | | | IRLFDAPIYILNSNSMDFLGFSARVHK |
| | | | | | | | | MMVSLTINTDGLLYVNDVLKPTGAYLA |
| | | | | | | | | SAIGNIKDFKLEDFMASQQYKMYISAT |
| | | | | | | | | EYVPSECQDCIWRNPCSGGALQNRYSK |
| | | | | | | | | ENGFSNKTIYCGTNRSILSRVSEYLII |
| | | | | | | | | KGVDESKIMSNIGL (SEQ ID 186) |
| | KPGEGWVNFTWNKSF | 1792.97 | Photorhabdus | CDE | WP_172911276.1 | MKELQKAIQ | WP_172911275.1 | MPKIKHFEVILKISERCNLNCSYCYVF |
| | | | | | | KNSANLKNQ | | NMGSELALNSAPVISHNTIIELKYFLE |
| | | | | | | KAKEASNLL | | RVAEETTPDVIQIDLHGGEPLMLKKER |
| | | | | | | DAVRGGKPG | | FVYLCETLRSGDYKNAEFRLGLQTNAT |
| | | | | | | EGWVNFTWN | | LIDDEWIEIFEKFEVAVSISIDGPKHI |
| | | | | | | KSF (SEQ | | NDKYRIDHKGRSSYEATLNGYQALYTA |
| | | | | | | ID 131) | | AKKRNILPLPPVLSVIDPEANGKELFE |
| | | | | | | | | HLYHDMQCRKFDFLLPDYNYENPTNTE |
| | | | | | | | | GIKRFLTAICDAWFEQNDPACDVRILS |
| | | | | | | | | AHLTRLMGTTGHVILGVTPQIESYKAV |
| | | | | | | | | AITVTSTGDIYIDDSLRSTLSKIFTPI |
| | | | | | | | | GNIKNTSYAQIVNSPPMRELSKIEASL |
| | | | | | | | | PDDCQGCIWKTICAGGRPINRYSRDNA |
| | | | | | | | | FNNKTIYCDAMQAFLGRGAAYLVELGL |
| | | | | | | | | SENEIEKNIGIAEHE (SEQ ID |
| | | | | | | | | 187) |
| | WVNAFANRTMGFLFKL | 1911.25 | Erwinia | CDE | WP_168428711.1 | MSKLQREIT | WP_168428712.1 | MRLIKGEKIKHLEIIFQVSKRCNISCS |
| | | | | | | SNKAQLVNA | | YCQVFIMGNTLAADSHPTKSLNNVIAL |
| | | | | | | DVRKMQRKV | | RGFFERSTAENEIEVIQVDFHGGKPLM |
| | | | | | | FVDSLLDTV | | MKKDRFDQMCHILLQGDYGNSRIELAL |
| | | | | | | SGGWVNAFA | | QTHGILVDEEWITLFEKYKVQASIPVD |
| | | | | | | NRTMGFLFK | | GLRHSNNRHRPDRTGESTYKGTINGLR |
| | | | | | | L (SEQ ID | | LLQNAWQQGRLPAEPGILSVANAKANG |
| | | | | | | 132) | | ADIYHHFVDVLKCQRFDFLIPDDHHDD |
| | | | | | | | | ITDSEGIGRFLNEALDAWFADGRPELF |
| | | | | | | | | VRIFNTYLGTLLDKQFSRVLGMSANVE |
| | | | | | | | | SAYAFTVTADGLLRIDDTLRSTSDEIF |
| | | | | | | | | NPVGHVRDLSLAGVLKNTAVEEYLSLS |
| | | | | | | | | NTLPEGCKDCVWNNVCHGGRLVNRFSQ |
| | | | | | | | | ANRFNNKTVFCSSMRIFLSRGASHLMA |
| | | | | | | | | TGIDERTIMANIQG (SEQ ID 188) |
| | ASTAETWFKLDWKKSF | 1941.17 | Xenorhabdus | DEC | WP_189757993.1 | MKELQKIIH | WP_189757994.1 | MNKINHLEVILKISERCNLNCSYCYVF |
| | | | | | | ENSANLKNQ | | NMGSDIALNSAPVISHNTIIGLKGFLE |
| | | | | | | KGQKASELL | | RVAEDVNPDVIQIDLHGGEPLMLKKER |
| | | | | | | DFVRGGAST | | LIYLCETLNSGDYKGAELRFALQTNAT |
| | | | | | | AETWFKLDW | | LINNEWIAIFEKFNISVNISIDGPKHI |
| | | | | | | KKSF (SEQ | | NDKYRIDHKGRSSYEATLNGYKALCTA |
| | | | | | | ID 133) | | AKERNILNYPSILSVIDPEASGKELFD |
| | | | | | | | | HFYHDMQCKRFDFLLPDSNYENTTNTE |
| | | | | | | | | GVKRFLIDVCDAWFEQSDPNCDVRILS |
| | | | | | | | | SYFTRLAGSSKYIVLGVTPPTEGFEAL |
| | | | | | | | | AITVTSTGDIYIDDTLRSTVSEIFTPI |
| | | | | | | | | GNIADATYAQIVNSQPMREFHKIESSL |
| | | | | | | | | PVDCQGCIWQKICAGGKPVNRYSRDNA |
| | | | | | | | | FNNKTIYCDTMAALLGRGAAYLVELGL |
| | | | | | | | | SENELAKNIGIAEL (SEQ ID 189) |
| | SSDDDGIFFKTTWDRR | 1942.03 | Xenorhabdus | DEC | WP_189757997.1 | MKELQKVIQ | WP_189757994.1 | MNKINHLEVILKISERCNLNCSYCYVF |
| | | | | | | ENSANLKNQ | | NMGSDIALNSAPVISHNTIIGLKGFLE |
| | | | | | | KGQKASELL | | RVAEDVNPDVIQIDLHGGEPLMLKKER |
| | | | | | | DAVRGGSSD | | LIYLCETINSGDYKGAELRFALQTNAT |
| | | | | | | DDGIFFKTT | | LINNEWIAIFEKFNISVNISIDGPKHI |
| | | | | | | WDRR (SEQ | | NDKYRIDHKGRSSYEATINGYKALCTA |
| | | | | | | ID 134) | | AKERNILNYPSILSVIDPEASGKELFD |
| | | | | | | | | HFYHDMQCKRFDFLLPDSNYENTTNTE |
| | | | | | | | | GVKRFLIDVCDAWFEQSDPNCDVRILS |
| | | | | | | | | SYFTRLAGSSKYIVLGVTPPTEGFEAL |
| | | | | | | | | AITVTSTGDIYIDDTLRSTVSEIFTPI |
| | | | | | | | | GNIADATYAQIVNSQPMREFHKIESSL |
| | | | | | | | | PVDCQGCIWQKICAGGKPVNRYSRDNA |
| | | | | | | | | FNNKTIYCDTMAALLGRGAAYLVELGL |
| | | | | | | | | SENELAKNIGIAEL (SEQ ID 190) |
| | ADSQPKARAWFANASFSKRF | 2281.52 | Burkholderia | CDE | WP_175425513.1 | MDLHVFKKE | WP_175425514.1 | MIEHDKINRLEVILKVTERCNIDCTYC |
| | | | | | | MMAGAQQEE | | YYFNGNNRDYMGQPPYLTVDTAKSLAV |
| | | | | | | RELLAEIDP | | YLRNAACSHSIDEIRIDLHGGEPLLMK |
| | | | | | | ELLALVGGG | | KAKMSAVLEILRSGVADFTDLTICIQT |
| | | | | | | ADSQPKARA | | NATLLDEEWISIFEKYSVSVGVSLDGS |
| | | | | | | WFANASFSK | | PDENDLYRVDKKGKGTHSVVVKAIELL |
| | | | | | | RF (SEQ | | KAANKKSEGIFAGIICVVNPDFDGKKI |
| | | | | | | ID 135) | | YRHFVDDLGVERIHFLKANQTRDGADI |
| | | | | | | | | KLVAGTRKFLLGALNEWINDGNFNIYV |
| | | | | | | | | RQFTEPLKOLCTSSAPSPCSDRYVAMT |
| | | | | | | | | VRANGDIAIDDDFRNTLPSLFNLGLNI |
| | | | | | | | | SDSALADFLDRPGVADFHRACGEVSPS |
| | | | | | | | | CLQCGAREICKNGTGLAESVLHRYSFI |
| | | | | | | | | NKFRNASLFCESHQAIIIRLGQFAISR |
| | | | | | | | | GVPWSTIERNMAGIRNN (SEQ ID |
| | | | | | | | | 191) |
| | VESQSKPRAWFANSSFSKRF | 2355.6 | Trinickia | CDE | WP_207004678.1 | MDLHVFKKE | WP_207004679.1 | MLIRLVIQKTPHFLVRNFRGCSTHQCF |
| | | | | | | MMAGAQQVE | | PKCIEPESSSCVLINNWRRNDGARKIN |
| | | | | | | REMPAELDP | | RLEVIVKVTERCNIDCTYCYYFNGENG |
| | | | | | | EFLALVGGG | | DYANQPPYLTVDTARSLAIYLHNASRS |
| | | | | | | VESQSKPRA | | HSIDEIRIDLHGGEPLLMKKTRMSVML |
| | | | | | | WFANSSFSK | | EIFRSSIPDSTDLTICIQTNAILLDEE |
| | | | | | | RF (SEQ | | WISIFAKYNVSVGVSLDGPPRENDLYR |
| | | | | | | ID 136) | | VDKKGRGTHSAIAKAIEMLKKANKKCA |
| | | | | | | | | GVFAGVICVVNPDFDGRKVYRHFVDDL |
| | | | | | | | | GIERIHFLKPNQTRDGADIKLVEGTSK |
| | | | | | | | | FLLDALNEWINDSNPNIYVRQFTDPIR |
| | | | | | | | | RLCASGPSSPFSDRYVAVTVRANGEIA |
| | | | | | | | | IDDDFRNTLPSLFNLELNVADSALADF |
| | | | | | | | | LNHPGVFDFHQACAEVPPSCLQCGANG |
| | | | | | | | | ICQSGIGLNESVLHRYSFINKFRNASL |
| | | | | | | | | FCQSHQAIIIRLGQFAISHGVPWSTIE |
| | | | | | | | | KNMIRIHDN (SEQ ID 192) |
| | ASSQANSRGWFANATWSKAWR | 2378.55 | Burkholderia | CDE | WP_162999177.1 | MDLHAFKNE | WP_121856868.1 | MFISFSTKSHVTSLLARKLAPRNDASL |
| | | | | | | MMVGAQQVE | | GHQFWTESTLLKISKEMKNIDKINRLE |
| | | | | | | REAPVELDS | | VILKVTERCNIDCTYCYYFNGSNHDYT |
| | | | | | | ELLALVGGG | | SQPPYLNIDTAKSLAGYLRDATRAHSI |
| | | | | | | ASSQANSRG | | DEIQIDLHGGEPLLMKKSRMSDMLEIF |
| | | | | | | WFANATWSK | | RNSISDQTDLRISIQTNATLLDEEWLS |
| | | | | | | AWR (SEQ | | IFAKYNVSVGVSLDGPPRENDLHRVDK |
| | | | | | | ID 137) | | KGNGTHSAVSKAIAMLIEKNKTCEGVF |
| | | | | | | | | AGVICVINPDFDGSKTYRHFVDDLGIE |
| | | | | | | | | RIHFLKPNQTRDAADIKLTEGTSKFLL |
| | | | | | | | | DTLSEWINDSDRNIYVRQFTDPLKRIC |
| | | | | | | | | ASDASESPPHRFVAMTVRANGEIAVDD |
| | | | | | | | | DFRNTLPSLFNLGLNVSNSTLADFINH |
| | | | | | | | | PKVADFHRACDEVPPFCSQCGAKGICQ |
| | | | | | | | | SGAGLGESVLHRYSFINKFRNASLFCT |
| | | | | | | | | SHQAVIIELGKFALSHGMPWATIEENM |
| | | | | | | | | TGNRI (SEQ ID 193) |

^{a}C-terminal residues after the GG motif.
^{b}Molecular weight of the fully modified core peptide.
^{c}Topology of xyeCDE genes in the biosynthetic gene cluster.
^{d}Protein ID and sequence for a representative pair of precursor and rSAM are shown.

The protease, transporter and protease/transporter may be fused or may be separately expressed. In some embodiments, the protease, transporter and the protease/transporter are encoded by the same nucleic acid molecule. In some embodiments, the protease, transporter and protease/transporter are derived from Xenorhabdus nematophila (Xnc).

In some embodiments, an amino acid sequence of the protease is at least 70% identical to the amino acid sequence of SEQ ID NO: [XncC]. In some embodiments, an amino acid sequence of the transporter is at least 70% identical to the amino acid sequence of, SEQ ID NO: [XncD]. In some embodiments, an amino acid sequence of the protease/transporter is at least 70% identical to the amino acid sequence of SEQ ID NO: [XncE].

In some embodiments, the protease and/or the protease/transporter is capable of cleaving the modified precursor polypeptide to form the polypeptide. In some embodiments, the protease and/or the protease/transporter is capable of cleaving the modified precursor polypeptide at a Gly-Gly motif.

In some embodiments, the transporter and/or the protease/transporter is capable of transporting the polypeptide out from of a host cell.

In some embodiments, the nucleic acid sequence is provided to the host cell via a phage.

In some embodiments, the method comprises b) isolating the cleaved modified polypeptides that are exported out from the host cell. In some embodiments, the method comprises isolating the polypeptide from the culture medium.

The method may be performed under anaerobic or oxygen-free conditions.

Table 8 shows a list of precursor polypeptide and rSAM sequences, and protease, transporter and protease/transporter sequences that may be used.

TABLE 8
Precursor polypeptide, rSAM, protease, transporter and protease/transporter

| | | Restriction | |
|---|---|---|---|
| Gene | Vector | Sites | Insert Sequenceª |
| xncAB | pET-28a(+) | NdeI_XhoI | AGCAAATTACAGCGTGAAATTGCAGCAAACAAAGCTCAACTGAGC |
| (Protein ID: | | | CATGAAGACAAGAAGAAAACGCAGCACAAAGAGCTTGTTGACAG |
| WP_ | | | CCTGCTGGATACTGTCTCTGGTGGTTGGATAAACGCTTTTGGAAA |
| 010848441.1, | | | CTGGGAGAGAGCCTTTCATTAAtacactgccgggggaggttttcttccccctt |
| WP_ | | | ctctttcttcattctggcgaataATGATAATGACGACATCAAAGAGTGAGA |
| 010848442.1) | | | TTGAGGGGATTCTTTGAGCGCTCCGCAGCAGAAAACGAGATTGA |
| | | | CTATAGCGGTTCCCGGCTTGAATTAGCATTACAGACTAACGGTAT |
| | | | CATGCCAGCATATCAATCGATGGACCAAAACATATCAATGACCGC |
| | | | TATCGGTTGGACCGAAAAGGAAAAAGCACTTACGAAGGAACAATT |
| | | | AGATCAAACATCTTGAGATCATTCTCAAAATTAGTGAACGATGCAA |
| | | | TATCAATTGCTCCTATTGCTATGTATTCAATATGGGTAACTCACTG |
| | | | AGTTATCCAAGTCGATTTTCACGGTGGTGAACCACTGATGATGAA |
| | | | AAAAGACCGTTTCGATCAAATGTGTGACATTCTTCGGCAGGGTGA |
| | | | TCTGATTGATGATGAATGGATTTCACTGTTTGAAAAACATAAAGTC |
| | | | TGATGGCATAGGTATTGGCAGATTCATGAATGAAGCGCTTGACGC |
| | | | GCTACCGATAGTCCTCCGGTCATATCGCTTGATAACGTGCTGGCG |
| | | | CCCGGGAGAGCCCGGCATTCTCTCTGTGGCAAACCCCACAGCGA |
| | | | AGCACTTCGATTTCCTCATACCCGACGCTCACCATGATGATGATAT |
| | | | GGCATGAGCGCGAATGTAGAATCTGCTTATGCTTTCACGGTAACT |
| | | | GCCGACGGCCTGCTCCGTATTGATGATACTTTGCGTTCCACCTCT |
| | | | CCGGCGTACTCAATTCACCTAATGTCAAAGAATATCTTTCACTAAA |
| | | | TAGTGAACTGCCAAGTGATTGTGCAGATTGTGTGTGGAACAAAAT |
| | | | ATGGTGCAGAGATTTATCACCACTTTGCAAACGTCCTCAAATGTC |
| | | | ATGGTTTGCTGACGGTCGGTCAGAGATTTTTGTTCGAATCTTTAAC |
| | | | ACATACCTTGGCACGATGCTAAGTAACCAGTTTTACCGGGTTATT |
| | | | GATGAAATATTCAATGCCATTGGGCATCTCAGTGAATTGTCACTCT |
| | | | CACGGCTTGCGCATGCTCCAGAATGCGTGGAAGCAAGGGCGACT |
| | | | CTGTCACGGTGGCCGCTTGGTCAATCGCTTTTCACGGGCAAACCG |
| | | | TTTCAATAATAAAACCGTGTTCTGTTCATCAATGAGGCTTTTCCTT |
| | | | AGTCGCGCGGCTTCACACCTGATTACGGCTGGTATTGATGAAGAA |
| | | | ACAATAATGAAAAATATTCAGAAATAG |
| | | | (SEQ ID 194) |
| xncCDE | pCDFDuet-1 | NdeI_XhoI | GAAAAAATCAATTTCTGGTTATCAAAGTTTTCATGTGCCGCCCTCG |
| (Protein ID: | | | CTATTTGTTGTACATCTTGCCTTGCTGACTCGGGAAATTCGGTAAC |
| WP_ | | | ACTTAAGCTGAATTATGACAAATATTTCACGCCTCATGCAACTTTC |
| 013185693.1, | | | ATCATTAATGGCCACCCGGTAAATATGATGATTGATACAGGTTCTT |
| WP_ | | | CGAAGGGCTTTTATCTTCAAGAGCCTCAACTAAAAAAAATACAAG |
| 013185694.1, | | | GCCTCAAAAAAGAAAGCACTTATTACAGTACTAATATCACCGGGA |
| WP_ | | | AAAGACAGGAGAACACAGAGTATCTCGCCGCTTCTCTCGACATGA |
| 013185695.1) | | | ATGGCCTTAAATTAAAAAACGTAACCGTGATCCCATTTAAACAATG |
| | | | GGGAGCGCTGATTTCTAACACAGGTAAATTGCCGGATGGCCCTGT |
| | | | TGTCGGTCTCGATGCGTTTAAAGATAAACAAATTATGCTGGATTTT |
| | | | GTGTCTCATTCATTCACGATGAGCGACAGTTTTATCCATAACATGC |
| | | | CGGTTCCGAAAGGCTTTAACGCATTCACTTTCCATATGTCTCCTGA |
| | | | TAAGCCGCCTGCGGTATCACTGATTGCACAAAGCAGTGGAATCAT |
| | | | TACGCATTCACTGGCATTAGAGCAAACAAGAGTTAAGCGCAACGA |
| | | | TGGCATGGTTTTTGATGTTGATCAGTCTGGACACACATACCATTTG |
| | | | TGGTTCGTACAGTGAACGGATAAATGTCATCGGAACCGTGGTTTA |
| | | | TTCCTCAGAAATCGAAAGGTACTTATAGACTTTAAAAACAAGAAG |
| | | | AATCTTTCGTGCCGAGGCTTTGCAACACAAACGAGAAGGTTGGCT |
| | | | TGGTTCGAGAAAAAATCAAACCTGTATGCGAATTTTAAGAAGAAA |
| | | | TACGCATCCACATTAAGCATTTCTTCTGCAAAGGTCAAAGTGATAG |
| | | | AGTATTTAATCGTCGCGCCGTTTGATGGAATGATAACCAGTGTTA |
| | | | GTTTTTATTTCCGATGAGCACCGAAACAGAAAAGAATGACAACTC |
| | | | GGCATTGCGCTTGATGCTGAATGGATAAACAGAAAGAAAGATTAT |
| | | | TAGCCGATATAGCACAAAAAATACTGATTACAGAAAAACAAAAAG |
| | | | AATGAGAGTCTCGGCATACCCTTACCAGTGGTATGGAAAGATTGC |
| | | | ATTCTGGACACCGGTGCCACTGCGTCTGTGATTTGGCGTGAAAGA |
| | | | CGGCGCTTCTCGTTTGCATATACCGTCAGCGCTCTCTATTTGTTGC |
| | | | GAGCATTTTTTCTATCAGTGGTGACACTCAGACAAATCTGGGTGC |
| | | | CACCAATGTTGAAACGGTAGAACTTTTAAATAAGCAACGTAACGC |
| | | | GCTGTCTAAAAAGCTTGATATTGCGGCCAATGAATCAAAAGCAAA |
| | | | ATGGATAACGAAGGATGCCAGGCCACTCTGCTCACAATTAAATCA |
| | | | AAAACTGGAAATCCCCAGCATTTTGGTGCGGTTGTTGTTGTCGGA |
| | | | AATTTTAAACACATGGGCAACGTTGATGGCCTTTTAGGGAATAAC |
| | | | GAAAGTCTGCAAAACCTGATAGAAACTTCAGAAAAACAGCAAGCG |
| | | | CCCTGCTGGGAGAGTTGCAGGATCTGAAAAATGACGTTTCGGTTA |
| | | | TCGACAGGAAACTCGACAAAGAAACAGCATCTCTCACTGTCGAAA |
| | | | CAGCCCATATCGGTGAAAGAGTGACTGCCGGCCAGCAAATAGCC |
| | | | CTTAAACAGTATGAACCCAAAAGCTGCCTGCTGGTCGATCCGAAG |
| | | | CTGACAATCCTTGTTATTTTCTTTTTCATCATATTGATAATTGCATT |
| | | | CAAGATTTATCTCAGCGAAAAAATTAAAAATAAACAACAGGAAATA |
| | | | GTGCTGATACCACAAGGTGCGACAGAAAAGGTTGAGTTGTTTTCA |
| | | | CCGTCTGATTCTCTCGGTGAAGTGACCAGCGGACAGCAAGTCAG |
| | | | AGGCATCATAGAAACGATATCGGCAGCACCGGTCAATGTCACCTC |
| | | | ACAGATGCAGATGAAAGGTGAAGAGGTAAAAAAGGGGCTTTTTC |
| | | | GGATTGTCGTACAACCAAAATTGACCGGACAACAAACAAACATTT |
| | | | CCCTTCTACCCGGCATGGAAGTGGAAACAGAGATCTATGTGAAAA |
| | | | CCCGAAAATTGTACGAATGGTTATTTATCCCCATTAAAGGGGCAT |
| | | | ATGAACGGGCGACAGACAGTACGGAATAAatATGCAGTATAAGAT |
| | | | GAGTGATTTTTTCGAGTTTTTCGTCAAAAAACTCCCGGTGATAATA |
| | | | CAAACAGAGACCACAGAATGCGGGTTGGCATGTCTGGCCATGAT |
| | | | TGCTGCCTGGTATGGCCGTGAGACTGATATCTACAGCATGAGAAA |
| | | | GGTTTTTGACGTGTCAAACAATGGCATGACATTAAGGCAGATCAT |
| | | | CACGGCGGCCGGGCGAATAAACATGAATACCAGAGCTGTGCGGC |
| | | | TGGAACTCAACGAACTCAGCAGTGTCAGGCTTCCGTGCATCTTGC |
| | | | ACTGGTCCTTTAATCATTTTGTCGTGTTAAAAAAATTCACAAAAAA |
| | | | AGGGGCAGTCATCCATGATCCCGCCTTGGGAAAAAGAACTGTCA |
| | | | CTCTGAAAGAACTCTCAAATAAGTTTACGGGCATCGCTCTGGAAG |
| | | | TCTGGCCCCAGACGGAGTTTAAAAAGGAAAAGGTCAGTGAAAGC |
| | | | ATAACCATCACGGATATGTTTCGCGGTGTTGCCGGCCTTAAGAAT |
| | | | ACGCTGTTTAAAATCATTCTGTTGTCGCTCTTTATTGAAGTACTGG |
| | | | CACTTTCCATCCCTCTCAGCTCTCAATTCATTATTGATGTTGTTCTA |
| | | | CGGTCCAGTGACCTCAGTATGCTGAATTTCATTGTCATTGGAATC |
| | | | GTTCTTCTGCTCTCCCTGCGCGCTGCTTTCAGTATTGTGCGCGCC |
| | | | TGGGCTCTTATGGCAATGCGTTACTCACTTGGCATACAGTGGAGT |
| | | | TCCGGTTTTTTTAACCGGTTACTCAGATTGCCGGTCACTTTTTTTG |
| | | | AAAAACGTCACGTAGGTGATATCGCCTCCAGATTGACATCGTTGA |
| | | | GCGAAGTTCAAGAAGCCTTTACAGCAGAAATGCTGACTTCGTTAC |
| | | | TTGATGTACTTATTCTCATAACGCTGGCTGTGCTCATGTTCTGTTA |
| | | | CAGCCCTCTTCTGACCCTTCTCCCGCTACTCATGACTACCGTTTAT |
| | | | CTTGGGGTCAAATTTGCTTTTTATGACAGATACATGGGAGCAAAA |
| | | | GTAGAAGCAATTACGCATGAAGCGCAGCAATCATCCTACTTTCTC |
| | | | GAAACAATACGAGGCGTAGCGTGCGTGAAAGTATTTGGCCTGAC |
| | | | AGAATTCCGACGTATCACATGGCTTAACCGGGTGATTGATACTGC |
| | | | CAATGCCCGGGCCCATTTATTTAAGATAGACCTCATCAGCCAAAC |
| | | | GCTTTCAGGTTTCCTGACGGGGCTATCATCGGCGGCCATTTTGTT |
| | | | TATGGGGAGTCATCTCACAGAACGCGGCCTGATCACTGCCGGCA |
| | | | TTCTGTTTGCTTTTCTGCTCTATACCGATATGTTTCTGACACGTTCA |
| | | | GTGAAGGTAATAAATTCACTGTTTGCTTTTCGCCTTATTTCGATAC |
| | | | ACACGCACCGATTGACCGATATTGCAACAGCCCAGACAGAAAATG |
| | | | CATGGAACCCGGAAGATCCCGTCACACTCGATAATGTAAAAGGCC |
| | | | GGATAACACTGAACAATCTCACATATCGGTACGGAGAAACTGAAC |
| | | | CCTGTATTTTCGACTGTATCGACATGGAAATTAATGCTGGTGAGA |
| | | | GTGTGGCGATCGTAGGTCCGTCAGGTTGCGGTAAATCGACACTT |
| | | | CTCCGGGTCATGGCCGGCCTGGTTCTCCCTCAGTCAGGCGATGT |
| | | | GTCAATTGATGATGTCAGTGTGAAAAAAATGGGTATTGACGAATA |
| | | | TCGCAGACACACGGCGTTTGTCATGCAAGATGATAAGCTTTTTGC |
| | | | TGCCTCATTGATGGATAACATATCCGCTTTTGATCCACAGCCAAAT |
| | | | ATTGATTGGATACATGAATGCGCTAAGGCGGCGGCAATACACGAT |
| | | | GAAATTATGACTATGCCGATGCAGTACGAAACCATGGTGGGTGAC |
| | | | ATGGGGAGCATTCTTTCAGGCGGACAAAAACAGCGTGTATCCCTT |
| | | | GCACGGGCACTTTACAAGTGTCCGCGTATCCTCTTTCTTGATGAG |
| | | | GCCACCAGCCATCTCGACGTTTTTAATGAACGCAAGATAAATGAG |
| | | | GCTGTAAAGCAGATGCCGATTACGCGTGTATTTGTGGCTCATCGG |
| | | | CCAGAAATGATCGCTGTCGCAGACCGAGTTTATAACCTGAGGGAT |
| | | | AAGACCTTTACAACGTAA |
| | | | (SEQ ID 195) |
| xnCBCDE | pCDFDuet-1 | NdeI_XhoI | ATGACGACATCAAAGAGTGAGAAGATCAAACATCTTGAGATCATT |
| | | | CTCAAAATTAGTGAACGATGCAATATCAATTGCTCCTATTGCTATG |
| | | | TATTCAATATGGGTAACTCACTGGCTACCGATAGTCCTCCGGTCA |
| | | | TATCGCTTGATAACGTGCTGGCGTTGAGGGGATTCTTTGAGCGCT |
| | | | CCGCAGCAGAAAACGAGATTGAAGTTATCCAAGTCGATTTTCACG |
| | | | GTGGTGAACCACTGATGATGAAAAAAGACCGTTTCGATCAAATGT |
| | | | GTGACATTCTTCGGCAGGGTGACTATAGCGGTTCCCGGCTTGAAT |
| | | | TAGCATTACAGACTAACGGTATTCTGATTGATGATGAATGGATTTC |
| | | | ACTGTTTGAAAAACATAAAGTCCATGCCAGCATATCAATCGATGG |
| | | | ACCAAAACATATCAATGACCGCTATCGGTTGGACCGAAAAGGAAA |
| | | | AAGCACTTACGAAGGAACAATTCACGGCTTGCGCATGCTCCAGAA |
| | | | TGCGTGGAAGCAAGGGCGACTCCCGGGAGAGCCCGGCATTCTCT |
| | | | CTGTGGCAAACCCCACAGCGAATGGTGCAGAGATTTATCACCACT |
| | | | TTGCAAACGTCCTCAAATGTCAGCACTTCGATTTCCTCATACCCGA |
| | | | CGCTCACCATGATGATGATATTGATGGCATAGGTATTGGCAGATT |
| | | | CATGAATGAAGCGCTTGACGCATGGTTTGCTGACGGTCGGTCAG |
| | | | AGATTTTTGTTCGAATCTTTAACACATACCTTGGCACGATGCTAAG |
| | | | TAACCAGTTTTACCGGGTTATTGGCATGAGCGCGAATGTAGAATC |
| | | | TGCTTATGCTTTCACGGTAACTGCCGACGGCCTGCTCCGTATTGA |
| | | | TGATACTTTGCGTTCCACCTCTGATGAAATATTCAATGCCATTGGG |
| | | | CATCTCAGTGAATTGTCACTCTCCGGCGTACTCAATTCACCTAATG |
| | | | TCAAAGAATATCTTTCACTAAATAGTGAACTGCCAAGTGATTGTGC |
| | | | AGATTGTGTGTGGAACAAAATCTGTCACGGTGGCCGCTTGGTCAA |
| | | | TCGCTTTTCACGGGCAAACCGTTTCAATAATAAAACCGTGTTCTGT |
| | | | TCATCAATGAGGCTTTTCCTTAGTCGCGCGGCTTCACACCTGATTA |
| | | | CGGCTGGTATTGATGAAGAAACAATAATGAAAAATATTCAGAAAT |
| | | | AGtggagccggacaATGGAAAAAATCAATTTCTGGTTATCAAAGTTTT |
| | | | CATGTGCCGCCCTCGCTATTTGTTGTACATCTTGCCTTGCTGACTC |
| | | | GGGAAATTCGGTAACACTTAAGCTGAATTATGACAAATATTTCAC |
| | | | GCCTCATGCAACTTTCATCATTAATGGCCACCCGGTAAATATGAT |
| | | | GATTGATACAGGTTCTTCGAAGGGCTTTTATCTTCAAGAGCCTCA |
| | | | ACTAAAAAAAATACAAGGCCTCAAAAAAGAAAGCACTTATTACAG |
| | | | TACTAATATCACCGGGAAAAGACAGGAGAACACAGAGTATCTCGC |
| | | | CGCTTCTCTCGACATGAATGGCCTTAAATTAAAAAACGTAACCGT |
| | | | GATCCCATTTAAACAATGGGGAGCGCTGATTTCTAACACAGGTAA |
| | | | ATTGCCGGATGGCCCTGTTGTCGGTCTCGATGCGTTTAAAGATAA |
| | | | ACAAATTATGCTGGATTTTGTGTCTCATTCATTCACGATGAGCGAC |
| | | | AGTTTTATCCATAACATGCCGGTTCCGAAAGGCTTT |
| | | | 33 |
| | | | AACGCATTCACTTTCCATATGTCTCCTGATGGCATGGTTTTTGATG |
| | | | TTGATCAGTCTGGACACACATACCATTTGATTCTGGACACCGGTG |
| | | | CCACTGCGTCTGTGATTTGGCGTGAAAGACTTAAACAGTATGAAC |
| | | | CCAAAAGCTGCCTGCTGGTCGATCCGAAGATGGATAACGAAGGA |
| | | | TGCCAGGCCACTCTGCTCACAATTAAATCAAAAACTGGAAATCCC |
| | | | CAGCATTTTGGTGCGGTTGTTGTTGTCGGAAATTTTAAACACATG |
| | | | GGCAACGTTGATGGCCTTTTAGGGAATAACTTCCTCAGAAATCGA |
| | | | AAGGTACTTATAGACTTTAAAAACAAGAAGGTTTTTATTTCCGATG |
| | | | AGCACCGAAACAGAAAAGAATGACAACTCAATCTTTCGTGCCGAG |
| | | | GCTTTGCAACACAAACGAGAAGGTTGGCTCGGCGCTTCTCGTTTG |
| | | | CATATACCGTCAGCGCTCTCTATTTGTTGCCTGACAATCCTTGTTA |
| | | | TTTTCTTTTTCATCATATTGATAATTGCATTTGGTTCGTACAGTGAA |
| | | | CGGATAAATGTCATCGGAACCGTGGTTTATAAGCCGCCTGCGGTA |
| | | | TCACTGATTGCACAAAGCAGTGGAATCATTACGCATTCACTGGCA |
| | | | TTAGAGCAAACAAGAGTTAAGCGCAACGAGAGCATTTTTTCTATC |
| | | | AGTGGTGACACTCAGACAAATCTGGGTGCCACCAATGTTGAAACG |
| | | | GTAGAACTTTTAAATAAGCAACGTAACGCGCTGTCTAAAAAGCTT |
| | | | GATATTGCGGCCAATGAATCAAAAGCAAACAAGATTTATCTCAGC |
| | | | GAAAAAATTAAAAATAAACAACAGGAAATAGAAAGTCTGCAAAAC |
| | | | CTGATAGAAACTTCAGAAAAACAGCAAGCGTGGTTCGAGAAAAAA |
| | | | TCAAACCTGTATGCGAATTTTAAGAAGAAAGGCATTGCGCTTGAT |
| | | | GCTGAATGGATAAACAGAAAGAAAGATTATTACGCATCCACATTA |
| | | | AGCATTTCTTCTGCAAAGGTCAAAGTGATAGCCCTGCTGGGAGAG |
| | | | TTGCAGGATCTGAAAAATGACGTTTCGGTTATCGACAGGAAACTC |
| | | | GACAAAGAAACAGCATCTCTCACTGTCGAAATAGCCGATATAGCA |
| | | | CAAAAAATACTGATTACAGAAAAACAAAAAGAGTATTTAATCGTCG |
| | | | CGCCGTTTGATGGAATGATAACCAGTGTTACAGCCCATATCGGTG |
| | | | AAAGAGTGACTGCCGGCCAGCAAATAGCCGTGCTGATACCACAA |
| | | | GGTGCGACAGAAAAGGTTGAGTTGTTTTCACCGTCTGATTCTCTC |
| | | | GGTGAAGTGACCAGCGGACAGCAAGTCAGAATGAGAGTCTCGGC |
| | | | ATACCCTTACCAGTGGTATGGAAAGATTGCAGGCATCATAGAAAC |
| | | | GATATCGGCAGCACCGGTCAATGTCACCTCACAGATGCAGATGAA |
| | | | AGGTGAAGAGGTAAAAAAGGGGCTTTTTCGGATTGTCGTACAACC |
| | | | AAAATTGACCGGACAACAAACAAACATTTCCCTTCTACCCGGCAT |
| | | | GGAAGTGGAAACAGAGATCTATGTGAAAACCCGAAAATTGTACGA |
| | | | ATGGTTATTTATCCCCATTAAAGGGGCATATGAACGGGCGACAGA |
| | | | CAGTACGGAATAAatATGCAGTATAAGATGAGTGATTTTTTCGAGT |
| | | | TTTTCGTCAAAAAACTCCCGGTGATAATACAAACAGAGACCACAG |
| | | | AATGCGGGTTGGCATGTCTGGCCATGATTGCTGCCTGGTATGGC |
| | | | CGTGAGACTGATATCTACAGCATGAGAAAGGTTTTTGACGTGTCA |
| | | | AACAATGGCATGACATTAAGGCAGATCATCACGGCGGCCGGGCG |
| | | | AATAAACATGAATACCAGAGCTGTGCGGCTGGAACTCAACGAACT |
| | | | CAGCAGTGTCAGGCTTCCGTGCATCTTGCACTGGTCCTTTAATCA |
| | | | TTTTGTCGTGTTAAAAAAATTCACAAAAAAAGGGGCAGTCATCCAT |
| | | | GATCCCGCCTTGGGAAAAAGAACTGTCACTCTGAAAGAACTCTCA |
| | | | AATAAGTTTACGGGCATCGCTCTGGAAGTCTGGCCCCAGACGGA |
| | | | GTTTAAAAAGGAAAAGGTCAGTGAAAGCATAACCATCACGGATAT |
| | | | GTTTCGCGGTGTTGCCGGCCTTAAGAATACGCTGTTTAAAATCAT |
| | | | TCTGTTGTCGCTCTTTATTGAAGTACTGGCACTTTCCATCCCTCTC |
| | | | AGCTCTCAATTCATTATTGATGTTGTTCTACGGTCCAGTGACCTCA |
| | | | GTATGCTGAATTTCATTGTCATTGGAATCGTTCTTCTGCTCTCCCT |
| | | | GCGCGCTGCTTTCAGTATTGTGCGCGCCTGGGCTCTTATGGCAAT |
| | | | GCGTTACTCACTTGGCATACAGTGGAGTTCCGGTTTTTTTAACCG |
| | | | GTTACTCAGATTGCCGGTCACTTTTTTTGAAAAACGTCACGTAGGT |
| | | | GATATCGCCTCCAGATTGACATCGTTGAGCGAAGTTCAAGAAGCC |
| | | | TTTACAGCAGAAATGCTGACTTCGTTACTTGA |
| | | | 34 |
| | | | TGTACTTATTCTCATAACGCTGGCTGTGCTCATGTTCTGTTACAGC |
| | | | CCTCTTCTGACCCTTCTCCCGCTACTCATGACTACCGTTTATCTTG |
| | | | GGGTCAAATTTGCTTTTTATGACAGATACATGGGAGCAAAAGTAG |
| | | | AAGCAATTACGCATGAAGCGCAGCAATCATCCTACTTTCTCGAAA |
| | | | CAATACGAGGCGTAGCGTGCGTGAAAGTATTTGGCCTGACAGAA |
| | | | TTCCGACGTATCACATGGCTTAACCGGGTGATTGATACTGCCAAT |
| | | | GCCCGGGCCCATTTATTTAAGATAGACCTCATCAGCCAAACGCTT |
| | | | TCAGGTTTCCTGACGGGGCTATCATCGGCGGCCATTTTGTTTATG |
| | | | GGGAGTCATCTCACAGAACGCGGCCTGATCACTGCCGGCATTCT |
| | | | GTTTGCTTTTCTGCTCTATACCGATATGTTTCTGACACGTTCAGTG |
| | | | AAGGTAATAAATTCACTGTTTGCTTTTCGCCTTATTTCGATACACA |
| | | | CGCACCGATTGACCGATATTGCAACAGCCCAGACAGAAAATGCAT |
| | | | GGAACCCGGAAGATCCCGTCACACTCGATAATGTAAAAGGCCGG |
| | | | ATAACACTGAACAATCTCACATATCGGTACGGAGAAACTGAACCC |
| | | | TGTATTTTCGACTGTATCGACATGGAAATTAATGCTGGTGAGAGT |
| | | | GTGGCGATCGTAGGTCCGTCAGGTTGCGGTAAATCGACACTTCTC |
| | | | CGGGTCATGGCCGGCCTGGTTCTCCCTCAGTCAGGCGATGTGTC |
| | | | AATTGATGATGTCAGTGTGAAAAAAATGGGTATTGACGAATATCG |
| | | | CAGACACACGGCGTTTGTCATGCAAGATGATAAGCTTTTTGCTGC |
| | | | CTCATTGATGGATAACATATCCGCTTTTGATCCACAGCCAAATATT |
| | | | GATTGGATACATGAATGCGCTAAGGCGGCGGCAATACACGATGA |
| | | | AATTATGACTATGCCGATGCAGTACGAAACCATGGTGGGTGACAT |
| | | | GGGGAGCATTCTTTCAGGCGGACAAAAACAGCGTGTATCCCTTGC |
| | | | ACGGGCACTTTACAAGTGTCCGCGTATCCTCTTTCTTGATGAGGC |
| | | | CACCAGCCATCTCGACGTTTTTAATGAACGCAAGATAAATGAGGC |
| | | | TGTAAAGCAGATGCCGATTACGCGTGTATTTGTGGCTCATCGGCC |
| | | | AGAAATGATCGCTGTCGCAGACCGAGTTTATAACCTGAGGGATAA |
| | | | GACCTTTACAACGTAA |
| | | | (SEQ ID 196) |
| smcAB | PET-28a(+) | NdeI_XhoI | TCTAAATTAGCCAAAGAAATTAACATGAATAAAGCAGCCGTCACC |
| (Protein ID: | | | GTTGCAGCTGATAAAAAAGACGCACGAAAAGCACTGGCTCAATCT |
| WP_ | | | ATGCTGGATAGCGTTTCTGGCGGTTGGGTCAACGCCTTTGCGCGT |
| 071845309.1, | | | TGGTCCAAAAGCTTCTAAttgaccttggtgcagggtgggagaccgccctgcac |
| WP_ | | | tttctcctttgttgaacagtggtacgggcaATGACGAATAAGAAAAAAATAAA |
| 047728930.1) | | | GCATCTTGAAATAATTTTAAAGGTTAGTGAACGATGCAACATTAAC |
| | | | TGCACGTATTGCTATGTATTCAACCTGGGCAATGATTTGGCAATA |
| | | | AATTCAAAACCAATTATTTCTCATAAAATCATTGAAGATTTGAGAG |
| | | | GTTTTTTCGAGCGGGCCTGCCAGGAGTATGAAATAGAAACGGTTC |
| | | | AGGTTGACTTTCATGGCGGCGAACCGTTAATGATGGGGAAAGAG |
| | | | CGTTTCGACAATGCCTGCAAAGAGCTTATCTCAGGTGACTATAAT |
| | | | GGCGCCAGGCTCAACCTTGCCTGTCAGACAAACGCTATCCTTATT |
| | | | GATAATGAGTGGATTGATATTTTCTCGAAATATAATATCAGCGTGG |
| | | | GGATTTCTATTGATGGCCCCAAGCACATTAACGACAGGCACCGCC |
| | | | TGGATAGAAAGGGACGCAGCACCTACGAAGGTACGGTAAAAGGG |
| | | | CTGGAGATGCTGCAGGTTGCCTGGAAAGCGGGCCGATTGATCGA |
| | | | TGAACCCGGCATCCTGTGCGTCGCCAATCCTTCGGTAAAAGGCG |
| | | | CTGAAATCTATCGTCATTTTGTCGATGTACTGAAATGCAAAAAATT |
| | | | TGATTTCCTCATTCCGGATGAAAGCCATGACACCTGCACGGATCC |
| | | | GGACGGACTGGCGGATTTTTATTGCTCGGCGCTGGACGAGTTCTT |
| | | | TTTGGACGCGGATAAAGAGGTGTATGTGCGCTACTTCCATACGCA |
| | | | CATCCAATCCATGTTGAGTTCAGAATTCAATCCGGTAATGGGAGT |
| | | | AAGCAAAGCCGGGAACGATACTCTCGCTTTCACGGTGAGTTCCGA |
| | | | TGGTGAACTGTATGTGGATGATACGCTGAGAGCAACCAATGACCC |
| | | | TATATTTACGCCTATTGGTAATATTCAACATTTAATACTGTCAGAC |
| | | | ACTCTCGCCTCATGGCAGATGACAAAGTATATGGCTGTGAATAGT |
| | | | CAGCTTCCTACCGTTTGCGGTGACTGTGTCTGGCAAAAAGTTTGT |
| | | | GGCGGAGGGCGTCATATTCAGCGTTATTCTACAGCCGATGATTTT |
| | | | AACCGTGAAACCGTTTTTTGTCCGTCGGTAAGAAAGATCATGAGC |
| | | | CGTGCGGCTTCGCATTTGATTGAATCGGGCGTGGCAGAGGATAT |
| | | | AATCATGAAAAACTTAGAGGTTAACTCATGA |
| | | | (SEQ ID 197) |
| smcCDE | pCDFDuet-1 | NdeI_XhoI | ATCAAGCGGCTATCCTTATTGGCGTTCTTGTTTTCCGGCATCAGC |
| (Protein ID: | | | ATGGCGAGTCTTCCCGCTGATTTTGGGCGGTTGCGGTATGATGAA |
| WP_ | | | CGTGGACTGCCGTTAATTGATGTCCGGATCGATAATCGTCTTCAT |
| 047728928.1, | | | ACCTTAATGTTGGATACCGGCAGCGGGGAGGGGATGCATCTTTAT |
| WP_ | | | AAACACGATCTTGACAACTTAGTGGCTAATCCTGGCCTGCAGGCG |
| 080490739.1, | | | ACCGAACAAGCCCCTCGCCGGTTGATGGATGTTTCAGGGGGTGA |
| WP_ | | | AAATAAAGTTTCCTCATGGAAGATTAATCGATTACTTATTTCCAAT |
| 047728923.1) | | | ATTCCTTTCGATAATGTTGAAGCGGTAAGTTTTAAACCATGGGGA |
| | | | TTAAGCATCGGCGGTGATGTCCCTATGAATGAAGTGATGGGGTTG |
| | | | GGGCTTTTTCGAGAACGCAGAGTGCTGATGGATTTTAAAAACGAT |
| | | | CGGTTAAAAATATTGGCCGACTTGCCATCTGACATAAAGAAATGG |
| | | | TCATCGTACCCCATCGAACCAACCGCATCGGGATTGCGCGTTACC |
| | | | GCCTCCGCAGGCGGTATGCCTTTGCATTTGATTGTCGATACTGCG |
| | | | GCCAGCCATTCTCTGCTGTTTTCAGACCGTTTGCCGCCGGGCCTC |
| | | | CTTTTCTCTGGGTGCCGCGACATTGAGCCGGAAGCGTCGAATCTG |
| | | | GATTGCCGGGTGACAAAAATCGCTTTTACGGATCGCGAAGGTAA |
| | | | GGCTCGTGATGACCAGGCCGTCGTTGCCTCTGGTGCCACGCCCC |
| | | | CGGAACTGGATTTTGACGGTCTTTTGGGGATGAAGTTTATGCGGG |
| | | | GACATCAGGTGATCATCGATATGCCTGAACGCCTGCTCTATATCA |
| | | | GCCGTTAGcgtgATGGACAAAGAAAACTCGTTTTTCCGCCAGGAG |
| | | | GCGTTGCAGCATAAAAAAAAGCCTGGCTGGGCGATTTTACCGTT |
| | | | TCGGCGCCATCAGTGTTGCCCATCGCGTTATGGAGCGCCGTTGG |
| | | | CGTTTTGCTGTTGGCTACCCTTCTGTTATTCACCACTTATGCCAAA |
| | | | AGAGTCCCCGTGACCGGGCGAGTCATCTATACGCCTTCCGCTGCT |
| | | | GAGGCGGTGTTTAACCATGACGGGATTATCGGCCGCATCGAAGT |
| | | | GCACCAAGGGGAAAGGGTTAAGAAAGGGGATGTCATCGCGACGT |
| | | | TTTCACGCGATGTCGCCTATGTCGGGGGAGGCATGAATCAGGCA |
| | | | TTGCAAGATGCGGCGCAGCGCCAGCTTACCGAGTTGCAAAAGCG |
| | | | CGCGGGAGAGCGGCGTAAAGAGGGAGAAGAAGAGCGCTTGCGT |
| | | | TTACGTGAGAAAGTCAGCGCCAAAGAACGGGAAATGGTGGCGAT |
| | | | TCAAGCTGCGGCCGAAGCCGAATCGGAGCACATCGTCGGTTTGA |
| | | | AGAAGCGGATGGCGCTTTATCAACAGCTGTTACTGAAAGGTATTA |
| | | | CGACCGTACAAGAGAAAATTGAGCGGGAGAACGAATATCATAATT |
| | | | CTATTGCACAGCTGAACACGCATCGAATCAATATCGCGCGGGTGA |
| | | | AAGGAGAGCTGCTGCAATTCGAGGATGAGCTGGCTCGCTCTGAA |
| | | | TCGCAAGAAAAACAGTCTATTACTGACATTCAACAGCAGAAGGTC |
| | | | ACGCTGCAACAGCAGGTGATTAATGCCTCTGCGGTCGTGGAGTC |
| | | | TCGGGTTGTGGCTCCGCTTGATGGCGTCGTCGCTTCAATGAGCAT |
| | | | TTTGGAAGGACAGAGAGTGACCGCCGGCGCAGTTGCCGCAGTGG |
| | | | TGGTGCCGGAAAATGCACGTCCGTTCGTTGAAATGTGGATCCCG |
| | | | CCCTCTGCGCTGCAGGAGGTGAAAGCGGGTCAGCATGTTTTCAT |
| | | | GCGCGTCGCATCCTTGCCGTGGGAGTGGTTTGGGAAAGTGTCCG |
| | | | GCACGGTTGCCGCCGTCAGCGAGAGTCCTGAGGCGCTGACGGG |
| | | | AAATAATCGACGTTTTCGCGTGCTGATCGCGCCCGATGTCGGAAC |
| | | | GCGAGCGCTGCCTGCGGGAGTGGACGTTGAGGCCGACATATTGA |
| | | | CGACGCATCGGCGCATCTGGGAATGGCTCTTCTTACCATTAAAAC |
| | | | AAAGTATTAACCGCATGACGGCTGAGAGTTGAcacATGCTTTTTTC |
| | | | CTGGCAAAAAACACCGCTGATTCTACAGTCGGAAACGAATGAGTG |
| | | | TGGGTTGGCCTGTTTGGCCATGATGGCCGGTTATTTCGGCAAACG |
| | | | CATCGATCTTGCTTCGGCGCGTACCCTTCACGGGATCGGCAGCCA |
| | | | CGGGATGACGCTGCGAGATCTCATTACGGCGTTTGAACGTGTGG |
| | | | GGATGACGGCTCGTGCTTCGCGCGTAGAGCTGGATGAACTGCGT |
| | | | TCTCTCAGCCGCCCTGCGATTCTTCACTGGTCATTCAATCATTTCG |
| | | | TGGTGCTGGTGAAAGTGACGCGBTCGGGGCGCGGTGATCCTGGAT |
| | | | CCTGCCATTGGTCGCCGCAGCATTTCATTGCGTGAACTGTCGGAT |
| | | | AAATTTACCGGCGTTTTGGTGGAAGCATGGCCTGCGGAGACCTTC |
| | | | GATAAGAAAGCGCTGGAAATGAATGTCACCGTATCCGATCTTTTT |
| | | | CGTGGCGTACGGGGCTTAAGACGCATTTTTACCGGCGTTCTGATG |
| | | | CTTTCGGTCTTGGTGGAACTGCTCTCCATTGCGGTACCCGCCGCG |
| | | | TCACAATTTACTATCGATACGTTAGTGCGTTCATCAGACCGCGAA |
| | | | GGAATATTTTTTGTCGGTATCGTGGTCATTTCCGCATTGCTGATTA |
| | | | AGTCCGCCTTTTCGGTGGTGCGTGCCTGGATTTTGATGAATCTGC |
| | | | GCTATACGCTCGGCGTGAAATGGGCTGAAATGTTCTTTAACCGGC |
| | | | TTATCAAACTTACGCTGTCATTTTTTGAGAAGCGGCACACCGGCG |
| | | | ATATCGCGTCGCGCTTCCAGTCGTTGACCGCCATTCAGGAAGCGT |
| | | | TTACGGCCGATATGGTTGCCTCTCTCTTGGATGCGATTGTGATTG |
| | | | TCATTTCAATGGCGATCATTTTTACCTATTCACCTGTGCTGGCCAT |
| | | | CGGCCCCCTGATCGCCGCCTGCGCCTATGCCGCCTTGAAGGCGG |
| | | | GCCTGTTCTCGACCTACCGCAATCGTAAAATTGAACATATCGCCTT |
| | | | CGAAGCGGTGCAATCCTCCCACTTCCTTGAAACCGTCAGAGCGAT |
| | | | CGGCGCGATCAAAATGTTGAACCTGACGCCGGTTCGTCGGCGCG |
| | | | AATGGGTCAACCATGTGGTCAACAGCACGCATGCGGGGAACCAG |
| | | | CTGTTTAAACTCGATCTGCTGACCAACACGGCGGCCGTGCTGCTG |
| | | | GTGGGATTTTCCGGGATTTTCGTGCTTAGCGTCGGGGCCATCGG |
| | | | ATTTGATAAAGGCATTACGACTGGCGCCTTGCTGGCCGTGATGCT |
| | | | GTATGCCGATATGGTGATTACCCGCACGGTGAAGTTAGTCAATGC |
| | | | GGTTTCTGATTTTTGCCTGGTATCCATGCACAGTCAGCGTTTGACT |
| | | | GACGTGGCTGTTTCACCCGTGGAACGGGATGAGGGAGAACAAGT |
| | | | GTCGCCACAGCTGAATGGGCATATCGTGATCCGCAACTTAGCGTT |
| | | | CCGCCATTCCCAGACCGAACGCAACATCTTCGAGGGGATCAATCT |
| | | | TGAGATCATGCCAGGGGAAAACGTCGCGATCGTCGGGCCGTCCG |
| | | | GGTGTGGTAAGTCAACATTCCTCCATGTGCTGGCGGGGTTGTAC |
| | | | GAATCTACCGAAGGGGATGTTTTCATTAACAACGTGGGGATGTCT |
| | | | GGCATGGGCAAACGAGACATTCGTGAACATGTCGCTTTTGTCATG |
| | | | CAGGACGACAAACTCTTGGCTGGAACCATACAGCAGAATATTACC |
| | | | GGTTTTACCGCGTCCCCCGATGTGGAACGCATGGCTGAATGCGC |
| | | | CAATCATGCCGCGATTGACGAAGAAATCAGCGCATTTCCACAGGG |
| | | | ATATGAGTCGATGATCGGTGATATTGGTAGCACGCTTTCTGGCGG |
| | | | GCAACGCCAGCGTATTTCTATCGCCAGAGCGCTATACCGGCAACC |
| | | | TCGTGTGCTGCTGCTTGATGAGGCAACCAGCGATCTTGATATCGA |
| | | | TAACGAGAAAAAGATCACTCGCGCCATCGGGCAATTGCCGATAAC |
| | | | CCGCATTTTTGTTGCTCATCGCCCAGAAATGATCAAGTCAGCGGA |
| | | | TCGGGTCTTTAATCTTCATCTGAATGCCTGGGTGAAGCAGGAAAA |
| | | | TCGGGGGGGCGCTACAATGTTGATCGCCGACAAGGTTCACATAA |
| | | | GCTGA |
| | | | (SEQ ID 198) |
| etcAB | PET-28a(+) | NdeI_XhoI | AGCAAATTACAGCATGAAATCGCGTCAAACAAAGCCCGCCTGAAT |
| (Protein ID: | | | AATGCTGACGATAAAAAAGCACAGCGTAAAATCCTTGTTGATAGC |
| WP_ | | | CTGCTGGATACTGTCTCTGGCGGCTGGATAAATGCCTTTGCTAAC |
| 017801003.1, | | | TGGACTAAGCGTATCTAAttgagactgcacgggggagatttccacccccgtgt |
| WP_ | | | tttcccatggaggaggatacacATGACACAGTTAAAAGGCGAAAAAATAA |
| 017801004.1) | | | AGCATCTTGAAATAATTTTAAAAATTAGTGAACGCTGCAATATTAA |
| | | | TTGTACTTACTGCTATGTATTCAATATGGGTAATACACTGGCAACC |
| | | | GATAGCACGCCGGTAATTTCTCTGGATAACGTATACGCGCTGAGG |
| | | | GGATTTTTTGAACGATCGGCTGCCGAAAATGACATTGAGGTTATT |
| | | | CAGGTAGACTTTCACGGTGGCGAACCGCTGATGATGAAAAAAGA |
| | | | CCGTTTCGATCGCATGTGCCAGATTCTCTTGCAGGGTAACTACCG |
| | | | CAGTTCAAAATTTGAACTGGCATTACAAACCAATGGCATTTTGATT |
| | | | GATGACGAGTGGATTGCGCTTTTTGAAAAACATCAGGTGCATGCC |
| | | | AGTATATCGGTCGACGGACCAAAACATATCAATGACCGTCATCGG |
| | | | TTAGACCGTAAGGGGAAGAGCACTTACGAGGGCACAATTACCGG |
| | | | TTTACGCCTGCTGCAAAATGCGTGGCAGCAAGGGCGTCTGCCAG |
| | | | GTGAACCAGGCATACTTTCAGTGGCCAACGCCAATGCAAATGGTG |
| | | | CGGAGATTTATCGCCACTTTGCCGATACTCTCCAGTGCCAGCGTT |
| | | | TCGATTTTCTTATACCAGACGATCATCACGACGATAGCCCTGATG |
| | | | GCGAAGGTGTAGGCCGATTTCTGAACGAGGCACTGGATGCATGG |
| | | | TTTGCTGATGGGCGGCCAGAAATCTTTATTCGAATCTTTAATACTT |
| | | | ATCTCGGCACCATGCTAAACAGCCAGTTTAATCGGGTGCTTGGTA |
| | | | TGAGTGCTAATGTTGAGTCCGCCTATGCCTTTACAGTAACAGCCG |
| | | | ACGGCATGCTGCGTATTGATGACACATTGCGTTCGACATCTGATG |
| | | | AGATATTCAATGCCGTTGGGCATGTCAGTGAATTATCGCTGGCGA |
| | | | GGGTACTTGAAACATCTTGTGTTAAAGAATATCTCGCGTTAAGCA |
| | | | GCAATCTGCCGACAGTGTGCGCAGAATGCGTATGGAATAATATCT |
| | | | GCCACGGCGGCCGTCTGGTAAATCGTTTTTCACGCACTAATCGTT |
| | | | TCAACAATAAAACCGTTTTCTGCAAATCGATGAGATTATTTCTTAG |
| | | | TCGCGCTGCATCGCATCTTATGGCATCGGGCGTGGATGAAAAAG |
| | | | AAATCATGAAAAACATTCAAAAATAG |
| | | | (SEQ ID 199) |
| etcCDE | pCDFDuet-1 | NdeI_XhoI | AAGATGATAATAACCTGGTTATTAAACCGCTTATATTTTGTATTCG |
| (Protein ID: | | | CCTTTAGCACGACACTATCCTTTGCTGATATGGAAAAATCCGTAAC |
| WP_ | | | CTTAACGCTGAGCTTTGATCAGCTTGCCACCCCGCATGCAAATTT |
| 017801005.1, | | | CGTCATCAATGGCACCCCGGTCTATGCCATGGTTGATACGGGTTC |
| WP_ | | | TTCATTTGGTTTCCATCTTTATCAAAATCAACTTAATAAAATCAAAG |
| 017801006.1, | | | GATTAAAAAAAGAACGTACATATCGTAGTACTGATGGAAAAGGTA |
| WP_ | | | AAGTTCAGGAAAATATAGCGTATCTGGCTAAATCTCTCGATATGA |
| 026111678.1) | | | ATGGGTTGAAATTAAGAGATGTCCCCGTCACTCCATTTAAGCAGT |
| | | | GGGGGCTGATGATCTCTGGCGAAGGTGAATTGCCGCAGAGCCAG |
| | | | GTCGTGGGGTTAGGTGCATTTAAAGATAAACAAATATTACTGGAT |
| | | | TATAAGGGGAAATCACTCACCATTGGCGACAACATCGCTTCTGAA |
| | | | TCGCAAATCAAAGAAAATTTTCAGGAATATTCTTTTCAAATGTCTT |
| | | | CCGATGGCATGATCTTTCAAGCCGAGCAATCCGGGCATAAGTATC |
| | | | ATCTGATTATGGATACAGGTTCCACCGTTTCCATAATCTGGCGTG |
| | | | AGAGACTTAAATCCAGACAACCTGAGAGCTGTCTTATTGTCGATC |
| | | | CTGAGATGGATAATGAAGGATGCGAGGCACTGATGCTGGAAACG |
| | | | AAATCGAAGAATGGCAAAATCGAGCATTTTGGCGCGGTCATTGTA |
| | | | GCCGGTGACTTTGAACATATGGGCAATATTGATGGACTTATAGGT |
| | | | AACAACTTCCTCAAAAGCAGAAAGCTATTGATAGATTTTAAAAATA |
| | | | ATAAGGTTTTTATTTCCGATGACAACAGAAAAGGATGATGAGTCA |
| | | | GTCTTTCGTGCCGAGGCATTGCAACATAAGCGTGAGGGATGGTTT |
| | | | GGCCCTTCCCGTCTGCATGTCCCGTCAGGTCTCACTATTTTTCTGA |
| | | | TAACCGGCCTGATAACCGGCATTTTCACTGTATCCATTATTACGTT |
| | | | TGGTTCGTACAGCGAACGGATAAACGTCACCGGAATGGTGGCTT |
| | | | ATGATCCTCCAGCGGTGGCGTTAATGGCACTACGTGATGGGATAA |
| | | | TAACCCGTTCCTCTGCATTTGAGGGAACAATCATAAAACGCGGCC |
| | | | AGCTGGTTTTCACGGTAAGCAGTGATATTCATACCAACCTTGGCC |
| | | | CTGCCAACGTTGAAATGATGGCGCTGTTAAAAAAGCAACGTGATG |
| | | | CACTGTCTAAAAAGCTTGAGATCACCATTAGCAATGCTCAAAAAA |
| | | | ATAGTCTCTATCTGGCCAGTAAAACTAAAATAAAACAGCAGGAAA |
| | | | TTAACAGCCTGGAAGCGTTGATACAAGAAAGCGAAATTCAGAAGG |
| | | | AATGGTTCGCAGAAAAATCCAGGCTGTATACCCACTTAAGAAAAA |
| | | | AAGGCATCGCGCTTGATTCGGATCTGATAGACAGGCGAAAAGATT |
| | | | ATTATTTATCAGCAGAAAGTTTATCTTCATCGAAGGTAAGGCGGAT |
| | | | CACTCTGCAAGGTGAGTTGCTGGAGTTACAGAAACAAGCGTCATC |
| | | | TGTAGACAGGGATTTAAATGAAAAAAAAGAATCCTTTATTATAGAA |
| | | | CTGGCAACCATTGATCAAAGGATTCTTGATGCTGAGAAAAACAAA |
| | | | GAATATTTAATTGTCGCCCCCTTTGATGGCGTCATAACCAGCGTA |
| | | | AGCGCACATATTGGTGAAAGGGTAACAGCTGGACAGAGAATAGC |
| | | | TGTGCTTGTGCCGCAAGGCGCAACGGCAAAAGTTGAGCTACTTTC |
| | | | GCCTTCTGATTCAATTGGTGAAGTCGTCAGAGGGTTGCAAGTAAA |
| | | | AATGAGAGTGGCCGCATACCCTTATCAGTGGTATGGGAAAATCCG |
| | | | TGGCGCGATAGAAGCGATATCGGTAGCACCAGTCAATATGACATC |
| | | | CCCGGCACAGGCAAAGAGTGATTATAGCGGCAAAGGACTTTTTC |
| | | | GCATCATTGTCACACCAGAGCTGACAGAGCAGCAATTGAATATTT |
| | | | CGCTTTTACCTGGCATGGAGGTCGAAGCGGAAATATATGTTAAAA |
| | | | CCAGAAAAGTTTACCAATGGTTATTTATACCTGTCAGGCGGGCAT |
| | | | ATGAACGTGCAACGGACAGCATGGAATAGagATGCAATATAATAT |
| | | | CAGCGCATTTTTTCAGTCTTTTAGCAAAAGGCTACCGGTAATAATG |
| | | | CAAACAGAGGTTACTGAGTGCGGATTAGCTTGCCTGGCAATGATA |
| | | | GCCGCATGGTATGGTCGCAAGACAGATATTTACGGGATGCGAAA |
| | | | ACTTTTTGACGTCTCAAGTAACGGCATGACATTAAGGCAAATAAT |
| | | | GACAGCCGCAGGACGAATAAACCTGAATGCCCGTGCAGTGCGGC |
| | | | TTGAGCTGGAGGAGCTGAGCAGCACATAAACTTCCGTGTATTTTGC |
| | | | ACTGGTCATTCAACCATTTCGTGGTGTTGAAAAAGATAAGCAAAA |
| | | | AAGGCGCTATCATCCATGACCCCGCATCCGGAAAGAGAATTATCA |
| | | | GCATCAATGAACTGTCCAATAAATTTACCGGCATCGCTCTGGAAG |
| | | | TGTGGCCTCAGGCCGAATTTAAAAAAGAAAAAATCAGCGAGAGTA |
| | | | TTACTGTCAGCGATATGTTTCGCGGCGTAGACGGACTTGGGCGT |
| | | | GTGCTGTGTAAAATTCTTCTGTTATCACTGTTTATCGAGATTCTGG |
| | | | CCCTTTCTGTTCCTCTTGCCTCTCAATTTATTATTGATATTGCGTTA |
| | | | AAGGCAAGCGACCTCAACATGTTGAATTTTATTATAACTGGCGTC |
| | | | GTTTTTCTGCTTATCCTGCGTGCGATTCTTAGTATGGTTCGCGCCT |
| | | | GGACGCTTATGGCGATACGTTATTCACTTGGCATCCAGTGGAGCG |
| | | | CCGGATTTTTTAACCGCCTGCTAAAGCTGCCGGTGGCCTTTTTTG |
| | | | AAAAGCGCCATGTCGGAGATATTGCCTCGAGGCTGACTTCGCTAA |
| | | | ATGAGGTGCAGGAAGCATTTACGGCAGAAATGCTTACTTCTCTGC |
| | | | TCGACGTACTTATTCTGCTGGCGCTGATCGCGCTGATGTTCGCTT |
| | | | ACAGCCCATTTTTGGCCATCATATCCCTGCTGATGGCCGCTGTTT |
| | | | ATCTGGGGGTGAAATTAATGTTCTATGACACCTGCATGGGGGCGA |
| | | | AAGTTGAGGCGATAGCGCATGAAGCCCAGCAATCATCCCACTTTC |
| | | | TGGAGACTGTGCGCGGCGTGGCAGCGGTAAAAGTGTTTGATTTA |
| | | | GCTGAATACCGGCGTAACGCATGGCTTAACCGGGTTATTGATACC |
| | | | GCGAATGCACGCGCTCATCTGTTAAAGATAGATCTTATTAACCAG |
| | | | ACGCTTTCGGCTCTGCTGACGGGTCTCTCATCGGCAGCGATCCTG |
| | | | TTTATCGGCGGCAGCCTGATGGAAGCGGGCATAATGACCGCGGG |
| | | | TATTCTGTTGGCTTTTCTGCTCTATGCAGATATGTTCCTTACCCGT |
| | | | TCAGTGAAGGTGATAAATTCGCTGTTTGATTTTCGTCTGATCTCGA |
| | | | TCCACACGCAGCGCCTGACAGATATTGCTGCAACCGAAACAGAAA |
| | | | GTGCATGGAATCCGCTAAATCCTGTACGGCTTGAGAACGTATCCG |
| | | | GCCAGCTAACCCTGAGTGCGCTTTCATTTCGCTACAGTGAGGCGG |
| | | | AACCCTTTATTTTCGAAGGGATAGATATGGAGATCAAACCGGGCG |
| | | | AGAGCGTAGCGATTATCGGCCCATCAGGCTGTGGTAAATCGACG |
| | | | CTTCTCAATGTTATGGGGGGTCTGACTCTTCCGCATTCAGGAGAG |
| | | | ATATTTATTGATGGCGTTAGTGTCCGCCAGACTGGTATTGACGAA |
| | | | TACCGTCGGCACACGGCGTTTGTCATGCAGGATGATAAATTATTT |
| | | | GCAGCCTCACTCATGGATAACATCACTTCTTTTACCCCACAGCCTG |
| | | | ATATTGACTGGATGCATGAATGCGCCACGGCAGCGGCAATCCAT |
| | | | GATGAGATTATGGCGATGCCGATGCAATACGAAACGATGGTGGG |
| | | | TGACATGGGAAGTATTCTTTCTAGCGGACAAAAACAGCGCGTGTC |
| | | | GCTCGCCAGGGCGCTGTACAAGCGTCCCCGCATTCTGTTTCTTGA |
| | | | TGAGGCCACCAGTGACCTGGACGTTATTAACGAGCGGAAGATCA |
| | | | ATGAAGCGGTAAAACAGATGCCTGTTACACGGGTATTCGTGGCTC |
| | | | ACCGGCCAGAGATGATTGCTGTCGCCGATCGGGTTTATAACCTGA |
| | | | GAGATAAAACTTTTGTGCCATCAGGCTATGAGGTTACAGATTAA |
| | | | (SEQ ID 200) |
| pacAB | PET-28a(+) | NdeI_XhoI | TCTAACTTGAAAAAAGAAATCGCTGAAACTAAAACTGAAATTAAAG |
| (Protein ID: | | | GTACTAAAGTTAAAAATAATCAACCTCAACCTCTAACAGAAGATCT |
| WP_ | | | GCTCGACCAAATCTCTGGTGGTTGGGTGAATGCTTACGCAAGATG |
| 072023203.1, | | | GACAAACCGCTTTTAAattcagtagattaaagtcagggggcttaattgccccca |
| WP_ | | | tttgattctttcgagctgagcaatgttcgtagttggaacttaacctgccattttcgtattac |
| 036768348.1) | | | tggcatagggtctaacaaagtaaaaaATGGAGCTTCGAGTGATGGTTAAT |
| | | | TCATTAGTTAAGAAAAAAATTCAACATCTTGAAGTAATATTAAAGA |
| | | | TAAGCGAGCGATGTAATATCAATTGTGACTATTGTTACGTATTCAA |
| | | | TAGAGGAAATTCAGCGGCTAATGATAGCCCCGCCAGGATCTCTCA |
| | | | TGCGAATATTGATTACCTGGTGGATTTCTTTCAGCGGGGAAGTCA |
| | | | AGAATATGATATTGACACTCTGCAAATTGATTTTCATGGAGGAGA |
| | | | ACCTCTCATGATGAAAAAGCCGCAGTTTGCCAGTATGTGTGAGCG |
| | | | ACTAGCCTCAGGTAATTACCATGGTTCGAAAATCAGATTTGCATTA |
| | | | CAGACTAATGGCATCCTTATTGATGATGAATGGATATCTTTATTCG |
| | | | AAAAATATTCTGTCAGTGTGAGTGTCTCCATTGATGGACCGAAGC |
| | | | ATATTAATGATCGTCATCGCTTAGACAGAAAAGGGCGTAGTACTT |
| | | | ACGAAGGTACTATACGGGGTCTCCGTAAACTTCAAGAAGCTTATC |
| | | | AAGCAGGTCGGCTGCCGTCAGATCCGGGTATTTTGTGTGTCGCG |
| | | | AATGCTAAAGCAAGCGGGGCTGAAATATATCGACACTTTGTTGAT |
| | | | AACCTGGGCGTTTATGGCTTTGATTTTCTGGTACCTGACGACTGT |
| | | | TACACTGATGCCCAGGTTGATCCAGATGGCGTTGGACGTTTCCTA |
| | | | AATGAGGCGTTAGATGAATGGGTGAATGACAATAACCCCAAGATT |
| | | | TTTGTGCGTCTTTTTAATACCCATATTGCCAGTCTTCTTGGCGCGG |
| | | | AAAATGCGGGGTTTTTGGGGCATAACCCAAGCGTAGCTGGAATAT |
| | | | ATGCATTTACCATTGGTTCAGATGGTTTTGTCCGTGTCGATGATAC |
| | | | CTTGAGATCGACATCTGACCGTATTTTCGACATCATTGGTCACATT |
| | | | TCTGAAATCAGCCTATCTGAAGTATTAAATAGCCCACAGTTTCAGG |
| | | | AATATGCGTCTATAGGGGAATCGTTACCAACAGAATGTGAAGACT |
| | | | GTATTTGGGCAAAAGTTTGTGCCGGTGGGCGCATAGTTAATCGCT |
| | | | TCTCGCATGAAGAGAGATTTAAACGCAAGTCAGTATATTGTTATTC |
| | | | AATGAGAAGCCTTCTTAGCCGCGTTTCAGCTCATCTTCTCAATATG |
| | | | GGGATTGAGGAAGATCGCATTATGAAAGCGATTGGCCGGTAA |
| | | | (SEQ ID 201) |
| pacDEC | pCDFDuet-1 | NdeI_XhoI | CCAGTAGGCGCCTCAGTTTGGACAATAATAGCGCTTGTTATTATT |
| (Protein ID: | | | GTCAGCCTTGTTGTGTTCATGATAATAGGCACTTACACACAGAAG |
| WP_ | | | GTTCGGCTAATGGGGGAAATTATCTACGAGCCTGCGGTTGCGAG |
| 051690838.1, | | | AATAGAAGCAACGGGTAACGGAACCATTGTCCGTAGTTTTGCTGT |
| WP_ | | | TGAAGGGAAAGAAGTTCGCGCTGGAGATGTTATTTTTATCGTTAA |
| 036768349.1, | | | CATGGAAACTCAAACCGAATATGGGCGTACAAGTCATGAAATTAC |
| WP_ | | | TTCTGCCCTCAAGTCACAAAAAACCGCTATTGAACGAGAGATCAT |
| 110882651.1) | | | GCTGAAATCAGAGGCGTCTGATCAAGAAAGTGATTTTCTTACCCA |
| | | | GCGTCTTAAGAATAAGGAAGCGGAAATTCAAGAATTAGACAACCT |
| | | | GATCACAAAATCAACCGAACAAGTCGCGTGGCTATTTGACAAAGC |
| | | | TCAGCTTTTCAATAAATTAGTTGGGAAAGGAATCGCACTTGAAATA |
| | | | GATCATATAGAACGCCGCTCTGATTATTATACTGCTTCTGTTCAAC |
| | | | TGGCGGCTTACAAACGAGAAAAGGTTAAGTTACAGGGTGAATCTC |
| | | | TCGATATCAGGGCGAGGTTGGCGACAATCCACATTGGACTTGAAA |
| | | | CTTCACGTGAAACATTACGTCGAGATATTGCACGGCTAGATCAAG |
| | | | ACTTAGTCTCTACGGCAGAACGAAGGGAACTCTATATAACGTCTC |
| | | | CAATTGACGGTAAGTTAACGGGAATTACTGGATTAGTTGGCAAAA |
| | | | GAATTCGCTCGTCCCAGGAATTAGCGAGTGTTGTACCTACTTCGG |
| | | | GCCGCCCCAAAGTAGAAATCTTTTCCACTTCTGAAGTTATTGGAG |
| | | | AATTACGCGAGGGACAATCTGTAAAATTACGGTTTGATGCTTATC |
| | | | CATACCAGTGGTTTGGGCAGCATGATGGTATTGTTACTGCAATTT |
| | | | CCACGACTTCAGTTGAAGGGAGTTTAGGAATAAAGGATGAAAATA |
| | | | ATCAGCAACAGAAACGGTATTTTCAGGTTCATATCCGTCCTAAAA |
| | | | GCGACGGTGTACTCTTAGCGGGAAATATGCATCCTTTACGGCCCG |
| | | | GAATGGGGGTCGAAACAGACATTTTTATAAGAAAAAGGCCAATCT |
| | | | ACGAATGGATTTTGTTACCTCTAAAAAGAATTCATGTCGCGACTCA |
| | | | AGGTAAACCTGGAGATGATGTATGAATGTCACAATGAAAGGCTAC |
| | | | TTTGAAGCATTCAGGCACCATCTTCCTGTAGTGATGCAAACAGAG |
| | | | GCTACGGAATGTGGACTCGCTTGTGTCGCTATGATTGCAGGTTAT |
| | | | TATGGACTTAATATGGATCTGCAAGCGCTTCGCAAATATTATCAG |
| | | | GTGTCTTTAAAAGGTATGAACCTGCGCGATATTATCGTATTAGCT |
| | | | GATCGCCTCTCATTAGCGTCTCGTCCAATTCGAGCTGATCTTGATT |
| | | | CTTTAAGTCAGGTAAAAACGCCTTGTATTTTGCATTGGTCTTTTAA |
| | | | TCACTTTGTTGTATTAAAGAAATTTTCACGCCGTGGGGTCGTTATT |
| | | | CACGATCCGGCAAAAGGCGAGAGAAGAATTTCTATCGATGAGTTA |
| | | | TCTAAAAAATTTACGGGTATTGCACTTGAGCTTTGGCCAAATAAAG |
| | | | ACTTTCAGAAACGTACTGAAAAGAAAACAATTCGACTGCTGGATA |
| | | | TGTTTAAAAACGTTTCTGGATTATCTCGGGCTTTAGTTCAAGTATT |
| | | | GGCTTTATCATTTTGTATTGACTTCTTGCTATGGCCGTGCCGATG |
| | | | GCAGCTCAATTCACGATAGATATGGCTTTGAGGTCTAGCGATATT |
| | | | GATCTTGTCTCTGTGATTGTGTGCGGAATTATTGGCTTATTAATAT |
| | | | GATCGCCTCTCATTAGCGTCTCGTCCAATTCGAGCTGATCTTGATT |
| | | | TAAGTATACTTTGGGTATTCAATGGAGCTCTGGGCTTTTTAGTCAT |
| | | | ATGATCCGATTACCTACTTCATACTTTGAAAAGCGTCATATTGGTG |
| | | | ACGTCACTTCGCGATTTAACTCTTTATCGGCAGTACAAGATGCCTT |
| | | | CACCGCGGATATGATAGCTTCACTCTTAGACATTGTTGTGGTGAT |
| | | | TGGACTCTTCTTTTTAATGTGGGTTTACAATGGTTATCTTGCTGTC |
| | | | GTGGTCATTTCGATATCCATTGTATACGCATCGCTAAAATTCTTTC |
| | | | TTTTTCGAGCCTATCGTTCGGCTAATCTCGAGGCGATAGCCCATG |
| | | | AATCTCAGCAACAGTCACACTTCCTTGAAACAGTACGCGGCATCA |
| | | | CTTGCGTTAAAATTTTTGACTTAGCCGATCGCAGACGATCCGATT |
| | | | GGCTCAATCTTGTTATTGATGAAGCCAATGCAAAAATATACCTCTT |
| | | | TAAAATTGACCTGGTGACACAGACTGCGGCACAGCTTTTAATTGG |
| | | | TCTTACTTCTGCATCCATATTATGGTTAGGCGCTAAATTGATTGAT |
| | | | GGCGGCGCGTTAACCACAGGTATGCTTTTTGCCTTCTTGATTTAC |
| | | | TCTGATATGTACGTAAATCGAACCATACGAGTGGTTGACTCGATT |
| | | | ATTAAACTTCGCTTGATCGATATGCATAGCGAACGACTGTCAGAA |
| | | | GTGGCTTTAGCCGAACCTGAACATAATGAAGGGGATGCTGTTCTA |
| | | | TCATGTCCTGAAACAATTTCAGGCAGTATTGAAATTAAAAGCCTGA |
| | | | GTTATCGTTATGGCGATGGCGAACCCGCTATATTTGAGAATGTTT |
| | | | TTCTGTCTATTAAGGCTGGTGAAAGTATCGCTATAGTTGGGCCGT |
| | | | CAGGTTGTGGTAAATCGACACTGCTTAAGACAATCGGTGGATTAG |
| | | | TCTCGCCAGAAAGTGGCTTTATTTATTTGGACGGAGTTGATGTGC |
| | | | GGAGATTAGGACTTGGGGCCTACCGTAGCCATATCGCTTGTGTCT |
| | | | TACAAGAGGACAGATTATTTGCGGGATCGCTATTGGATAATATTA |
| | | | GTTCATTCGACGTTAAGCCTGACCATGAATGGGTATATGAGTGTG |
| | | | CTCGTCTTGCTTCAATTCACGCTGAAATAGAAGAGATGCCAATGA |
| | | | AATATGAAACAATGGTTGGAGACATGGGCAGTGCTCTGTCAGGT |
| | | | GGACAACGGCAGCGTATTTCTCTTGCCAGGGCATTGTACAAACGT |
| | | | CCAAAGATATTATTTCTTGATGAAGCAACGAGTGATCTGGATATC |
| | | | GATAACGAAGCAAAAATTAATGACTCAATACGAGAACTAAAGATT |
| | | | ACCAGGGTATTTGTAGCCCATCGTCCGACAATGATCGCAATGGCG |
| | | | GATAGGGTTTTTGATCTAAGTATGAACGCAGAAGTGGAGAACCCC |
| | | | CATGCATTTTTCTCTAAGTAAACATATCAAGGTGACCGCATTTGTT |
| | | | GCTTTTTCTTCCATGATGTCATTATTTGTTGCAAATTCTATGGCCG |
| | | | CTGAAAAAGTCATGCATATCAATTTTCAATTTGATGAATTTGCTCT |
| | | | ACCGATAGCAAATCTTGAAATTGATGGAAAAACTCAAAATCTTATG |
| | | | ATCGATACGGGTTCAACTATAGGTCTCCATTTATCTAAAAACCTGA |
| | | | TGTCGAAAATTTCCGGCTTAGTTATCGAACCTGAAAAAGCGCGTT |
| | | | CTACTGACCTTACGGGTAAGACTTTTTTAAATGACAAATTTAATAT |
| | | | TCCACGGCTTTCGATAAATGGCATGATGTTTAAAGATGTTAAAGG |
| | | | GGTTTCATTAACACCATGGGGAATGAAATTAATTGGAGACAATGA |
| | | | TCTTCCTTCCTCAATGGTAATTGGCCTTGATTTATTCAAGGGAAAG |
| | | | GTGGTTCTTATTGATTATAAAAGCCGGAAATTATCAGTTTCTGATC |
| | | | GTTTGCAAGCGTTGGGAGTCAATGTGGATAATGGTTGGATAAAAT |
| | | | TGCCGCTGAGACTGACTAAAGAAGGCATTGCTGTCAAAGTTTCAC |
| | | | AAAACTTTAAAAGCTACAACATGGTATTGGATACTGGCGCATCGG |
| | | | TTTCGATTTTTTGGAAAGAAAGATTGAAATCTCCTCCGGTTAACAT |
| | | | TTCTTGCCAGGCTGTGGTTAAAGAGATGGACAATGAAGGGTGTGT |
| | | | TGCATCGACGTTTCAGCTTGACGAAATGGGCGTTAAGGGAGTTAA |
| | | | GCTGAATTCGGTATTGGTTGATGGGGGATTTAATCAGTTAAATAC |
| | | | TGATGGATTAATCGGGAATAATTTCTTTAATAAATACGCAGTATTA |
| | | | ATCGACTTCCCTGGTAAGAGATTATTCATTAAAGAGAACTCGTAG |
| | | | (SEQ ID 202) |
| xyeB₂₄-xncCDE | pCDFDuet-1 | NdeI_XhoI | GCTAACAAAGAAAAAATCAAACACCTGGAAATCATCCTGAAAGTT |
| (Protein ID: | | | TCTGAACGTTGCAACATCAACTGCACCTACTGCTACGTTTTCAACC |
| WP_ | | | TGGGTAACGACCTGGCTATCAACTCTAAACCGATCATCTCTCACG |
| 103774053.1, | | | GTACCATCAAAAACCTGCGTGGTTTCTTCGAACGTGCTTGCCAGG |
| WP_ | | | AATACGAAATCGAAACCGTTCAGGTTGACTTCCACGGTGGTGAAC |
| 013185693.1, | | | CGCTGATGATCGGTAAAGACCGTTTCGACAACGCTTGCAAAGAAC |
| WP_ | | | TGGTTTCTGGTGACTACAACGGTACCCGTCTGAACCTGGCTTGCC |
| 013185694.1, | | | AGACCAACGCTATCCTGATCGACAACGAATGGATCGACATCTTCT |
| WP_ | | | CTAAACACAACATCTCTGTTGGTATCTCTATCGACGGTCCGAAAC |
| 013185695.1) | | | ACATCAACGACCGTCACCGTCTGGACCGTAAAGGTCGTTCTACCT |
| | | | ACGAAGGTACCGTTAAAGGTCTGGAAATGCTGCAGGCTGCTTGG |
| | | | CGTGCTGGTCGTCTGATCGACGAACCGGGTATCCTGTGCGTTGCT |
| | | | AACCCGTCTGTTAAAGGTGCTGAAATCTACCGTCACTTCGTTGAC |
| | | | GTTCTGAAATGCAAAAAATTCGACTTCCTGATCCCGGACGAATCT |
| | | | CACGACACCTGCACCGACCCGGAAGGTCTGTCTGACTTCTACTGC |
| | | | TCTGCTCTGGACGAATTCTTCCTGGACGCTGACAAAGAAGTTTAC |
| | | | GTTCGTTACTTCCACACCCACATCCAGTCTATGCTGTCTCTGGAAT |
| | | | TCTCTCCGGTTATGGGTGTTTCTAAAGCTGGTTCTGACACCCTGG |
| | | | CTTTCACCGTTTCTTCTGACGGTGAACTGTACGTTGACGACACCC |
| | | | TGCGTTCTACCAACGACTCTATCTTCACCCGATCGGTCACATCCA |
| | | | GTCTCTGACCCTGTCTGAAGCTCTGACCTCTTGGCAGATGCAGAA |
| | | | ATACCTGTCTGTTGACAACCAGCTGCCGGAAGTTTGCATCGACTG |
| | | | CATCTGGAAAAAACTGTGCGGTGGTGGTCGTCACATCCAGCGTTA |
| | | | CTCTTCTGCTGACGACTTCAACCGTGAAACCGTTTTCTGCCCGTCT |
| | | | ATCCGTAAAATCATGTCTCGTGCTGCTTCTCACCTGATCGAATCTG |
| | | | GTGTTACCGAAGACATCATCATGAAAAACCTGGAAGTTAACTCTT |
| | | | AATGGAGCCGGACAATGGAAAAAATCAATTTCTGGTTATCAAAGT |
| | | | TTTCATGTGCCGCCCTCGCTATTTGTTGTACATCTTGCCTTGCTGA |
| | | | CTCGGGAAATTCGGTAACACTTAAGCTGAATTATGACAAATATTTC |
| | | | ACGCCTCATGCAACTTTCATCATTAATGGCCACCCGGTAAATATG |
| | | | ATGATTGATACAGGTTCTTCGAAGGGCTTTTATCTTCAAGAGCCTC |
| | | | AACTAAAAAAAATACAAGGCCTCAAAAAAGAAAGCACTTATTACA |
| | | | GTACTAATATCACCGGGAAAAGACAGGAGAACACAGAGTATCTCG |
| | | | CCGCTTCTCTCGACATGAATGGCCTTAAATTAAAAAACGTAACCGT |
| | | | GATCCCATTTAAACAATGGGGAGCGCTGATTTCTAACACAGGTAA |
| | | | ATTGCCGGATGGCCCTGTTGTCGGTCTCGATGCGTTTAAAGATAA |
| | | | ACAAATTATGCTGGATTTTGTGTCTCATTCATTCACGATGAGCGAC |
| | | | AGTTTTATCCATAACATGCCGGTTCCGAAAGGCTTTAACGCATTCA |
| | | | CTTTCCATATGTCTCCTGATGGCATGGTTTTTGATGTTGATCAGTC |
| | | | TGGACACATACCATTTGATTCTGGACACCGGTGCCACTGCGTC |
| | | | TGTGATTTGGCGTGAAAGACTTAAACAGTATGAACCCAAAAGCTG |
| | | | CCTGCTGGTCGATCCGAAGATGGATAACGAAGGATGCCAGGCCA |
| | | | CTCTGCTCACAATTAAATCAAAAACTGGAAATCCCCAGCATTTTGG |
| | | | TGCGGTTGTTGTTGTCGGAAATTTTAAACACATGGGCAACGTTGA |
| | | | TGGCCTTTTAGGGAATAACTTCCTCAGAAATCGAAAGGTACTTATA |
| | | | GACTTTAAAAACAAGAAGGTTTTTATTTCCGATGAGCACCGAAAC |
| | | | AGAAAAGAATGACAACTCAATCTTTCGTGCCGAGGCTTTGCAACA |
| | | | CAAACGAGAAGGTTGGCTCGGCGCTTCTCGTTTGCATATACCGTC |
| | | | AGCGCTCTCTATTTGTTGCCTGACAATCCTTGTTATTTTCTTTTTCA |
| | | | TCATATTGATAATTGCATTTGGTTCGTACAGTGAACGGATAAATGT |
| | | | CATCGGAACCGTGGTTTATAAGCCGCCTGCGGTATCACTGATTGC |
| | | | ACAAAGCAGTGGAATCATTACGCATTCACTGGCATTAGAGCAAAC |
| | | | AAGAGTTAAGCGCAACGAGAGCATTTTTTCTATCAGTGGTGACAC |
| | | | TCAGACAAATCTGGGTGCCACCAATGTTGAAACGGTAGAACTTTT |
| | | | AAATAAGCAACGTAACGCGCTGTCTAAAAAGCTTGATATTGCGGC |
| | | | CAATGAATCAAAAGCAAACAAGATTTATCTCAGCGAAAAAATTAAA |
| | | | AATAAACAACAGGAAATAGAAAGTCTGCAAAACCTGATAGAAACT |
| | | | TCAGAAAAACAGCAAGCGTGGTTCGAGAAAAAATCAAACCTGTAT |
| | | | GCGAATTTTAAGAAGAAAGGCATTGCGCTTGATGCTGAATGGATA |
| | | | AACAGAAAGAAAGATTATTACGCATCCACATTAAGCATTTCTTCTG |
| | | | CAAAGGTCAAAGTGATAGCCCTGCTGGGAGAGTTGCAGGATCTG |
| | | | AAAAATGACGTTTCGGTTATCGACAGGAAACTCGACAAAGAAACA |
| | | | GCATCTCTCACTGTCGAAATAGCCGATATAGCACAAAAAATACTG |
| | | | ATTACAGAAAAACAAAAAGAGTATTTAATCGTCGCGCCGTTTGAT |
| | | | GGAATGATAACCAGTGTTACAGCCCATATCGGTGAAAGAGTGACT |
| | | | GCCGGCCAGCAAATAGCCGTGCTGATACCACAAGGTGCGACAGA |
| | | | AAAGGTTGAGTTGTTTTCACCGTCTGATTCTCTCGGTGAAGTGAC |
| | | | CAGCGGACAGCAAGTCAGAATGAGAGTCTCGGCATACCCTTACC |
| | | | AGTGGTATGGAAAGATTGCAGGCATCATAGAAACGATATCGGCA |
| | | | GCACCGGTCAATGTCACCTCACAGATGCAGATGAAAGGTGAAGA |
| | | | GGTAAAAAAGGGGCTTTTTCGGATTGTCGTACAACCAAAATTGAC |
| | | | CGGACAACAAACAAACATTTCCCTTCTACCCGGCATGGAAGTGGA |
| | | | AACAGAGATCTATGTGAAAACCCGAAAATTGTACGAATGGTTATT |
| | | | TATCCCCATTAAAGGGGCATATGAACGGGCGACAGACAGTACGG |
| | | | AATAAATATGCAGTATAAGATGAGTGATTTTTTCGAGTTTTTCGTC |
| | | | AAAAAACTCCCGGTGATAATACAAACAGAGACCACAGAATGCGG |
| | | | GTTGGCATGTCTGGCCATGATTGCTGCCTGGTATGGCCGTGAGA |
| | | | CTGATATCTACAGCATGAGAAAGGTTTTTGACGTGTCAAACAATG |
| | | | GCATGACATTAAGGCAGATCATCACGGCGGCCGGGCGAATAAAC |
| | | | ATGAATACCAGAGCTGTGCGGCTGGAACTCAACGAACTCAGCAG |
| | | | TGTCAGGCTTCCGTGCATCTTGCACTGGTCCTTTAATCATTTTGTC |
| | | | GTGTTAAAAAAATTCACAAAAAAAGGGGCAGTCATCCATGATCCC |
| | | | GCCTTGGGAAAAAGAACTGTCACTCTGAAAGAACTCTCAAATAAG |
| | | | TTTACGGGCATCGCTCTGGAAGTCTGGCCCCAGACGGAGTTTAAA |
| | | | AAGGAAAAGGTCAGTGAAAGCATAACCATCACGGATATGTTTCGC |
| | | | GGTGTTGCCGGCCTTAAGAATACGCTGTTTAAAATCATTCTGTTGT |
| | | | CGCTCTTTATTGAAGTACTGGCACTTTCCATCCCTCTCAGCTCTCA |
| | | | ATTCATTATTGATGTTGTTCTACGGTCCAGTGACCTCAGTATGCTG |
| | | | AATTTCATTGTCATTGGAATCGTTCTTCTGCTCTCCCTGCGCGCTG |
| | | | CTTTCAGTATTGTGCGCGCCTGGGCTCTTATGGCAATGCGTTACT |
| | | | CACTTGGCATACAGTGGAGTTCCGGTTTTTTTAACCGGTTACTCA |
| | | | GATTGCCGGTCACTTTTTTTGAAAAACGTCACGTAGGTGATATCG |
| | | | CCTCCAGATTGACATCGTTGAGCGAAGTTCAAGAAGCCTTTACAG |
| | | | CAGAAATGCTGACTTCGTTACTTGATGTACTTATTCTCATAACGCT |
| | | | GGCTGTGCTCATGTTCTGTTACAGCCCTCTTCTGACCCTTCTCCCG |
| | | | CTACTCATGACTACCGTTTATCTTGGGGTCAAATTTGCTTTTTATG |
| | | | ACAGATACATGGGAGCAAAAGTAGAAGCAATTACGCATGAAGCG |
| | | | CAGCAATCATCCTACTTTCTCGAAACAATACGAGGCGTAGCGTGC |
| | | | GTGAAAGTATTTGGCCTGACAGAATTCCGACGTATCACATGGCTT |
| | | | AACCGGGTGATTGATACTGCCAATGCCCGGGCCCATTTATTTAAG |
| | | | ATAGACCTCATCAGCCAAACGCTTTCAGGTTTCCTGACGGGGCTA |
| | | | TCATCGGCGGCCATTTTGTTTATGGGGAGTCATCTCACAGAACGC |
| | | | GGCCTGATCACTGCCGGCATTCTGTTTGCTTTTCTGCTCTATACCG |
| | | | ATATGTTTCTGACACGTTCAGTGAAGGTAATAAATTCACTGTTTGC |
| | | | TTTTCGCCTTATTTCGATACACACGCACCGATTGACCGATATTGCA |
| | | | ACAGCCCAGACAGAAAATGCATGGAACCCGGAAGATCCCGTCAC |
| | | | ACTCGATAATGTAAAAGGCCGGATAACACTGAACAATCTCACATA |
| | | | GGAAATTAATGCTGGTGAGAGTGTGGCGATCGTAGGTCCGTCAG |
| | | | GTTGCGGTAAATCGACACTTCTCCGGGTCATGGCCGGCCTGGTTC |
| | | | TCCCTCAGTCAGGCGATGTGTCAATTGATGATGTCAGTGTGAAAA |
| | | | AAATGGGTATTGACGAATATCGCAGACACACGGCGTTTGTCATGC |
| | | | AAGATGATAAGCTTTTTGCTGCCTCATTGATGGATAACATATCCGC |
| | | | TTTTGATCCACAGCCAAATATTGATTGGATACATGAATGCGCTAAG |
| | | | GCGGCGGCAATACACGATGAAATTATGACTATGCCGATGCAGTAC |
| | | | GAAACCATGGTGGGTGACATGGGGAGCATTCTTTCAGGCGGACA |
| | | | AAAACAGCGTGTATCCCTTGCACGGGCACTTTACAAGTGTCCGCG |
| | | | TATCCTCTTTCTTGATGAGGCCACCAGCCATCTCGACGTTTTTAAT |
| | | | GAACGCAAGATAAATGAGGCTGTAAAGCAGATGCCGATTACGCG |
| | | | TGTATTTGTGGCTCATCGGCCAGAAATGATCGCTGTCGCAGACCG |
| | | | AGTTTATAACCTGAGGGA |
| | | | (SEQ ID 203) |
| xyeA₂₄₋₁ | PET-28a(+) | NdeI_Xhol | TCTAAACTGGCTAAAGAAATCTCTATGAACAAAGCTGCTGTTATCA |
| engineered | | | TCGACGGTGACAAAAAAGACGTTCGTCGTGCTCTGACCCAGTCTA |
| | | | TGCTGGACTCTGTTTCTGGTGGTTGGGTTAACgcaTTCGCTCGTTG |
| | | | GTCTaaaCGTTGGTAAAATTCGAGCTCGGCGCGCCTGCAGGTCGA |
| | | | CAAGCTTGCGGCCGCATAATGCTTAAGTCGAACAGAACCCAAGAC |
| | | | CAGGGGGGCTCGCCACGTTGGCTAATCCTGGTACATCTTGTAATC |
| | | | AATATTCAGTAGAAAATTTGTGTTAGA |
| | | | (SEQ ID 204) |
| xyeA₂₄₋₂ | pET-28a(+) | NdeI_Xhol | TCTAAACTGGCTAAAGAAATCTCTATGAACAAAGCTGCTGTTATCA |
| engineered | | | TCGACGGTGACAAAAAAGACGTTCGTCGTGCTCTGACCCAGTCTA |
| | | | TGCTGGACTCTGTTTCTGGTGGTTGGGTTAACgcaTTCGCTCGTTG |
| | | | GTCTaaaCGTttcTAAAATTCGAGCTCGGCGCGCCTGCAGGTCGAC |
| | | | AAGCTTGCGGCCGCATAATGCTTAAGTCGAACAGAACCCAAGACC |
| | | | AGGGGGGCTCGCCACGTTGGCTAATCCTGGTACATCTTGTAATCA |
| | | | ATATTCAGTAGAAAATTTGTGTTAGAA |
| | | | (SEQ ID 205) |
| His6-ykcA + | pRSFDuet-1 | NcoI_XhoI | GGTCATCACATCATCATCATCATCACAGCTCTGGATTAGTGCCGC |
| ykcB | | | GCGGTAGTCATATGTCTCGCTTACAAAAAGAAATCAATGAAACTA |
| (Protein ID: | | | AGACAGTCATTAACATTTGTAATACTAAAAAGAGTCAACCTCAGCA |
| WP_ | | | TCTTGCAGACAGTATTCTCGACAAGATAGCAGGCGGTTGGGTGAA |
| 072082693.1, | | | TGCTTTTGTAAACTGGCCAAAAAGTTTTTAAgaattcgagctcggegcgc |
| WP_ | | | ctgcaggtcgacaagcttgcggccgcataatgcttaagtcgaacagaaagtaatcgt |
| 050115763.1) | | | attgtacacggccgcataatcgaaattaatacgactcactataggggaattgtgagcg |
| | | | gataacaattccccatcttagtatattagttaagtataagaaggagatatacatATGG |
| | | | TCAATCAATTAAACATTCAAAGCATCCAACACCTTGAAATAATATT |
| | | | AAAAATAAGCGAACGCTGTAATATTAATTGTGATTATTGCTATGTA |
| | | | TTCAATAAAGGTAATCCGGCGGCTAATAACAGCCCCGCCAGATTG |
| | | | TCAGATAGAAACATTAATGACTTAGCTGAATTTCTTCACACAGCAT |
| | | | GTCGGGAATATAAAATCGGTACCCTACAAATTGATTTCCACGGGG |
| | | | GGGAACCGTTATTGATGAAAAAAGAAAACTTCGCCAAAATGTGTG |
| | | | AGCGATTACTGACAGGAAGATACTCGAAGACTAATATCAGATTCG |
| | | | CATTGCAAACTAACGGCACACTTATTGATGAAGAATGGATATCAC |
| | | | TATTTGAAAAATATTCTGTGAACGCAAGTATTTCTATTGATGGCCC |
| | | | GAAACATATTAATGACAGGCATCGTTTAGATACCAAAGGGCGTAG |
| | | | CACTTACGAGGCGACAGTGCGTGGTTTGCGTATACTCCAACATGC |
| | | | TCATAAGCAAGGCCGTATTCCATCGGCACCGGGGGTTTTATGTGT |
| | | | CGCGAATGCTCAAGCAAATGGTGCTGAGATATATCGTCATTTTGT |
| | | | GGACGAATTAAAGGTTTATGGTTTTGATTTTCTGGTGCCAGACGA |
| | | | TTGTTATCATGACACTAATATTGACCCTGTTGGTATTAGCCGCTTC |
| | | | CTAAATGAAGCTTTGGATGAATGGTTCAAGGACAGCAACCCTAAT |
| | | | ATTTTTGTCCGCCTTTTTCAAACACACTTAGCTCATTTGCTCGGTA |
| | | | CAAAGCATCAAGGAATTTTAGGGCATTCACCCAGCGCCACTGGG |
| | | | GCATACGCATTCACCGTGGGTTCAGATGGTTTTATTCGTGTGGAT |
| | | | GATACCTTACGCGCCACATCAGACAGAATTTTCAATCCCATTGGT |
| | | | CATGTTTCTGAAATCAGCCTAACTGATGCACTTAATAGCCCTCAGT |
| | | | TCCAGGAGTACGCGTCAGTCGGCCAAGCTCTGCCCCATGAATGC |
| | | | AACGGTTGCATTTGGGAAAACGTCTGTGCTGGAGGTCGTATTATG |
| | | | AATCGTTTTTCACCTGAAACCCGCTTCGACCGCAAGTCTGTTTATT |
| | | | GCTATTCCATGAGAAGTTTCCTCAGCCGCGCCGCTGCACACCTAC |
| | | | TCAATATGGGCATCAAGGAAGAGCGCATTATGACAGCAATTGGG |
| | | | CGATAA |
| | | | (SEQ ID 206) |
| xncA_{L}-ykcA_{C} | PET-28a(+) | NdeI_XhoI | AGCAAATTACAGCGTGAAATTGCAGCAAACAAAGCTCAACTGAGC |
| | | | CATGAAGACAAGAAGAAAACGCAGCACAAAGAGCTTGTTGACAG |
| | | | CCTGC |
| | | | 42 |
| | | | TGGATACTGTCTCTGGTGGTTGGGTTAACGCTTTCGTTAACTGGC |
| | | | CGAAATCTTTCTAA |
| | | | (SEQ ID 207) |
| XnCA_{L}-xecA_{C} | PET- | NdeI_XhoI | AGCAAATTACAGCGTGAAATTGCAGCAAACAAAGCTCAACTGAGC |
| | 28a(+) | | CATGAAGACAAGAAGAAAACGCAGCACAAAGAGCTTGTTGACAG |
| | | | CCTGCTGGATACTGTCTCTGGTGGTTGGGTTAACGCTTTCGCTAA |
| | | | CTGGTCTAAATCTTTCTAA |
| | | | (SEQ ID 208) |
| xnCA_{L}-socA_{C} | PET- | NdeI_XhoI | AGCAAATTACAGCGTGAAATTGCAGCAAACAAAGCTCAACTGAGC |
| | 28a(+) | | CATGAAGACAAGAAGAAAACGCAGCACAAAGAGCTTGTTGACAG |
| | | | CCTGCTGGATACTGTCTCTGGTGGTTGGGTTAACGCTTTCGCTCG |
| | | | TTGGGACAAAAAATTCTAA |
| | | | (SEQ ID 209) |
| xncA_{L}-phcA_{C} | pET- | NdeI_XhoI | AGCAAATTACAGCGTGAAATTGCAGCAAACAAAGCTCAACTGAGC |
| | 28a(+) | | CATGAAGACAAGAAGAAAACGCAGCACAAAGAGCTTGTTGACAG |
| | | | CCTGCTGGATACTGTCTCTGGTGGTTGGGTTAACGCTTTCGCTAA |
| | | | CTGGACCAAACGTTTCTAA |
| | | | (SEQ ID 210) |
| xncA_{L}-ajcA_{C} | pET- | NdeI_XhoI | AGCAAATTACAGCGTGAAATTGCAGCAAACAAAGCTCAACTGAGC |
| | 28a(+) | | CATGAAGACAAGAAGAAAACGCAGCACAAAGAGCTTGTTGACAG |
| | | | CCTGCTGGATACTGTCTCTGGTGGTTGGGTTAACGTTTTCGCTCG |
| | | | TTGGGACAAACAGATCTAA |
| | | | (SEQ ID 211) |
| xncA_{L}-vscA_{C} | pET- | NdeI_XhoI | AGCAAATTACAGCGTGAAATTGCAGCAAACAAAGCTCAACTGAGC |
| | 28a(+) | | CATGAAGACAAGAAGAAAACGCAGCACAAAGAGCTTGTTGACAG |
| | | | CCTGCTGGATACTGTCTCTGGTGGTTGGGTAAACGCCTTCGCACG |
| | | | CTTCACGAAGCGCTTCTGA |
| | | | (SEQ ID 212) |

^{a}Small letters indicate untranslated region.

In some embodiments, the nucleic acid molecules are introduced into the host cell via a pET28a(+) vector and/or pCDFduet-1 vector. In some embodiments, the nucleic acid molecules are introduced into the host cell via a pET28a(+) vector, pCDFduet-1 vector, pACYCDuet-1 vector, pETDuet-1 vector, pCOLADuet-1 vector, pRSFDuet-1 vector, pBAD vector, or a combination thereof.

In some embodiments, the host cell is E. coli NiCo21(DE3) cell. In some embodiments, the host cell is E. coli NiCo21(DE3), BL21(DE3), BL21-AI, BL21 Star™ (DE3) pLysS, Rosetta™ (DE3), or a combination thereof.

Through the method described above, the polypeptides obtained may be distinct from each other. These polypeptides are then tested for the desired properties. In this way, resources can be preserved as polypeptides having the same chemical structure is not tested.

The present invention also provides a method of producing a polypeptide, the method comprising:
a) expressing a precursor polypeptide and a rSAM/SPASM maturase;
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide to form a polypeptide with a cyclophane moiety connecting the X₁ and X₃ residues in each motif.

In some embodiments, the method further comprises contacting the polypeptide of step a) with a protease.

The present invention also provides a method of producing a polypeptide, the method comprising:
a) expressing a precursor polypeptide and a rSAM/SPASM maturase in order to form a modified precursor polypeptide; and
b) cleaving the modified precursor polypeptide from the rSAM/SPASM maturase using a protease to form a cleaved modified polypeptide;
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide to form a modified precursor polypeptide with a cyclophane moiety connecting the X₁ and X₃ residues in each motif.

This allows the method to be more versatile as a commercial protease can be used to cleave the modified precursor polypeptide in vitro.

In some embodiments, the protease is derived from Xenorhabdus Spp. In some embodiments, only the protease is derived from Xenorhabdus Spp.

In some embodiments, at least one motif comprises X₁ and X₃ connected via phenylene to form a cyclophane moiety. In some embodiments, at least one motif comprises X₁ and X₃ connected via indolylene to form a cyclophane moiety. In some embodiments, the two motifs separately comprises phenylene and indolylene. In some embodiments, the X₁ and X₃ in the second motif are connected via phenylene to form a cyclophane moiety.

The present invention also provides a method of synthesising a polypeptide as disclosed herein, the method comprising:
(a) coupling a pre-sequence peptide to a support, wherein said pre-sequence peptide comprises amino acid residues having side chain functionalities which are, if necessary, protected during the synthesis;
(b) coupling one or more N-protected amino acids to the N-terminus of the pre-sequence peptide to form a precursor polypeptide, wherein each coupling is performed in stepwise fashion and under conditions in which each of the amino acids of the target peptide is coupled and subsequently N-deprotected;
c) cleaving said precursor polypeptide from the support; and
d) synthetically or enzymatically connecting the X₁ and X₃ in each motif to form a cyclophane moiety.

The step of d) connecting the X₁ and X₃ in each motif to form a cyclophane moiety can occur before the cleaving step c). In this regard, the modification of the precursor polypeptide can occur on the support.

The step of d) may be performed synthetically. For example, the precursor peptide may comprise an alkyne moiety and an ortho-iodoaniline moiety. A Larock indole synthesis may be performed to form an indolyene containing cyclophane. Alternatively, the precursor peptide may comprise a halophenyl moiety such that a halo substitution may be performed to form a phenylene containing cyclophane.

The support may be a solid phase material or resin (for example, low cross-linked polystyrene beads) which may form a covalent bond between the carbonyl group and the resin, most often an amido or an ester bond. Alternatively, the synthetic method may be performed without the use of a support.

Accordingly, the method may comprise:
(a) synthesising a precursor polypeptide, the precursor polypeptide comprising a first three residue motif (from a N-terminus) and a second three residue motif, the first and second three residue motif optionally separated by 1 to 3 amino acid residue, and at least two C-terminus residues, wherein the three residue motif is each represented by X₁-X₂-X₃; and
b) synthetically or enzymatically connecting the X₁ and X₃ in each motif to form a cyclophane moiety.

The present invention also provides a method of modifying a precursor polypeptide, the precursor polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is an amino acid residue, the amino acid independently selected from tryptophan, phenylalanine, tyrosine, histidine, an unnatural aromatic amino acid or a derivative thereof;
wherein each X₂ and X₃ are independently any amino acid residue; and
wherein at least one of the two C-terminus residues is an aromatic residue; the method comprising:
enzymatically connecting the X₁ and X₃ residues in each motif to form a cyclophane moiety.

In some embodiments, at least one motif comprises X₁ and X₃ connected via phenylene to form a cyclophane moiety. In some embodiments, at least one motif comprises X₁ and X₃ connected via indolylene to form a cyclophane moiety. In some embodiments, the two motifs separately comprises phenylene and indolylene. In some embodiments, the X₁ and X₃ in the second motif are connected via phenylene to form a cyclophane moiety.

In some embodiments, the enzyme is rSAM/SPASM maturase.

The present invention also provides a composition comprising a polypeptide as disclosed herein.

In one embodiment, there is provided a pharmaceutical composition comprising a polypeptide as defined herein. The pharmaceutical composition may comprise a pharmaceutically acceptable carrier. By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like. Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The present invention also provides a use and/or method of treating a disease. In one embodiment, there is provided a method of treating a disease in a subject, comprising administering an effective amount of a polypeptide or composition as defined herein to the subject in need thereof. Provided herein is also a modified polypeptide or composition as defined herein for use in treating a disease. Also provided herein is the use of the modified polypeptide or composition in the manufacture of a medicament for the treatment in a subject. The disease may, for example, an infectious disease. The disease may be caused by a bacteria, or a bacterial infection.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender. The term “administering” refers to contacting, applying, injecting, transfusing or providing a composition of the present invention to a subject.

In some embodiments, the bacterial infection is caused by a Gram-negative bacteria. In other embodiments, the Gram-negative bacteria is selected from Escherichia coli, Pseudomonas aeruginosa, Candidatus Liberibacter, Agrobacterium tumefaciens, Acinetobactor baumannii, Moraxella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Actinobacillus actinomycetencomitans, Aggregatibacter actinomycetemcomitans, Porphyromonas gingivalis, Helicobacter pylori, Francisella tularensis, Yersinia pestis, Vibrio cholera, Morganella morganii, Edwardsiella tarda, Campylobacter jejuni, Haemophilus influenza, Enterobacter cloacae, or a combination thereof.

Examples of polypeptides and their MIC values are shown in Table 3.

The present disclosure also concerns a method of killing and/or inhibiting proliferation of bacteria, comprising contacting the bacteria with an effective amount of a polypeptide as disclosed herein.

The present disclosure also concerns a method of disinfecting a surface, comprising contacting the surface with an effective amount of a polypeptide as disclosed herein.

The surface may be a medical device or implant.

In the embodiments that follows, the invention is described in relation to some conditions for consistency to showcase the present invention. However, the skilled person would understand that the invention is not limited to such.

### Example 1: Methodology

A three-step approach for antibiotic discovery was envisioned. In step 1, genomic enzymology is used to identify and assign function to proteins that define a natural product family. In step 2, the natural products are produced using synthetic biology—BGCs are synthesized and expressed in a heterologous host producing the natural products. In step 3, the products are tested for bioactivities against a panel of pathogenic bacteria. Historically, typical bioactivity-guided platforms utilize crude or partially purified extracts, which leads to identification of only the most potent natural products while the minor components or those with less potent activities are overlooked.

This workflow is problematic, leads to rediscovery of known compounds, and led pharmaceutical companies to abandon natural product drug discovery programs in the 1980s and 1990s. In the present strategy, chemistry is prioritized so that only molecules which have not been characterized or tested for bioactivity are obtained. This approach yields that targeted compound directly and subsequent MIC values can be obtained for each molecule produced. This workflow solves the problems associated with isolation of known compounds, laborious de-replication, bioactive but minor constituents, and cryptic metabolites.

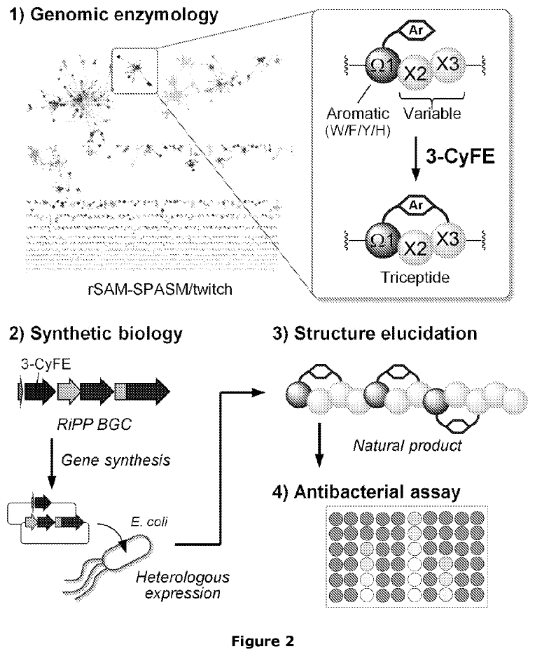

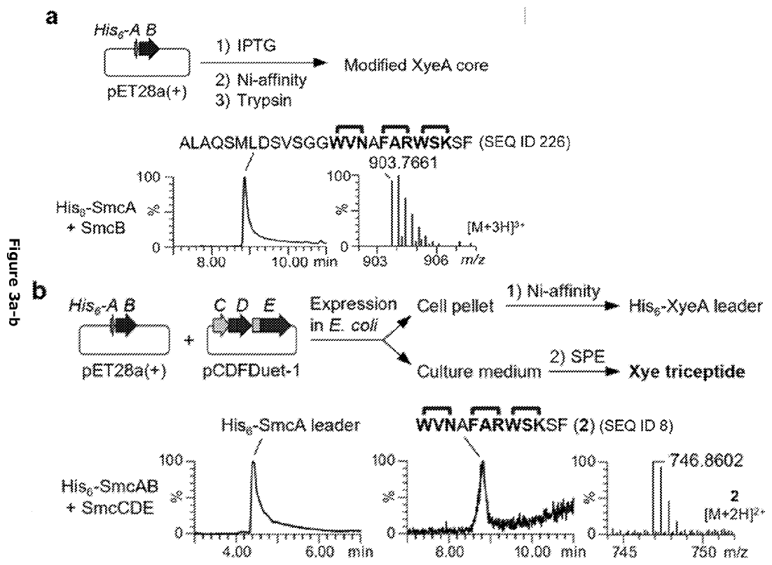

For example, a chemically-guided workflow is disclosed herein to reveal antibiotic activity for Series A xenorceptides, which are named xenorceptides A1-A10. Fundamentally, this workflow starts from a posttranslational modifying enzyme sequence and ends with a peptide antibiotic (FIG. 2). This workflow is demonstrated on triceptides, a relatively new RiPP family with no known bioactivity. In particular, the chemically-guided workflow, named GEnSyBER-A herein, can be used to discover ribosomally synthesized and posttranslationally modified peptide (RiPP) antibiotics. This approach starts from radical SAM enzyme sequence-function space enriched in 3-residue cyclophane forming enzymes. Synthetic biology enabled the production of xenorceptides A1-A10, RiPP natural products associated with the Xye maturase system. Xenorceptides are 12-mer triceptides that contain three separate three-residue cyclophanes. Xenorceptide A2 was found to selectively kill several carbapenamase-resistant Enterobacteriaceae (CRE) with MIC values between 4-8 μg/ml. This workflow can provide unique peptide antibiotics with activities against priority pathogens of interest.

### Example 2: Xye Maturase System (ABCDE)

For example, the Xye maturase systems encode a precursor (XyeA), rSAM/SPASM maturase (XyeB), protease (XyeC), transporter (XyeD), and protease/transporter (XyeE) (FIG. 1a). Bioinformatic analysis revealed 81 XyeA precursors with 56 encoding unique core sequences. The latter represents the total number of different xenorceptides that could be produced. The core peptides contain two or three Ωxx motifs (Ω=Trp, Phe or Tyr) downstream of the conserved GG motif and are classified into 4 types (FIG. 1b). Type A is the most prevalent and all Q residues in the conserved ΩxxxΩxxΩxx sequence are involved in the 3-residue cyclophanes. Xenorceptide A1 (1) is a representative of Type 1. Although antibacterial activity was not detected for 1, it is hypothesized that the diversity in bacterial sources and core sequences within XyeA precursors had the potential to generate peptide antibiotics.

The Xye nucleic acid sequence is encoded by a 5-gene cassette containing precursor (XyeA), radical SAM enzyme (XyeB), protease (XyeC), transporter (XyeD), and fused protease transporter (XyeE). The radical SAM enzyme (XyeB) introduces the 3 rings and the protease-transporter (XyeE) cleaves the modified precursor. All genetic components to produce the antibiotic have been identified and functionally validated (substrate, enzymes, protease, and transporter). This opens up opportunities for applying these enzymes to modify non-cognate core peptide sequences, hence their relative flexibility in antibiotic discovery. This allows for a more efficient way of producing the natural products. The polypeptides are also stable to heat, proteolytic degradation, and low pH. The polypeptides may also be effective against Gram-negative bacteria, including clinical strains which are resistant to last-line antibiotics. Only a limited number of antibiotics have been approved that selectively target Gram-negative bacteria.

In contrast, Darobactin, which is the most comparable antibiotic is produced from by the dar gene cluster, contains 5 genes (precursor, radical SAM enzyme, and 3× transporters). The radical SAM enzyme (DarE) is responsible for the 2-rings in the natural product. The protease responsible for cleavage has not been identified. To obtain the darobactin, an undefined protease in E. coli is used.

### Example 3: xncAB and xncCDE

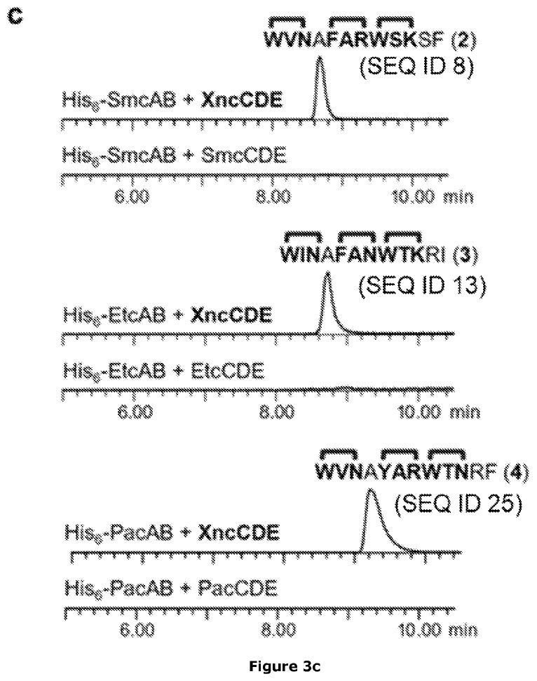

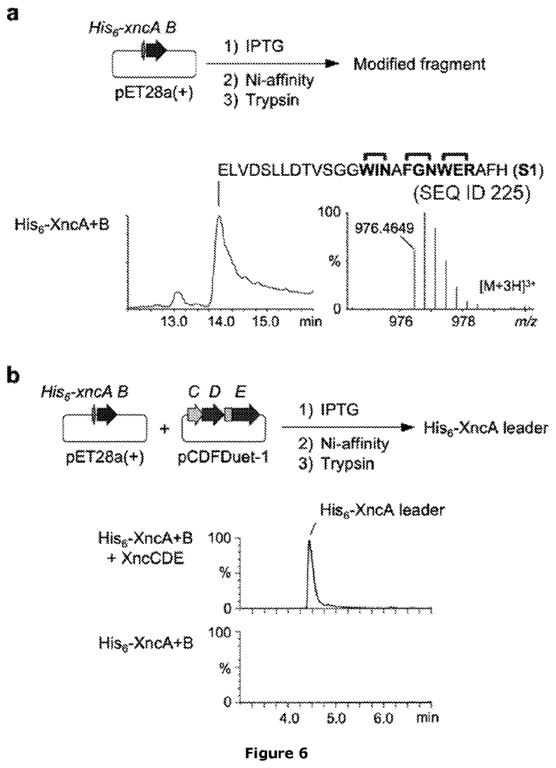

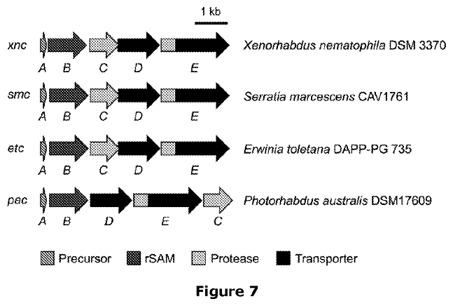

For the production of xenorceptides, it was first established that 1 can be produced in E. coli by expressing the xnc BGC split into two vectors: His₆-xncAB in pET28a(+) and xncCDE in pCDFDuet-1. The xncA gene was expressed with as an N-terminal His x 6 tag (His₆) so that the precursor could be purified, and the modifications detected (FIG. 6). This two-vector system allows testing of His₆-xyeAB expressions first to ensure maturation by the rSAM/SPASM enzyme then xyeCDE in a second vector can be expressed in a subsequent expression to facilitate cleavage and export (FIGS. 3a and 3b). 3 BGCs named smc, etc, and pac from Serratia marcescens, Erwinia toletana, and Photorhabdus australis, respectively, were selected for heterologous expression (FIG. 7).

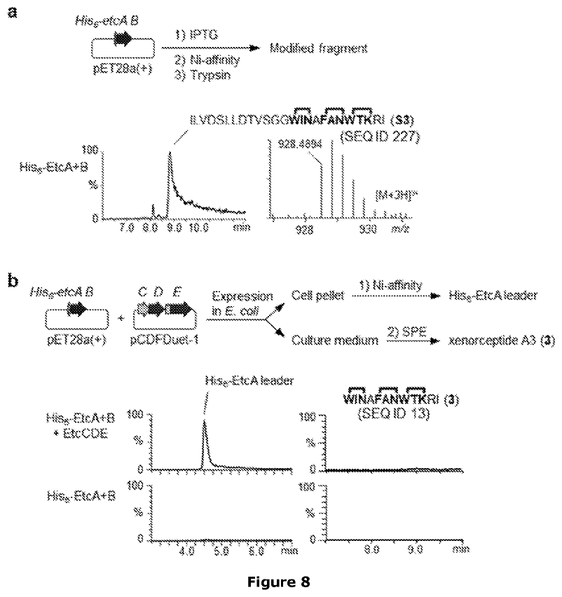

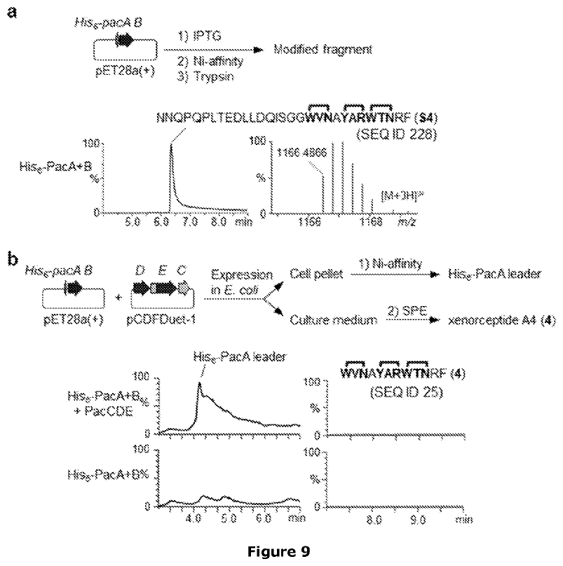

To initiate heterologous expression, native AB constructs were synthesized and inserted into pET28a vector. The three constructs containing His₆-AB were expressed in E. coli NiCo2l(DE3) cells. The precursors were purified by Ni-affinity chromatography, digested with trypsin and subjected to LC-MS. As demonstrated in FIG. 3a, the digest obtained from the His₆-SmcAB construct included a triply-charged fragment at m/z 903.7661, corresponding to −6 Da mass loss from the C-terminal region of SmcA (ALAQSMLDSVSGGWVNAFAR-WSKSF, m/z 905.7831 [M+3H]³⁺). Expressions of His₆-EtcAB and His₆-PacAB constructs also resulted in detecting similar modified fragments (FIGS. 8 and 9). These experiments showed efficient modification by rSAM enzymes in E. coli and we proceeded with full cluster expression.

The remaining genes (CDE) for each cluster were synthesized and inserted into pCDFduet-1. Native His₆-XyeAB constructs were co-expressed with native XyeCDE constructs in E. coli. Both the cell biomass and the medium were analyzed separately by two methods. First, the cell pellet was processed as above to detect whether the precursor peptide was cleaved. Purified His₆-PacA, His₆-SmcA, and His₆-EtcA were detected as truncated leaders losing C-terminal residues after the GG motif, implying the protease is functioning (FIGS. 3b, 8, and 9). Second, the products were extracted from the culture medium using solid-phase extraction. The desired end products from smc, etc, and pac clusters were either undetectable or detectable in trace amounts. This result suggested D or E transporters are not functioning efficiently for native His₆-AB+CDE expressions (FIGS. 3b, 8, and 9). To increase the yields of end products, nonnative combinations of His₆-AB+CDE were tested. As shown in FIG. 3c, Smc, Etc, and Pac products (2-4) could be efficiently produced using combinations of native His₆-XyeAB+XncCDE at a yield of 1.0-4.6 mg per liter. Tandem mass spectrometry (MSMS) analysis of these products confirmed the primary amino acid sequence and localized −2 Da losses to each of the three Ω1-X₂-X₃ motifs.

### Example 4: Characterisation

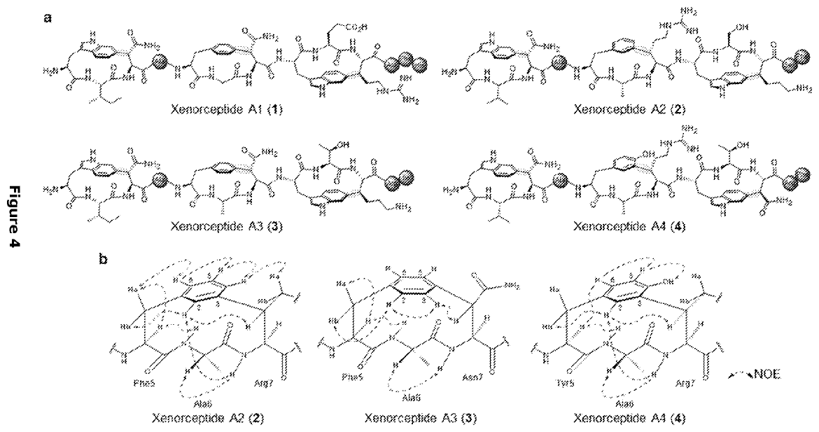

The structures of products 2-4 were characterized by NMR to understand whether the XyeB maturases from different Genera catalyze cyclophane formation with identical substitution pattern and the planar chirality with respect to the indole. Products 2-4 were characterized analogous to xenorceptide A1 reported previously. In all cases, the XyeB maturases carry out the same crosslinking of Trp as in 1 (FIG. 4a). The Phe residue in 3 was assigned as para-substituted analogous to 1 (FIG. 4b). However, 2 was elucidated as meta-substituted based on 2D NMR. Phe5-H2 (δ 6.91 ppm) appears as a singlet and has NOESY correlations with both Phe5-Hβb (δ 2.73 ppm) and Arg7-Hβ (δ 2.87 ppm). The remaining three aromatic protons within the same spin system (H4, δ 7.17 ppm; H5, δ 7.25 ppm; H6, δ 7.09 ppm) exhibit NOESY correlations with Phe5-Hβa (δ 2.96 ppm) and Arg7-Hγ (δ 2.10, δ 1.94 ppm), suggesting these protons lie on the same face and the new C(sp²)-C(sp³) bond is formed between Phe5-C3 with Arg7-Cβ (FIG. 4b). The Pac product (4) encodes a Tyr5 instead of Phe5, and the Tyr is crosslinked at C3 of Tyr (FIG. 4b). This substitution pattern has been observed by triceptide maturases reported previously. The relative conformations of the cyclophane rings were assigned by NOESY and coupling constant analysis, which showed the orientation of the indole in the Trp-derived cyclophanes are identical for 1-4. The absolute configuration of X2 residues were assigned by advanced Marfey's method in addition to guanidine isothiocyanate derivatization. These analyses led to all α-positions to be of the natural L-configuration and the remaining amino acids to be as shown. The planar chirality of the Trp was assigned as Sp. The Smc, Etc, and Pac products were named xenorceptide A2 (2), xenorceptide A3 (3), and xenorceptide A4 (4), respectively (FIG. 4).

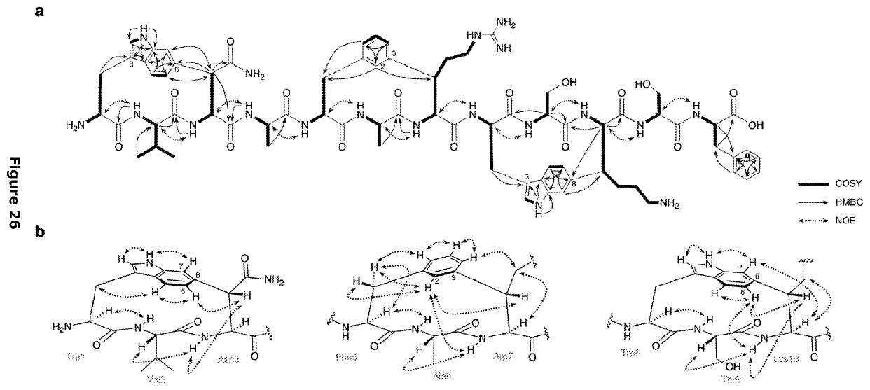

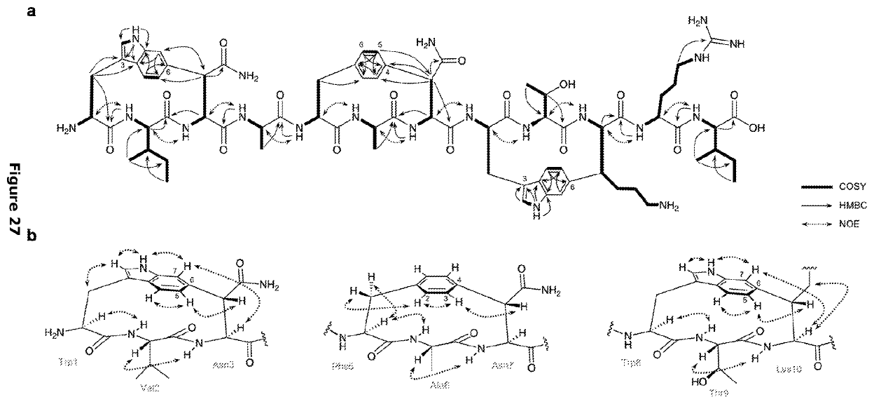

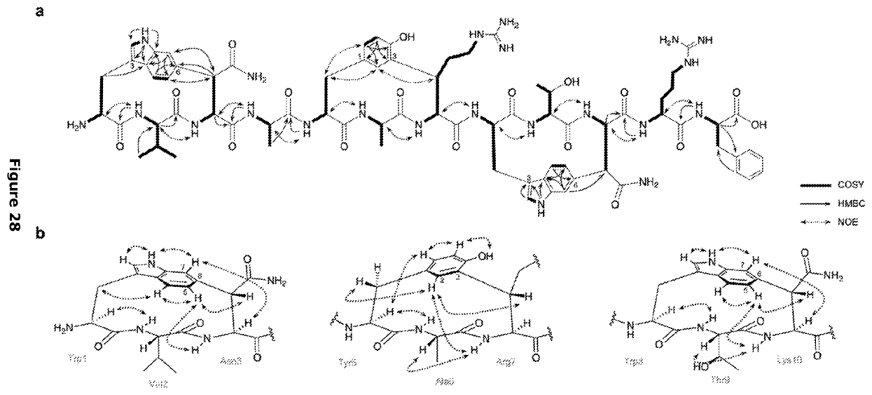

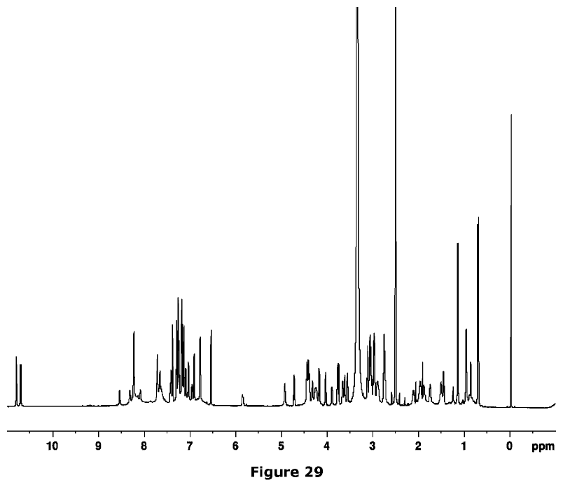

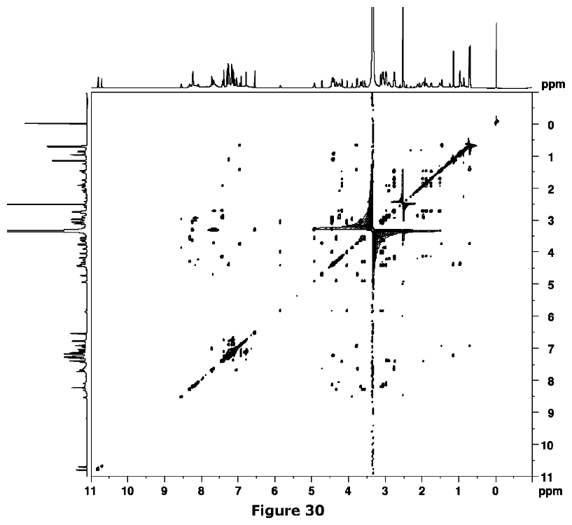

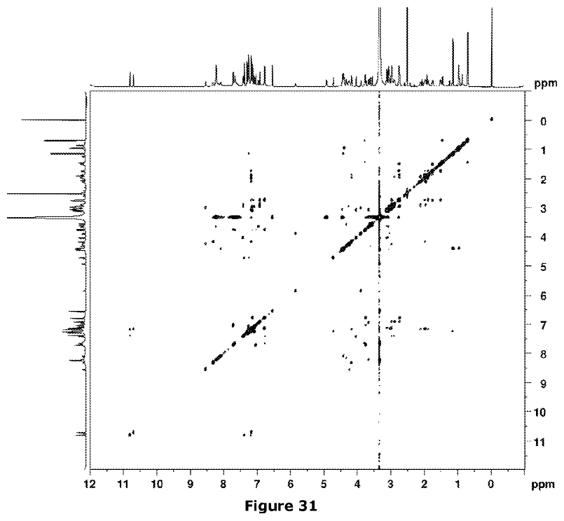

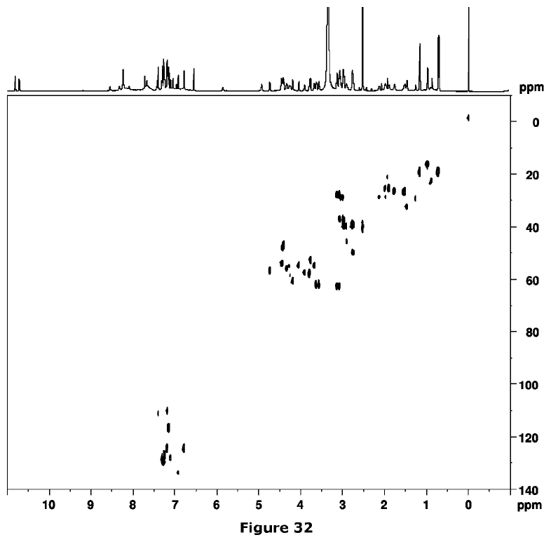

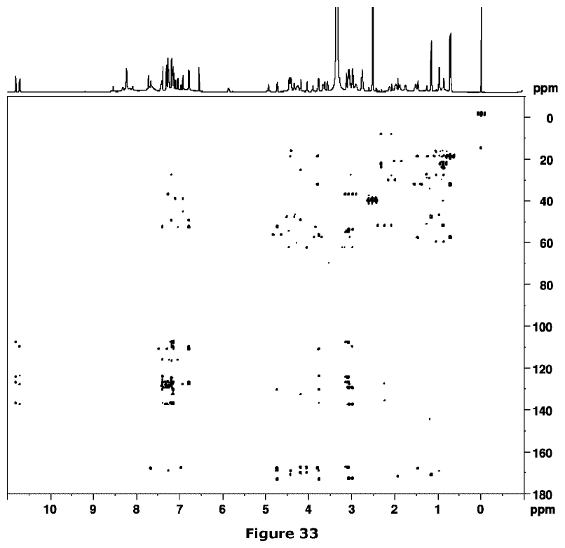

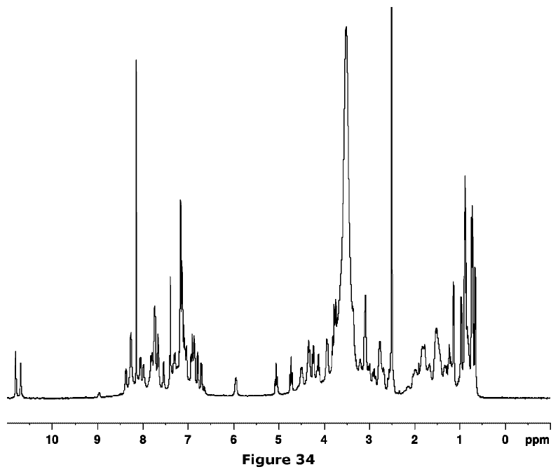

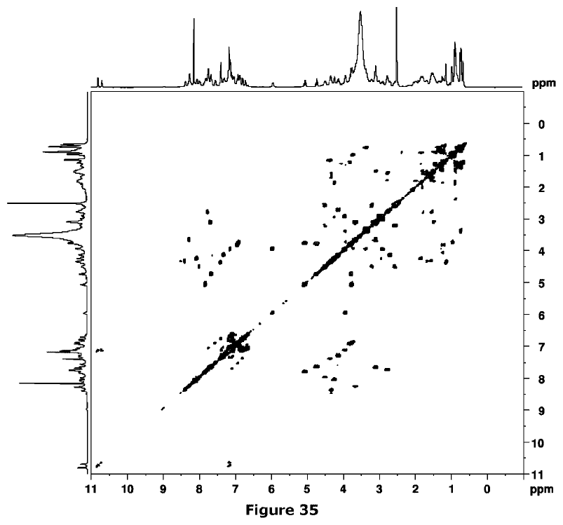

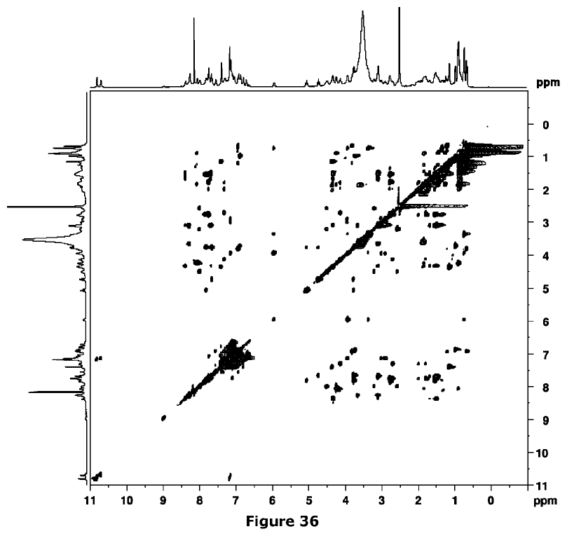

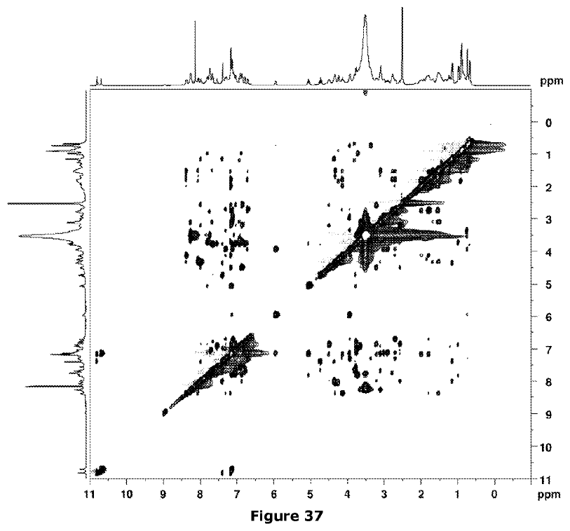

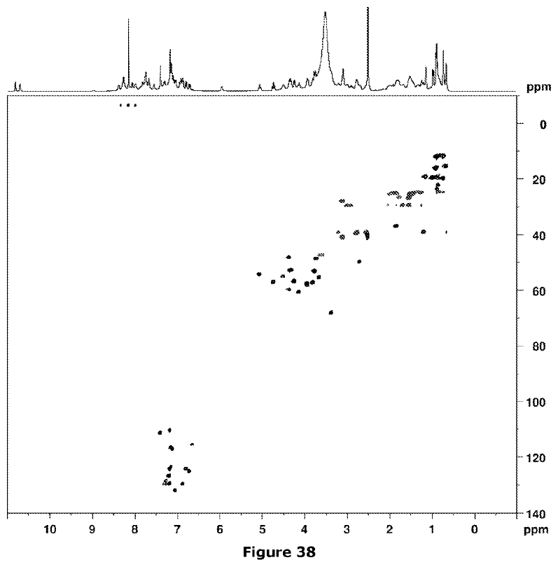

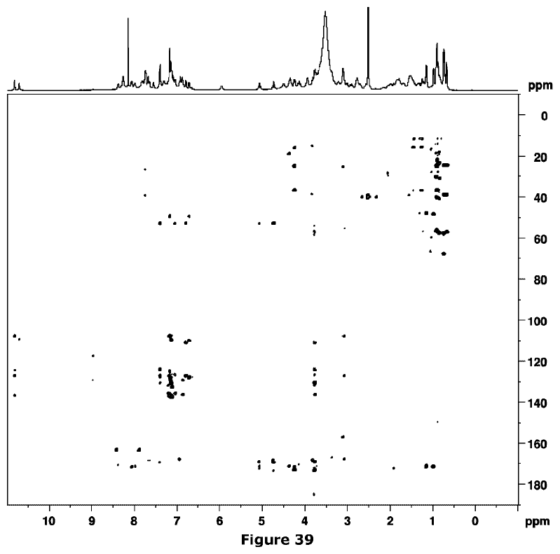

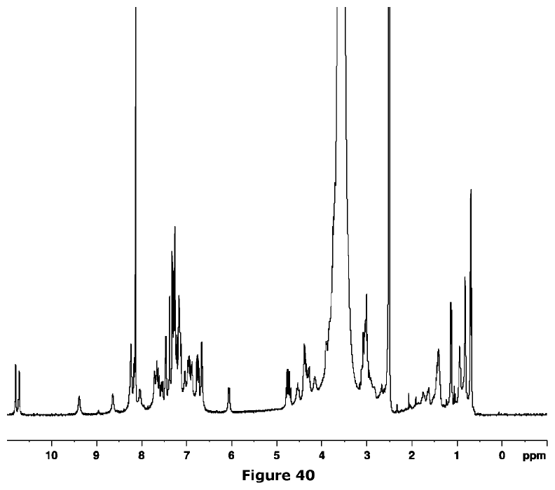

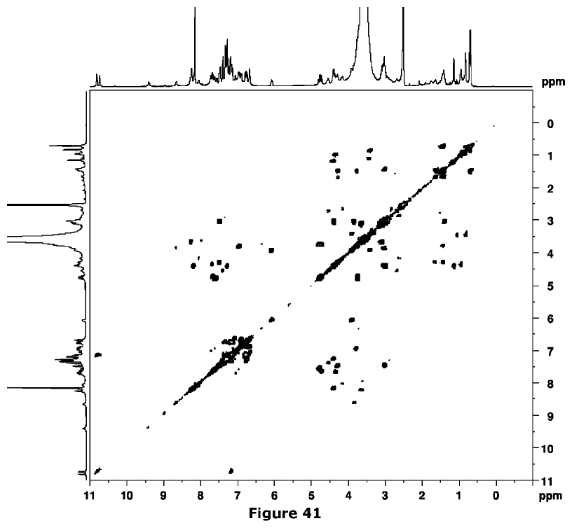

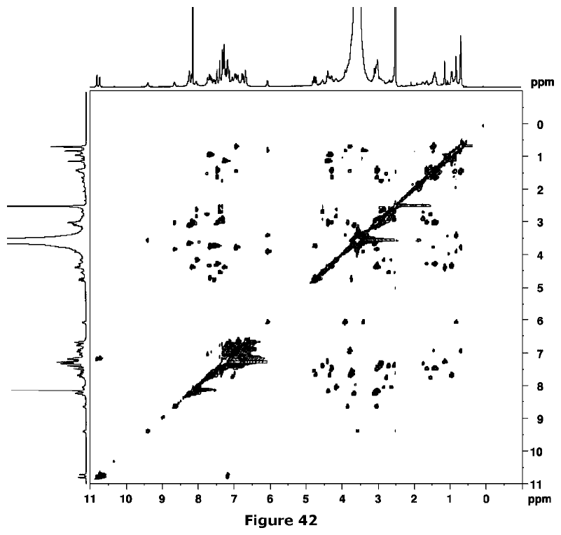

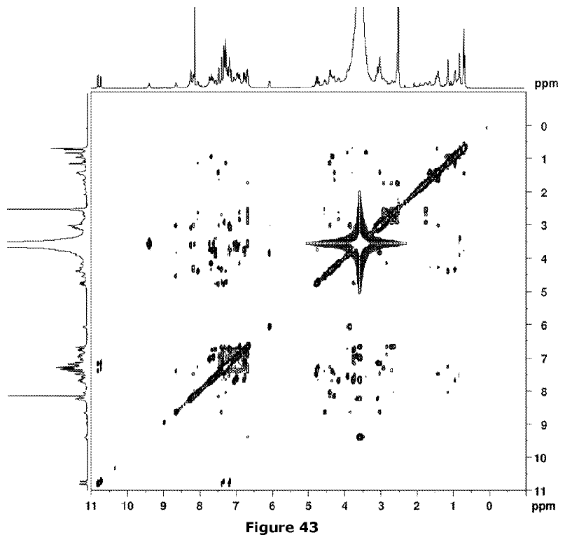

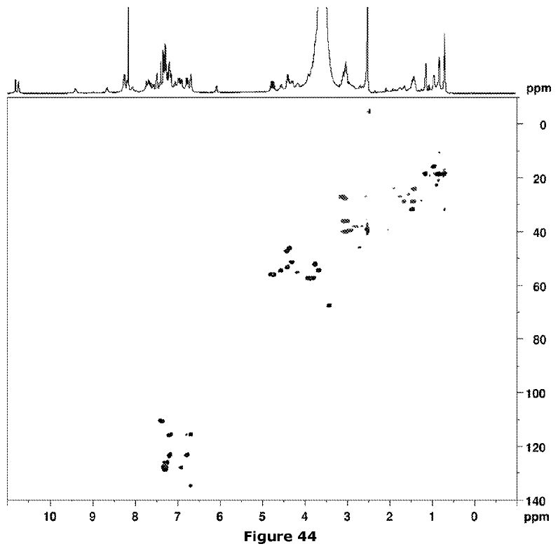

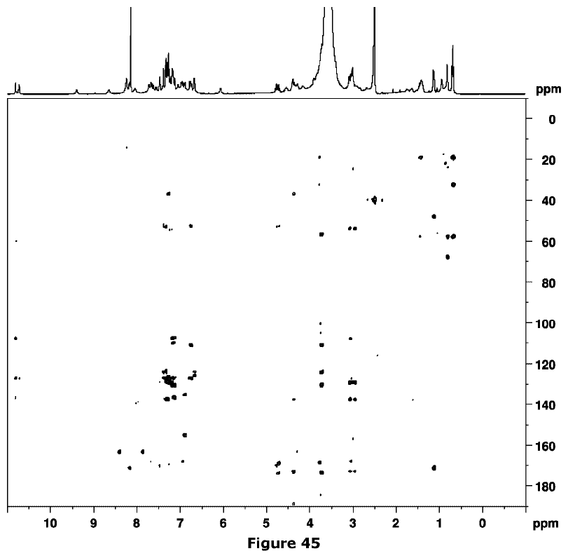

Structural eludication of xenorceptide A2 (2), xenorceptide A3 (3) and xenorceptide A4 (4) are shown in FIG. 26-28. FIG. 29-45 shows the NMR spectra used to derive the xenorceptide structures. Table 18-20 shows the summarised NMR data for these xenorceptides.

### Example 5: Antibacterial Activity

The four xenorceptides (1-4) along with unmodified sequences were screened for antibacterial activity. Minimal inhibitory concentrations (MICs) were obtained for 1-4 using microbroth dilution assays against Gram-positive and Gram-negative bacteria (Table 10). 2-4 showed selective activity against Gram-negative pathogens, E. coli ATCC 25922 and K. pneumoniae ATCC 700603 (Table 10). No activity was observed against Gram-positive bacteria (B. subtilis ATCC 6633 and S. aureus ATCC 29737) for any of the products tested. Encouraged by the activity of xenorceptide A2 (2) further testing was carried out on a broader panel including multi-drug resistant pathogens.

TABLE 9
MIC values (μg/mL) of xenorceptide
A2 (2) against Enterobacteriaceae.

| | | Xenorceptide |
|---|---|---|
| Species | Strain^{a} | A2 (2) |
| Escherichia coli | M6 | 8 |
| | M10 | 4 |
| | M11 | 4 |
| | CRE1006 | 4 |
| | ATCC 25922 | 4 |
| Klebsiella pneumoniae | CRE 1007 | 8 |
| | CRE1008 | 8 |
| | CRE1011 | 8 |
| | CRE1012 | 8 |
| | ATCC 700603 | 8 |
| Enterobacter cloacae | CRE1010 | 4 |
| | CRE1014 | 16 |
| | CRE1015 | 32 |
| | CRE1016 | 16 |
| | CRE1017 | 32 |
| Salmonella typhimurium | ATCC 14028 | 8 |
| Salmonella entereditis | ATCC 13076 | 8 |
| Shigella flexneri | M90T | 2 |

^{a}CRE strains are carbapenem-resistant clinical isolates. M6, M10, and M11 strains are carbapenem- and colistin-resistant clinical isolates.

TABLE 10
Antimicrobial activity of 1-4.

| | MIC (μg/mL) | | | | |
|---|---|---|---|---|---|
| | Xenorceptide | xenorceptide | xenorceptide | xenorceptide | xenorceptide |
| Strain | A1 (1) | A2 (2) | A3 (3) | A4 (4) | A8 (8) |

Gram-negative bacteria

| | | | | | |
|---|---|---|---|---|---|
| Escherichia coli | 64 | 4 | 8 | 8 | 2 |
| ATCC 25922 | | | | | |
| Klebsiella | 64 | 8 | 8 | 16 | 4 |
| pneumoniae | | | | | |
| ATC 700603 | | | | | |
| Morganella | >64 | 32 | 64 | 64 | — |
| morganii | | | | | |
| ATCC 25830 | | | | | |
| Pseudomonas | >64 | 64 | 64 | >64 | 64 |
| aeruginosa | | | | | |
| ATCC 9027 | | | | | |
| Acinetobacter | >64 | >64 | >64 | >64 | >64 |
| baumanii | | | | | |
| ATCC 19606 | | | | | |

Gram-positive bacteria

| | | | | | |
|---|---|---|---|---|---|
| Bacillus subtilis | >64 | >64 | >64 | >64 | — |
| ATCC 6633 | | | | | |
| Staphylococcus | >64 | >64 | >64 | >64 | >64 |
| aureus | | | | | |
| ATCC 29737 | | | | | |

TABLE 11
MIC value of xenorceptide A2 (2) against bacterial pathogens.

| | | MIC | | | MIC |
|---|---|---|---|---|---|
| Species | Strain | (μg/ml) | Species | Strain | (μg/mL) |
| Gram-negative bacteria | | | Gram-negative bacteria | | |
| (Enterobacteriaceae) | | | (Other families) | | |
| Escherichia coli | M6 | 8 | Acinetobacter | ACBA1001 | 32 |
| | M10 | 4 | baumannii | ACBA1002 | 32 |
| | M11 | 4 | | ACBA1003 | 32 |
| | CRE1006 | 4 | | ACBA1004 | 64 |
| | ATCC 25922 | 4 | | ATCC 19606 | >64 |
| Klebsiella | CRE 1007 | 8 | Pseudomonas | DR4877/07 | 64 |
| pneumoniae | CRE1008 | 8 | aeruginosa | DR5790/07 | 64 |
| | CRE1011 | 8 | | DM4150R | 64 |
| | CRE1012 | 8 | | DM23376 | >64 |
| | ATCC 700603 | 8 | | ATCC 9027 | 64 |
| Enterobacter | CRE1010 | 4 | Morganella | CRE1001 | 32 |
| cloacae | CRE1014 | 16 | morganii | ATCC 25830 | 32 |
| | CRE1015 | 32 | Gram-positive bacteria | | |
| | CRE1016 | 16 | Staphylococcus | ATCC 29737 | >64 |
| | CRE1017 | 32 | aureus | ATCC 43300 | >64 |
| Salmonella | ATCC 14028 | 8 | Bacillus cereus | ATCC 11778 | >64 |
| typhimurium | | | Bacillus subtilis | ATCC 6633 | >64 |
| Salmonella | ATCC 13076 | 8 | | | |
| entereditis | | | | | |
| Shigella flexneri | M90T | 2 | | | |

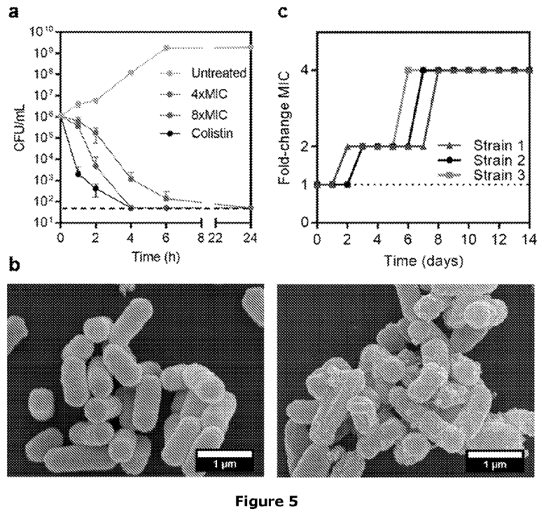

Xenorceptide A2 (2) was tested against a larger panel of drug-resistant clinical isolates. These results are summarized in Table 9 and confirm the selective activity against Gram-negative Enterobacteriaceae, several of which are carbapenem-resistant Enterobacteriaceae (CRE) pathogens. Next, time-kill assays against the colistin-resistant strain E. coli M6 was carried out which showed that xenorceptide A2 (2) has a bactericidal effect over 24 h at both 4× and 8×MIC, causing 3-log reduction in bacteria count (FIG. 5a). To further understand the killing effect of xenorceptide A2 (2), we imaged the morphology of E. coli M6 in the presence of xenorceptide A2 (2) by scanning electron microscopy (FIG. 5b). These images show significant disruption of the bacteria membranes within 2 h of treatment, followed by cell lysis and death (FIG. 5b). Xenorceptide A2 (2) did not exhibit any cytotoxicity against HepG2 human cells up to a concentration of 256 μg/ml. Finally, we incubated xenorceptide A2 (2) at sub-inhibitory concentrations with E. coli M6 to test if resistance developed. Over the course of two weeks, we obtained strains that were ˜4-fold resistant to xenorceptide A2 (2) with an MIC of 32 μg/ml (FIG. 5c).

### Example 6: Discussion

Natural products have been the main source of currently used antibiotics but no new classes of antibiotics have been introduced since the 1980s. Over the last few decades, bioactivity-guided isolation discovery has suffered from rediscovery of known compounds. The fundamental difference between the present invention and bioactivity-guided isolation is the former prioritizes chemistry while the latter prioritizes the bioactivity. In the present invention, only unknown molecules are screened, and MIC values are obtained directly. To the best of the inventors' knowledge, a natural product of a new chemotype able to selectively kill CRE pathogens has not been identified using a chemically-guided approach.

Using bioactivity-guided approaches, promising antibiotics against Gram-negative pathogens have been isolated from the entomopathogenic bacteria, Xenorhabdus and Photorhabdus. Odilorhabdins are broad spectrum peptide antibiotics that bind to a new ribosome site. Previous work has identified darobactin from strains of Photorhabdus by testing of concentrated extracts (20×). Recently, this concept was developed further to assay HPLC fractions of Xenorhabdus and Photorhabdus extracts representing a 200× fold increase in concentrations, which led to the antibiotic, 3′-amino-3′-deoxyguanosine, a pro-drug with selective activity against E. coli.

Structural similarities and differences are apparent in xenorceptide A2 and darobactin. The C-terminal pentapeptide of both share an identical Trp-derived cyclophane appended to Ser-Phe. Differences are in the N-terminus. Xenorceptide A2 has two three-residue cyclophanes separated by an Ala residue. Darobactin contains a second ether crosslinked cyclophane that is fused to a central Trp residue. Darobactin has broad spectrum activity against Gram-negative pathogens and the mechanism of action was shown to bind to the bacterial insertase BamA20, an essential outer membrane protein in Gram-negative bacteria. Significantly, it is shown that xenorceptide A2 composed of non-fused three-residue cyclophanes has activity against specific Gram-negative bacteria. While the mechanism of action for xenorceptide A2 remains to be elucidated, the N-terminal cyclophanes appear to confer a greater selectivity for Enterobacteriaceae vs other bacteria.

In conclusion, GEnSyBER-A as an end to end workflow for the discovery of RiPP antibiotics is presented. This work-flow was applied to identify Xenorceptide A2 from radical SAM sequence function space. Xenorceptide A2 has promising activity against priority pathogens for which antibiotics are urgently needed. The strains of Serratia from which xenorceptide A2 is encoded are clinical isolates which may represent important and understudied sources for antibiotics.

### Example 7: Bioinformatic Mapping of Xye BGCs

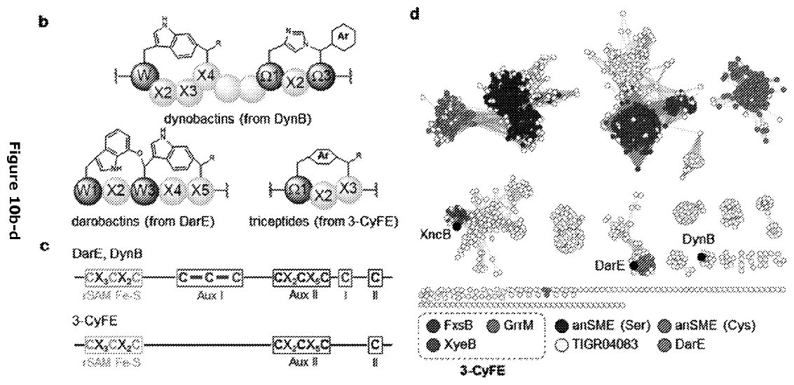

The Xye maturase systems encode a precursor (XyeA), rSAM/SPASM maturase (XyeB), protease (XyeC), transporter (XyeD), and protease/transporter (XyeE). The XyeA precursors are ˜55 AA in length with the core sequences being typically 13-16 residues. Core peptides contain a ΩxxxΩxxΩxx motif (Ω1=Trp, Phe or Tyr) where all Q residues are involved in a 3-residue cyclophane. The Gly-Gly motif XyeA indicates the end of the leader sequence. In our bioinformatic analysis, we identified 81 XyeA precursors with 37 encoding unique core sequences (Table 3; Type A). The latter represents the total number of different xenorceptides that could be produced. In addition to the canonical type described above, three additional core types are readily identified based on homology to rSAM/SPASM XyeB maturases in the RefSeq database. The second, third, and fourth types contain ΩxxΩxx (Type B, n=2 unique core sequences), ΩxxxΩxx (Type C, n=1 unique core sequence), and ΩxxxxΩxx (Type D, n=16 unique core sequences) motifs, respectively. We suggest that precursor types B-D are classified under xenorceptides (Table 3) because all precursors contain the Gly-Gly motif, BGCs typically conserve the characteristic five genes (xyeABCDE), and several maturases are identified by the cut-off defined for annotating XyeB radical SAM/SPASM proteins (TIGR04496) (FIG. 10d). We predict that maturases from types B-D will also catalyze formation of triceptide macrocycles. The main source bacteria belong to the order Enterobacterales and a phylogentic tree based on the gene sequences for xyeB from Type A precursors was constructed (FIG. 11a). The 5 predominant genera that encode xye BGCs are Erwinia, Xenorhabdus, Serratia, Yersinia, and Photorhabdus. The source microbiomes of the bacteria are plants, nematode, and animals. Representative BGCs and core sequences from different genera are shown in FIG. 11b. With bioinformatic mapping of the Xye maturase system complete, we proceeded to produce selected xenorceptides using synthetic biology.

### Example 8: Heterologous Expression of Xenorceptides in E. coli

For production of xenorceptides, we used two different expression systems that allowed systematic production of xenorceptides from different bacterial genera. We first established that 1 can be produced in E. coli by expressing the xnc BGC split into two vectors: His6-xncAB in pET28a(+) and xncCDE in pCDFDuet-1. The xncA gene was expressed with as an N-terminal His×6 tag (His6) so that the precursor could be purified, and the modifications were detected (FIG. 6). This two-vector system allows testing of His6-xyeAB expressions first to ensure maturation by the rSAM/SPASM enzyme then xyeCDE in a second vector can be expressed in a subsequent expression to facilitate cleavage and export (FIGS. 3a and 3b).

To initiate heterologous expression, native AB constructs were synthesized and inserted into pET28a(+) vector (Table 8). The three constructs containing His6-A+B were coexpressed in E. coli NiCo21(DE3) cells. The precursors were purified by Ni-affinity chromatography, digested with trypsin and subjected to LC-MS. As demonstrated in FIG. 3a, the digest obtained from the smcAB construct included a double-charged fragment at m/z 1389.6797, corresponding to −6 Da mass loss from the C-terminal region of SmcA (ELVDSLLDTVSGGWVNAFARWSKSF (SEQ ID 235), m/z 1392.7032 [M+2H]²⁺). Expressions of etcAB and pacAB constructs also resulted in detecting similar modified fragments. These experiments showed efficient modification by rSAM enzymes in E. coli and we proceeded with full cluster expression.

The remaining genes (CDE) for each cluster were synthesized and inserted into pCDFduet-1. Native His6-A+B constructs were coexpressed with native XyeCDE constructs in E. coli Nico21(DE3). Both the cell biomass and the medium were analyzed separately by two methods. First, the cell pellet was processed as above to detect whether the precursor peptide was cleaved. Purified His6-SmcA, His6-EtcA, and His6-PacA were detected as truncated leaders losing C-terminal residues after the GG motif, implying the protease (C or E) are functioning (FIG. 3b). The products were extracted and purified from the culture medium by solid-phase extraction using a reversed-phase polymeric resin. The desired end products from smc, etc, and pac clusters were either undetectable or detectable in trace amounts (FIG. 3b). This result suggested D or E transporters are not functioning efficiently for native His6-AB+CDE expressions. To increase the yields of end products, we tested nonnative combinations of His6-AB+CDE; i.e. AB is from one species and CDE is from another species. As shown in FIG. 3c, Smc, Etc, and Pac products could be efficiently produced using combinations of native His6-XyeAB+XncCDE. In this case, XyeAB are selected from SmcAB, EtcAB and PacAB. Tandem mass spectrometry (MSMS) analysis of these products confirmed the primary amino acid sequence and localized −2 Da losses to each of the three Ω1-X2-X3 motifs. Using these combinations, we proceeded with production of the Smc, Etc, and Pac products by larger scale fermentation, solid-phase extraction (polymeric resin), and preparative reversed phase HPLC which provided sufficient material for biological testing.

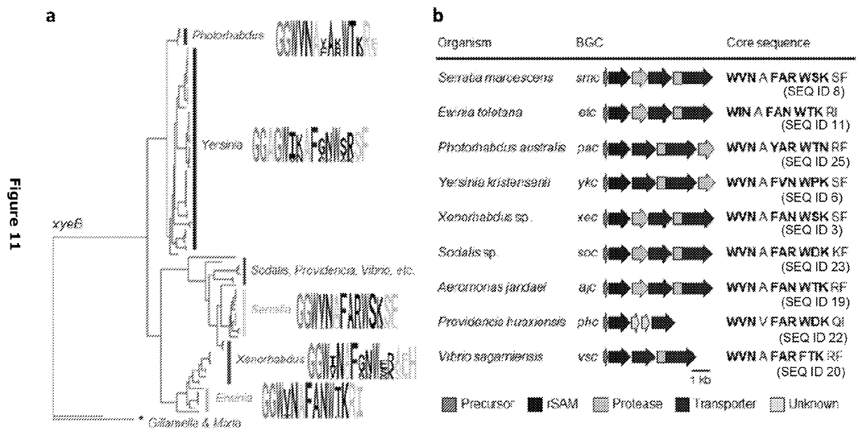

The second approach used to produce xenorceptides was expression of chimeric leader-core hybrids with the Xnc maturation and export machinery. These constructs were composed of His6-XncA leader (His6-XncAL) fused to the XyeA core of the target natural product inserted in pET28a(+). This precursor construct was coexpressed with XncBCDE encoded in pCDFDuet-1. This combination of genetic components allows a small gene fragment for the precursor to be synthesized and avoids the costly synthesis of the transport machinery. Using these constructs we pursued production of the products from different bacterial genera including: Yersinia kristensenii (ykc), Xenorhabdus sp. (xec), Sodalis sp. (soc), Aeromonas jandaei (ajc), Provedencia huaxiensis (phc), and Vibrio sagamiensis (vsc) (FIGS. 12a and 12b). Upon fermentation and extraction all of these products could be detected and analyzed −2 Da mass losses localized to the expected motifs. However, the products from phc and vsc were not produced in sufficient amounts for biological evaluation. With suitable constructs in hand, we proceeded with larger scale production of 5-8 for biological evaluation.

### Example 9: Antibacterial Activity of Xenorceptides

The eight xenorceptides along with synthetic versions of the unmodified peptide sequences were screened for antibacterial activity. Our initial panel for testing consisted of quality control strains representing Gram-positive and Gram-negative bacteria (Table 10). Minimal inhibitory concentration (MIC) values were obtained for 1-8 using broth microdilution assays. While 1 showed weak or no activity, we were encouraged that 2-4, and 8 showed selective activity for Gram-negative pathogens (E. coli ATCC 25922 and K. pneumoniae ATCC 700603). No activity was observed against Gram-positive bacteria (B. subtilis ATCC 6633 and S. aureus ATCC 29737) for any of the products tested, and suggests the bioactive products are selective against Gram-negative strains. The unmodified synthetic peptides representing the core sequences from 2-4 also did not show any bioactivity against Gram-negative and Gram-positive bacteria, which confirms that the cyclophane rings are critical to the bioactivity of the Xye peptides. Encouraged by the activity exhibited by 2-4, we carried out structure elucidation and further biological evaluation.

### Example 10: Structure Elucidation of Xenorceptides

The structures of products 2-4 were characterized by NMR spectroscopy to understand whether the XyeB maturases from different genera catalyze cyclophane formation with identical substitution pattern and the planar chirality with respect to the indole, using NMR spectra, assigned chemical shifts, and key correlations. Products 2-4 were characterized analogous to xenorceptide A1. In all cases, the XyeB maturases carry out the same crosslinking of Trp as in 1 (FIG. 4a). The Phe residue in 3 was assigned as para-substituted analogous to 1 (FIG. 4b). However, 2 was elucidated as meta-substituted based on 2D NMR. Phe5-H2 (δ 6.91 ppm) appears as a singlet and has NOESY correlations with both Phe5-Hpb (δ 2.73 ppm) and Arg7-Hβ 195 (δ 2.87 ppm). The remaining three aromatic protons within the same spin system (H4, δ 7.17 ppm; H5, δ 7.25 ppm; H6, δ 7.09 ppm) exhibit NOESY correlations with Phe5-Hβa (δ 2.96 ppm) and Arg7-Hβ (δ 2.10, δ 1.94 ppm), suggesting these protons lie on the same face and the new C(sp2)-C(sp3) bond is formed between Phe5-C3 with Arg7-Cy (FIG. 4). The Pac product (4) encodes a Tyr5 instead of Phe5, and the Tyr is crosslinked at C3 of Tyr (FIG. 4). This substitution pattern has been observed by triceptide maturases reported previously. The relative conformations of the cyclophane rings were assigned by NOESY and coupling constant analysis, which showed the orientation of the indole in the Trp-derived cyclophanes are identical for 1-4. The absolute configuration of X2 residues were assigned by advanced Marfey's method in addition to guanidine isothiocyanate derivatization. These analyses led to all α-positions to be of the natural L-configuration and the remaining amino acids to be as shown. The planar chirality of the Trp was assigned as Sp. The Smc, Etc, and Pac products were named xenorceptide A2 (2), xenorceptide A3 (3), and xenorceptide A4 (4), respectively (FIG. 4).

Structural eludication of xenorceptide A2 (2), xenorceptide A3 (3) and xenorceptide A4 (4) are shown in FIG. 26-28. FIG. 29-45 shows the NMR spectra used to derive the xenorceptide structures. Table 18-20 shows the summarised NMR data for these xenorceptides.

### Example 11: Biological Evaluation of Xenorceptide A2

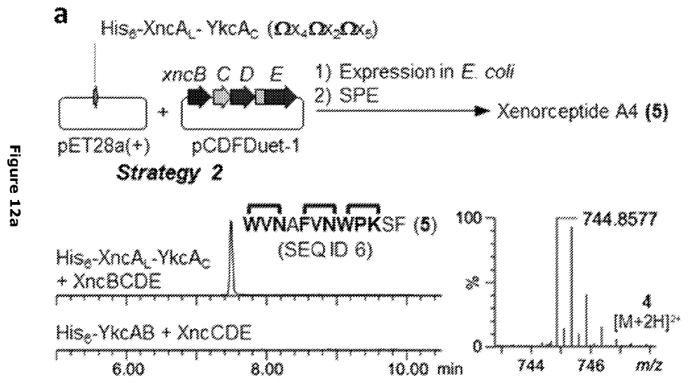

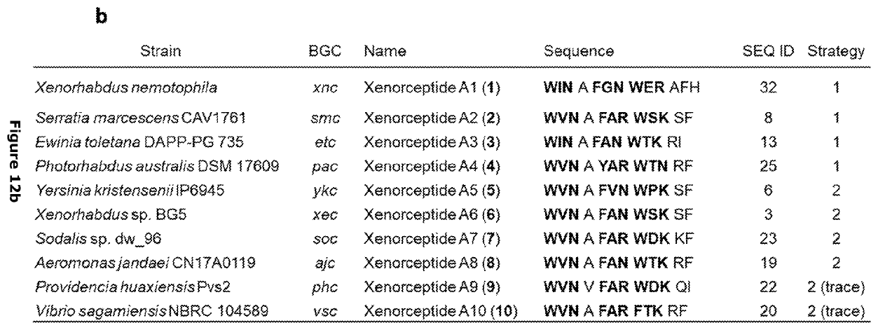

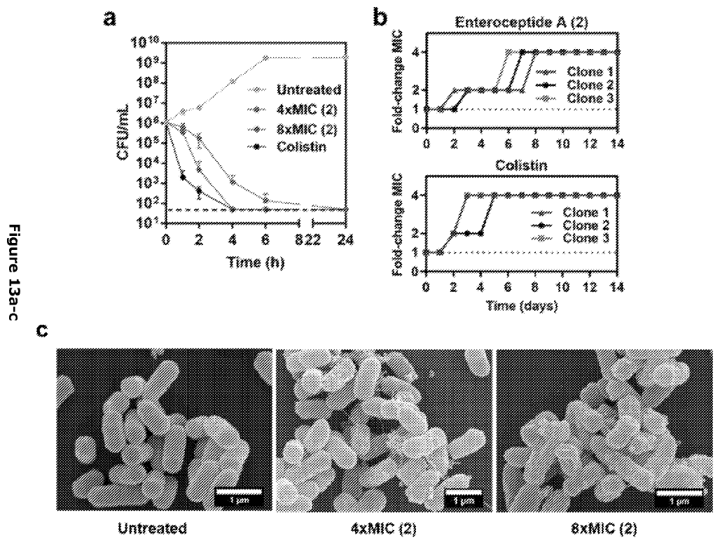

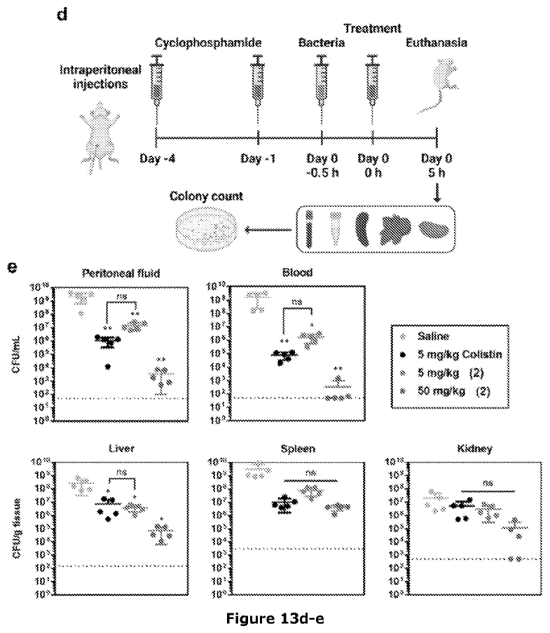

Xenorceptide A2 (2) was tested against a larger panel of clinical drug-resistant isolates. These results are summarized in Table 11 and confirm the selective activity (2-8 g/ml MICs) against Gram-negative Enterobacteriaceae, several of which are carbapenem-resistant Enterobacterales (CRE) pathogens. Next, we carried out time-kill assays against E. coli M6 (a carbapenem- and colistin-resistant clinical isolate) which showed that xenorceptide A2 (2) has a bactericidal effect over 24 h at 8×MIC, causing 3-log reduction in bacteria count (FIG. 13a). To further understand the killing effect of xenorceptide A2 (2), we imaged the morphology of E. coli MG in the presence of xenorceptide A2 (2) by scanning electron microscopy. Within 4 h of peptide treatment, the cells showed clear membrane damage and surface blebbing, followed by cell lysis and death (FIG. 13c). Xenorceptide A2 did not show any cytotoxicity against HepG2 human cells up to a concentration of 256 μg/ml. To understand resistance development, we incubated xenorceptide A2 at sub-inhibitory concentrations with E. 221 coli M6. Over the course of two weeks we obtained strains that were ˜4-fold resistant to xenorceptide A2 (2) with an MIC of 32 μg/ml (FIG. 13b). In contrast, E. coli M6 readily became less susceptible to colistin at an earlier time point than xenorceptide A2 (2). After extensive in vitro biological evaluations, we evaluated the in vivo antimicrobial efficacy of xenorceptide A2 (2) using a peritonitis model in neutropenic mice (FIG. 13d). After 30 min of inoculation with E. coli M6, mice (n=5 per group) were given a single intraperitoneal injection of treatment or saline. At 5 h post-treatment, the mice were euthanized for collection of peritoneal fluid, blood, and organs for quantification of bacteria burden using colony counting method. Xenorceptide A2 (2) displayed concentration-dependent antimicrobial effect in peritoneal fluid, blood, and liver where 50 mg/kg dose caused a 6-, 7-, and 4-log decrease in colony count relative to saline control results, respectively (FIG. 13e). While weaker effect was observed in spleen and kidney, 50 mg/kg xenorceptide A2 (2) still achieved 2-log reduction in bacteria burden. At the same dose of 5 mg/kg, the peptide displayed comparable efficacy to colistin.

### Example 12: Discussion

Antibiotics against Gram-negative pathogens are urgently needed. Natural products have been the main source of currently used antibiotics but no new classes of antibiotics have been introduced since the 1980s. Of the bacterial pathogens, Gram-negative are challenging for antibiotic discovery due to their dual membrane envelope. At current, there are two approaches for identifying natural product derived antibiotics. The first is using bioactivity-guided isolation. These platforms typically start with in vitro cell based assays where activity from a crude or partially purified extract is prioritized. A series of purification and retesting steps are carried out until the active component is isolated and characterized. This process was and remains the key process for which antibiotics have been discovered. However, over the last few decades, bioactivity-guided isolation discovery has suffered from rediscovery of known compounds. The second method is by producing targeted products directly for their chemical novelty—a chemically guided or chemistry first approach. The novelty may vary from as little as a functional group (congener of a known natural product) or could be a new and unpredictable scaffold. In this approach, the natural products are obtained by heterologous expression, host organism (native or engineered), or by chemical synthesis. We demonstrate the second approach to yield the targeted compounds directly and MIC values were obtained for each molecule produced.

In recent years promising antibiotics against Gram-negative pathogens have been described using bioactivity-guided approaches by exploiting unique bacterial sources, in particular the entomopathogenic bacteria, Xenorhabdus and Photorhabdus. While these organisms have been studied for their natural products, several antibiotics that target Gram-negative pathogens have been reported in recent years. Using a combination of different strategies (culturing under various conditions, co-culturing with other microorganisms, and mutations to the host RNA polymerase) led to the identification of odilorhabdins, broad spectrum peptide antibiotics from Xenorhabdus and Photorhabdus. In a separate study, darobactin was identified from strains of Photorhabdus by testing of 20× concentrated extracts. This concept was developed further to assay HPLC fractions representing 200× fold increase in concentrations, which led to the antibiotic, 3′-amino-3′-deoxyguanosine, a pro-drug with selective activity against E. coli and dynobactin, a second RiPP natural product able to target Gram-negative bacteria by inhibition of BamA.

Genome mining and synthetic biology have reinvigorated drug discovery from natural products and enabled chemistry-first approaches to advance. However, the discovery of selective inhibitors of Gram-negative bacteria using this approach has been less successful. One drawback is the need to treat each BGC on a case-by-case basis and requires specific manipulation for heterologous expression or activation of the pathway in host strains. We addressed some of these difficulties by developing two systems to access several natural products from different BGCs. Another approach independent of a producing microorganism has been to chemically synthesis natural products directly based on BGC-predicted compounds. This has been demonstrated by Wang and coworkers to identify macolacins, that show promising activity against Gram-negative bacteria. This methodology is most suited when the structures can be accurately predicted and the natural products are amenable to synthesis. For xenorceptide A2, bioinformatic prediction would have predicted the para-substituted Phe-derived cyclophane possibly resulting in a less or inactive product. The recent total synthesis of darobactin demonstrates the difficulty and complexity of synthesizing this class of molecules and represents a significant challenge. In this scenario, heterologous production has clear advantages over other methods for production.

Another potential drawback of chemistry first approaches is that the bioactivity of the target compounds cannot be predicted with certainty. However, some clues to what bioactivity can be expected using the composition of the BGC as a rudimentary guide.

In this example, xye BGCs are reminiscent of microcin or bacteriocin BGCs so we suspected the products may contain bactericidal activity. During the course of our work, the discovery of darobactins and dynobactins supported that xenorceptides possessing antibiotic activity likely existed. We proved our hypothesis to be valid for selected products obtained. This result was encouraging and supports that further production and testing of the remaining genetically encoded xenorceptides or variants may lead to products with higher potency, selectivity for other pathogenic bacteria, or have broader spectrum activity.

The C-terminal pentapeptide of xenorceptide A2 (2) including the 3-residue cyclophane is identical in sequence and configuration compared to darobactin. Darobactin has broad spectrum activity against Gram-negative pathogens and the mechanism of action was shown to bind to the bacterial insertase BamA, an essential outer membrane protein in Gram-negative bacteria. The N-terminus of xenorceptide A2 carries two distinct three-residue cyclophanes separated by a single amino acid. This feature differentiates xenorceptide A2 from both daroactin and dynobactin. Of significance with regard to the structures of dynobactin and xenorceptide A2 is that non-fused three-residue cyclophanes are able to inhibit selected Gram-negative bacteria. Xenorceptide A2 is more potent than dynobactin and has comparable potency to darobactin against Enterobactericeae. Another notable effect for xenorceptide A2 is that resistance development halted at 4×MIC and occurred over a period of 6-8 days. This shows that E. coli are less resistant to xenorceptide A2 compared to darobactin. While the mode of action for xenorceptide A2 remains to be elucidated, the two N-terminal cyclophanes appear to confer a greater selectivity for specific genera within Enterobacteriaceae. The producers of xenorceptides A2 (Serratia species) and G (Aeromonas jandaei) that have the highest potency against Gram-negative bacteria are derived from human samples while the other host strains are from other animals or plants. RiPP cyclophanes are among the most promising chemotypes for antibiotic development against Gram-negative pathogens. Their advantages include resistance to proteases, water solubility, first in class potential, and possess a unique mode of action. The discovery of darobactin, dynobactin, and xenorceptides also demonstrate efficacy of the two existing techniques to identify natural product antibiotics. Darobactins and dynobactins were identified using host strains and innovative bioactive guided fractionation. The discovery of xenorceptide A was identified by producing a series within a natural product class then screening for activity. We used synthetic genes and cross-combinations of genetic components (hybrid BGCs) to enable the production of the desired natural products. We envisage a similar or optimized approach using different combinations of genetic components will allow access to the remaining xenorceptides. The systematic production and testing of natural product families will hopefully become more routine to identify new and potent antibiotics to control antibiotic resistance pathogens.

### Example 13: Heterologous Expression of Xenorceptides A11 (11) A12-1 (12) and A12-2 (13) in E. coli

For the production of xenorceptides A11 (11), A12-1 (12) and A12-2 (13), they were produced in E. coli by expressing the Smc2A/pET28a(+), Smc3A-1/pET28a(+) or Smc3A-2/pET28a(+)+Smc3B-XncCDE/pCDFDuet-1. The Smc2A, Smc3A-1 or Smc3A-2 gene was expressed as an N-terminal His x 6 tag (Hiss) so that the precursor could be purified, and the modifications detected (FIGS. 14-16). This two-vector system allows His₆-xyeA precursor peptides modified by the rSAM/SPASM enzyme xyeB followed by xncCDE to cleave and export that is in a similar manner as above mentioned xenorceptides (FIGS. 3a and 3b).

The His₆-Smc2A/pET28a(+), His₆-Smc3A-1/pET28a(+) or His₆-Smc3A-2/pET28a(+) construct was co-expressed with Smc3B-XncCDE/pCDFDuet-1 construct in E. coli. The cell medium was analyzed by extraction of the culture medium using solid-phase extraction (SPE). The desired end products, xenorceptide All (11), xenorceptide A12-1 (12) and xenorceptide A12-2 (13) from Smc2A, Smc3A-1 and Smc3A-2 precursors, respectively were detected from LCMS and confirmed by MSMS analysis to localized −2 Da losses to each of the three Ω1-X2-X3 motifs (FIGS. 14-16). To sufficiently produce the end products 11-13 for antimicrobial assays, large scale culture was carried out. Total 10 liter of Smc2A, 6 liter of Smc3A-1 and S liter of Smc3A-2 were cultured, SPE extracted and HPLC purified to yield 11 (8.5 mg, 0.85 mg per liter), 11 (3.6 mg, 0.60 mg per liter) and 11 (5.5 mg, 0.68 mg per liter). Xenorceptide All (11), xenorceptide A12-1 (12) and xenorceptide A12-2 (13) were tested against a panel of clinical drug-resistant isolates. These results are summarized in Table 15.

### Example 14: Full Cluster Expression of Type B and Type D Xenorceptides

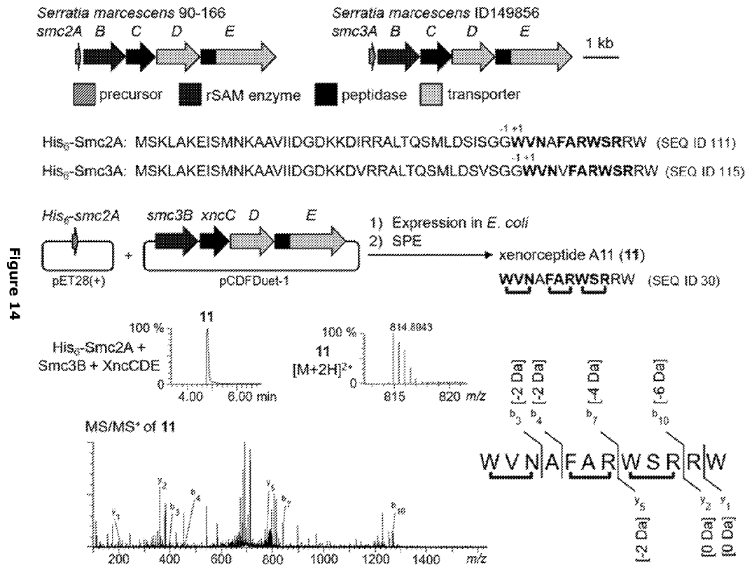

The Xye maturase system (GenProp1090) is derived from the names of three bacterial genera where it is commonly found: Xenorhabdus, Yersinia, and Erwinia. The substrate precursors are collectively referred to as XyeA, the rSAM proteins as XyeB, the proteases as XyeC, the transporters as XyeD, and the proteases/transporters as XyeE. Type B XyeA precursors containing ΩxxΩxxxx (n=2) and type D precursors containing ΩxxxxΩxxxx (n=16) through homology searches of rSAM/SPASM XyeB maturases in the RefSeq database. Subsequently, we screened the function of all the rSAM through co-expression of the precursor-rSAM pairs in E. coli. Based on these screening results, we have selected certain type B and type D family BGCs for full-gene cluster expression, specifically xgc, psc, poc, phc, kcc2, bbc, kcc1 and plc (as shown in FIG. 17). These three-letter short name to the gene clusters were given from the strain Xenorhabdus griffiniae VH1 (xgc), Pandoraea sp. PE-S2R-1 (psc), Pandoraea oxalativorans DSM 23570 (pol), Photorhabdus heterorhabditis Q614 (phc), Kosakonia cowanii pasteuri (kcc2 and kcc1), Bordetella bronchialis AU17976 (bbc) and Photorhabdus laumondii BOJ-47 (plc). For the xgc cluster, which contains two precursor genes, we named these two precursors XgcA1 and XgcA2. Additionally, the kcc2 and kcc1 clusters share the same protease and transporter, so both kcc2AB and kcc1AB were coexpressed with the protease and transporter genes labeled kcc2CDE.

To investigate whether XyeCDE can function on corresponding Xye precursor in E. coli, type B and type D family His6-tagged precursor and rSAM genes constructs were synthesized and inserted into pRSFDuet-1 vector, along with the relevant protease, transporter genes were cloned onto pCDFDuet-1 vector. These pairs of plasmids were then transformed into E. coli NiCo (DE3) host cells. The two-vector system enables testing of His6-xyeAB expression to ensure proper maturation by the rSAM enzyme, followed by expression of xyeCDE in a second vector to facilitate cleavage and export.

Each gene cluster was fermented in a small scale of 200 mL in LB media firstly, then the truncated leader and modified full-length peptides were purified using Nickel-affinity chromatography and digested with trypsin; the end products were purified by solid phase extraction (SPE) from culture media. The full-length peptides, truncated precursors, trypsin digested fragments and end products were then detected through LC-MS analysis.

Similarly, genes of each cluster's His6-tagged precursor and rSAM enzyme were cloned into pRSFDuet-1 plasmid, while the relevant protease, transporter genes were cloned into pCDFDuet-1 plasmid. These pairs of plasmids were then transformed into E. coli NiCo21 host cells. The two-vector system enables testing of His6-xyeAB expression to ensure proper maturation by the rSAM/SPASM enzyme, followed by expression of xyeCDE in a second vector to facilitate cleavage and export. Each gene cluster was fermented in a small scale of 200 mL, then the full-length precursors were purified by nickel affinity chromatography, digested with trypsin and subjected to LCMS, the end products were purified by SPE form culture media.

TABLE 12
Summary of Xye Type B and Type D full-cluster
expression screening

| | | Detection by LC-MS | | |
|---|---|---|---|---|
| | | SEQ | Truncated | Modified |
| BGC | Core sequence | ID | Leader | Core |
| xgCA1 | ASTAETWFKLDWKKSF | 54 | Yes | Yes |
| xgCA2 | SSDDDGIFFKTTWDRR | 55 | Yes | Yes |
| kcc2 | RGEGWVRAYWAKRF | 50 | Yes | Yes |
| kcc1 | DGRWLQWIKNH | 41 | Yes | Yes |
| phc | KPGEGWVNFTWNKSF | 52 | Yes | Yes |
| plc | GDRWLKWIKNH | 40 | Yes | No |
| poc | NVFVNATWSRAM | 47 | No | No |
| psc | GNAFVNATWSRAM | 234 | No | No |
| bbc | FANATWSKSF | 233 | No | No |

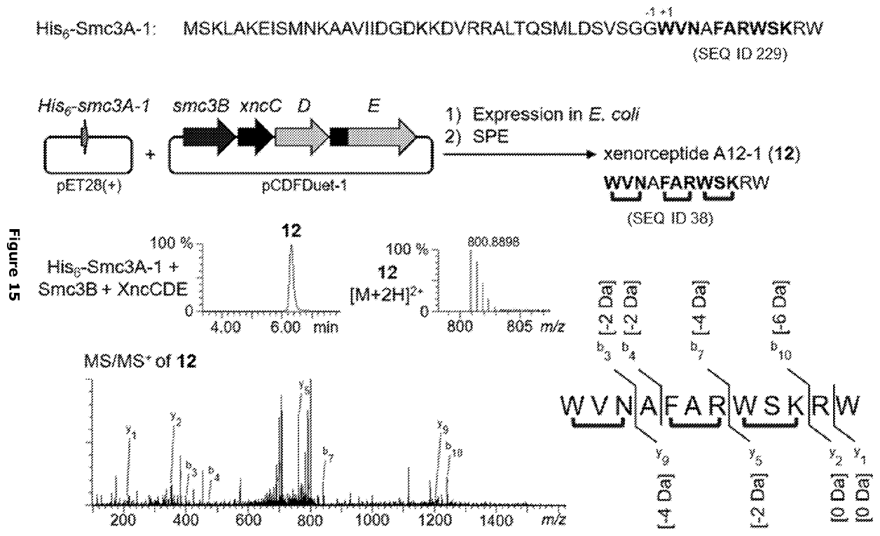

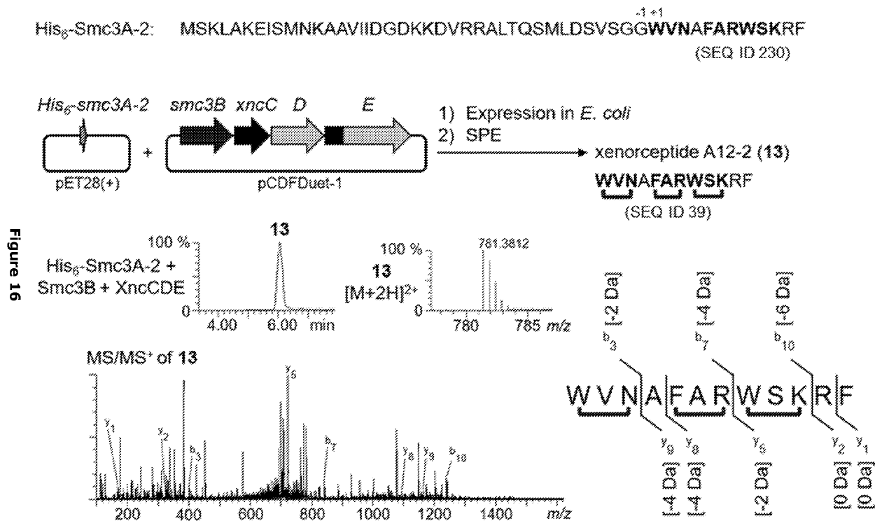

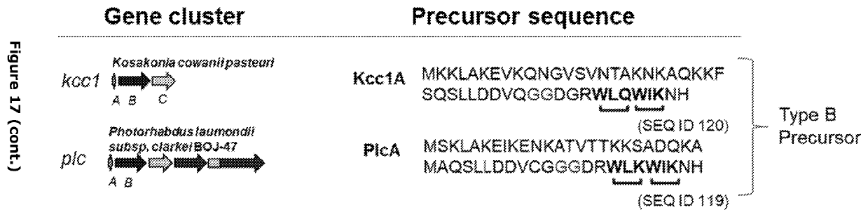

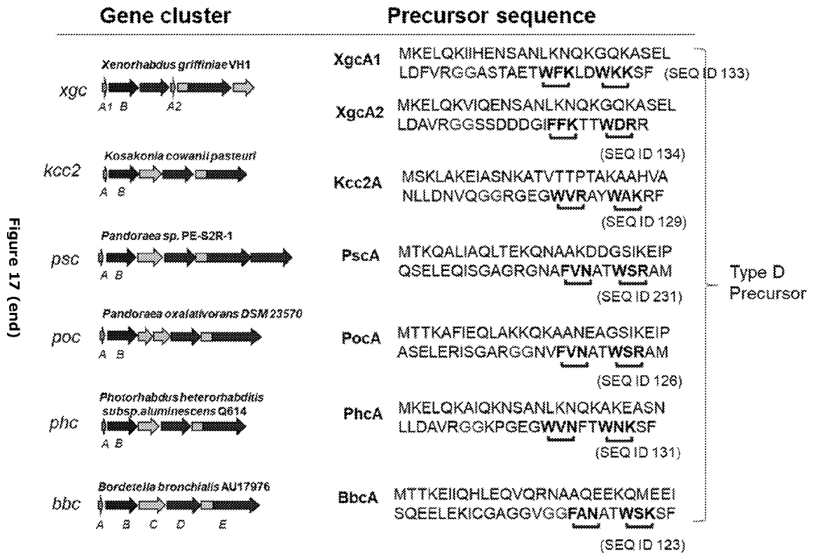

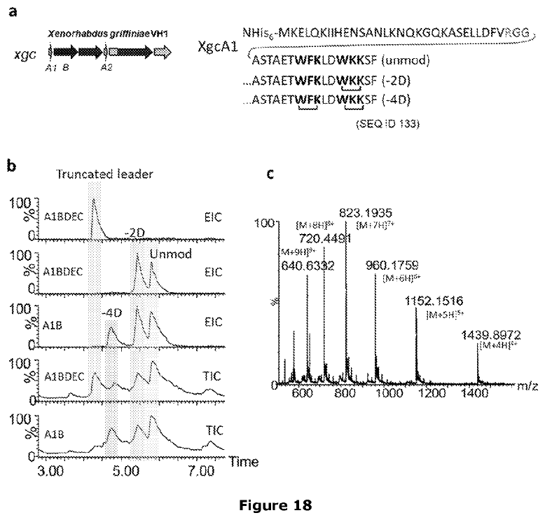

The clear peaks of truncated leaders from LC-MS data suggested that protease from xgc, phc, kcc2 and phc clusters can work well in E. coli for their corresponding precursors, and the cleavage site of these cluster are the GG motif as predicted. In the precursors XgcA1, XgcA2 and PhcA, there is an arginine located at the C-terminal immediately adjacent to Gly-Gly, which serves as the cleavage site of trypsin. Therefore, only full-length data for these three precursors are presented. (FIG. 18) Taking XgcA1 as an example, the LC-MS data shows that both mono-modified (−2D) and bi-modified (−4D) full-length precursors can be detected in both XgcA1B and XgcA1B+XgcDEC expression systems. However, the truncated leader that cleaves at the GG motif is only present in the full-cluster expression system. This suggests that the presence of protease is necessary for the successful cleavage of the XgcA1 precursor at the Gly-Gly motif. (FIG. 18)

In the case of kcc2 and kcc1, truncated leader is detectable in full-length, but in small quantities, so only the relatively clear digested fragment is shown. The characteristic fragment “AAHVANLLDNVQGG” (SEQ ID 236) ([M+H]⁺, m/z 1378.3395) is only detectable in Kcc2AB+Kcc2CDE expression, and similarly characteristic fragment “FSQSLLDDVQGG” (SEQ ID 237) ([M+H]⁺, m/z 1151.5164)” is only detectable in kcc1 full-cluster expression.

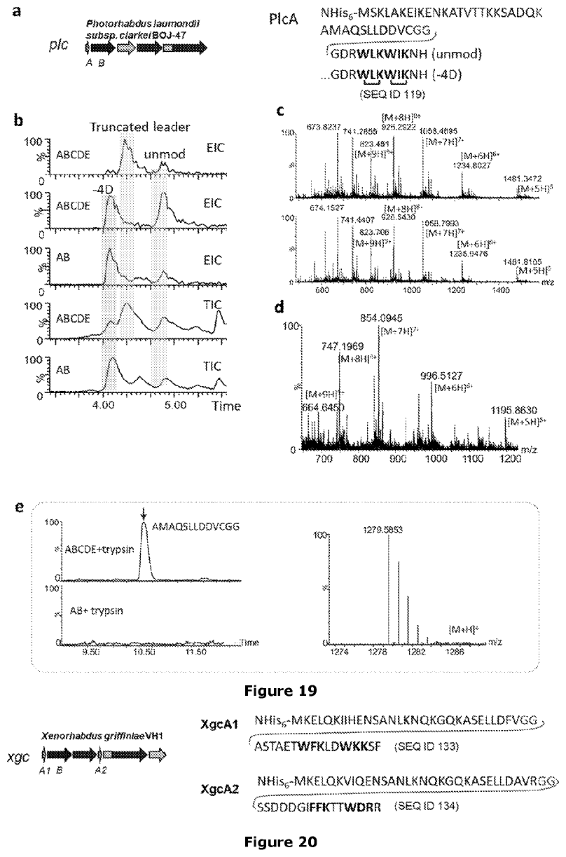

Observations have revealed that the plc precursor contains three consecutive Gly motifs at its C-terminal. (FIG. 19a) In full-length LCMS samples, significantly truncated precursors were detected from the first two GG motifs, (FIG. 19b, c) and similarly, trypsin-digested samples also showed clear evidence of cleavage at the first two GG motifs in the Plc precursors, supporting that these motifs act as a cleavage site. However, no product was detected in the supernatant, which suggests that the plc protease can function in E. coli, but the transporter is not operational in this organism. (FIG. 19). The other three clusters psc, bbc and poc, we attempted to use various combinations of proteases and transporters, but no desired compound was detected. Alternative strategy would be utilized on these clusters.

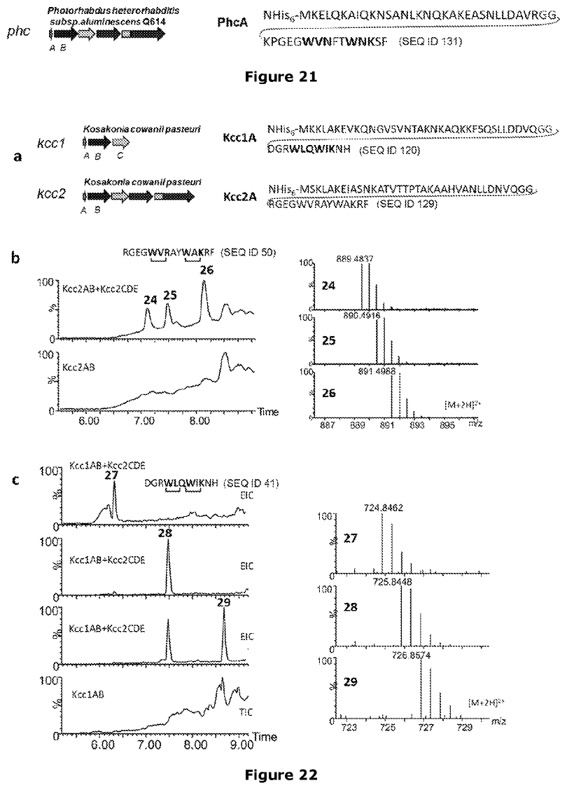

LC-MS data from small-scale SPE experiments revealed that full gene cluster expression of kcc2, kcc1, phc, xgc (A1 and A2) led to the detection of their respective end products, as compared to only His6-XyeAB expression. As demonstrated in FIG. 21, the products obtained from the kcc2AB+kcc2CDE construct included a double-charged fragment at m/z 889.4837, corresponding to −4 Da mass loss from the C-terminal core region of Kcc2A (RGEGWVRAYWAKRF, m/z 891.4710 [M+2H]²⁺), as well as a double-charged fragment at m/z 890.4916, corresponding to −2 Da mass loss of the core fragment, and an unmodified fragment at m/z 891.4988. Similarly, expression of kcc1 constructs resulted in the detection of −4 Da and −2 Da mass losses modified and unmodified core peptide fragments, which were displayed using an extracted ion chromatogram (EIC) in FIG. 10c because they were trace amounts. Tandem mass spectrometry (MS/MS) was conducted to locate the modifications to specific residues. MSMS analysis localized the −2 Da modifications to the first Ω1×2×3 motif for Kcc2A core peptide and the second Ω1×2×3 motif for −2 Da Kcc1 product. For phc and xgc (Aland A2), only fully modified end products were detected. In comparing the precursor A1 and A2 of Xgc, the efficiency of the Xgc transporter for XgcA1 is higher than that for XgcA2, evidenced by the significantly larger amount of XgcA1 end product detected in the supernatant compared to XgcA2. These results are summarized in Table 14 and illustrated in FIG. 20-22.

Large scale fermentation followed by SPE and preparative reversed phase HPLC was carried out for xgc(A1), phc and kcc2 clusters based on their good yield in small-scale experiments, to obtain a sufficient amount of compound from xgcA1, kcc2, kcc1, phc, plc. However, the yields of compounds from xgcA2, poc, psc and bbc were relatively low, making it difficult to obtain sufficient quantities for biological evaluation by SPE. Therefore, we designed several variants and utilize alternative strategies for xgcA2 and kcc1, as well those clusters that failed in full cluster expression.

### Example 15. In Vitro Cleavage of Leader Peptide from Modified Precursors

For the precursors that cannot be produced using the full-cluster expression strategy, we designed G-to-K/R/E variants in an attempt to obtain the predicted natural products via peptidase digestion. The core peptides are composed of 10-16 amino acids, which we have labelled with positive numbers starting from the first residue of the predicted core sequence. We were initially interested in the bbc cluster due to the presence of two Gly-Gly motifs at the C-terminal region (FIG. 17), with the GG closer to the C-terminal adjacent to the first Ω, which is a unique feature of type A Xye precursors. However, it was found that the rSAM BbcB can only catalyze the formation of one ring, which different from previous screening results. To determine which GG motif is the boundary between leader and core peptide and investigate the possibility of using another rSAM to form two rings, we designed a fusion precursor consisting of the BbcA leader and Kcc2A core and co-expressed it with BbcB. The purified product was trypsin-digested and analyzed via LCMS, revealing that only the longer leader helped to produce −2D modification in the Kcc2A core. These results suggest that the boundary between the precursor and core is located at the second GG motif.

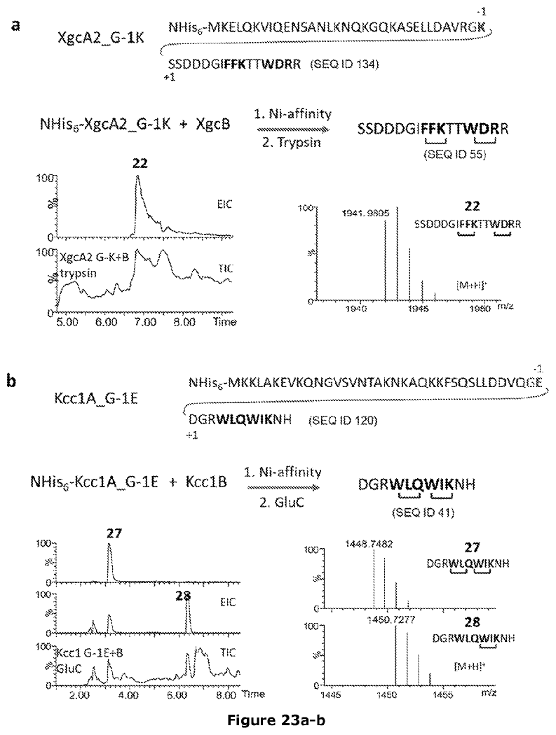

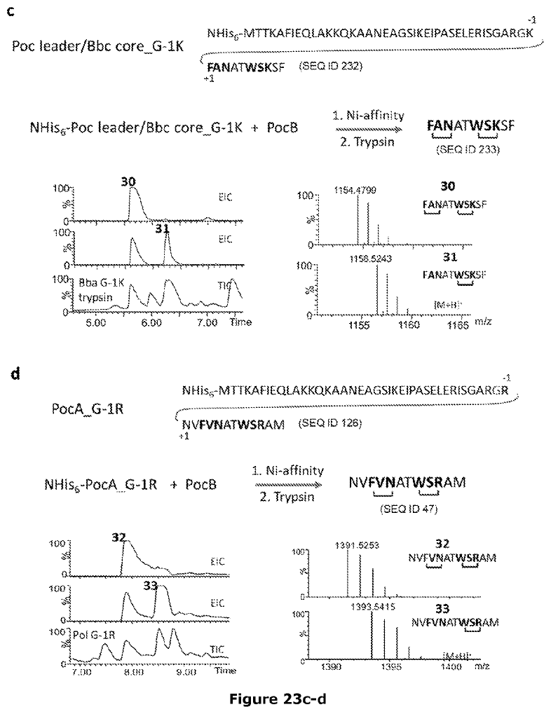

We investigated whether PocB rSAM could assist BbcA in forming two rings, as PocB has a high conversion rate to modify PocA, and the PocA core peptide is similar to the BbcA core. We also designed the Gly(−1) to Lys variant of PocA leader to generate the expected BbcA core peptide after trypsin cleavage. The results showed that PocB could indeed assist in the production of ˜4D and −2D modified BbcA core peptides, labelled compound 30 and 31, respectively. (FIG. 23c) We also designed variants of XgcA2(G-1K), Kcc1A(G-1E), and PocA(G-1R) to co-express their corresponding rSAM and then digested with appropriate peptidases to produce the predicted natural products. FIG. 23 a, b, d shows that the yield of these targeted fragments was good. The core peptides of PlcA and PscA have similarities with Kcc1A and PocA, respectively.

After the large-scale fermentation of 14-18 L of each variant, nickel affinity chromatography was used for purification, followed by semi-preparative HPLC to obtain a certain amount of compound 22, 27, 28, 30 and 31.

TABLE 13
Xye Type B and Type D core peptides

| Compound | Sequence |
|---|---|
| 21 | ASTAETWFKLDWKKSF (SEQ ID 54) |
| 22 | SSDDDGIFFKTTWDRR (SEQ ID 55) |
| 23 | KPGEGWVNFTWNKSF (SEQ ID 52) |
| 24 | RGEGWVRAYWAKRF (SEQ ID 50) |
| 25 | RGEGWVRAYWAKRF (SEQ ID 50) |
| 26 | RGEGWVRAYWAKRF (SEQ ID 50) |
| 27 | DGRWLQWIKNH (SEQ ID 41) |
| 28 | DGRWLQWIKNH (SEQ ID 41) |
| 29 | DGRWLQWIKNH (SEQ ID 41) |
| 30 | FANATWSKSF (SEQ ID 233) |
| 31 | FANATWSKSF (SEQ ID 233) |
| 32 | NVFVNATWSRAM (SEQ ID 47) |
| 33 | NVFVNATWSRAM (SEQ ID 47) |

* Bold residues refer to X₁ of the three-amino acid motif, where a cyclophane is formed between X₁ and X₃.

### Example 16. Antibacterial Activity

To assess the antibacterial activity of the compounds under investigation and determine their minimum inhibitory concentration (MIC), we purchased linear core peptides as internal standards and employed a spectroscopic method to quantify the samples for preliminary screening. Promising compounds will be produced in larger quantities and subjected to a more accurate MIC measurement. Our panel for testing consisted of E. coli, K. pneumoniae, E. cloacae, A. baumannii, E. faecalis and S. aureus (Table 14). MIC values were obtained for the compounds 21-29 and 30, 31, using broth microdilution assays. XgcA1 (21), XgcA2 (22), and both −4D and −2D Bbc products (30 and 31) showed no activity against all the strains that we tested. But we were encouraged by Kcc2 (24-25), Phc (23) and Kcc1 (27), 27 only had selective activity against K. pneumoniae with MIC value 8 μg/mL, 23 had some activity against E. coli, F. cloacae, A. baurmannii and K. pneumoniae, with MIC value range from 8-32 μg/mL. Notably, fully modified kcc2 core peptide (24) showed reasonable activity against Gram-negative strains E. coli, E. cloacae, A. baumannii, and K. pneumoniae with MIC value range from 1-4 μg/mL. From this result, it seems that the antibacterial activity of 24 is stronger but more narrow-spectrum than Darobactin, and selectively kills Gram-negative bacteria. Secondly, 25, which is single modified Kcc2 product, was also active against these test bacteria, but weaker than 24 that is fully modified, the unmodified product 26 was not active against any of the test bacteria, which confirms that the cyclophane rings are critical to the bioactivity of the Xye peptides.

TABLE 14
Antimicrobial activity

| | MIC (μg/mL) | | | | | | | | | | |
|---|---|---|---|---|---|---|---|---|---|---|---|
| Strain | 21 | 22 | 23 | 24 | 25 | 26 | 27 | 28 | 29 | 30 | 31 |
| Gram-negative bacteria | | | | | | | | | | | |
| Escherichia coli | >64 | >64 | 16 | 1 | 8 | >64 | >64 | — | >64 | >64 | >64 |
| ATCC 25922 | | | | | | | | | | | |
| Klebsiella pneumoniae | >64 | >64 | 32 | 2 | 16 | >64 | 8 | — | >64 | >64 | >64 |
| ATC 700603 | | | | | | | | | | | |
| Enterobacter cloacae | >64 | >64 | 32 | 4 | 16 | >64 | >64 | — | >64 | >64 | >64 |
| Acinetobacter baumanii | >64 | >64 | 64 | 2 | 16 | >64 | >64 | — | >64 | >64 | >64 |
| ATCC 19606 | | | | | | | | | | | |
| Gram-positive bacteria | | | | | | | | | | | |
| Enterococcus faecalis | >64 | >64 | >64 | 64 | >64 | >64 | >64 | — | >64 | >64 | >64 |
| Staphylococcus aureus | >64 | >64 | >64 | >64 | >64 | >64 | >64 | — | >64 | >64 | >64 |
| ATCC 29737 | | | | | | | | | | | |

TABLE 15
MIC value of xenorceptides A11, A12-1, A12-2,
D1 and B1 against bacterial pathogens

| | Xenorceptide | | | | | |
|---|---|---|---|---|---|---|
| Strain | Subtype | A11 | A12-1 | A12-2 | D1 | B1 |
| Escherichia | M2 | 8 | 8 | 4 | 4 | >32 |
| coli | M6 | 4 | 2 | 2 | 2 | >32 |
| | M10 | 2 | 2 | 2 | 2 | >32 |
| | M11 | 4 | 2 | 4 | 2 | >32 |
| Klebsiella | CRE1006 | 4 | 2 | 2 | 2 | >32 |
| pneumoniae | ATCC | 1 | 2 | 1 | 1 | >32 |
| | 25922 | | | | | |
| | CRE 1007 | 4 | 2 | 4 | 4 | >32 |
| | CRE1008 | 4 | 4 | 4 | 4 | >32 |
| | CRE1011 | 4 | 4 | 8 | 2 | >32 |
| | CRE1012 | 4 | 4 | 4 | 4 | >32 |
| | ATCC | — | — | — | 2 | — |
| | 700603 | | | | | |
| Pseudomonas | DR4877/07 | 32 | 32 | 32 | 16 | >32 |
| aeruginosa | DR5790/07 | 32 | 32 | 32 | 16 | >32 |
| | DM4150R | 16 | 32 | 32 | 32 | >32 |
| | DM23376 | 16 | >32 | 32 | 16 | >32 |
| Acinetobacter | ACβA1001 | 16 | 8 | 16 | 4 | >32 |
| baumanii | ACβA1002 | 16 | 8 | 8 | 4 | >32 |
| | ACβA1003 | 16 | 8 | 16 | 4 | >32 |
| | ACβA1004 | 16 | 8 | 16 | 4 | >32 |
| | ATCC | — | — | — | 2 | >32 |
| | 19606 | | | | | |
| Enterobacter | CRE1010 | 4 | 2 | 2 | 4 | >32 |
| cloacae | CRE1014 | 8 | 8 | 32 | 8 | >32 |
| | CRE1015 | 16 | 16 | 16 | 8 | >32 |
| | CRE1016 | 8 | 8 | 16 | 8 | >32 |
| | CRE1017 | 16 | 16 | 32 | 8 | >32 |
| | ATCC | — | — | — | 4 | >32 |
| | 13047 | | | | | |

Xenorceptide D1: SEQ ID 50;
Xenorceptide B1: SEQ ID 40

### Example 17. Structure Elucidation

Compound 24 has the strongest and broadest spectrum of anti-microbial activity among all the type A, type B and type D xenorceptides we have obtained so far, so we decided to prioritize the production of sufficient amounts of 24 for structure analysis. Concentrated SPE elute fraction from 40 L culture of Kcc2AB coexpressed with Kcc2CDE was subjected to reverse phase preparative HPLC using a C18 column followed by a Luna PFP column to get ˜6.8 mg of pure product.

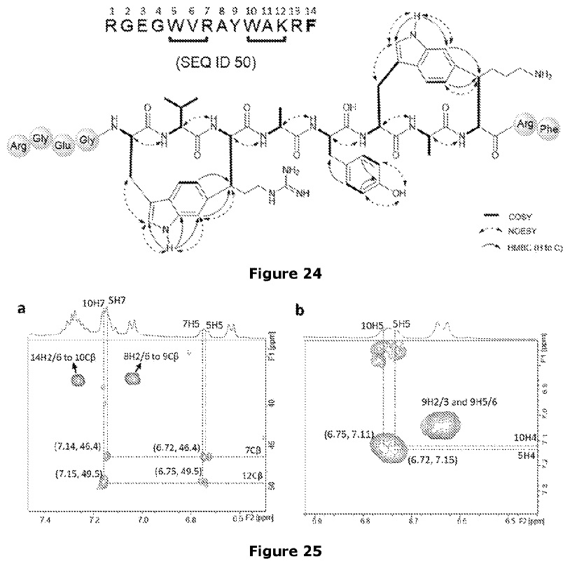

Compound 24 is composed of 14 amino acids, which we have labelled with positive numbers starting from the first residue of the predicted core sequence (FIG. 24). Sequential assignment of backbone NHs and their corresponding spin systems was performed using MS/MS and 2D NMR analysis, which confirmed the N-terminal (RGEG) and C-terminal (RF) sequences were unmodified. MS/MS of compound 24 showed −2 Da mass shifts localized to each of the WVR and WAK motifs within the predicted core peptide fragmentation, indicating that cyclization may have occurred within the two motifs.

Chemical shifts of side chain protons were assigned using COSY and TOSCY spectra. COSY and TOCSY correlations were observed between Ha and methyl group (Ala8 and Ala11) and through the spin system of iso-propyl side chain of Val6. The chemical shifts of Hβ/Cβ of Arg7 (δ 2.82 ppm/46.38 ppm) and Lys12 (δ 2.70 ppm/49.60 ppm) were assigned by TOCSY, COSY, and HSQC correlations starting from NH signals. 1H and 13C chemical shifts of the Trp5 and Trp10 were assigned starting from Arg7 Hβ/Cβ and Lys12 Hβ/Cβ respectively.

For the first macrocyclic ring, 2D NMR analysis indicated that Trp5 was now substituted at Trp5-C6, based on the following observations: Trp5-H4 (δ 7.15 ppm) and Trp5-H5 (δ 6.72 ppm) were assigned adjacent based on 3JHH coupling. The location of Trp5-H5 was supported by HMBC correlations to Arg7Cβ and a NOESY correlation to Arg7Hβ, 1H signals of Trp5-H5 appeared as a doublet. Trp5-H7 (δ 7.14 ppm) was assigned based on HMBC correlations to Arg7Cβ, a NOESY correlation to Arg7Hβ, Arg7Hγ (δ 2.13 ppm) and Trp5-indole NH (δ 10.74 ppm). The assignment of Trp5-H2 (δ 7.14 ppm) was supported by 3JHH coupling with Trp5-indole NH and a NOESY correlation to Trp5Hβ (δ 2.94 ppm). The indole NH gave correlations to C2, C3, C7, C7a. The protons for H1, H2, H4, H5, and H7 of Trp10 could be assigned while H6 was not observed. Collectively, these observations supported a new C—C bond between Trp5C6 and Arg7Cβ. Determination of the newly formed bond in the WAK motif was carried out in a similar fashion. FIG. 25 revealed key correlations that allowed assignment of the newly formed bonds.

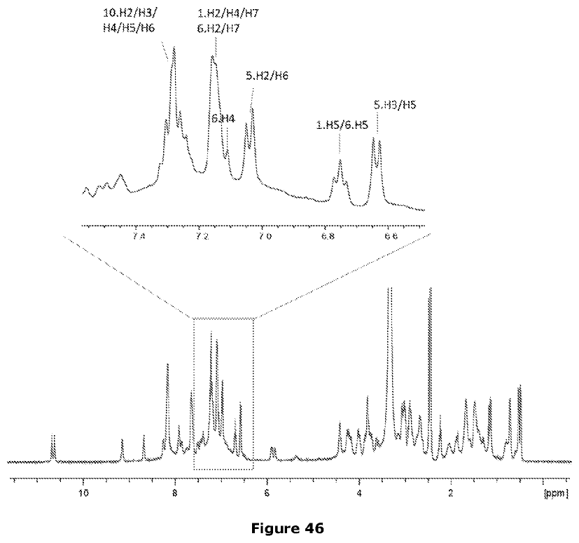

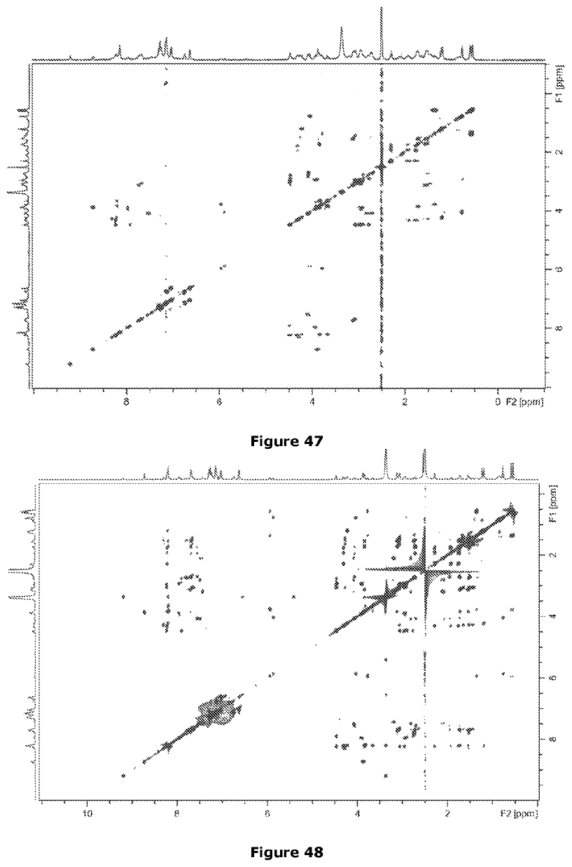

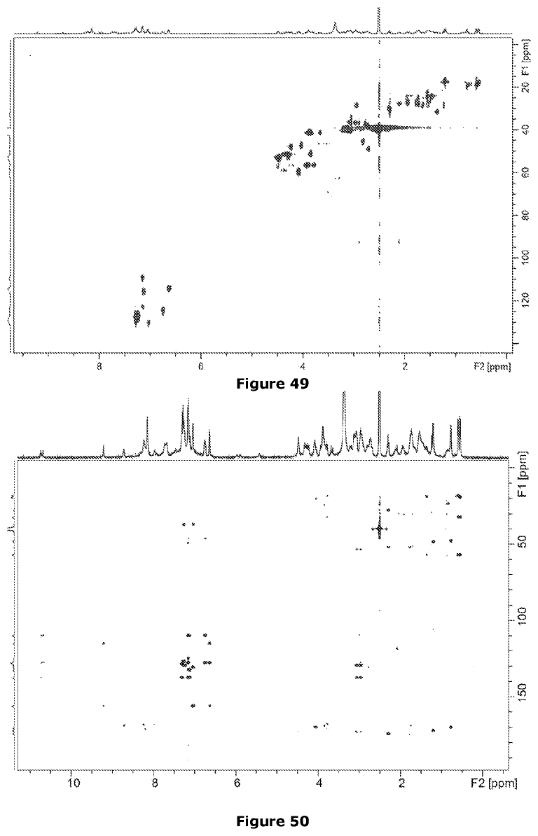

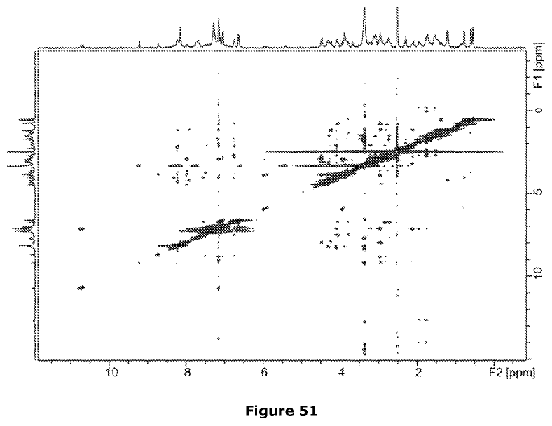

FIG. 46-51 shows the NMR spectra used to derive the structure of xenorceptide D1 (24). Table 21 shows the summarised NMR data for xenorceptide D1 (24).

### Materials, Equipment, and General Experimental Procedures.

Chemicals and reagents were purchased from the following suppliers: Acetonitrile from Tedia (USA); Isopropanol and methanol from Thermo Fisher Scientific (USA); Kanamycin and spectinomycin from GoldBio; Isopropyl β-D-1-thiogalactopyranoside (IPTG) from Combi-Blocks; and Strata-X® Polymeric Solid Phase Extraction (SPE) Sorbent (33 μm) from Phenomenex (USA); NMR solvent DMSO-d6 from Cambridge Isotope Labs (USA). Other chemicals and reagents were purchased from either Sigma (USA) or Bio Basic (Canada). Synthetic genes inserted into expression vectors were purchased from Twist Bioscience (USA). Escherichia coli NiCo21(DE3) cells were purchased from New England Biolabs (USA). Electroporation was carried out using mode p2 (2.5 kV, 5.6 ms) on a MicroPulser Electroporator (Bio-Rad, USA). Ultrasonication was carried out using an Ultrasonic Cleaner 142-0307 (VWR, USA). Centrifugation was carried out using either an Eppendorf® Centrifuge 5424R or 581CR (Germany), or an Avanti JXN-26 Ultracentrifuge (Beckman Coulter, USA). SPE was performed using either 12-Position Vacuum Manifold Set (Phenomenex, USA) or Vac-Man® Vacuum Manifold (Promega, USA). Sample solutions were concentrated using either a rotary evaporator (Rotavapor® R-210, Büchi, Switzerland), centrifugal evaporator (Genevac EZ-2 Elite, SP Scientific, UK), or freeze dryer (ScanVac CoolSafe, LaboGene, Denmark). LC-MS experiments were performed on a Waters Acquity UPLC System coupled to Xevo G1 QToF Mass Spectrometer (USA) and data was analyzed using MassLynx v.4.1. Preparative HPLC was carried out on a Shimadzu Nexera Prep System. NMR spectra were acquired at 298 K using a Bruker 400 MHz Avance Neo Nanobay NMR Spectrometer (USA) with a Bruker iProbe 5 mm SmartProbe or a Bruker 800 MHz Avance Neo NMR Spectrometer (USA) with a Bruker 5 mm CPTXI Cryoprobe and data was analyzed using Bruker Topspin v3.6.

Transformation of Plasmids into E. coli Cells.

Plasmids containing precursor (xyeA) and rSAM (xyeB) genes or those containing peptidase and transporter (xyeCDE) genes were synthesized by Twist Bioscience. The plasmids were reconstituted in autoclaved Milli-Q grade 1 water to a final concentration of 10 ng/μL. For full-length gene cluster expression, 1 μL of plasmid DNA was added to 70 μL of E. coli electrocompetent cells and transformed in a 2 mm electroporation cuvette. For coexpression, 1 μL of each plasmid DNA containing the appropriate genes was added to 70 μL of E. coli electrocompetent cells and transformed in a 2 mm electroporation cuvette. 1 mL of lysogeny broth (LB) was subsequently added to the transformed cells in an Eppendorf tube and incubated in the shaker at 37° C., 200 rpm for 1 h. Following this, the bacteria cells were centrifuged at 4,000 rpm for 10 min at 25° C. and the cell pellet obtained by disposing the supernatant. The cell pellet was then resuspended with the residual supernatant and streaked on LB agar supplemented with appropriate antibiotics to be grown overnight at 37° C.

Expression and purification of His₆-precursors.

An overnight culture of the transformant was inoculated into LB medium in an Ultra Yield® flask (Thomson) at a ratio of 1:100 v/v with appropriate antibiotics. The flask was shaken at 250 rpm and 37° C. until OD₆₀₀ reaches 1.5-3.0. The culture was cooled in an ice bath for 30 min. Protein expression was induced in the presence of 1 mM IPTG at 16° C. and shaken at 250 rpm for 16 to 24 h. The cells harvested by centrifugation were reconstituted in denaturing lysis buffer (100 mM NaH₂PO₄, 10 mM Tris, 9 M urea, 10 mM imidazole, pH 8.0) and then lysed by ultrasonication. The His₆-precursor in the supernatant was captured on HisPur Ni-NTA resin (Thermo Scientific, 625 mL per 20 mL supernatant) and purified according to the instructions provided by the manufacturer. The protein was eluted using NPI-250 (50 mM NaH₂PO₄, 300 mM NaCl, 250 mM imidazole, pH 8.0) and the buffer was exchanged into 50 mM Tris-HCl (pH 7.5) using a PD Minitrap G-10 column (GE Healthcare). When XyeAB were expressed, the purified protein was digested by trypsin (10 μg per 1 mL eluate) at 37° C. for 16 h, or by GluC (10 μg per 1 mL eluate) at 25° C. for 16 h. Digested precursors were analyzed by LC-MS using the following conditions: column=Phenomenex Kinetex XB-C18, 5 μm, 150×4.6 mm; mobile phase/gradient=solvent A: H2O (+0.1% formic acid, FA), solvent B: CH₃CN (+0.1% FA), isocratic 4% B for 2 min, followed by a linear gradient to 60% B over 10 min; flow rate=0.5 mL/min; column temp.=50° C. When XyeAB and XyeCDE were coexpressed, the purified protein was directly analyzed by LC-MS using the following conditions: column=Phenomenex Aeris WIDEPORE C4, 3.6 μm, 150×4.6 mm; mobile phase/gradient=solvent A: H2O (+0.1% formic acid, FA), solvent B: 1:1 CH₃CN/i-PrOH (+0.1% FA), isocratic 4% B for 2 min, followed by a linear gradient to 60% B over 12 min; flow rate=0.5 mL/min; column temp.=50° C.

### Purification of Full-Gene Cluster Expression by SPE and Preparative HPLC

After the overnight protein expression by IPTG, cells were removed by centrifugation at 4,000 rpm for 15 min at 4° C. 1 L supernatant was combined with 5.5 g of free-standing Strata-X® resin in a 2 L conical flask and shaken at 16° C., 160 rpm to allow binding of the core peptide to the resin. Peptide-bound resin was then washed twice with 60% methanol (55 mL), 100% methanol (55 mL), and finally eluted with 60% CH3CN with 0.1% FA (55 mL). The elution fraction was concentrated in vacuo, reconstituted in 20% CH3CN with 0.1% FA, and subjected to purification by preparative HPLC at the following conditions: solvent A: H2O (+0.1% TFA), solvent B: CH3CN (+0.1% TFA) Kinetex XB-C18, 5 μm, 250×21.2 mm: isocratic 4% B for 1 min, followed by a linear gradient to 30% B over 22 min; flow rate=20 mL/min; UV detection=280 nm; column temp.=room temperature.

### Purification of Xenorceptides.

After the overnight protein expression by IPTG, cells were removed by centrifugation at 4,000 rpm for 15 min at 4° C. 1 L supernatant was combined with 5.5 g of free-standing Strata-X® resin in a 2 L conical flask and shaken at 16° C., 160 rpm to allow binding of the core peptide to the resin. Peptide-bound resin was then washed twice with 60% methanol (55 mL), 100% methanol (55 mL), and finally eluted with 60% acetonitrile with 0.1% FA (55 mL). The elution fraction was concentrated in vacuo, reconstituted in 20% acetonitrile with 0.1% FA, and subjected to purification by preparative HPLC at the following conditions: column=Imtakt, Cadenza 5CD-C18, 5 μm, 250×20 mm; mobile phase/gradient=solvent A: H2O (+0.1% FA), solvent B: CH₃CN (+0.1% FA), isocratic 5% B for 1 min, followed by a linear gradient to 25% B over 17 min; flow rate=21.2 mL/min; UV detection=220 nm; column temp.=room temperature.

Yields of xenorceptides. Xenorceptide A1 (1) was obtained with yield of 5.0 mg/L of culture as a white powder. Xenorceptide A2 (2) was obtained with yield of 4.6 mg/L of culture as a white powder. Xenorceptide A3 (3) was obtained with yield of 1 mg/L of culture as a slightly yellow powder. Xenorceptide A4 (4) was obtained with yield of 3.3 mg/L of culture as slightly yellow powder.

### Minimum Inhibitory Concentration (MIC) Determination.

MIC screening of the peptides against a panel of ATCC and clinical strains was performed using broth microdilution method.¹ Briefly, peptides stock solutions in DMSO (0.1/G TFA) were diluted into Mueller Hinton Broth (MHB), followed by two-fold serial dilution in a 96-well plate. Bacteria culture in mid-log phase was diluted into MHB to yield 106 colony-forming units (CFU)/mL. Equal volume of the starting inoculum was added to the peptide samples, then incubated for 18-20 h (37° C., 120 rpm). OD₆₀₀ of the samples was then measured using Tecan Infinite M200 (TECAN, Männedorf, Switzerland). MIC is defined as the lowest peptide concentration to achieve more than 90% reduction in OD₆₀₀ relative to the drug-free control. The experiments were repeated three times. Colistin-resistant clinical isolates are a kind gift from Dr. Jeanette Koh (National University Hospital, Singapore). Multidrug-resistant clinical isolates are a kind gift from Dr. Lakshminarayanan Rajamani (Singapore Eye Research Institute, Singapore).

### Killing Kinetics Determination.

Peptides stock solutions were diluted into MHB to desired concentrations. Bacteria culture in mid-log phase was diluted into MHB to yield 10⁶ CFU/mL. The mixture was incubated at 37° C. with shaking. At each time point, 10 μL of the sample was drawn out and subjected to ten-fold serial dilution. 20 μL of relevant dilutions was dropped onto MHA plate using the drop plate method. The plate was incubated for 18-20 h at 37° C. Colony number was counted, and used for calculating the CFU/mL according to the equation:

CFU/mL=Colony count×50×dilution factor

### Field-Emission Scanning Electron Microscopy (FE-SEM) Microscopy.

E. coli M6 culture at mid-log phase was diluted to an OD₆₀₀ of 0.1. After incubating the bacteria with the peptide at 8×MIC for 1 h, 2 h, or 4 h at 37° C. with shaking, the samples were washed thrice in PBS. After overnight fixation with 2.5% glutaraldehyde (in PBS) at 4° C., the samples were washed twice in PBS, and then re-suspended in 500 μL of PBS. Sample was dropped onto cover slips pre-treated with poly-l-lysine. After 30 min, unbound cells were washed away with PBS. Following post-fixation with 1% OSO₄ for 30 min, OsO₄ was removed, and the cover slips were washed twice with distilled water. Samples were dehydrated using a series of ethanol solutions (50%, 75%, 95%, 3×100%). They were then subjected to critical point drying using Leica EM CPD300 (Wetzlar, Germany), followed by sputter gold coating using Leica EM ACE200 (Wetzlar, Germany). Viewing of the samples was performed using JEOL JSM-6701F (Tokyo, Japan). Images were processed using ImageJ (National Institutes of Health, Bethesda, MD).

### Serial Passage.

Resistance development of E. coli M6 against xenorceptide A2 was assessed by serial passaging of the bacteria in broth containing subinhibitory concentrations of the peptide. In brief, bacteria culture at mid-log phase was diluted to 10⁵-10⁶ CFU/mL in MHB containing 0.25×, 0.5×, 1×, 2×, and 4×MIC of the peptide. After 24h of incubation (37° C., 120 rpm shaking), the new visually observed MIC value was recorded, and the culture at highest peptide concentration showing visible growth was diluted to 105-106 CFU/mL in MHB. A new set of peptide concentration range was added to the cultures based on the latest MIC. This process was repeated over 14 days for three independent starting cultures.

### Advanced Marfey's Analysis.

100 μg each of product was hydrolyzed in 6 M HCl (1 mL) at 110° C. for 18 h. The hydrolysate was concentrated using a centrifugal evaporator and reconstituted in water (100 μL), followed by addition of 1 M NaHCO₃ (40 μL) and 1% w/v of Nα-(2,4-dinitro-5-fluorophenyl)-L-valinamide (L-FDVA) in acetone (200 μL). The mixture was incubated at 42° C. for 1 h and quenched with 2 M HCl (20 μL). L-Amino acid standards were derivatized in the same manner using L- and D-FDVA. The sample was diluted with CH₃CN/H₂O (1:1 v/v) and analyzed by LC-MS using negative ion mode. Retention times of the derivatized samples and standards are summarized in Table 15 with detailed LC conditions.

TABLE 15
Retention times of Marfey's type analysis of Xenorceptides.

| | Retention time (min)^{a} | | | | |
|---|---|---|---|---|---|
| Amino | L-DVA- | D-DVA- | Hydroly- | Hydroly- | Hydroly- |
| acid | std | std | sate of 2^{b} | sate of 3^{b} | sate of 4^{b} |
| L-Ala | 9.13 | 10.57 | 9.13 | 9.13 | 9.13 |
| L-Arg | 4.28 | 3.92 | n.d.^{c} | 4.28 | 4.28 |
| L-Asp | 7.63 | 7.98 | n.d.^{c} | n.d.^{c} | n.d.^{c} |
| L-Ile | 11.66 | 14.32 | — | 11.64 | — |
| L-Lys | 4.01 | 3.64 | n.d.^{c} | n.d.^{c} | — |
| L-Phe | 11.93 | 13.87 | 11.93 | n.d.^{c} | 11.92 |
| L-Ser | 7.31 | 7.66 | 11.31 | — | — |
| L-Thr | 7.41 | 9.10 | — | 7.43 | 7.42 |
| D-allo- | 7.66 | 8.44 | — | — | — |
| Thr | | | | | |
| L-Trp | 11.53 | 12.77 | n.d.^{c} | n.d.^{c} | n.d.^{c} |
| L-Tyr | 9.54 | 10.33 | — | — | n.d.^{c} |
| L-Val | 10.60 | 13.04 | n.d.^{c} | — | n.d.^{c} |

^{a}Analytical condition: MS polarity = negative; column: Kinetex XB-C18, 2.6 μm, 150 × 4.6 mm; flow rate: 0.50 mL/min; column temperature: 50° C.; mobile phase/gradient: 30% H₂O/CH₃CN + 0.1% FA isocratic for 2 min followed by linear gradient to 70% H₂O/CH₃CN + 0.1% FA over 17 min.
^{b}Derivatized with L-FDVA.
^{c}Not detected.

Derivatization of the hydrolysate of peptide 3 with GITC to resolve L-Ile and L-allo-Ile.

100 μg of hydrolysate of 3, L-Ile, and L-allo-Ile were derivatized with 2,3,4,6-tetra-O-acetyl-β-D-glucopyranosyl isothiocyanate (GITC) using the same protocol as Marfey's type analysis described above except that GITC (200 μL, 1% in acetone) was used instead of L-FDVA and the reaction was placed at room temperature for 1 h. The samples were then diluted with 1:1 ACN/H₂O and analyzed by LCMS using negative mode. The retention times are given in Table 16 with detailed LC condition.

TABLE 16
Retention times of GITC derivatization of 3.

| | Retention time (min)^{a} | | |
|---|---|---|---|
| Amino | | L-allo- | Hydrolysate |
| acid | L-std^{b} | std^{b} | of 3^{b} |
| Ile | 10.32 | 10.26 | 10.31 |

^{a}Analytical condition: MS polarity = negative; column: Kinetex XB-C18, 2.6 μm, 150 × 4.6 mm; flow rate: 0.50 mL/min; column temperature: 50° C.; mobile phase/gradient: 30% H₂O/CH₃CN + 0.1% FA isocratic for 2 min followed by linear gradient to 70% H₂O/CH₃CN + 0.1% FA over 17 min.
^{b}Derivatized with GITC.

TABLE 17
High-resolution MS data of modified peptide products identified in this study.

| | | | | Calculated | Observed | |
|---|---|---|---|---|---|---|
| | Compound | | Charge | mass | mass | |
| SEQ ID | # | Sequenceª | State | (monoisotopic) | (monoisotopic) | Δppm |
| 32 | 1 | WINAFGNWERAFH | [M + 2H]²⁺ | 821.3709 | 821.3721 | 1.5 |
| 8 | 2 | WVNAFARWSKSF | [M + 2H]²⁺ | 746.8597 | 746.8602 | 0.7 |
| 13 | 3 | WINAFANWTKRI | [M + 2H]²⁺ | 757.3886 | 757.3889 | 0.4 |
| 25 | 4 | WVNAYARWTNRF | [M + 2H]²⁺ | 789.3735 | 789.3741 | 0.8 |
| 225 | S1 | ELVDSLLDTVSGGWI | [M + 3H]³⁺ | 976.4631 | 976.4649 | 1.8 |
| | | NAFGNWERAFH | | | | |
| 226 | S2 | ALAQSMLDSVSGGW | [M + 3H]³⁺ | 903.7675 | 903.7661 | −1.5 |
| | | VNAFARWSKSF | | | | |
| 227 | $3 | ILVDSLLDTVSGGWI | [M + 3H]³⁺ | 928.4887 | 928.4896 | 1.0 |
| | | NAFANWTKRI | | | | |
| 228 | S4 | NNQPQPLTEDLLDQI | [M + 3H]³⁺ | 1166.5589 | 1166.5593 | 0.3 |
| | | SGGWVNAYARWTN | | | | |
| | | RF | | | | |

^{a}Cyclized three-residue motifs are indicated in red.

In vivo efficacy in peritonitis model.

All animal procedures were performed in accordance with protocols approved by the Institutional Animal Care and Use Committee (IACUC) at National University of Singapore (Singapore). Female C57BL/6NTac mice aged 6-8 weeks were acquired from InVivos Pte Ltd (Singapore, Singapore). Solutions for injections were prepared fresh in pharmaceutical grade saline and filter-sterilized. Murine peritonitis model was established according to literature. Briefly, healthy mice were rendered neutropenic by administering i.p. injection (0.5 mL) of cyclophosphamide on day −4 (150 mg/kg) and day −1 (100 mg/kg). On day 0, mice were infected with E. coli M6 (109 CFU/mL) through i.p. injection (0.1 mL). At 30 min post-inoculation, mice were given i.p. injection (0.5 mL) of a single dose of Smc (5 or 50 mg/kg), colistin (5 mg/kg), or saline control (n=5 mice per treatment group). At 2 h post-treatment, mice were humanely euthanized by carbon dioxide asphyxiation and cervical dislocation. Sterile PBS (3 mL) was injected into the peritoneal cavity, followed by abdominal massage and collection of peritoneal fluid (1-2 mL). Blood (0.3-0.5 mL) was collected through cardiac puncture. Liver, spleen, and kidney were surgically removed and stored in 0.1% Triton X-100 (in PBS). Tissue homogenization was performed using gentleMACS dissociator (Miltenyi Biotec, Germany) by following a published protocol. Cell aggregates were removed using a 30 μm mesh MACS SmartStrainer (Miltenyi Biotec). Blood, peritoneal fluid, and tissue homogenates were plated on LB agar and incubated overnight for colony counting.

### LC-MS Experiments

Mobile phases used are as follows: (A1) H2O+0.1% formic acid; (B1) CH3CN+0.1% formic acid; (B2) 1:1 CH3CN/isopropanol+0.1% formic acid. Details of conditions used for various samples are listed below:

For full-length precursors analyses, 10 μL of sample was injected into the system and left to run with the Phenomenex® Aeris Widepore 3.6 μm C4 column (150×4.6 mm) as stationary phase and mobile phases of A1 and B2 were used at a flow rate of 0.5 mL/min for 20 minutes and 10-75% B2 gradient over 12.5 minutes.

For digested fragment analyses, 40 μL of sample was injected into the system and left to run with Phenomenex Kinetex XB-C18, 5 μm, 150×4.6 mm column (150×4.6 mm) as stationary phase and mobile phases of A1 and B1 were used at a flow rate of 0.5 mL/min for 25 minutes and 4-60% B1 gradient over 17 minutes.

For SPE fractions, 40 μL of sample was injected into the system and left to run with Phenomenex Kinetex XB-C18, 5 μm, 150×4.6 mm column (150×4.6 mm) as stationary phase and mobile phases of A1 and B1 were used at a flow rate of 0.5 mL/min for 15 minutes and 4-32% B1 gradient over 7 minutes.

For subsequent MS/MS of fragmentation of selected ions, a collision energy of 30-45 eV was used. MassLynx v.4.1 was finally used to analyze the data collected.

### Antimicrobial Assays

MIC values for compounds (1-11) were assessed using 96-well plate format with Mueller Hinton (MH) broth, using the two-fold dilution method, previously reported in standard methods provided by Clinical and Laboratory Standards S8 Institute (CLSI). Kanamycin and ampicillin were used as antibacterial control agents. According to the reference, the compounds (1-11) were first dissolved in DMSO+0.1% TFA at a concentration of 3.2 mg/mL and 4 μL was serially diluted in 96 μL of MH broth. Then, sequential 2-fold serial dilutions of the mix were diluted in 50 μL MH broth and 50 μL cell cultures were added to wells. After incubation at 37° C. for 18 h, the lowest concentrations that completely inhibited the growth of bacteria in microdilution wells were detected by microplate reader for each tested compound, the values were recorded in Table 14. All assays were carried out in triplicate.

### General Cyclophane Synthetic Protocol

Precursor peptide containing alkyne moiety and 2-bromoacetanilide moiety (1.00 g, 1.04 mmol, 1.0 equiv) and Pd(PtBu₃)₂ (180 mg, 0.347 mmol, 0.3 equiv) were added to a flame-dried round bottom flask. The flask was evacuated and backfilled with argon (3×). Dry dioxane (100 mL) and DIPEA (0.99 mL, 5.20 mmol, 5.0 equiv) were added and the mixture was heated to 85° C. After 1.5 h, the reaction solution was cooled to ambient temperature then evaporated under vacuum. The crude solid may be purified via flash column chromatography using a gradient of 30% to 90% EtOAc in DCM.

TABLE 18
NMR data for xenorceptide A2.

| Residue | Position | ¹H^{a} | ¹³C^{a, b} | COSY | HMBC (H to C) | NOESY |
|---|---|---|---|---|---|---|
| Trp1 | C═O | | 168.3 | | | |
| | NH₂ | 8.22 | | Hα | | Trp1-Hα |
| | α | 3.65 | 54.5 | NH₂, Hβ | | Trp1-NH₂, Trp1-Hβa, |
| | | | | | | Tryp1-Hβb, Val2-NH |
| | β | 3.10 (Ha) | 27.0 | Hα | Trp1-Ca, Trp1-C2, | Trp1-Hα, Trp1-H4 |
| | | 3.06 (Hb) | | | Trp1-C3, Trp1-C3a | Trp1-Hα, Trp1-H2 |
| | 1 | 10.80 | | H2 | Trp1-C2, Trp1-C3, | Trp1-H2, Trp1-H7 |
| | | | | | Trp1-C3a, Trp1-C7a | |
| | 2 | 7.18 | 124.6 | H1 | Trp1-C3a, Trp1-C7a | Trp1-H1, Tryp1-Hβb |
| | 3 | | 108.0 | | | |
| | 3a | | 127.2 | | | |
| | 4 | 7.13 | 116.4 | H5 | Trp1-C3, Trp1-C3a, | Trp1-Hβa, Trp1-H5 |
| | | | | | Trp1-C6, Trp1-C7a | |
| | 5 | 6.77 | 124.2 | H4, H7 | Trp1-C3a, Trp1-C7 | Trp1-H4, Asn3-NH, |
| | | | | | | Asn3-Hβ |
| | 6 | | 130.9 | | | |
| | 7 | 7.38 | 110.7 | H5 | Trp1-C3a, | Trp1-H1 |
| | | | | | Trp1-C5, Asn3-Cb | |
| | 7a | | 137.1 | | | |
| Val2 | C═O | | 168.5 | | | |
| | NH | 6.94 | | Hα | Trp1-C═O | Trp1-Hα, Val2-Hβ |
| | α | 3.77 | 57.0 | NH, Hβ | Val2-C═O, Val2- | Val2-Hβ, |
| | | | | | Cβ, Val2-Cγ-M1 | Val2-Hγ-M1, Asn3-NH |
| | β | 1.45 | 31.9 | Hα, Hγ, | Val2-C═O, Val2- | Val2-Hγ-M1, Val2-Hγ-M2 |
| | | | | Hγ-M1, | Cα, Val2-Cγ-M1 | |
| | | | | Hγ-M2 | | |
| | γ-M1 | 0.70 | 18.4 | Hβ | Val2-Cα, Val2-Cβ | Val2-Hβ |
| | γ-M2 | 0.68 | 18.4 | Hβ | Val2-Cα, Val2-Cβ | Val2-Hβ |
| Asn3 | C═O | | 169.6 | | | |
| | NH | 7.67 | | Hα | Val2-C═O | Trp-H5, Val2-Hα |
| | α | 4.71 | 55.9 | NH, Hβ | Val2-C═O, Asn3-Cβ, | Ala4-NH |
| | | | | | Asn3-CONH₂, | |
| | | | | | Asn3-C═O | |
| | β | 3.74 | 52.0 | Hα | Trp1-C5, Trp1-C6, | Trp1-H5 |
| | | | | | Trp1-C7, Asn3-CONH₂, | |
| | | | | | Asn3-Cα, Asn3-C═O | |
| | CONH₂ | | 173.8 | | | |
| Ala4 | C═O | | 171.7 | | | |
| | NH | 7.24 | | Hα | Asn3-C═O | Asn3-Hα, Ala4-Hα, |
| | | | | | | Ala4-Hβ |
| | α | 4.40 | 48.1 | NH, Hβ | Ala4-Cβ | Ala4-NH, Ala4-Hβ, |
| | | | | | | Phe5-NH |
| | β | 1.13 | 18.4 | Hα, Hγ | Ala4-Cα, Ala4-C═O | Ala4-NH, Ala4-Hα |
| | | | | | | Phe5-NH |
| Phe5 | C═O | | n.d.^{c} | | | |
| | NH | 8.08 | | Hα | | Ala4-Hα, Ala4-Hβ, |
| | | | | | | Phe5-Hα, Phe5-Hβ |
| | α | 4.26 | 54.5 | NH, Hβ | | Phe5-Hα, Phe5-Hβ, |
| | | | | | | Phe5-H6, Ala6-NH |
| | β | 2.96 (Ha) | 39.5 | Hα | | Phe5-NH, Phe5-H2, |
| | | | | | | Phe5-H6 |
| | | 2.73 (Hb) | | | | Phe5-NH, Phe5-H2 |
| | 1 | | n.d.^{c} | | | |
| | 2 | 6.91 | 133.3 | H5 | Phe5-Cβ, Phe2-C6, | Phe5-Hβa, Phe5-Hβb, |
| | | | | | Arg7-Cβ | Arg7-NH, Arg7-Hβ |
| | 3 | | n.d.^{c} | | | |
| | 4 | 7.17 | 123.4 | H6 | Phe2-C2, Phe2-C6 | Arg7-Hγ |
| | 5 | 7.25 | 129.1 | H2 | | Phe5-H4, Phe5-H6 |
| | 6 | 7.09 | 127.6 | H3 | | Phe5-H5, Phe5-Hα, |
| | | | | | | Phe5-Hβa |
| Ala6 | C═O | | 169.9 | | | |
| | NH | 7.86 | | Hα | | Phe5-Hα |
| | α | 4.38 | 46.4 | NH, Hβ | Ala6-Cβ | Ala6-Hβ, Arg7-NH |
| | β | 0.95 | 15.8 | Hα | Ala6-Cα, Ala6-C═O | Ala6-Hα |
| Arg7 | C═O | | n.d.^{c} | | | |
| | NH | 7.58 | | Hα | | Phe5-H2, Ala6-Hα |
| | α | 4.23 | 58.3 | NH, Hβ | | Arg7-Hβ, Arg7-Hγ, |
| | | | | | | Trp8-NH |
| | β | 2.87 | 45.7 | Hα | Arg7-Cδ | Phe5-H2, Arg7-Hα, |
| | | | | | | Trp8-NH |
| | γ | 2.10 (Ha) | 28.3 | | | Phe5-H4, Arg7-Hα |
| | | 1.94 (Hb) | | | | Phe5-H4, Arg7-Hα |
| | δ | 2.96 | 37.2 | | | |
| | C | | n.d.^{c} | | | |
| | (guanidine) | | | | | |
| Trp8 | C═O | | 170.6 | | | |
| | NH | 8.53 | | Hα | | Arg7-Hα, Arg7-Hβ, |
| | | | | | | Trp8-Hβ |
| | α | 3.89 | 57.0 | NH, Hβ | | Trp8-Hβ, Thr9-NH |
| | β | 3.02 (Ha) | 28.3 | Hα | Trp8-C3 | Trp8-NH, Trp8-Hα |
| | | 2.98 (Hb) | | | | |
| | 1 | 10.70 | | H2 | Trp8-C2, Trp8-C3, | Trp8-H2, Trp8-H7 |
| | | | | | Trp8-C3a, Trp8-C7a | |
| | 2 | 7.16 | 123.9 | H1 | Trp8-C7a | Trp8-NH |
| | 3 | | 110.3 | | | |
| | 3a | | 128.2 | | | |
| | 4 | 7.14 | 115.9 | H5 | Trp8-C6, Trp8-C7α | Trp8-H5 |
| | 5 | 6.77 | 124.6 | H4 | Trp8-C3a, Trp8-C7 | Trp8-H4, Lys10-NH, |
| | | | | | | Lys10-Hβ |
| | 6 | | 132.9 | | | |
| | 7 | 7.17 | 110.4 | | Arg10-Cβ | Trp8-H1, Lys10-Hα |
| | 7a | | 137.8 | | | |
| Ser9 | C═O | | 167.9 | | | |
| | NH | 5.84 | | Hα | | Trp8-Hβ |
| | α | 4.03 | 54.5 | NH, Hβ | Trp8-C═O, Ser9-Cβ, | Ser9-Hβ, Lys10-NH |
| | | | | | Ser9-C═O | |
| | β | 3.09 | 62.0 | Hα | Ser9-C═O | Ser9-NH, Lys10-NH |
| Lys10 | C═O | | 170.7 | | | |
| | NH | 7.42 | | Hα | | Trp8-H5, Ser9-Hα, |
| | | | | | | Lys10-Hα, Lys10-Hβ |
| | α | 4.16 | 60.7 | NH, Hβ | Trp8-C6, Ser9-C═O, | Trp8-H7, Lys10-NH, |
| | | | | | Lys10-C═O, Lys10-Cβ, | Lys10-Hγa, Lys10-Hγb, |
| | | | | | Lys10-Cγ | Ser11-NH |
| | β | 2.73 | 49.5 | Hα, Hγ | | Trp8-H5, Lys10-Hα, |
| | | | | | | Lys10-Hγa, Lys10-Hgb, |
| | | | | | | Lys10-Hδa, Lys10-Hδb |
| | γ | 1.97 (Ha) | 24.5 | Hβ, Hδ | | Lys10-Hα, Lys10-Hβ |
| | | 1.86 (Hb) | | | | Lys10-Hα, Lys10-Hβ |
| | δ | 1.74 (Ha) | 25.7 | Hγ, Hε | | Lys10-Hβ |
| | | 1.50 (Hb) | | | | Lys10-Hβ |
| | ε | 2.75 | 39.4 | NH₂, Hδ | | Lys10-NH₂ |
| | NH₂ | 7.64 | | Hε | | Lys10-Hε |
| Ser11 | C═O | | n.d.^{c} | | | |
| | NH | 8.31 | | Hα | | Lys10-Cα, Ser11-Hβ |
| | α | 4.32 | 55.7 | NH, Hβ | | Ser11-Hβ, Phe12-NH |
| | β | 3.58 | 61.9 | Hα, Hγ | | Ser11-NH |
| Phe12 | C═O | | 173.2 | | | |
| | NH | 8.15 | | Hα | | Ser11-Hα, Phe12-Hβb |
| | α | 4.42 | 53.3 | NH, Hβ | | Phe12-NH |
| | β | 3.05 | 36.9 | | Phe12-Cα, Phe12-C1, | |
| | | 2.96 | | | Phe12-C2, Phe12-C═O | Phe12-NH |
| | 1 | | 137.3 | Hα, Hγ | | |
| | 2 | 7.26 | 129.2 | Hβ, Hδ | Phe12-Cβ, Phe12-C4, | |
| | | | | | Phe12-C6 | |
| | 3 | 7.29 | 128.8 | Hβ | Phe12-C1, Phe12-C5 | |
| | 4 | 7.24 | 127.0 | Hγ | Phe12-C2, Phe12-C6 | |
| | 5 | 7.29 | 128.7 | | Phe12-C1, Phe12-C5 | |
| | 6 | 7.26 | 129.2 | | Phe12-Cβ, Phe12-C4, | |
| | | | | | Phe12-C6 | |

^{a}800 MHz in DMSO-d₆ at 298 K.
^{b}Assigned by HSQC and HMBC.
^{c}Not detected.

TABLE 19
NMR data for xenorceptide A3.

| Residue | Position | ¹H^{a} | ¹³C^{a, b} | COSY | HMBC (H to C) | NOESY |
|---|---|---|---|---|---|---|
| Trp1 | C═O | | 167.7 | | | |
| | NH₂ | 8.26 | | Hα | | Trp1-Hβ |
| | α | 3.65 | 54.8 | NH₂, Hβ | | Ile2-NH |
| | β | 3.08 | 27.4 | Hα | Trp1-C3, Trp1-C3a, | Trp1-NH₂, Trp1-Hα, |
| | | | | | Trp1-C═O | Trp1-H2 |
| | 1 | 10.80 | | H2 | Trp1-C2, Trp1-C3, | Trp1-H2, Trp1-H7 |
| | | | | | Trp1-C3a, Trp1-C7a | |
| | 2 | 7.16 | 123.9 | H1 | Trp1-C3, Trp1-C3a, | Trp1-Hβ, Trp1-H1 |
| | | | | | Trp1-C7a | |
| | 3 | | 107.5 | | | |
| | 3a | | 126.8 | | | |
| | 4 | 7.13 | 116.0 | H5 | Trp1-C6, Trp1-C7a | Trp1-H5 |
| | 5 | 6.78 | 123.9 | H4, H7 | Trp1-C3a, Trp1-C7, | Trp1-H4, Asn3-Hβ |
| | | | | | Asn3-Cβ | |
| | 6 | | 130.3 | | | |
| | 7 | 7.39 | 110.8 | H5 | Trp1-C3a, Trp1-C5, | Trp1-H1, Asn3-Hα |
| | | | | | Asn3-Cβ | |
| | 7a | | 136.5 | | | |
| Ile2 | C═O | | 167.8 | | | |
| | NH | 6.92 | | Hα | Trp1-C═O | Trp1-Hα |
| | α | 3.80 | 56.7 | NH, Hβ | Ile2-Cβ, Ile2-Cγ-ε | Asn3-NH, |
| | β | 1.19 | 38.5 | Hα, Hγ | | Ile2-Hγ-Mε |
| | γ | 1.32 | 24.1 | Hβ, Hδ | | Ile2-Hδ |
| | γ-Mε | 0.66 | 14.8 | Hβ | Ile2-Cα, Ile2-Cb, | Ile2-Hα, Ile2-Hβ |
| | | | | | Ile2-Cγ | |
| | δ | 0.72 | 11.0 | Hγ | Ile2-Cβ, Ile2-Cγ | Ile2-Hγ |
| Asn3 | C═O | | 169.2 | | | |
| | NH | 7.65 | | Hα | | Ile2-Hα |
| | α | 4.72 | 56.4 | NH, Hβ | Ile2-CO, Asn3-Cβ, | Trp1-H7, Ala4-NH, |
| | | | | | Asn3-CONH₂, | |
| | | | | | Asn3-C═O | |
| | β | 3.77 | 52.5 | Hα | Trp1-C5, Trp1-C6, | Trp1-H5 |
| | | | | | Trp1-C7, | |
| | | | | | Asn3-CONH₂, | |
| | | | | | Asn3-Cα | |
| | CONH₂ | | 173.1 | | | |
| Ala4 | C═O | | 171.1 | | | |
| | NH | 7.40 | | Hα | Asn3-C═O | Asn3-Hα |
| | α | 4.37 | 47.7 | NH, Hβ | Ala4-Cβ, Ala4-C═O | Ala4-Hβ, Phe5-NH |
| | β | 1.13 | 18.6 | Hα, Hγ | Ala4-Cα, Ala4-C═O | Ala4-Hα |
| Phe5 | C═O | | n.d.^{c} | | | |
| | NH | 7.98 | | Hα | Ala4-C═O | Ala4-Hα |
| | α | 4.50 | 54.6 | NH, Hβ | | Ala6-NH, |
| | β | 3.20 (Ha) | 38.6 | Hα | | Phe5-Hβb, Phe5-H6 |
| | | 2.56 (Hb) | | | | Phe5-Hβa, Phe5-H6 |
| | 1 | | 135.6 | | | |
| | 2 | 6.85 | 129.2 | H3 | Phe5-C4, Phe5-C6 | Phe5-Hβa, |
| | | | | | | Phe5-Hβb, Phe5-H3 |
| | 3 | 7.03 | 131.5 | H2 | Phe5-C1, Phe5-C3, | Phe5-H2, Asn7-Hβ |
| | | | | | Asn7-Cβ | |
| | 4 | | 136.2 | | | |
| | 5 | 7.19 | 126.2 | | Phe5-C1, Phe5-C3 | |
| | 6 | 7.16 | 129.0 | | | |
| Ala6 | C═O | | 171.2 | | | |
| | NH | 6.88 | | Hα | | Phe5-Hα |
| | α | 3.72 | 48.2 | NH, Hβ | | Asn7-NH |
| | β | 0.96 | 19.0 | Hα | Ala6-Cα, | |
| | | | | | Ala6-C═O | |
| Asn7 | C═O | | 172.4 | | | |
| | NH | 7.81 | | Hα | | Ala6-Hα, Asn7-Hβ |
| | α | 5.05 | 53.8 | NH, Hβ | Ala6-C═O, Asn7-Cβ, | Trp8-NH |
| | | | | | Asn7-CONH₂, | |
| | | | | | Asn7-C═O | |
| | β | 3.75 | 52.5 | Hα | Phe5-C3, Phe5-C4, | Phe5-H5, Asn7-NH |
| | | | | | Phe5-C5, | |
| | | | | | Asn7-CONH₂, | |
| | | | | | Asn7-C═O | |
| | CONH₂ | | | | | |
| Trp8 | C═O | | n.d.^{c} | | | |
| | NH | 7.12 | | Hα | | Asn7-Hα, Trp8-Hα |
| | α | 3.94 | 56.9 | NH, Hβ | | Trp8-NH, Thr9-NH |
| | β | 3.00 (Ha) | 29.1 | Hα | | Trp8-H2 |
| | | 2.88 (Hb) | | | | Trp8-H2 |
| | 1 | 10.69 | | H2 | Trp8-C3, Trp8-C3a, | |
| | | | | | Trp8-C7a | |
| | 2 | 7.12 | 123.1 | H1 | Trp8-C3, Trp8-C4, | Trp8-Hβa, Trp8-Hβb |
| | | | | | Trp8-C7a | |
| | 3 | | 109.3 | | | |
| | 3a | | 127.5 | | | |
| | 4 | 7.10 | 116.3 | H5 | Trp8-C7a, Trp8-C6 | Trp8-H5 |
| | 5 | 6.70 | 124.7 | H4 | Trp8-C3a, Trp8-C7, | Trp8-H4, |
| | | | | | Lys10-Cβ | Lys10-Hβ |
| | 6 | | 132.3 | | | |
| | 7 | 7.16 | 109.8 | | Trp8-C5, Lys10-Cβ | Lys10-Hα, Lys10-Hγa, |
| | | | | | | Lys10-Hγb |
| | 7a | | 137.1 | | | |
| Thr9 | C═O | | 166.8 | | | |
| | NH | 5.95 | | Hα | | Trp8-Hα |
| | α | 3.93 | 57.6 | NH, Hβ | Thr9-C═O | Thr9-Hβ, Thr9-Hγ, |
| | | | | | | Lys10-NH |
| | β | 3.35 | 67.5 | Hα | Thr9-C═O | Thr9-Hα, Thr9-Hγ |
| | γ | 0.72 | 19.2 | | Thr9-Cα, Thr9-Cβ | Thr9-Hα, Thr9-Hβ |
| Lys10 | C═O | | 170.2 | | | |
| | NH | 7.30 | | Hα | | Thr9-Hα |
| | α | 4.12 | 60.0 | NH, Hβ | Lys10-C═O | Trp8-H7, Lys10-Hγ, |
| | | | | | | Arg11-NH |
| | β | 2.68 | 49.2 | Hα, Hγ | | Trp8-H5 |
| | | 1.98 (Ha) | 24.9 | Hβ, Hδ | | Lys10-Hγb, Trp8-H7, |
| | | | | | | Lys10-Hα |
| | γ | 1.78 (Hb) | | | | Lys10-Hγa, Trp8-H7, |
| | | | | | | Lys10-Hα |
| | δ | 1.53 | 26.2 | Hγ, Hε | Lys10-Cε | |
| | ε | 2.78 | 38.7 | NH₂, Hδ | | Lys10-NH₂ |
| | NH₂ | 7.74 | | Hε | | Lys10-Hε |
| Arg11 | C═O | | 171.4 | | | |
| | NH | 8.38 | | Hα | Lys10-C═O | Lys10-Hα, Arg11-Hα, |
| | | | | | | Arg11-Hβ |
| | α | 4.32 | 52.3 | NH, Hβ | | Arg11-NH, Arg11-Hβ, |
| | | | | | | Arg11-Hγ, Ile12-NH, |
| | β | 1.66 (Ha) | 28.8 | Hα, Hγ | | Arg11-NH |
| | | 1.52 (Hb) | | | | |
| | γ | 1.50 | 25.6 | Hβ, Hd | | Arg11-Hα, Arg11-Hδ |
| | δ | 3.09 | 40.4 | Hγ | Arg11-C | Arg11-Hγ |
| | | | | | (guanidine) | |
| | C | | 156.8 | | | |
| | (guanidine) | | | | | |
| Ile12 | C═O | | 172.8 | | | |
| | NH | 8.06 | | Hα | Arg11-C═O | Arg11-Hα |
| | α | 4.23 | 56.2 | NH, Hβ | Arg11-C═O, | Ile12-NH, Ile12-Hβ |
| | | | | | Ile12-Cβ, Ile12-Cγ, | |
| | | | | | Ile12-Cγ-Mε, | |
| | | | | | Ile12-C═O | |
| | β | 1.83 | 36.4 | Hα, Hγ | | Ile12-Ha, Ile12-Hδ, |
| | | | | | | Ile12-Hγ-Mε |
| | γ | 1.23 | 24.3 | Hβ, Hδ | Ile12-Cβ, | |
| | | | | | Ile12-Cγ-Mε, | |
| | | | | | Ile12-Cδ | |
| | γ-Mε | 0.89 | 15.5 | Hβ | Ile12-Cα, Ile12-Cβ, | Ile12-Hβ |
| | | | | | Ile12-Cγ | |
| | δ | 0.86 | 11.1 | Hγ | Ile12-Cβ, Ile12-Cγ | Ile12-Hβ |

^{a}400 MHz in DMSO-d₆ + 0.3% TFA-d at 298 K.
^{b}Assigned by HSQC and HMBC.
^{c}Not detected.

TABLE 20
NMR data for xenorceptide A4.

| Residue | Position | ¹H^{a} | ¹³C^{a, b} | COSY | HMBC (H to C) | NOESY |
|---|---|---|---|---|---|---|
| Trp1 | C═O | | 167.7 | | | |
| | NH₂ | 8.24 | | Hα | | Trp1-Hα, Trp1-Hβ |
| | α | 3.65 | 54.6 | NH₂, Hβ | | Trp1-NH₂, Val2-NH |
| | β | 3.09 | 27.3 | Hα | | Trp1-NH₂, Trp1-H4 |
| | 1 | 10.80 | | H2 | Trp1-C3, Trp1-C3a, | Trp1-H2, Trp1-H7 |
| | | | | | Trp1-C7a | |
| | 2 | 7.17 | 123.6 | H1 | Trp1-C3, Trp1-C3a | Trp1-H1 |
| | 3 | | 107.3 | | | |
| | 3a | | 126.5 | | | |
| | 4 | 7.13 | 115.8 | H5 | Trp1-C6, Trp1-C7a | Trp1-Hb, Trp1-H5 |
| | 5 | 6.77 | 123.7 | H4 | Trp1-C3a, Trp1-C7, | Trp1-H4, Asn3-Hβ, |
| | | | | | Asn3-Cβ | Asn3-NH |
| | 6 | | 130.1 | | | |
| | 7 | 7.38 | 110.6 | | Trp1-C3a, Trp1-C5, | Trp1-H1, Asn3-Hα |
| | | | | | Asn3-Cβ | |
| | 7a | | 136.6 | | | |
| Val2 | C═O | | 167.8 | | | |
| | NH | 6.95 | | Hα | Trp1-C═O | Trp1-Hα |
| | α | 3.77 | 57.3 | NH, Hβ | Val2-C═O | Asn3-NH |
| | β | 1.45 | 32.0 | Hα, Hγ-M1, | Val2-Cγ-M1 | Val2-Hγ-M1, |
| | | | | Hγ-M2 | Val2-Cγ-M2 | Val2-Hγ-M2 |
| | γ-M1 | 0.69 | 18.9 | Hβ, Hδ | Val2-Cα, Val2-Cβ, | Val2-Hβ |
| | | | | | Val2-Cγ-M2 | |
| | γ-M2 | 0.68 | 18.4 | Hβ | Val2-Cα, Val2-Cβ, | Val2-Hβ |
| | | | | | Val2-Cγ-M1 | |
| Asn3 | C═O | | 168.5 | | | |
| | NH | 7.65 | | Hα | Val2-Cα | Val2-Hα, Trp1-H5 |
| | α | 4.73 | 56.1 | NH, Hβ | Asn3-C═O | Trp1-H7, Ala4-NH |
| | β | 3.74 | 52.4 | Hα | Trp1-C5, Trp1-C6, | Trp1-H5 |
| | | | | | Trp1-C7, Asn3-Cα | |
| | CONH₂ | | | | | |
| Ala4 | C═O | | 170.8 | | | |
| | NH | 7.27 | | Hα | | Asn3-Hα |
| | α | 4.39 | 47.4 | NH, Hβ | | Ala4-Hβ, Tyr5-NH |
| | β | 1.13 | 18.6 | Hα, Hγ | Ala4-Cα, | Ala4-Hα, Tyr5-NH |
| | | | | | Ala4-C═O | |
| Tyr5 | C═O | | n.d.^{d} | | | |
| | NH | 8.04 | | Hα | | Ala4-Hα, Ala4-Hβ, |
| | | | | | | Tyr5-Hβa, Tyr5-Hβb |
| | α | 4.16 | 55.3 | NH, Hβ | | Ala6-NH |
| | β | 2.84 (Ha) | 38.1 | Hα | | Tyr5-NH, Tyr5-Hβb, |
| | | | | | | Tyr5-H2, Tyr5-H6 |
| | | 2.62 (Hb) | | | | Tyr5-NH, Tyr5-Hβa, |
| | | | | | | Tyr5-H2, Tyr5-H6 |
| | 1 | | 125.6^{c} | | | |
| | 2 | 6.67 | 135.3 | | | Tyr5-Hβa, Tyr5-Hβb, |
| | | | | | | Arg3-Hβ |
| | 3 | | 123.6^{c} | | | |
| | 4 | | 154.9 | | | |
| | 5 | 6.66 | 115.8 | H6 | Tyr5-C1, Tyr5-C3 | Tyr5-H6, Tyr5-OH |
| | 6 | 6.89 | 128.2 | H5 | Tyr5-C2, Tyr5-C4 | Tyr5-Hba, Tyr5-Hβb, |
| | | | | | | Tyr5-H5 |
| | OH | 9.39 | | | | Tyr5-H5 |
| Ala6 | C═O | | n.d.^{d} | | | |
| | NH | 7.68 | | Hα | | Tyr5-Hα, Ala6-Hβ |
| | α | 4.34 | 46.3 | NH, Hβ | | Ala6-Hβ, Asn7-NH |
| | β | 0.93 | 15.9 | Hα | | Ala6-NH |
| Arg7 | C═O | | n.d.^{d} | | | |
| | NH | 7.39 | | Hα | | Ala6-Hα, Trp8-NH |
| | α | 4.54 | 54.7 | NH, Hβ | | Trp8-NH |
| | β | 2.69 | 46.2 | Hα | | Arg7-Hγ |
| | γ | 2.54 (Ha) | 27.3 | | | Arg7-Hβ, Arg7-Hδ |
| | | 1.75 (Hb) | | | | |
| | δ | 2.91 | 39.7 | | | Arg7-Hγ |
| | C | | n.d. | | | |
| | (guanidine) | | | | | |
| Trp8 | C═O | | n.d.^{d} | | | |
| | NH | 8.64 | | Hα | | Arg7-NH, Arg7-Hα, |
| | | | | | | Trp8-Hβ |
| | α | 3.85 | 57.7 | NH, Hβ | | Trp8-Hβ, Thr9-NH |
| | β | 3.01 | 28.1 | Hα | | Trp8-NH, Trp8-Hα, |
| | | | | | | Trp8-H2, Trp8-H4 |
| | 1 | 10.72 | | H2 | Trp8-C3, Trp8-C3a | Trp8-H2, Trp8-H7 |
| | 2 | 7.15 | 123.3 | H1 | Trp8-C3, Trp8-C7a | Trp8-NH |
| | 3 | | 109.7 | | | |
| | 3a | | 126.9 | | | |
| | 4 | 7.18 | 116.2 | H5 | Trp8-C6 | Trp8-Hβ, Trp8-H5 |
| | 5 | 6.73 | 123.5 | H4 | Trp8-C3a | Trp8-H4, Lys10-NH, |
| | | | | | | Lys10-Hβ |
| | 6 | | 130.0 | | | |
| | 7 | 7.32 | 110.8 | | Trp8-C3a, Trp8-C5, | Trp8-NH, Lys10-Hα |
| | | | | | Asn10-Cβ | |
| | 7a | | 136.4 | | | |
| Thr9 | C═O | | 167.2 | | | |
| | NH | 6.06 | | Hα | | Trp8-Hα |
| | α | 3.90 | 57.5 | NH, Hβ | | Asn10-NH |
| | β | 3.41 | 67.5 | Hα, Hγ | | Thr9-Hγ, Asn10-NH |
| | γ | 0.81 | 18.7 | Hβ | Thr9-Cα, Thr9-Cβ | Thr9-Hβ |
| Asn10 | C═O | | 169.5 | | | |
| | NH | 7.55 | | Hα | | Trp8-H5, Thr9-Hα, |
| | | | | | | Thr9-Hβ |
| | α | 4.77 | 56.0 | NH, Hβ | Asn10-C═O | Trp8-H7, Arg11-NH |
| | β | 3.73 | 52.5 | Hα, Hγ | | Trp8-H5 |
| | CONH₂ | | n.d.^{d} | | | |
| Arg11 | C═O | | 170.8 | | | |
| | NH | 7.48 | | Hα | Asn10-C═O | Asn10-Cα, Arg11-Hα, |
| | | | | | | Arg11-Hβ |
| | α | 4.29 | 51.4 | NH, Hβ | | Arg11-NH, Arg11-Hβ, |
| | | | | | | Phe12-NH |
| | β | 1.63 (Ha) | 29.0 | Hα, Hγ | | Arg11-NH, Arg11-Hα, |
| | | 1.42 (Hb) | | | | Phe12-NH |
| | γ | 1.40 | 24.3 | Hβ, Hδ | | Arg11-Hδ |
| | δ | 3.01 | 40.3 | Hγ | | Arg11-Hγ |
| | C | | n.d.^{d} | | | |
| | (guanidine) | | | | | |
| Phe12 | C═O | | 172.4 | | | |
| | NH | 8.16 | | Hα | Arg11-C═O | Arg11-Hα, Arg11-Hβ, |
| | | | | | | Phe12-Hα, Phe12-Hβ |
| | α | 4.38 | 53.4 | NH, Hβ | Phe12-Cβ, Phe12-C1, | Phe12-NH |
| | | | | | Phe12-C═O | |
| | | 3.06 | 36.4 | | Phe12-C═O | Phe12-NH |
| | β | 3.00 | | Hα | | |
| | 1 | 137.2 | | | | |
| | 2 | 128.9 | 7.27 | | Phe12-Cβ, Phe12-C4, | |
| | | | | | Phe12-C6 | |
| | 3 | 128.1 | 7.29 | H4 | Phe12-C1, Phe12-C5 | |
| | 4 | 126.2 | 7.21 | H3, H5 | Phe12-C2, Phe12-C6 | |
| | 5 | 128.1 | 7.29 | H4 | Phe12-C1, Phe12-C5 | |
| | 6 | 128.9 | 7.27 | | Phe12-Cβ, Phe12-C4, | |
| | | | | | Phe12-C6 | |

^{a}400 MHz in DMSO-d₆ + 0.2% TFA-d at 298 K.
^{b}Assigned by HSQC and HMBC.
^{c}The assignment of Tyr5-C1 and Tyr5-C3 are interchangeable.
^{d}Not detected.

TABLE 21
NMR data for xenorceptide D1.

| Residue | Position | ¹H^{a} | ¹³C^{b} | COSY | HMBC (H to C) | NOESY |
|---|---|---|---|---|---|---|
| Arg(−4) | C═O | | 18.9 | | | |
| | NH | 8.22 | | Hα | Arg(−4)-CO | |
| | α | 3.86 | 42.2 | NH, Hβ | | |
| | β | 3.20 | 40.2 | Hα, Hγ | | |
| | γ | 1.53 (Ha) | 26.6 | Hβ, Hδ | | |
| | | 1.72 | | | | |
| | | (Hb) | | | | |
| | δ | 2.70 | 39.2 | Hγ | | |
| Gly(−3) | C═O | | 168.8 | | | |
| | NH | 8.71 | | Hα | | |
| | α | 3.88 | 42.18 | NH, Hβ | | |
| Glu(−2) | C═O | | 172.1 | | | |
| | NH | 8.20 | | Hα | | |
| | α | 4.30 | 52.5 | NH, Hβ | | |
| | β | 1.78 (Ha) | 28.0 | Hα, Hγ, | | |
| | | 1.93 | | OH | | |
| | | (Hb) | | | | |
| | γ | 2.28 (Ha) | 30.5 | Hβ | | |
| | | 2.30 | | | | |
| | | (Hb) | | | | |
| Gly(−1) | C═O | | 168.2 | | | |
| | NH | 8.20 | | Hα | Gly(−1)-CO | |
| | α | 3.86 | 42.2 | NH, Hβ | | Trp1-NH |
| Trp1 | C═O | | 168.2 | | | |
| | NH | 7.98 | | Hα | Gly(−1)-CO | Gly(−1)-Hα, Trp1-Hα, |
| | | | | | | Trp1-Hβ |
| | α | 3.94 | 57.4 | Hβ, NH | | Val2-NH, Trp1-Hβ, |
| | | | | | | Trp1-H4 |
| | β | 2.94 | 29.4 | Hα | Trp1-C3a | Val2-NH, Trp1-Hα, |
| | | | | | | Trp1-H2, Trp1-H4 |
| | 4 | 7.15 | 116.7 | H5 | Trp1-C3, Trp1-C3a, | Trp1-Hβ, Trp1-H5 |
| | | | | | Trp1-C5, Trp1-C6, | |
| | | | | | Trp1-C7a | |
| | 5 | 6.72 | 125.1 | H4 | Arg3-Cβ, Trp1-C3a, | Arg3-Hβ, Trp1-H7 |
| | | | | | Trp1-C7 | |
| | 6 | | 132.4 | | | |
| | 7 | 7.14 | 110.0 | | Arg3-Cβ, Trp1-C3, | Arg3-Hβ, Trp1-H5 |
| | | | | | Trp1-C3a,Trp1-C5, | |
| | | | | | Trp1-C6, Trp1-C7 | |
| | 7a | 137.5 | | | | |
| | 1 | 10.74 | | H2 | Trp1-C2, Trp1-C7, | Trp1-H2 |
| | | | | | Trp1-C7a | |
| | 2 | 7.16 | 123.7 | NH | Trp1-C3, Trp1-C3a, | Trp1-Hβ, Trp1-NH |
| | | | | | Trp1-C7a | |
| | 3 | | 110.1 | | | |
| | 3a | | 128.2 | | | |
| Val2 | C═O | | 171.7 | | | |
| | NH | 5.96 | | Hα | | Trp1-Hα, Val2-Hγ1, |
| | | | | | | Val2-Hγ2 |
| | α | 3.77 | 57.2 | NH, Hβ | Val2-CO, Arg3-CO, | Val2-Hβ, Val2-Hγ1, |
| | | | | | Val2-Cβ | Val2-Hγ2, Arg3-Hα |
| | β | 1.36 | 32.5 | Hα, | Val2-Cα, Val2-Cγ1, | Val2-NH, Val2-Hα, |
| | | | | Hγ1, | Val2-Cγ2, | Val2-Hγ1, Val2-Hγ2, |
| | | | | Hγ2 | | Arg3-NH |
| | γ1 | 0.54 | 19.3 | Hβ | Val2-Cα, Val2-Cβ, | Val2-Hα, Val2-Hβ |
| | | | | | Val2-Cγ2 | |
| | γ2 | 0.60 | 18.6 | Hβ | Val2-Cα, Val2-Cβ, | Val2-Hα, Val2-Hβ |
| | | | | | Val2-Cγ1 | |
| Arg3 | C═O | | 170.5 | | | |
| | NH | 7.49 | | Hα | | Val2-Hα, Val2-Hβ, |
| | | | | | | Arg3-Hβ |
| | α | 4.08 | 60.5 | NH, Hβ | | Ala4-NH |
| | β | 2.82 | 46.4 | Hα, Hγ | | Ala4-NH |
| | γ | 2.13 | 28.0 | Hβ, Hδ | | Arg3-Hα, Arg3-Hβ, |
| | | | | | | Arg3-Hδ, |
| | δ | 3.20 | 40.3 | NH | | Arg3-Hγ |
| | NH (side | 7.45 | | Hδ | | Arg3-Hδ |
| | chain) | | | | | |
| Ala4 | C═O | | 172.3 | | | |
| | NH | 8.20 | | Hα | Ala4-CO | Ala4-Hα, Ala4-Hβ |
| | α | 4.22 | 48.7 | NH, Hβ | Ala4-Cβ, Ala4-CO | Ala4-Hβ, Tyr5-NH |
| | β | 1.20 | 18.9 | Hα | Ala4-Cα, Ala4-CO | Ala4-Hα, Ala4-NH |
| Tyr5 | C═O | | 173.0 | | | |
| | NH | 7.75 | | Hα | | Tyr5-Hα, Tyr5-Hβ |
| | α | 4.57 | 51.6 | NH, Hβ | Tyr5-CO | |
| | β | 2.62 (Ha) | 35.0 | Hα | Tyr5-Cα, Tyr5-C1 | Tyr5-NH, Tyr5-H2, |
| | | 2.12 (Hb) | | | | Tyr5-H6 |
| | 1 | | 131.1 | | | |
| | 2 | 7.04 | 130.9 | H3 | Tyr5-Cβ, Tyr5-C1, | Tyr5-Hα, Tyr5-Hβ, |
| | | | | | Tyr5-C3, Tyr5-C5, | Tyr5-H3 |
| | | | | | Tyr5-C4, Tyr5-C6 | |
| | 3 | 6.63 | 115.37 | H2 | Tyr5-C2, Tyr5-C5, | Tyr5-H2 |
| | | | | | Tyr5-C6 | |
| | 4 | | 156.5 | | | |
| | 5 | 6.63 | 115.37 | H6 | Tyr5-C2, Tyr5-C3, | Tyr5-H6 |
| | | | | | Tyr5-C6 | |
| | 6 | 7.04 | 130.9 | H5 | Tyr5-Cβ, Tyr5-C1, | Tyr5-Hα, Tyr5-Hβ, |
| | | | | | Tyr5-C2, Tyr5-C3, | Tyr5-H5 |
| | | | | | Tyr5-C4, Tyr5-C5 | |
| | OH | 9.21 | | | Tyr5-C3, Tyr5-C4, | Tyr5-H3, Tyr5-H5 |
| | | | | | Tyr5-C5 | |
| Trp6 | C═O | | 169.0 | | | |
| | NH | 8.72 | | Hα | Trp6-CO | |
| | α | 3.88 | 42.1 | NH, | Trp6-CO | Ala7-NH |
| | | | | Hβ (Ha), | | |
| | | | | Hβ (Hb), | | |
| | β | 2.92 (Ha) | 29.4 | Hα | Trp6-Cα, Trp6-C3a | Trp6-H2 |
| | | 2.89 (Hb) | | | | |
| | 4 | 7.11 | 116.9 | H5 | Trp6-C3a, Trp6-C3a, | Trp6-Hβ(Hb) |
| | | | | | Trp6-C6, Trp6-C7, | |
| | | | | | Trp6- C7a | |
| | 5 | 6.75 | 125.1 | H4 | Lys8-Cβ, Trp6-C3a, | Trp6-H4, Lys8-Hα, |
| | | | | | Trp6-C7 | Lys8-Hβ |
| | 6 | | 132.6 | | | |
| | 7 | 7.15 | 110.2 | | Lys8-Cβ, Trp6-C3a, | Trp6-H5, |
| | | | | | Trp6-C5, Lys8-C6, | Lys8-Hα, |
| | | | | | Trp6-C7a | Lys8-Hβ |
| | 7a | | 137.5 | | | |
| | 1 | 10.68 | | H2 | Trp6-C2, Trp6-C7 | Trp6-H2, Trp6-H7 |
| | 2 | 7.14 | 123.7 | H1 | Trp6-C3, Trp6-C3a, | Trp6-H1, Trp6-Hβ |
| | | | | | Trp6-C7a | |
| | 3 | | 110.1 | | | |
| | 3a | | 127.9 | | | |
| Ala7 | C═O | | 170.3 | | | |
| | NH | 5.88 | | Hα | | Trp6-Hα, Ala7-Hβ, |
| | α | 4.05 | 48.2 | NH, Hβ | Ala7-CO, Ala7-Cβ | Ala7-Hβ, Lys8-NH |
| | β | 0.77 | 20.6 | Hα | Ala7-CO, Ala7-Cα | Ala7-Hα, Ala7-NH |
| Lys8 | C═O | | 170.2 | | | |
| | NH | 7.56 | | Hα | | Lys8-Hα, Lys8-Hβ, |
| | | | | | | Ala7-Hβ |
| | α | 4.05 | 48.1 | NH, Hβ | Lys8-CO | Lys8-Hβ, Lys8-NH, |
| | | | | | | Arg9-NH |
| | β | 2.7 | 49.6 | Hα, Hγ | | Trp6-H5, Trp6-H7 |
| | γ | 1.75 (Ha) | 28.1 | Hβ, Hδ | Lys8-Cδ | Trp6-H7, Lys8-Hβ |
| | | 1.94 (Hb) | | | | |
| | δ | 2.29 | 30.6 | Hγ, Hε | | Lys8-Hγ (Ha), |
| | | | | | | Lys8-Hγ (Hb) |
| | ε | 3.07 | 40.8 | Hδ, NH | | Lys8-Hδ |
| | | | | (side | | |
| | | | | chain) | | |
| | NH (side | 7.73 | | Hε | | |
| | chain) | | | | | |
| Arg9 | C═O | | 168.7 | | | |
| | NH | 8.23 | | Hα | | |
| | α | 4.09 | 60.5 | NH, Hβ | | |
| | β | 2.77 (Ha) | 37.0 | | | |
| | | 2.82 (Hb) | | Hα, Hγ | | |
| | γ | 1.72 (Ha) | 25.4 | Hβ, Hδ | | |
| | | 1.92 (Hb) | | | | |
| | δ | 2.31 | 30.6 | Hγ | | |
| | NH (side | 7.51 | | | Arg9-C | |
| | chain) | | | | (guanidine) | |
| | C | | 154.4 | | | |
| | (guanidine) | | | | | |
| Phe10 | C═O | | 172.7 | | | |
| | NH | 8.22 | | Hα | | |
| | α | 4.45 | 53.9 | NH, Hβ | | Phe10-Hβ |
| | β | 2.96 (Ha) | 29.5 | Hα | Phe10-Cα, Phe10-C2, | Phe10-Hα |
| | | 3.05(Hb) | | | Phe10-C6 | |
| | 1 | | 137.6 | | | |
| | 2 | 7.25 | 129.7 | H3 | Phe10-Cβ, Phe10-C3, | |
| | | | | | Phe10-C5, Phe10-C6 | |
| | 3 | 7.29 | 128.9 | H2 | Phe10-C1, Phe10-C5 | |
| | 4 | 7.23 | 126.9 | | Phe10-C2, Phe10-C6 | |
| | 5 | 7.29 | 128.9 | H6 | Phe10-C1, Phe10-C3 | |
| | 6 | 7.25 | 129.7 | H5 | Phe10-Cβ, Phe10-C3, | |
| | | | | | Phe10-C5, Phe10-C6 | |

^{a}400 MHz in DMSO-d₆ at 298 K.
^{b}Assigned by HSQC and HMBC.
^{c}not detected

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein X₁ and X₃ in each motif are connected to form a cyclophane moiety;
wherein at least one of the two C-terminus residues is an aromatic residue.

2. The polypeptide according to claim 1, wherein the first and second three residue motifs are separated by 1 to 3 amino acid residue.

3. The polypeptide according to claim 1 or 2, wherein the first three residue motif is not fused with the second three residue motif via the cyclophane moieties.

4. The polypeptide according to any one of claims 1 to 3, wherein the first X₁ is a residue selected from tryptophan, phenylalanine or a derivative thereof and the second X₁ is a residue selected from phenylalanine, tyrosine or a derivative thereof.

5. The polypeptide according to any one of claims 1 to 43, wherein X₂ is an amino acid residue, the amino acid independently selected from I, G, E, Y, V, L, A, D, S, T, N or Q.

6. The polypeptide according to any one of claims 1 to 5, wherein X₃ is an amino acid residue, the amino acid independently selected from N, R, S, D, Q or K.

7. The polypeptide according to any one of claims 1 to 6, wherein at least one of the two C-terminus residues is a polar and/or basic residue.

8. The polypeptide according to any one of claims 1 to 7, wherein at least one of the two C-terminus residues is an aromatic residue.

9. The polypeptide according to any one of claims 1 to 8, wherein the polypeptide comprises a third three residue motifs.

10. The polypeptide according to any one of claims 1 to 9, wherein when the polypeptide comprises a third three residue motif, X₃ of the first motif and X₁ of the second motif are separated by 1 amino acid residue, and X₃ of the second motif and X₁ of the third motif are covalently bonded to each other via an amide bond.

11. The polypeptide according to any one of claims 1 to 10, wherein the third X₁ is a residue independently selected from tryptophan, phenylalanine or a derivative thereof.

12. The polypeptide according to any one of claims 1 to 11, wherein the polypeptide is represented by Formula (I): 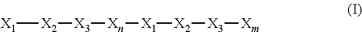
wherein each X₁ is an amino acid residue, the amino acid independently selected from tryptophan, phenylalanine, or a derivative thereof;
wherein each X₂ is an amino acid residue, the amino acid independently selected from leucine, isoleucine, valine, alanine, proline, serine, lysine, asparagine, phenylalanine, aspartic acid or a derivative thereof;
wherein each X₃ is an amino acid residue, the amino acid independently selected from lysine, glutamine, asparagine, arginine or a derivative thereof;
wherein Xₙ is an amide bond or 1 to 3 amino acid residue; and
wherein Xₘ is at least two C-terminus residues.

13. The polypeptide according to any one of claims 1 to 11, wherein the polypeptide is represented by Formula II): 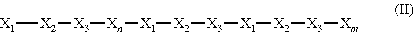
wherein each X₁ is an amino acid residue, the amino acid independently selected from tryptophan, phenylalanine, tyrosine, or a derivative thereof;
wherein each X₂ is an amino acid residue, the amino acid independently selected from valine, isoleucine, phenylalanine, tryptophan, alanine, leucine, glycine, serine, proline, threonine, aspartic acid, asparagine, glutamic acid, arginine or a derivative thereof;
wherein each X₃ is an amino acid residue, the amino acid independently selected from arginine, lysine, asparagine or a derivative thereof;
wherein Xₙ is an amide bond or 1 to 3 amino acid residue; and
wherein Xₘ is at least two C-terminus residues.

14. The polypeptide according to any one of claims 1 to 13, wherein X₁ and X₃ in the second motif are connected via phenylene to form a cyclophane moiety.

15. The polypeptide according to any one of claims 1 to 14, wherein the polypeptide is represented by Formula (Ia), (IIa), (Id) or (IId):

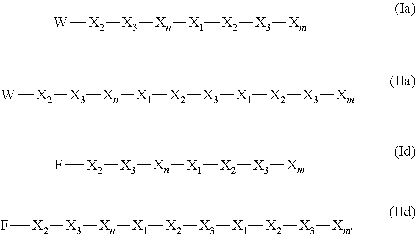

16. The polypeptide according to any one of claims 1 to 15, wherein the polypeptide is represented by Formula (Ib), (IIb), (Ie) or (IIe):

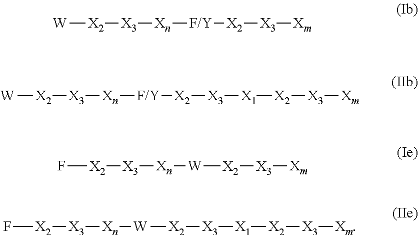

17. The polypeptide according to any one of claims 1 to 16, wherein when X₁ is W, X₁ is connected to X₃ via a 3,6 or 3,7 substituted indolylene moiety.

18. The polypeptide according to any one of claims 1 to 17, wherein when X₁ is F or Y, X₁ is connected to X₃ via a 1,3 or 1,4 disubstituted phenylene moiety.

19. The polypeptide according to any one of claims 1 to 18, wherein the polypeptide is represented by Formula (IIc):

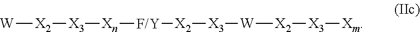

20. The polypeptide according to any one of claims 1 to 19, wherein the polypeptide is selected from:
(SEQ ID 19)
WVNAFANWTKRF
(SEQ ID 17)
WVNAFANWPKRF
(SEQ ID 13)
WINAFANWTKRI
(SEQ ID 37)
WWRAYARWRRSF
(SEQ ID 4)
WVNAFARWGKSF
(SEQ ID 36)
GWFRAYLRWSRSF
(SEQ ID 25)
WVNAYARWTNRF
(SEQ ID 14)
WVNAFAKWTKRI
(SEQ ID 26)
WVNAYARWTKRF
(SEQ ID 22)
WVNVFARWDKQI
(SEQ ID 15)
WVNFFAKFTKSF
(SEQ ID 30)
WVNAFARWSRRW
(SEQ ID 8)
WVNAFARWSKSF
(SEQ ID 34)
WVNVFARWSRRW
(SEQ ID 35)
AGWIRAFANWSRSF
(SEQ ID 23)
WVNAFARWDKKF
(SEQ ID 20)
WVNAFARFTKRF
(SEQ ID 10)
WVNVFARWDKAI
(SEQ ID 24)
WLNVFVRWDRAI
(SEQ ID 21)
WINVFARWNRAI
(SEQ ID 32)
WINAFGNWERAFH
(SEQ ID 3)
WVNAFANWSKSF
(SEQ ID 1)
WVNAFANWSKAL
(SEQ ID 2)
WVNAFGNWSKSL
(SEQ ID 16)
WVNAFLNWSRSF
(SEQ ID 12)
WVNAFLRWGKSF
(SEQ ID 7)
WINAFARWGRAF
(SEQ ID 33)
AGWIKVFGNWSRSF
(SEQ ID 9)
WVNAFVNWTKSF
(SEQ ID 18)
WVNAFLNWPRSF
(SEQ ID 29)
AGWIKAFGNWSRSF
(SEQ ID 6)
WVNAFVNWPKSF
(SEQ ID 28)
AGWINAFANWTKSF
(SEQ ID 31)
AGWINAFANWTRSF
(SEQ ID 27)
AGWINAFGNWTKSF
(SEQ ID 5)
WVNAFARWGRAF
(SEQ ID 38)
WVNAFARWSKRW
(SEQ ID 39)
WVNAFARWSKRF
(SEQ ID 50)
RGEGWVRAYWAKRF
(SEQ ID 52)
KPGEGWVNFTWNKSF
(SEQ ID 46)
KSEAAGGWVNFQWKNSW
(SEQ ID 49)
AGNDGWVKFGWKKKF
(SEQ ID 54)
ASTAETWFKLDWKKSF
(SEQ ID 41)
DGRWLQWIKNH
(SEQ ID 40)
GDRWLKWIKNH
(SEQ ID 44)
VGGFANATWSKSF
(SEQ ID 43)
VGGFANASWPKSF
(SEQ ID 45)
VGGFANATWPKSF
(SEQ ID 59)
NAFVNATWSRAM
(SEQ ID 47)
NVFVNATWSRAM
(SEQ ID 60)
NVFVNATWSRAI
(SEQ ID 55)
SSDDDGIFFKTTWDRR

21. The polypeptide according to any one of claims 1 to 20, wherein the polypeptide is selected from:

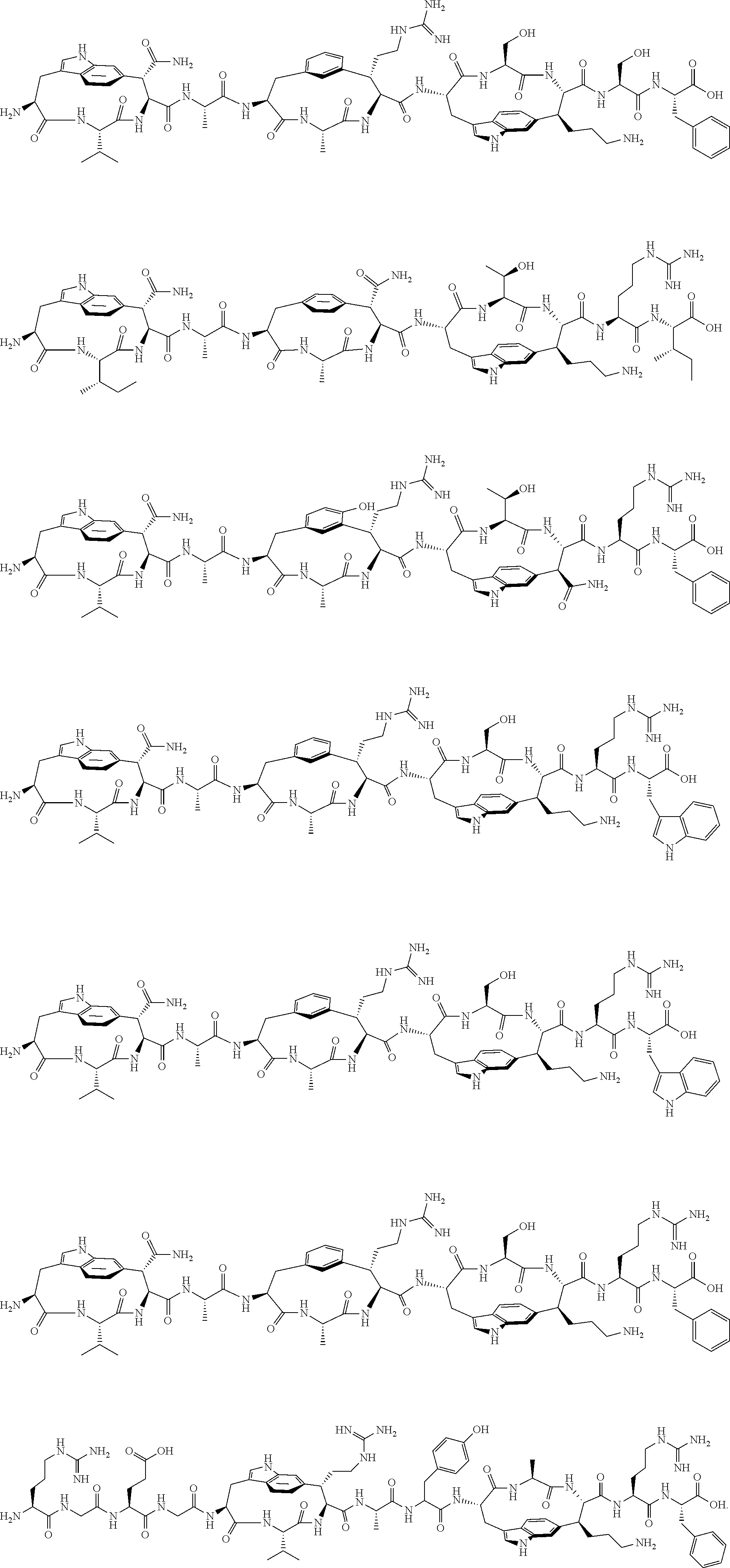

22. The polypeptide according to any one of claims 1 to 21, wherein the polypeptide is an isolated polypeptide.

23. The polypeptide according to any one of claims 1 to 22, wherein the polypeptide is characterised by an antibacterial activity.

24. The polypeptide according to any one of claims 1 to 23, wherein the polypeptide is characterised by a minimal inhibitory concentration (MIC) of about 2 μg/mL to about 10 μg/mL.

25. A composition comprising a polypeptide according to any one of claims 1 to 24.

26. A method of producing a polypeptide in a host cell, the method comprising:
a) introducing to the host cell one or more nucleic acid molecules, the nucleic acid molecules configured to express a precursor polypeptide (A), a rSAM/SPASM maturase (B), a protease (C), a transporter (D) and a protease/transporter (E);
wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide in the host cell to form a modified precursor polypeptide with a cyclophane moiety connecting the X₁ and X₃ residues in each motif;
wherein the protease, transporter and protease/transporter are capable of cleaving the modified precursor polypeptide from the rSAM/SPASM maturase to form a cleaved modified polypeptide and exporting the cleaved modified polypeptide out from the host cell.

27. The method according to claim 26, wherein at least the nucleic acid molecule configured to express A is derived from a Xye maturase system.

28. The method according to claim 26 or 27, wherein the nucleic acid molecules configured to express A and B are from one Xye species and the nucleic acid molecules configured to express C, D and E are from another Xye species.

29. The method according to any one of claims 26 to 28, wherein at least the nucleic acid molecules configured to express C, D and E are fused.

30. The method according to any one of claims 26 to 29, wherein the nucleic acid molecules configured to express A and B are fused.

31. The method according to claim 26 or 27, wherein the nucleic acid molecules configured to express B, C, D and E are fused.

32. The method according to any one of claims 26 to 31, wherein the nucleic acid molecules configured to express A, B, C, D and E are fused.

33. The method according to any one of claims 26 to 32, wherein the nucleic acid molecule configured to express A is at least 70% identical to and derived from a bacterial species selected from Serratia marcescens (smc), Erwinia toletana (etc), Photorhabdus australis (pac), Xenorhabdus nematophila (xnc), Xenorhabdus griffiniae VH1 (xgc), Pandoraea sp. PE-S2R-1 (psc), Pandoraea oxalativorans DSM 23570 (poc), Photorhabdus heterorhabditis Q614 (phc), Kosakonia cowanii pasteuri (kcc2 and kcc1), Bordetella bronchialis AU17976 (bbc) and Photorhabdus laumondii BOJ-47 (plc).

34. The method according to any one of claims 26 to 32, wherein the nucleic acid molecules configured to express C, D and E are at least 70% identical to and derived from Xenorhabdus nematophila (xnc).

35. The method according to any one of claims 26 to 34, wherein the rSAM/SPASM maturase has an amino acid sequence that is at least 70% identical to one of the following:
XncB:
(SEQ ID NO: 61)
MTTSKSEKIKHLEIILKISERCNINCSYCYVFNMGNSLATDSPPVISLDNVLALRGFFERSAAENEI
EVIQVDFHGGEPLMMKKDRFDQMCDILRQGDYSGSRLELALQTNGILIDDEWISLFEKHKVHASI
SIDGPKHINDRYRLDRKGKSTYEGTIHGLRMLQNAWKQGRLPGEPGILSVANPTANGAEIYHHFA
NVLKCQHFDFLIPDAHHDDDIDGIGIGRFMNEALDAWFADGRSEIFVRIFNTYLGTMLSNQFYRV
IGMSANVESAYAFTVTADGLLRIDDTLRSTSDEIFNAIGHLSELSLSGVLNSPNVKEYLSLNSELPS
DCADCVWNKICHGGRLVNRFSRANRFNNKTVFCSSMRLFLSRAASHLITAGIDEETIMKNIQK
YkcB:
(SEQ ID NO: 62)
MEVITGSEGRVMLNLLIEKNIRHLEIILKISERCNINCDYCYVFNKGNSAADDSPARLSNKNIHHLV
CFLQRACQEYKIGTVQIDFHGGEPLLMKKENFTDMCIQLISGNYCGSNIRLALQTNATLIDNEWIA
IFEKYSVNVSISIDGPKHINDRHRLDTKGRSTYESTVRGLRILQNAYQQGRLPSDPGILCVTNAQA
NGAEIYRHFVDELGVYSFDFLIPDDSYKDAHPDAVGIGRFLNEALDEWVKDNNAKIFVRLFQTHIA
SLLGQKNSGVLGHTPNITGVYALTVSSDGFVRVDDTLRSTSDRMFNPIGHLSEVNLSNVFASPQF
QEYSSIGQSLPTECEGCIWENICAGGRIVNRFSTEDRFKHKSIYCYSMRTFLSRSSAHLLNMGIKE
ERIMAAIRA
EtcB
(SEQ ID NO: 63)
MTQLKGEKIKHLEIILKISERCNINCTYCYVFNMGNTLATDSTPVISLDNVYALRGFFERSAAENDI
EVIQVDFHGGEPLMMKKDRFDRMCQILLQGNYRSSKFELALQTNGILIDDEWIALFEKHQVHASI
SVDGPKHINDRHRLDRKGKSTYEGTITGLRLLQNAWQQGRLPGEPGILSVANANANGAEIYRHF
ADTLQCQRFDFLIPDDHHDDSPDGEGVGRFLNEALDAWFADGRPEIFIRIFNTYLGTMLNSQFNR
VLGMSANVESAYAFTVTADGMLRIDDTLRSTSDEIFNAVGHVSELSLARVLETSCVKEYLALSSNL
PTVCAECVWNNICHGGRLVNRFSRTNRFNNKTVFCKSMRLFLSRAASHLMASGVDEKEIMKNIQ
K
MscB
(SEQ ID NO: 64)
MAPGPARAALTEFVLKVHARCDLACDHCYVYEHADQSWRRRPVRMTPEVLRTAAGRIAEHAAAH
DLPDVTVILHGGEPLLLGAERLGEVLADLRRVIDPVTRLRLGMQTNGVLLSERLCDLLAEHDVAVG
VSLDGDRAANDRHRRFRSGAGSYDQVLRAIGLLRRPAYRRIYSGLLCTVDVRNDPIAVYESLLTQ
EPPRIDFLLPHATWDDPPWRPAGGGTAYAGWLRAVYDRWLADGRPVSVRLFDSLLSTAAGGPS
GTEWLGLDPVDLAVVETDGEWEQADSLKTAYDGAPATGMTVFSHAADDVAASPLLARRRSGRA
GLSDECRRCPVVDQCGGGLFAHRYGAGHFDHPSVYCADLKELIVHVNENPPAPVRLDAGLPDDF
IDRLAALTGDRVAIGRLVEAQIAIVRALLAEVADRLPAGGAGADGWEALTALDRSAPESVARIAAH
PYVRAWAVDCLAGSGTGARQGPDYLSALAVAAALDAGTPVRLDVPVRSGRLHLPTVGTVLLPEV
GDGAARVETGPGSLRVAAGDVTVAIRPGTPGDAPRWWPTRVLAAPDVSVLLEDGDPHRDCHRL
PAGDRLDDAGAARWAETFAAAWQVIRDEVPGHAEELRAGLRAVVPLRRSGAGVSEASTARQAF
GGVAATETDAGSLAVLLVHEFQHSKMNALLDICDLVDGTRPIDITVGWRPDPRPAEAVLHGIYAH
AAVADIWRIRADRQVDGAQAVYRRYRDWTAEAIGALQRADALTPAGSRLVRQVARSMSGWPS
OscB:
(SEQ ID NO: 65)
MINPTLLNPEKIDISKFGPINLVVIQATSFCNLNCDYCYLPNRDLKNTLSLDLIEPIFKNIFNSPFVG
DEFTICWHAGEPLAVPISFYESAFQLIQAADQKYNQKQAKIWHSVQTNATYINQKWCDFIQEHNI
CVGVSLDGPEFIHDAHRQTRKGTGSHAQTMRGISFLQKNNIPFYVISVVTQDSLNYADEIFNFFR
ENGIYDVGFNLEEIEGVNQSSTLEAVGTSEKYRAFMQRFWELTSEVQGEFNLREFEAICGLIYSNT
RLTQTDMNNPFVLINIDYQGNFSTFDPELLSVNIKPYGNFILGNVLTDSFESVCDTEKFQKIYTDM
QEGIKLCRETCEYFGVCGGGAGSNKYWENGTFACSETMACRYRIKVVTDIILDKLENSLGLVENC
LscB:
(SEQ ID NO: 66)
MTISKMNLPVQTDNFRASSTLDLSAFGPINLVVIQSTSFCNLNCDYCYLRDRQSKNRLSLDLIEPIL
KTVLTSPFVGCDFTILWHAGEPLAMPISFYDSATALIREAERQYKTQPIQIFQSIQTNATLINQAWC
DCFRRNEIYVGVSLDGPAFLHDAHRQTYKGTGTHAATMRGISLLQKNEIPFNVICVLTQDSLDYP
DEIFNFFRSNRITEVGFNMEEAEGVHQHSTLDQQGTEERYRAFMQRFWDLTVQAKGEFKLREFE
TICTLAYTGDRLGYTDMNQPFVIVNFDHQGNFSTFDPELLSFKIKEYGDFVLGNVLHNTLESVCQT
EKFQKIYQDMAAGVVQCRQSCEYFGLCGGGAGSNKYWENGTFNCTETKACRYRIKVIADIVLEG
LENSLELANSIS
GscB
(SEQ ID NO: 67)
MSIVTSKPVINFKNTANFGPISLIIIQPNSFCNLDCDYCYLPDRHLQNKLSLDLIDPIFKSIFTSPFLG
CDFGVCWHAGEPLTMPVSFYKSAFQLIEEANTKYNKSEYSFYHSYQTNGTLINQGWCDLWQEYP
VHVGVSIDGPAFLHDVHRKNRKGGNSHDLTMRGIRYLQKNNIPYNTISVITEESLNYPDEMFNFF
AENEIYDLAFNMEETEGVNELTSLNGIEIEHKYSQFIKRFWQLVTESKLPFIVREFEILISLIYSGNR
LTNTDMNKPFVIVNFDYQGNFSTFDPELLSVKTDKYGDFIFGNVLKDSLESICETEKFKTIYKDIND
GVKLCSDNCSYFGICGGGAGSNKYWENGTFASMETQACRYRIKILTDVLVSTIENSLGL
MscB-375
(SEQ ID NO: 68)
MAPGPARAALTEFVLKVHARCDLACDHCYVYEHADQSWRRRPVRMTPEVLRTAAGRIAEHAAAH
DLPDVTVILHGGEPLLLGAERLGEVLADLRRVIDPVTRLRLGMQTNGVLLSERLCDLLAEHDVAVG
VSLDGDRAANDRHRRFRSGAGSYDQVLRAIGLLRRPAYRRIYSGLLCTVDVRNDPIAVYESLLTQ
EPPRIDFLLPHATWDDPPWRPAGGGTAYAGWLRAVYDRWLADGRPVSVRLFDSLLSTAAGGPS
GTEWLGLDPVDLAVVETDGEWEQADSLKTAYDGAPATGMTVFSHAADDVAASPLLARRRSGRA
GLSDECRRCPVVDQCGGGLFAHRYGAGHFDHPSVYCADLKELIVHVNENPPAPV.

36. The method according to any one of claims 26 to 35, wherein the rSAM/SPASM maturase is characterised by a rSAM domain and a SPASM domain;
wherein the rSAM domain is CNINCSYC (SEQ ID NO: 69); and
wherein the SPASM domain is CADCVWNKIC (SEQ ID NO: 70).

37. The method according to any one of claims 26 to 36, wherein the nucleic acid molecules are introduced into the host cell via a pET28a(+) vector, pCDFduet-1 vector, pACYCDuet-1 vector, pETDuet-1 vector, pCOLADuet-1 vector, pRSFDuet-1 vector, pBAD vector, or a combination thereof.

38. The method according to any one of claims 26 to 37, wherein the host cell is E. coli NiCo21(DE3), BL21(DE3), BL21-AI, BL21 Star™ (DE3) pLysS, Rosetta™ (DE3), or a combination thereof.

39. A method of producing a polypeptide, the method comprising:
a) expressing a precursor polypeptide and a rSAM/SPASM maturase; wherein the precursor polypeptide comprises a first three residue motif (from a N-terminus) and a second three residue motif, the first and second three residue motif optionally separated by 1 to 3 amino acid residue, and at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue;
wherein at least one of the two C-terminus residues is an aromatic residue;
wherein the rSAM/SPASM maturase is capable of modifying the precursor polypeptide to form a polypeptide with a cyclophane moiety connecting the X₁ and X₃ residues in each motif.

40. A method of synthesising a polypeptide according to any one of claims 1 to 24, the method comprising:
(a) coupling a pre-sequence peptide to a support, wherein said pre-sequence peptide comprises amino acid residues having side chain functionalities which are, if necessary, protected during the synthesis;
(b) coupling one or more N-protected amino acids to the N-terminus of the pre-sequence peptide to form a precursor polypeptide, wherein each coupling is performed in stepwise fashion and under conditions in which each of the amino acids of the target peptide is coupled and subsequently N-deprotected;
c) cleaving said precursor polypeptide from the support; and
d) synthetically or enzymatically connecting the X₁ and X₃ in each motif to form a cyclophane moiety.

41. A method of modifying a precursor polypeptide, the precursor polypeptide comprising:
a) a first three residue motif (from a N-terminus) and a second three residue motif, the first and second three residue motif optionally separated by 1 to 3 amino acid residue; and
b) at least two C-terminus residues;
wherein the three residue motif is each represented by X₁-X₂-X₃;
wherein each X₁ is a residue independently selected from tryptophan, phenylalanine, tyrosine, histidine, an unnatural aromatic amino acid residue or a derivative thereof;
wherein each X₂ and X₃ are independently any amino acid residue; and
wherein at least one of the two C-terminus residues is an aromatic residue;
the method comprising:
enzymatically connecting the X₁ and X₃ residues in each motif to form a cyclophane moiety.

42. The method according to claim 41, wherein the enzyme is rSAM/SPASM maturase.

43. A method of treating a bacterial infection in a subject in need thereof, comprising administering an effective amount of a polypeptide according to any one of claims 1 to 24 to the subject.

44. The method according to claim 43, wherein the bacterial infection is a Gram-negative bacterial infection.

45. The method according to claim 43 or 44, wherein the bacterial infection is characterised by a drug-resistance.

46. The method according to any one of claims 43 to 45, wherein the bacterial infection is caused by a Gram-negative bacteria selected from Escherichia coli, Pseudomonas aeruginosa, Candidatus Liberibacter, Agrobacterium tumefaciens, Acinetobactor baumannii, Moraxella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Actinobacillus actinomycetemcomitans, Aggregatibacter actinomycetemcomitans, Porphyromonas gingivalis, Helicobacter pylori, Francisella tularensis, Yersinia pestis, Vibrio cholera, Morganella morganii, Edwardsiella tarda, Campylobacter jejuni, Haemophilus influenza, Enterobacter cloacae, or a combination thereof.
